(19) United States
(12) Patent Application Publication
(10) Pub. No.: US 2023/0138393 A1
(43) Pub. Date: May 4, 2023

(54) LINKING AMINO ACID SEQUENCES, MANUFACTURING METHOD THEREOF, AND USE THEREOF

(71) Applicant: Temple University-Of The Commonwealth System of Higher Education, Philadelphia, PA (US)

(72) Inventors: Rongsheng E. Wang, Wynnewood, PA (US); Md Shafiqul Islam, Philadelphia, PA (US); Zhigang Lyu, Philadelphia, PA (US); Yue Zhao, Philadelphia, PA (US); Zakey Yusuf Buuh, Philadelphia, PA (US)

(21) Appl. No.: 17/904,229

(22) PCT Filed: Feb. 12, 2021
(86) PCT No.: PCT/US2021/017839

§ 371 Date: Aug. 15, 2022

Related U.S. Application Data
(60) Provisional application No. 62/976,599, filed on Feb. 14, 2020.

(51) Int. Cl. C07K 1/107; A61K 47/54

(52) U.S. Cl. CPC C07K 1/1077; A61K 47/545; A61K 47/542; A61K 47/54

(57) ABSTRACT

This invention provides compositions comprising linked amino acid sequences, pharmaceutical compositions comprising linked amino acid sequences, and methods of making thereof. This invention also provides methods of delivering said compositions to subjects and methods of treating various disorders and diseases using the said compositions.

### CROSS-REFERENCE TO RELATED APPLICATIONS

This application claims priority to U.S. Provisional Application No. 62/976,599, filed Feb. 14, 2020, which is hereby incorporated by reference herein in its entirety.

### STATEMENT REGARDING FEDERALLY SPONSORED RESEARCH OR DEVELOPMENT

This invention was made with government support under P30CA006927, R50CA221838, 2P50CA100632-16, and 1R35GM133468-01 awarded by the National Institute of Health. The government has certain rights in the invention.

### BACKGROUND OF THE INVENTION

Bioorthogonal reactions have greatly facilitated protein labeling in complex biological systems and led to widespread applications including imaging, enrichment, and identification, etc. (Devaraj N K, 2018, ACS Cent. Sci., 4:952-959; Elliott T S et al., 2016, Cell Chem. Biol., 23:805-815). Representative bioorthogonal reactions, such as azide-alkyne cycloaddition (Grammel M et al., 2013, Nat. Chem. Biol., 9:475-484), Staudinger ligation (Saxon E et al., 2000, Science, 287:2007-2010), and tetrazine cycloaddition (Elliott T S et al., 2016, Cell Chem. Biol., 23:805-815), have resulted in successful development of chemical reporters, which in conjunction with detection/affinity tags, have advanced the understanding of important biological pathways including post-translational modifications (PTMs) (Chuh K N et al., 2015, Curr. Opin. Chem. Biol., 24:27-37). Yet, current chemical reporters are bulky in length and size, thereby impeding their general applications. Even alkyne- or azide-based minimalist reporters for copper-catalyzed azide-alkyne cycloaddition (CuAAC) are still larger than the inherent carbon-hydrogen bond. This intrinsic steric hindrance has largely limited the application of chemical reporters to metabolic incorporation by enzymes possessing spacious active site pockets. For example, alkyne/azide-labelled PTM precursors or cofactors for acetylation (Yang Y Y et al., 2010, J. Am. Chem. Soc., 132:3640-3641; Yang C et al., 2013, J. Am. Chem. Soc., 135:7791-7794) and methylation (Binda O et al., 2011, ChemBioChem, 12:330-334; Grammel M et al., 20102, Chemical reporters of protein methylation and acetylation. Cambridge: Cambridge University Press) were too bulky to be incorporated by many cognate transferases. While “bump-hole” protein engineering strategy could work for a given enzyme, careful balances between mutations, structure folding, and function (potency, selectivity, etc.) are required (Runcie A C et al., 2016, Curr. Opin. Chem. Biol., 33:186-194). For many acetyltransferases that function as subunits in protein complexes, mutations in the “hole” may alter their substrate specificities, leading to results different from in vivo sub-acylome (Han Z et al., 2017, ACS Chem. Biol., 12:1547-1555), which undermines this approach's broad applications. Elucidating the molecular targets of these PTM transferases has been thereby compromised, despite being a key step towards the systematic dissection of PTMs and their roles in biological and pathology-related cellular signaling regulation (Yang C et al., 2013, J. Am. Chem. Soc., 135:7791-7794; Grammel M et al., 20102, Chemical reporters of protein methylation and acetylation. Cambridge: Cambridge University Press; Buuh Z Y, 2018, J. Med. Chem., 61:3239-3252).

Thus, there is a need in the art for a bioorthogonal reaction that can generate reporters for steric-free labeling of protein substrates and thereby allow for global profiling of molecular targets. The present invention addresses this unmet need in the art.

### SUMMARY OF THE INVENTION

In one aspect, the present invention relates, in part, to a method of stapling one or more amino acid sequences, the method comprising reacting a compound or salt thereof having the structure of Formula (I) and a compound or salt thereof having the structure of Formula (II)

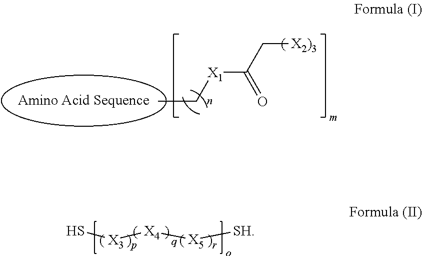

In some embodiments, each occurrence of X₁ is independently O, S, NR₁, CR₁R₂, or C(═R₃).

In some embodiments, each occurrence of X₂ is independently H, Br, Cl, F, or I.

In some embodiments, each occurrence of X₃, X₄, and X₅ is independently O, S, NR₁, CR₁R₂, C(═R₃), cycloalkyl, substituted cycloalkyl, heterocycloalkyl, substituted heterocycloalkyl, aryl, substituted aryl, heteroaryl, or substituted heteroaryl.

In some embodiments, each occurrence of R₁ and R₂ is independently hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, or substituted heteroaryl.

In some embodiments, each occurrence of R₃ is independently O, NR₁, or S.

In some embodiments, m is an integer from 1 to 10.

In some embodiments, o is an integer from 1 to 10.

In some embodiments, each occurrence of n, p, q, and r is independently an integer from 0 to 50.

In various embodiments, the compound having the structure of Formula (I) is a compound having the structure of Formula (III)

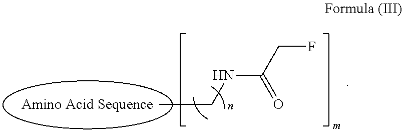

In some embodiments, the compound having the structure of Formula (II) is a compound having the structure of Formula (IV)

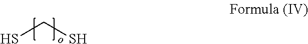

or a compound having the structure of Formula (V)

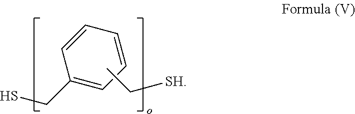

In some embodiments, the amino acid sequence comprises two or more amino acids. In some embodiments, the amino acid sequence is a protein or a fragment thereof, peptide or a fragment thereof, antigen or a fragment thereof, or any combination thereof. In some embodiments, the peptide or a fragment thereof is an axin peptide or a fragment thereof, HIV peptide or a fragment thereof, peptide or fragment thereof derived from one or more proteins related to neuron regeneration, peptide or fragment thereof derived from one or more proteins related to neuron degeneration, peptide or fragment thereof derived from one or more proteins related to immune signaling, or any combination thereof.

In various embodiments, the method of the present invention comprises reacting the compound or salt thereof having the structure of Formula (I) and the compound or salt thereof having the structure of Formula (II) in the presence of a base.

In some embodiments, the base is amidine compound, 1,8-Diazabicyclo[5.4.0]undec-7-ene (DBU), carbonate compound, K₂CO₃, Li₂CO₃, Na₂CO₃, Rb₂CO₃, Cs₂CO₃, K₂CO₃, KHCO₃, KOH, NaOH, LiOH, CsOH, RbOH, Ca(OH)₂, Sr(OH)₂, Ba(OH)₂, ammonia, methylamine, ethylamine, n-propylamine, isopropylamine, cyclohexylamine, dimethylamine, diethylamine, di-n-propylamine, trimethylamine, triethylamine, tri-n-propylamine, N,N-Diisopropylethylamine, aniline, N-methylaniline, N,N-dimethylaniline, p-bromoaniline, p-methoxyaniline, p-nitroaniline, pyrrole, pyrrolidine, imidazole, pyridine, piperidine, phosphazenes, or any combination thereof.

In various embodiments, the method of the present invention comprises reacting the compound or salt thereof having the structure of Formula (I) and the compound or salt thereof having the structure of Formula (II) in a solution having a pH around 7. In various embodiments, the method of the present invention comprises reacting the compound or salt thereof having the structure of Formula (I) and the compound or salt thereof having the structure of Formula (II) in a solution having a pH above around 7. In various embodiments, the method of the present invention comprises reacting the compound or salt thereof having the structure of Formula (I) and the compound or salt thereof having the structure of Formula (II) in a solution having a pH below around 7. For example, in one embodiment, the method comprises reacting the compound or salt thereof having the structure of Formula (I) and the compound or salt thereof having the structure of Formula (II) in an aqueous solution having a pH around 8.5. In one embodiment, the method comprises reacting the compound or salt thereof having the structure of Formula (I) and the compound or salt thereof having the structure of Formula (II) in an aqueous solution having a pH around 6. In some embodiments, the reacting the compound or salt thereof having the structure of Formula (I) and the compound or salt thereof having the structure of Formula (II) is faster in an aqueous solution having a pH above around 7.

In some embodiments, the method of the present invention comprises reacting the compound or salt thereof having the structure of Formula (I) and the compound or salt thereof having the structure of Formula (II) in the presence of a reducing agent. In some embodiments, the reducing agent is tris(2-carboxyethyl)phosphine (TCEP), tris(3-hydroxypropyl)phosphine (THPP), sodium cyanoborohydride, lithium aluminum hydride, sodium amalgam (Na(Hg)), sodium borohydride, sulfite reducing agent, dithionate reducing agent, Na₂S₂O₆, thiosulfate reducing agent, Na₂S₂O₃, KI, hydrazine, diisobutylaluminum hydride (DIBAL-H), oxalic acid, formic acid, ascorbic acid, reducing sugars, phosphites, hypophosphites, phosphorous acid, dithiothreitol (DTT), carbon monoxide, or any combination thereof.

In some embodiments, the compound or salt thereof having the structure of Formula (I) is reacted with the compound or salt thereof having the structure of Formula (II) in 1:1, 1:2, 1:5, 1:10, 1:50, or 1:100 molar ratio. In other embodiments, the compound or salt thereof having the structure of Formula (I) is reacted with the compound or salt thereof having the structure of Formula (II) in 2:1, 5:1, 10:1, 50:1, or 100:1 molar ratio.

In another aspect, the present invention relates, in part, to a method of linking one or more amino acid sequences and one or more compounds A, the method comprising reacting a compound or salt thereof having the structure of Formula (I) with a compound or salt thereof having the structure of Formula (VI)

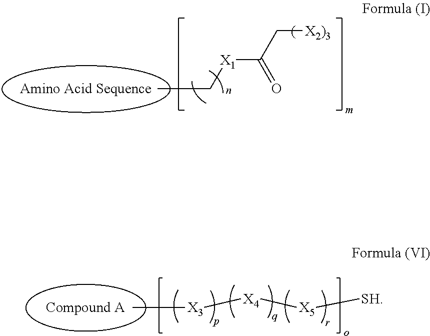

In some embodiments, the compound A is an antibody or a fragment thereof, antigen or a fragment thereof, protein or a fragment thereof, peptide or a fragment thereof, amino acid sequence or a fragment thereof, amino acid or a derivative thereof, small molecule or a derivative thereof, therapeutic agent or a derivative thereof, or any combination thereof.

In some embodiments, the compound having the structure of Formula (VI) is a compound having the structure of Formula (VII)

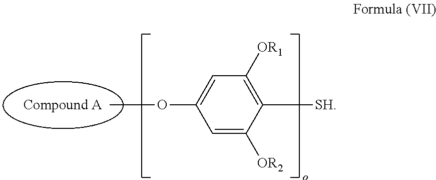

In some embodiments, the compound having the structure of Formula (VII) is a compound having the structure of Formula (VIII)

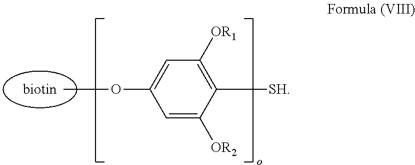

In some embodiments, the compound having the structure of Formula (VIII) is a compound having the structure of Formula (IX)

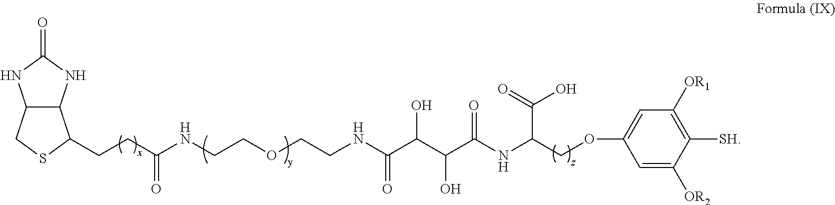

In some embodiments, each occurrence of x, y, and z is independently an integer from 1 to 10.

In another aspect, the present invention relates, in part, to compounds prepared by any of the herein-described methods of the present invention.

In some embodiments, the compound of the present invention is a compound or salt thereof having the structure of Formula (X)

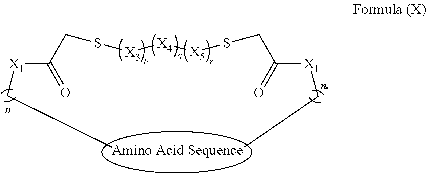

In some embodiments, the compound having the structure of Formula (X) is a compound having the structure of Formula (XI)

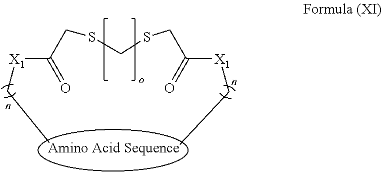

or a compound having the structure of Formula (XII)

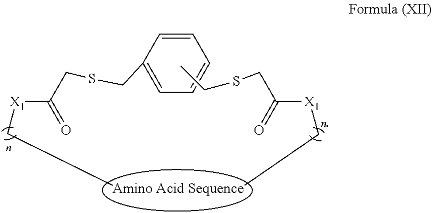

In other embodiments, the compound of the present invention is a compound or salt thereof having the structure of Formula (XIII)

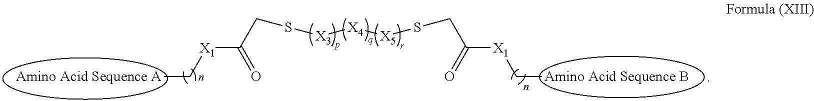

In some embodiments, the compound having the structure of Formula (XIII) is a compound having the structure of Formula (XIV)

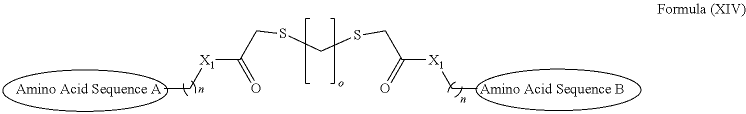

or a compound having the structure of Formula (XV)

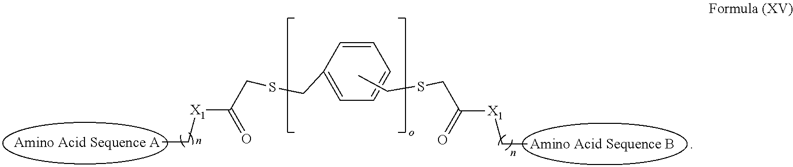

In some embodiments, each occurrence of the amino acid sequence A and amino acid sequence B is independently a protein or a fragment thereof, peptide or a fragment thereof, antigen or a fragment thereof, or any combination thereof. In some embodiments, the amino acid sequence A is identical to the amino acid sequence B.

In other embodiments, the compound of the present invention is a compound or salt thereof having the structure of

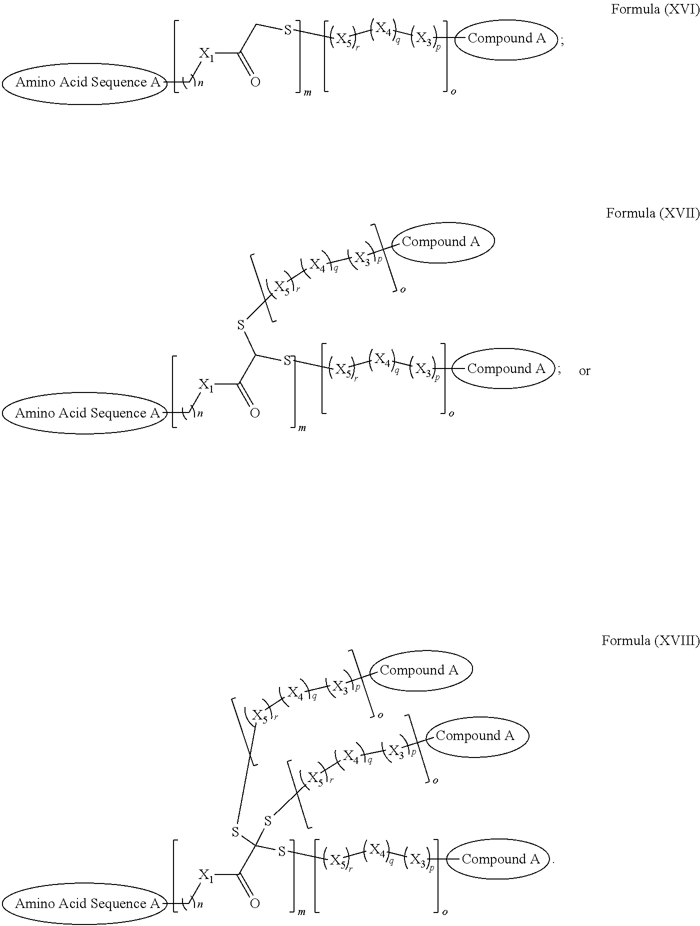

In some embodiments, the compound having the structure of Formula (XVI) is a compound having the structure of Formula (XIX)

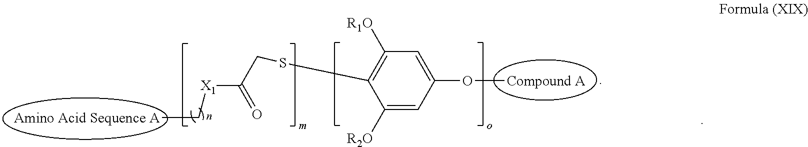

In some embodiments, the compound having the structure of Formula (XIX) is a compound having the structure of Formula (XX)

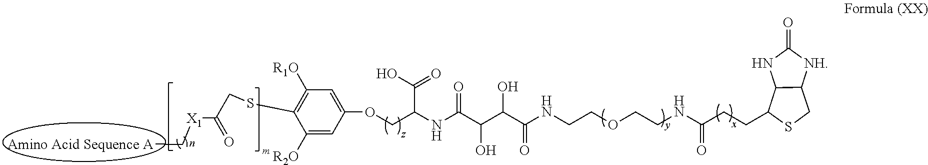

In various embodiments, the compounds of the present invention penetrate a cell.

In another aspect, the present invention relates, in part, to a method of delivering a stapled amino acid sequence into a subject in need thereof, the method comprising administering to the subject at least one compound of the present invention that penetrates a cell.

In yet another aspect, the present invention relates, in part, to a method of delivering an amino acid sequence, a compound A, or a combination thereof into a subject in need thereof, the method comprising administering to the subject at least one compound of the present invention that penetrates a cell.

In another aspect, the present invention relates, in part, to a method of treating a disease or disorder in a subject, the method comprising administering to the subject at least one compound of the present invention that penetrates a cell.

In another aspect, the present invention relates, in part, to a method of treating a disease or disorder in a subject, the method comprising administering to the subject at least one compound of the present invention that penetrates a cell.

In another aspect, the present invention relates, in part, to a method of labeling, imaging, and/or detecting a compound or salt thereof, the method comprising reacting the compound or salt thereof and a compound or salt thereof having the structure of Formula (XXI)

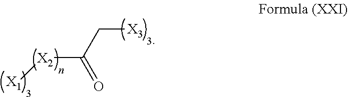

In some embodiment, each occurrence of X₁ and X₃ is independently hydrogen, halogen, OR₁, SR₁, NR₁R₂, CR₁(═R₃), alkyl, substituted alkyl, cycloalkyl, substituted cycloalkyl, heterocycloalkyl, substituted heterocycloalkyl, aryl, substituted aryl, heteroaryl, or substituted heteroaryl.

In some embodiment, each occurrence of X₂ is independently O, S, NR₁, CR₁R₂, C(═R₃), cycloalkyl, substituted cycloalkyl, heterocycloalkyl, substituted heterocycloalkyl, aryl, substituted aryl, heteroaryl, or substituted heteroaryl.

In some embodiment, each occurrence of R₁ and R₂ is independently hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, or substituted heteroaryl.

In some embodiment, each occurrence of R₃ is independently O, NR₁, and S.

In some embodiment, n is an integer from 0 to 10.

In another aspect, the present invention relates, in part, to a method of labeling, imaging, and/or detecting a compound or salt thereof having the structure of Formula (XXII)

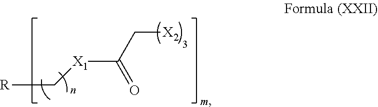

the method comprising reacting the compound or salt thereof having the structure of Formula (XXII) and a compound or salt thereof having the structure of Formula (II)

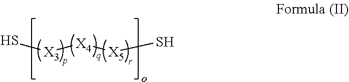

or
a compound or salt thereof having the structure of Formula (III)

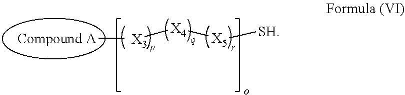

In some embodiments, R is an antibody or a fragment thereof, antigen or a fragment thereof, protein or a fragment thereof, peptide or a fragment thereof, amino acid sequence or a fragment thereof, amino acid or a derivative thereof, small molecule or a derivative thereof, therapeutic agent or a derivative thereof, or any combination thereof.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1D, depicts acetyl-CoA analogs to label KAT substrates through acetyltransferase assay. FIG. 1A depicts bond lengths of C—H, C—C≡CH, C—N₃, and C—F. FIG. 1B depicts an illustration of the acetyltransferase assay. FIG. 1C depicts a summary of the acetyltransferase assay results with Ac-CoA, 4-pentynoyl (4PY)-CoA, and F-Ac-CoA analogs, respectively. FIG. 1D depicts representative global profiling of lysine acetylation using anti-acetyl lysine antibodies that are commercially available from different vendors (#1-6). Top panel: Western blots of lysine acetylation in HEK 293 cell lysates with different anti-acetyl lysine antibodies (#1-6); Bottom panel: Coomassie blue staining of the 120 kDa area as loading controls.

FIG. 2, comprising FIG. 2A through FIG. 2C, depicts representative ESI-MS results for using acetyl-CoA analogs to label KAT substrates. Top row of MS spectra: results with GCN5 KAT and H3 (1-20) peptide. Middle row: results with MYST2 KAT and H4 (1-20) peptide. Bottom row: results with TIP60 KAT and H4 (1-20) peptide. Expected theoretical m/z were shown under each MS spectrum. “√” indicates the observed m/z matches to the expected values; “x” indicates not, which is the case for the assay with 4PY-CoA that only resulted in unmodified wild type substrates. FIG. 2A depicts Acetyl-CoA mixed with corresponding acetyl transferases (GCN5 or MYST2 or TIP60) and peptide substrates (H3-20: N-terminal 20-aa H3 peptide, exact mass 2182.2771 m/z; or H4-20: N-terminal 20-aa H4 peptide, exact mass 1990.1885 m/z). FIG. 2B depicts 4PY-CoA mixed with corresponding acetyl transferases (GCN5 or MYST2 or TIP60) and peptide substrates (H3-20: N-terminal 20-aa H3 peptide, exact mass 2182.2771 m/z; or H4-20: N-terminal 20-aa H4 peptide, exact mass 1990.1885 m/z). FIG. 2C depicts F-Ac-CoA mixed with corresponding acetyl transferases (GCN5 or MYST2 or TIP60) and peptide substrates (H3-20: N-terminal 20-aa H3 peptide, exact mass 2182.2771 m/z; or H4-20: N-terminal 20-aa H4 peptide, exact mass 1990.1885 m/z).

FIG. 3 depicts a schematic representation of fluorine-thiol displacement reaction (FTDR) using various fluorinated substrates.

FIG. 4, comprising FIG. 4A through FIG. 4C, depicts representative optimization of the FTDR by titrating the effects of reaction pH values. Standard nucleophile (thiophenol) and substrate (3) (2-Fluoro-N-phenethylacetamide) were used. FIG. 4A depicts a schematic representation of the FTDR using substrate (3) (2-Fluoro-N-phenethylacetamide and thiophenol. FIG. 4B depicts representative LC-MS spectra of reaction mixtures after 12 h of reaction under varied pH's. FIG. 4C depicts a summary plot of the product yields at different reaction pH's.

FIG. 5, comprising FIG. 5A through FIG. 5D, depicts representative results demonstrating that substrate (3) (2-Fluoro-N-phenethylacetamide) was stable upon incubation with glutathione. FIG. 5A depicts a schematic representation demonstrating that substrate (3) (2-fluoro-N-phenethylacetamide) is stable upon incubation with glutathione. FIG. 5B depicts a representative ¹H-NMR spectrum of (3) (2-fluoro-N-phenethylacetamide) (25 mM in 1:1 mix of deuterated sodium phosphate buffer and MeOD, pH 8.5). FIG. 5C depicts a representative ¹H-NMR spectrum of reduced glutathione (GSH) (25 mM in 1:1 mix of deuterated sodium phosphate buffer and MeOD, pH 8.5). FIG. 5D depicts a representative ¹H-NMR spectrum of the mixture of (3) (2-fluoro-N-phenethylacetamide) (25 mM) and GSH (25 mM) in the 1:1 mixed deuterated sodium phosphate buffer and MeOD, pH 8.5, after 24 h of incubation at 37° C.

FIG. 6, comprising FIG. 6A through FIG. 6D, depicts representative results demonstrating substrate (3) (2-fluoro-N-phenethylacetamide) is stable upon incubation with cysteine. FIG. 6A depicts a schematic representation demonstrating that (3) (2-fluoro-N-phenethylacetamide) is stable upon incubation with cysteine, FIG. 6B depicts a representative ¹H-NMR spectrum of (3) (2-fluoro-N-phenethylacetamide) (25 mM in 1:1 mix of deuterated sodium phosphate buffer and MeOD, pH 8.5). FIG. 6C depicts a representative ¹H-NMR spectrum of reduced glutathione (GSH) (25 mM in 1:1 mix of deuterated sodium phosphate buffer and MeOD, pH 8.5). FIG. 6D depicts a representative ¹H-NMR spectrum of the mixture of (3) (2-fluoro-N-phenethylacetamide) (25 mM) and GSH (25 mM) in the 1:1 mixed deuterated sodium phosphate buffer and MeOD, pH 8.5, after 24 h of incubation at 37° C.

FIG. 7, comprising FIG. 7A and FIG. 7B, depicts representative results obtained from structure-activity relationship study of benzenethiol derivatives.

FIG. 7A depicts a scheme of the benzenethiol derivatives. FIG. 7B depicts a plot of the conversion against reaction time.

FIG. 8 depicts a representative LC-MS spectra for analysis of the reaction mixtures undergoing general procedure B. The UV trace was for the reaction between substrate (3) (2-fluoro-N-phenethylacetamide) and the nucleophile 2,4,6-trimethoxybenzenethiol (13) after 13 h of incubation at 37° C. Identities of the peaks were confirmed by the corresponding ESI-MS analysis.

FIG. 9 depicts a summary of the pKa values of the benzenethiol derivatives. The data was referred from the SciFinder database.

FIG. 10 depicts representative results evaluating the second order rates of FTDR reactions between (3) (2-fluoro-N-phenethylacetamide) and (12) (3,4,5-trimethoxybenzenethiol), (3) (2-fluoro-N-phenethylacetamide) and (13) (2,4,6-trimethoxybenzenethiol), respectively. Equal concentrations of both reactants were used and the assays were repeated independently at three different concentrations (40 mM, 80 mM, and 160 mM). Plotting 1/[X]t (concentration of either reactant at time t) against time yielded the rate constant ((0.37±0.06)×10⁻³ M⁻¹ S⁻¹ for (3) (2-fluoro-N-phenethylacetamide) and (12) (3,4,5-trimethoxybenzenethiol), (1.03±0.06)×10⁻³ M⁻¹ S⁻¹ for (3) (2-fluoro-N-phenethylacetamide) and (13) (2,4,6-trimethoxybenzenethiol)). The reported values represented an average of the three independent experiments.

FIG. 11, comprising FIG. 11A through FIG. 11C, depicts biotinylation of fluorinated H3-20 peptide based on the FTDR. FIG. 11A depicts a reaction scheme for biotinylation of fluorinated H3-20 peptide based on the FTDR. FIG. 11B depicts a representative ESI-MS spectrum of the fluorinated H3-20 peptide before reacting with Biotin-SH during the process of biotinylation. H3-20 peptide sequence is “NH2-Ala-Arg-Thr-Lys-Gln-Thr-Ala-Arg-Lys-Ser-Thr-Gly-Gly-Lys-Ala-Pro-Arg-Lys-Gln-Leu-COOH”. FIG. 11C depicts a representative ESI-MS spectrum of the fluorinated H3-20 peptide after reacting with Biotin-SH during the process of biotinylation. H3-20 peptide sequence is “NH2-Ala-Arg-Thr-Lys-Gln-Thr-Ala-Arg-Lys-Ser-Thr-Gly-Gly-Lys-Ala-Pro-Arg-Lys-Gln-Leu-COOH”.

FIG. 12, comprising FIG. 12A through FIG. 12C, depicts representative results for FTDR-based tagging of protein substrates with Biotin-SH probe. FIG. 12A depicts a reaction scheme for tagging of protein substrates. The red star indicates IR dye. FIG. 12B depicts labeling of histone protein H3.1. The top panel is a gel image after CBB staining. The other images are for in-gel fluorescent detection of IR dye. FIG. 12C depicts labeling of nonhistone substrate EZH2 (1-500). The left panel is a gel image after CBB staining. The other images are for in-gel fluorescent detection of IR dye. The green band is the reference protein ladder under the 600 nm channel.

FIG. 13 depicts representative results demonstrating biotinylation of H4 protein substrates by MYST2 enzyme. Biotin was detected by streptavidin-IRDye 680RD under near infrared fluorescence scanning (Ex 685 nm/Em 730 nm).

FIG. 14 depicts representative characterization of EZH2 (1-500) protein by SDS-PAGE analysis.

FIG. 15, comprising FIG. 15A through FIG. 15C, depicts representative results of cellular evaluation of FTDR-based tagging with TAMRA-SH probe. FIG. 15A depicts a schematic representation of cellular pro-metabolite incorporation (1 mM, 6 h, at 37° C., step 1) and protein substrate detection (step 2). FIG. 15B depicts representative fluorescent microscopy images of fixed and permeabilized cells that were stained by Hoechst 33342 (blue) and TAMRA probes (red); Scale bars: 25 μm. FIG. 15C depicts representative results of cell lysate protein labeling by pro-metabolites and detection by TAMRA probes (red); Left panel: PAGE gel stained by CBB; Right panel: In-gel fluorescent detection. “C” is the positive control for CuAAC, which ran-domly labelled lysines on BSA with azide-NHS ester, followed by Cu-AAC mediated conjugation with TAMRA-Alkyne. “HATi” indicates the addition of HAT inhibitor (A-485) prior to step 1.

FIG. 16, comprising FIG. 16A and FIG. 16B, depicts representative in vitro cell cytotoxicity assay results to confirm the nontoxicity of ethyl ester pro-metabolites. FIG. 16A depicts representative results demonstrating relative cytotoxicity on HeLa cells by ethyl fluoroacetate or the control ethyl azidoacetate after 12 h of incubation were plotted. Error bars represent SD of three replicates. FIG. 16B depicts representative results demonstrating relative cytotoxicity on HEK293T cells by ethyl fluoroacetate or the control ethyl azidoacetate after 12 h of incubation were plotted. Error bars represent SD of three replicates.

FIG. 17 depicts a synthetic scheme of 2-fluoro-1-phenylethan-1-one.

FIG. 18 depicts a synthetic scheme of 1-fluoro-5-phenylpentan-2-one.

FIG. 19 depicts a synthetic scheme of 2-fluoro-N-phenethylacetamide.

FIG. 20 depicts a synthetic scheme of 2-fluoro-phenethylacetate.

FIG. 21 depicts a synthetic scheme of 3,4,5-trimethoxybenzenethiol.

FIG. 22 depicts a synthetic scheme of 2,4,6-trimethoxybenzenethiol.

FIG. 23 depicts a synthetic scheme of N,N-bis[(1,1-dimethylethoxy)carbonyl]-5-iodo-phenylmethyl ester.

FIG. 24 depicts a synthetic scheme of Benzyl 2-amino-5-(3,5-dimethoxy-4-thiocyanatophenoxy) pentanoate.

FIG. 25 depicts a synthetic scheme of the diol cleavable biotin linker conjugated with the 4-mercapto-3,5-dimethoxyphenoxy probe (Biotin-SH).

FIG. 26 depicts a synthetic scheme of the TAMRA dye conjugated 4-mercapto-3,5-dimethoxyphenoxy probe (TAMRA-SH).

FIG. 27 depicts a synthetic scheme of the TAMRA dye conjugated alkyne probe (TAMRA-Alkyne).

FIG. 28 depicts a schematic representation of general procedure A for Exploration of Fluorinated Substrate.

FIG. 29 depicts a schematic representation of optimizing reaction conditions—pH titration.

FIG. 30 depicts a schematic representation of bioorthogonality of the FTDR.

FIG. 31 depicts a schematic representation of general procedure B for structure-activity relationship studies of nucleophiles.

FIG. 32, comprising FIG. 32A through FIG. 32C, depicts representative results for validation of the FTDR-based labeling of acetylation substrates. FIG. 32A depicts a schematic representation of the cellular pro-metabolite incorporation (step 1), protein substrate labeling by TAMRA-SH (step 2), and extraction of the known acetylation substrates histones; or protein substrate labeling by Biotin-SH probe (step 2), enrichment with streptavidin beads, followed by western blot analysis of the proteins pulled down to examine the existence of alpha-tubulin, histone H3 and H4. FIG. 32B depicts a representative histone extraction results. Top panel: In-gel fluorescent detection; Bottom panel: CBB. FIG. 32C depicts representative western blotting results. “HATi” indicates the addition of anacardic acid and MG149 (Deng H et al., 2020, Chemosphere, 247:125825; Sun Y et al., 2006, FEBS Lett., 580:4353-4356; Simpson S et al., 2018, Front. Microbiol., 9:788).

FIG. 33, comprising FIG. 33A and FIG. 33B, depicts representative validation of the fluoroacetyl labeling sites on known protein substrates by proteomics analysis. FIG. 33A depicts a schematic representation of the cellular pro-metabolite incorporation, lysis, and the histone proteins extraction; or immunoprecipitation of alpha-tubulin after cellular pro-metabolite incorporation. FIG. 33B depicts representative summary of the F-Ac labeling sites (red) on representative histone proteins H2B, H4 (PDB: 1kx5), and alpha-tubulin (PDB: 1tub). Details of the proteomics results are available in the supporting information (FIG. 42 through FIG. 44).

FIG. 34 depicts a schematic representation of the steric-free bioorthogonal labeling of acetylation substrates based on a FTDR of the present invention.

FIG. 35, comprising FIG. 35A through FIG. 35C, depicts representative chemical reactivity of fluoroacetyl-CoA (F-Ac-CoA) and acetyl-CoA (Ac-CoA). FIG. 35A depicts representative comparison of rates of hydrolysis of 10 μM F-Ac-CoA and 10 μM Ac-CoA at 100 mM Tris buffer (pH 7.2). The pseudo-first-order rate constants were determined to be 7.5×10⁻³ s⁻¹ for F-Ac-CoA, and 2.0×10⁻⁴ s⁻¹ for Ac-CoA, respectively. FIG. 35B depicts representative non-enzymatic acetylation of F-Ac-CoA and Ac-CoA to the model protein bovine serum albumin (BSA) at pH 7.0 or pH 8.0, 37° C.; The positive control, “NHS-Acetate”, was tested in parallel, which was expected to readily modify lysines of BSA with acetates. Top row: western blot for acetyl-lysine or F-acetyl lysine residues on BSA; Bottom row: coomassie brilliant blue (CBB) staining as loading controls. The MultiMab™ antibody (Ac-K-100, Cell Signaling) used for western blots are a mixture of monoclonal antibodies that can recognize acetyl-lysine and F-acetyl lysine. FIG. 35C depicts representative characterization of the non-enzymatic F-acetylation using the TAMRA-SH probe based on FTDR. The positive control, “NHS-F-Acetate”, was prepared by modifying lysines on BSA with F-acetates.

FIG. 36, comprising FIG. 36A and FIG. 36B, depicts representative results from the stability and reactivity evaluations of the halo-acetamide tags and the probe (13) in mammalian cell lysates. The assays outlined in FIG. 36 were repeated in triplicate. FIG. 36A depicts representative model substrate (3), control (3-Cl), or the probe (13) that were each mixed with the corresponding internal standard, and incubated within the HEK293 cell lysates for 14 h under the same FTDR reaction conditions, respectively. The compounds were then recovered by extraction and analyzed by LC-MS, generating a summary plot of recovery yields on the basis of triplicate repeats. FIG. 36B depicts representative results of the peak area and m/z information on LC-MS for each group analyzed before and after incubation with the cell lysates.

FIG. 37, comprising FIG. 37A and FIG. 37B, depicts representative FTDR reaction in mammalian cell lysates. FIG. 37A depicts a schematic representation of the model substrate (3) and probe (13) that were mixed in the presence of HEK293 cell lysates. After incubation at 37° C. for 5 h, the mixture was extracted and analyzed by LC-MS/MS. FIG. 37B depicts representative LC-MS (top panel) and MS/MS (bottom panel) analysis of the cell lysate mixture identified the FTDR reaction product (47).

FIG. 38 depicts representative attempted FTDR reaction between probe 13 and the alpha-fluorinated model substrates of fatty acids including butyrate, malonic acid, succinic acid, myristic acid, and palmitic acid. Briefly, probe (13) (50 mM) and a model substrate (25 mM) were dissolved in 20 μL of Tris buffer/DMF (60 mM, pH 8.5) which also contained 100 mM TCEP. LC-MS analysis of the mixture after incubation at 37° C. for 12 h revealed no reaction product, presumably due to the steric hindrance of the secondary fluorides.

FIG. 39 depicts representative western blot assays for evaluating the removal of F-acetyl groups on histone substrate by different histone deacetylases (SIRT1 for fluoroacetylated histone H3, HDACs 1-3 and SIRT2 for fluoroacetylated histone H4). Negative control “−”: intact wild type H3 or H4; Positive control “+”: fluoroacetylated H3 or H4 after treatment with F-Ac-CoA and the corresponding acetyltransferases. Other samples lanes are the mixture of fluoroacetylated H3 or H4 with different histone deacetylases.

FIG. 40, comprising FIG. 40A through FIG. 40D, depicts representative LC-MS/MS analysis of fluoroacetyl-CoA formation in HEK293 cells treated with the pro-metabolite fluoroacetate. FIG. 40A depicts representative LC-MS of HEK293 cell extract demonstrating the eluted peak specific to fluoroacetyl-CoA (828.1265 m/z). The cells were treated with 1 mM ethyl fluoroacetate for 2 h. FIG. 40B depicts representative LC-MS of extracts from preheated HEK293 lysates that were incubated with 1 mM ethyl fluoroacetate for 2 h. FIG. 40C depicts representative product ions derived from fluoroacetyl-CoA for MS/MS analysis. FIG. 40D depicts representative LC-MS/MS fragmentation analysis of the eluted fluoroacetyl-CoA peak.

FIG. 41, comprising FIG. 41A and FIG. 41B, depicts representative FTDR-based imaging of acetylation after concurrent HDAC inhibition. FIG. 41A depicts representative results for HEK293 cells treated with the pro-metabolite ethyl fluoroacetate with or without the presence of the HDAC inhibitor cocktail (APExBIO) for 6 h or 12 h, before lysis and FTDR reaction. Left: CBB staining of all the cell lysate samples after FTDR reaction; Right: Fluorescent imaging. FIG. 41B depicts a schematic representation of the proposed hijacking of intrinsic acetylation. Under the dynamic acetylation-deacetylation equilibrium, the acetylated sites on protein substrates could be deacetylated to allow for modification by F-Ac (top row). With the equilibrium blocked by HDACi, the intrinsic acetylation may compete with F-acetylation.

FIG. 42 depicts representative proteomics analysis of histone H2B (SEQ ID NO: 1), which was extracted from HEK293 cells after treatment with pro-metabolite ethyl fluoroacetate. Pink legend: F-acetylation; Purple legend: wild type acetylation. The N-terminal amino acid is proline.

FIG. 43 depicts representative proteomics analysis of histone H4 (SEQ ID NO: 2), which was extracted from HEK293 cells after treatment with pro-metabolite ethyl fluoroacetate. Pink legend: F-acetylation; Purple legend: wild type acetylation. The N-terminal amino acid is serine.

FIG. 44 depicts representative proteomics analysis of alpha-tubulin (SEQ ID NO: 3), which was immunoenriched from HEK293 cells after treatment with pro-metabolite ethyl fluoroacetate. Pink legend: F-acetylation; Purple legend: wild type acetylation.

FIG. 45 depicts a schematic representation of the present invention that allowed the facile preparation of constrained macrocyclized peptides of different linker sizes, and led to the identification of stapled peptides that possessed improved target binding and cellular permeability compared to hydrocarbon stapled peptides.

FIG. 46 depicts representative reaction of the fluoroacetamide-containing model compound (1) with methyl hydrazine and cysteine, respectively. For the reaction with cysteine, the mixture contained 500 mM TCEP to ensure a reducing environment. The reaction progress was monitored by LC-MS after 12 h of incubation at 37° C.

FIG. 47 depicts representative time-dependent fluorine displacement reaction between the model compound (1) and benzyl thiol. The mixture contained 500 mM TCEP in the Tris/DMF solution. The reaction progress was monitored by LC-MS.

FIG. 48, comprising FIG. 48A and FIG. 48B, depicts representative FTDR between benzyl thiol and the α-fluoroacetamide containing amino acid building block (9). FIG. 48A depicts representative model FTDR between benzyl thiol and the α-fluoroacetamide containing amino acid building block (9). FIG. 48B depicts model macrocyclization carried out between unprotected model peptide (18) (SEQ ID NO: 4) and a commercially available linker 1,4-benzenedimethanethiol based on FTDR.

FIG. 49 depicts representative time-dependent stapling of the linear model peptide (18) with the linker 1,4-benzenedimethanethiol. The reaction progress was monitored by LC-MS. The peak I is starting peptide (18); peak II is the desired product (19) (SEQ ID NO: 4); peak III is the noncyclic byproduct modified by one equivalent of linker; peak IV is the noncyclic byproduct modified by two equivalents of linkers.

FIG. 50, comprising FIG. 50A through FIG. 50D, depicts representative FTDR coupling between an unprotected Axin peptide analogue (SEQ ID NO: 5) and various dithiol linkers. FIG. 50A depicts representative coupling with the unprotected analogue (20) (SEQ ID NO: 5) that has both L-fluoroacetamide substrates and the analogue (27) (SEQ ID NO: 5) that has both D-fluoroacetamide substrates, respectively. FIG. 50B depicts representative coupling with the unprotected analogue (34) (SEQ ID NO: 5) that has L-fluoroacetamide and D-fluoroacetamide (N-C direction), and analogue (38) (SEQ ID NO: 5) that has D-fluoroacetamide and L-fluoroacetamide (N-C direction), respectively. FIG. 50C depicts representative reported Axin analogue (42) (SEQ ID NO: 5) was prepared based on ring-closing metathesis. FIG. 50D depicts representative circular dichroism (CD) spectra of all peptide analogues.

FIG. 51, comprising FIG. 51A through FIG. 51D, depicts representative FTDR coupling between unprotected HIV C-CA binding peptide analogues (SEQ ID NO: 6) and 1,3-benzenedimethanethiol. FIG. 51A depicts representative FTDR coupling between unprotected HIV C-CA binding peptide analogues and 1,3-benzenedimethanethiol and HIV C-CA binding peptide (46) (SEQ ID NO: 6) that was stapled by RCM. FIG. 51B depicts representative CD spectra of the crosslinked peptide analogues. FIG. 51C depicts representative fluorescent confocal microscopy images of the HEK293T cells treated with peptides (43)-(46) (SEQ ID NO: 6). Blue: nucleus stained by Hoechst 33342; Green: FITC-labelled peptides. FIG. 51D depicts representative results of quantification analysis of the cell penetration of peptides (43)-(46) (SEQ ID NO: 6). The intracellular intensity of peptide (46) (SEQ ID NO: 6) was normalized as 1. “****” represents p<0.0001.

FIG. 52, comprising FIG. 52A through FIG. 52E, depicts representative results for 1,3-benzenedimethanethiol crosslinked Axin peptides (25), (32), (37), and (41). FIG. 52A depicts representative energy-minimized structures of 1,3-benzenedimethanethiol crosslinked Axin peptides (25), (32), (37), and (41). FIG. 52B depicts representative results demonstrating the slowest implied timescale of peptides (25) and (20) (no linker) shown as a function of MSM lag time. FIG. 52C depicts representative predicted per-residue helicity profiles of stapled and unstapled Axin peptides. Uncertainties were estimated from a bootstrap procedure where five MSMs were constructed by sampling the input trajectory data with replacement. FIG. 52D depicts representative predicted per-residue helicity profiles of HIV C-CA binding peptides (43), (44), and (45). FIG. 52E depicts representative comparison of experimental (CD) and predicted average helicities of peptides (43), (44), and (45).

FIG. 53 depicts representative Comparison of predicted and experimental helicities for stapled Axin peptides (21)-(26), (28)-(33), (35)-(37), and (39)-(41). Uncertainties (vertical bars) were calculated using a bootstrap procedure in which five different MSMs were constructed by sampling the input trajectory data with replacement. Peptides (25) and (37) are labeled with star makers to denote the highest experimentally measured helicities.

FIG. 54 depicts representative minimized structures of stapled Axin peptides crosslinked at the i, i+4 positions.

FIG. 55 depicts representative results demonstrating viability of cells after treatment with FITC-labelled peptide analogues. HEK293T cells were treated with HIV C-CA binding peptides, while DLD-1 cells were treated with Axin analogues at doses of 5, 10, or 15 μM. In each set, the cells treated with solvent only were normalized as the 100% control.

FIG. 56, comprising FIG. 56A through FIG. 56H, depicts representative results for a FTDR coupling between unprotected Axin-derived peptide analogues (SEQ ID NO: 7) (47) and (49) and 1,3-benzenedimethanethiol as well as the Axin analogue (51) (SEQ ID NO: 7) that was stapled by RCM and used for cell penetration studies. FIG. 56A depicts schematic representation of FTDR coupled peptide analogues (47) and (49) and the Axin analogue (51) that was stapled by RCM and used for cell penetration studies. FIG. 56B depicts representative fluorescent confocal microscopy images of the DLD-1 cells treated with peptides (47)-(51) (SEQ ID NO: 7) and FITC only as a negative control. Blue: nucleus stained by Hoechst 33342; Green: FITC. FIG. 56C depicts representative results for quantification analysis of the cell penetration of peptides (47)-(51) (SEQ ID NO: 7). The intracellular intensity of peptide (51) was normalized as 1. “****” represents p<0.0001. FIG. 56D depicts representative results for quantification analysis of the cell penetration of peptide (48) or (50) after the cells had been treated with blockers of endocytic pathways. The intracellular intensity of peptides in cells untreated with any blocker was normalized as 1. “****” represents p<0.0001. FIG. 56E depicts representative results demonstrating the binding affinity of unstapled (47) and stapled Axin analogues (48), (50), and (51) to the target β-catenin protein. Peptides (48) and (50) were stapled via FTDR, while peptide (51) was stapled by RCM. FIG. 56F depicts representative results demonstrating the stability of the aforementioned peptide analogues in 100% rat serum. FIG. 56G depicts representative results demonstrating the inhibition of these peptide analogues on DLD-1 cancer cell growth over 5 days. FIG. 56H depicts representative results demonstrating that the peptides stapled by the method described herein permeate mammalian cells using a significantly different mechanism from other known stapled peptides.

FIG. 57 depicts representative Raw imaging data for DLD-1 cells treated with 10 μM of FITC-labelled Axin analogues.

FIG. 58, comprising FIG. 58A through FIG. 58C, depicts representative cell selection for FITC-labelled Axin analogue (48) using the NIH ImageJ analyze particles function. FIG. 58A depicts representative single and multiple cell selection for the FITC-labeled peptide. FIG. 58B depicts representative extracellular particle selection and removal. FIG. 58C depicts representative removal of the cytoplasmic membrane from the mean intensity measurement.

FIG. 59, comprising FIG. 59A and FIG. 59B, depicts representative results demonstrating outlier exclusion for cells treated with FITC-labelled Axin analogue (48). FIG. 59A depicts representative histogram of the mean intensities of the FITC-labeled peptide with a normal distribution curve applied. With a normal distribution, four cells (yellow arrows) with values 31.13, 32.41, 35.64, and 42.38 is excluded from the data set. FIG. 59B depicts representative histogram of the mean intensities of the FITC-labeled peptide with the best fit, lognormal distribution curve applied. With a lognormal distribution, one cell (yellow arrow) with a value 42.38 is excluded from the data set.

FIG. 60 depicts representative fluorescent confocal microscopy images of the DLD-1 cells treated with the stapled Axin analogue (48) in the presence of different endocytotic pathway blockers (Nystatin, Chlorpromazine, Cytochalasin D, and NaClO₃). Green: FITC-labelled peptide (48); Blue: nucleus stained by Hoechst 33342. Control: cells pretreated with vehicle control and then incubated with peptide (48).

FIG. 61 depicts representative fluorescent confocal microscopy images of the DLD-1 cells treated with the stapled Axin analogue (50) in the presence of different endocytotic pathway blockers (Nystatin, Chlorpromazine, Cytochalasin D, and NaClO₃). Green: FITC-labelled peptide (50); Blue: nucleus stained by Hoechst 33342. Control: cells pretreated with vehicle control and then incubated with peptide (50).

FIG. 62 depicts representative results demonstrating viability of DLD-1 cells after imaging studies with representative stapled Axin analogues (48) or (50). Cells were pre-treated with different small molecule blockers of endocytic pathways before imaging studies. The viability of cells treated with only vehicle control were normalized as 100%.

FIG. 63 depicts representative generalized matrix Rayleigh quotient (GMRQ) scores, shown versus the number of states used to construct the MSMs, shown for MSMs of 1,3-benzenedimethanethiol stapled (L,D) Axin peptide (top) and unstapled (L,D) Axin peptide (bottom). Five-fold cross-validation was used, where the MSM was trained on ⅘ of the input data, and the GMRQ score computing using the remaining ⅕ of the data, in five separate trials. While the mean GMRQ score for the training data (blue) continues to increase with the number states, the mean score for the testing data reaches a peak at around 50 states (marked with a star). Based on the general consistency of these results for most of the peptide systems, MSMs was constructed using 50 states.

FIG. 64 depicts representative results demonstrating Slowest ten implied timescales for MSMs of 1,3-benzenedimethanethiol stapled (L,D) Axin peptide (top) and unstapled (L,D) Axin peptide (bottom), plotted as a function of lagtime, τ. Implied timescales were calculated as tᵢ=τ/ln λᵢ where λᵢ are the eigenvalues of the MSM transition matrix. Uncertainties were estimated using a per-trajectory bootstrap procedure.

FIG. 65 depicts representative examples of trajectory data projected on the first two tICs, with representative structures, for 1,3-benzenedimethanethiol stapled (L,D) Axin peptide (top), and unstapled (L,D) Axin peptide (bottom). The color scale represents regions of high (red) to low (blue) density.

FIG. 66 depicts representative comparison between the helicities of 1,3-benzenedimethanethiol stapled Axin peptides (blue circles) and unstapled Axin peptides (red circle). Uncertainties (vertical bars) were calculated using a bootstrap procedure in which five different MSMs were constructed by sampling the input trajectory data with replacement.

FIG. 67 depicts schematic representation of synthesis of 2-(9H-Fluoren-9-ylmethoxycarbonylamino)-3-(2-fluoro-acetylamino)-propionic acids (compounds (15)/(16).

### DETAILED DESCRIPTION

The present invention is based, in part, on the novel bioorthogonal reaction that can generate steric-free labeling of protein substrates and thereby allow for global profiling of molecular targets. Thus, the present invention provides, in part, methods of labeling, imaging, and/or detecting protein substrates using said bioorthogonal reaction. The present invention also relates, in part, to novel methods of linking amino acid sequences to various compounds (e.g., therapeutic agents, small molecules, etc.) as well as novel methods of making stapled amino acid sequences. The present invention also relates to the said amino acid sequences linked to various compounds and the said stapled amino acid sequences as well as compositions thereof. The present invention further provides methods of treating various disorders and diseases using the said compositions.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, such as an amount, a temporal duration, and the like, is meant to encompass variations of 20%, ±10%, +5%, +1%, or ±0.1% from the specified value, as such variations are appropriate to perform the disclosed methods.

As used herein, the term “alkyl,” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C—N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to aryl groups which contain at least one heteroatom selected from N, O, Si, P, and S; wherein the nitrogen and sulfur atoms may be optionally oxidized, and the nitrogen atom(s) may be optionally quaternized. Heteroaryl groups may be substituted or unsubstituted. A heteroaryl group may be attached to the remainder of the molecule through a heteroatom. A polycyclic heteroaryl may include one or more rings that are partially saturated. Examples include tetrahydroquinoline, 2,3-dihydrobenzofuryl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

The term “thiol” as used herein is represented by the formula —SH.

As used herein, the terms “amino acid”, “amino acidic monomer”, or “amino acid residue” refer to any of the twenty naturally occurring amino acids or pyrolysine or selenocysteine including synthetic amino acids with unnatural side chains and including both D and L optical isomers. The term “amino acid” includes, but is not limited to, proteinogenic amino acids.

As used herein, the term “amino acid sequence” refers to linked sequence of two or more amino acids and can be natural, synthetic, or a modification or combination of natural and synthetic. The term “amino acid sequence” includes, but is not limited to, a linear covalently bound amino acid sequence and/or a branched covalently bound amino acid sequence.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “recombinant polypeptide” as used herein is defined as a polypeptide produced by using recombinant DNA or RNA methods.

The term “stapling,” as used herein, refers a process by which two functional groups on different amino acids in a same amino acid sequence (e.g., amino sequence A) react with each in the presence of an appropriate linker to generate a cross-link between the two amino acids (a “staple”). The term “stapling,” as used herein, also refers a process by which two functional groups on different amino acids in different amino acid sequences (e.g., amino acid sequence A and amino acid sequence B) react with each in the presence of an appropriate linker to generate a cross-link between the two amino acid sequences (a “staple”). Stapling engenders constraint on a secondary structure, such as an alpha helical structure. The length and geometry of the cross-link can be optimized to improve the yield of the desired secondary structure content. The constraint provided can, for example, prevent the secondary structure to unfold and/or can reinforce the shape of the secondary structure, and thus makes the secondary structure more stable. Multiple stapling is also referred to herein as “stitching.” See, e.g., U.S. Pat. Nos. 7,192,713; 7,723,469; 7,786,072; U.S. Patent Application Publication Nos: 2010-0184645; 2010-0168388; 2010-0081611; 2009-0176964; 2009-0149630; 2006-0008848; PCT Application Publication Nos: WO 2010/011313; WO 2008/121767; WO 2008/095063; WO 2008/061192; and WO 2005/044839, which depict stapling and stitching of polypeptides. In certain embodiments, stapling may occur at i,i+2, i,i+3, i,i+4, i,i+7, i,i+8, i,i+10, and/or i,i+11 positions of the amino acid sequence.

As used herein, the term “stapled amino acid sequence” means that amino acid sequence regions are connected to each other. In one embodiment, in order to increase the chemical stability of alpha-helices, the i position and i+4 position (or i+7 and i+11 positions) of the alpha-helix can be stapled using various covalent bonding methods. Specifically, the amino acids at one or more positions selected from the group consisting of i, i+3, i+4, i+7, i+8, i+10 and i+11 (where i is an integer) may be stapled Amino acids may be stapled by a covalent bond to thereby increase the cell-penetrating ability. In some cases, two or more amino acid positions selected from the group consisting of i, i+3, i+4, i+7, i+8, i+10 and i+11 (where i is an integer) may be stapled. As used herein, the term “stapled amino acid sequence” refers to an amino acid sequence comprising at least one pair of functionalized amino acids, wherein the functionalized amino acids are joined by a staple. In the case of a helical stapled amino acid sequence, the plurality of amino acids joined by a plurality of peptide bonds and at least one staple forms a macrocyclic ring formed between the α-carbon of one amino acid and the α-carbon of another amino acid, which includes also any amino acid(s) between the functionalized amino acids.

As used herein, the term “linking” refers to joining of two or more components with a linker. For example, in one embodiment, “linking” amino acid sequences refers to “stapling” amino acid sequences. The term “linking”, as used herein, also refers to a formation of a covalent bond or non-covalent bond. For example, in one embodiment, the term “linking” refers to a formation of a covalent bond between a functional group on an amino acid in an amino acid sequence and a functional group on a small molecule.

As used herein, “linked” means to couple directly or indirectly one molecule with another by whatever means, e.g., by covalent bonding, by non-covalent bonding, by ionic bonding, or by non-ionic bonding. Covalent bonding includes bonding by various linkers such as thioether linkers or thioester linkers. Direct linking involves one molecule attached to the molecule of interest. Indirect linking involves one molecule attached to another molecule which in turn is attached directly or indirectly to the molecule of interest.

As used herein, the term “linkage” refers to bonds or chemical moiety formed from a chemical reaction between the functional group of a linker and another molecule. Such bonds may include, but are not limited to, covalent linkages and non-covalent bonds, while such chemical moieties may include, but are not limited to, esters, carbonates, imines phosphate esters, hydrazones, acetals, orthoesters, peptide linkages, and oligonucleotide linkages. Hydrolytically stable linkages means that the linkages are substantially stable in water and do not react with water at useful pH values, including but not limited to, under physiological conditions for an extended period of time, perhaps even indefinitely. Hydrolytically unstable or degradable linkages means that the linkages are degradable in water or in aqueous solutions, including for example, blood. Enzymatically unstable or degradable linkages means that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include, but are not limited to, ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

As used herein, the term “identical” refers to two or more sequences or subsequences which are the same.

In addition, the term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence.

As used herein, “fragment” is defined as at least a portion of a sequence. For example, in one embodiment, the term “fragment” refers to a portion of the variable region of the immunoglobulin molecule which binds to its target, i.e. the antigen binding region. Some of the constant region of the immunoglobulin may be included.

The term “DNA” as used herein is defined as deoxyribonucleic acid.

The term “RNA” as used herein is defined as ribonucleic acid.

The term “recombinant DNA” as used herein is defined as DNA produced by joining pieces of DNA from different sources.

The term “recombinant RNA” as used herein is defined as RNA produced by joining pieces of RNA from different sources.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides”. The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “antibody,” as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)₂ and F(ab′)₂, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen.

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

A “chimeric antibody” refers to a type of engineered antibody which contains a naturally-occurring variable region (light chain and heavy chains) derived from a donor antibody in association with light and heavy chain constant regions derived from an acceptor antibody.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

By the term “recombinant antibody” as used herein, is meant an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

As used herein, “antigen-binding domain” means that part of the antibody, recombinant molecule, the fusion protein, or the immunoconjugate of the invention which recognizes the target or portions thereof.

By the term “specifically binds,” as used herein, is meant a molecule, such as an antibody, which recognizes and binds to another molecule or feature, but does not substantially recognize or bind other molecules or features in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “effective amount” and “pharmaceutically effective amount” refer to a sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation. An “effective amount” or “therapeutically effective amount” of a compound is that amount of compound, which is sufficient to provide a beneficial effect to the subject to which the compound is administered.

As used herein, “pharmaceutically-acceptable” means that drugs, medicaments or inert ingredients which the term describes are suitable for use in contact with the tissues of humans and lower animals without undue toxicity, incompatibility, instability, irritation, allergic response, and the like, commensurate with a reasonable benefit/risk ratio.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The term “pharmaceutically acceptable salt” refers to any pharmaceutically acceptable salt, which upon administration to the subject is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, metabolites, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the microparticle cluster. The active agent-microparticle cluster combination may be coated further to encapsulate the agent-microparticle cluster combination and may be directed to a target by functionalizing the microparticle cluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

The term “solvate” in accordance with this invention should be understood as meaning any form of the active compound in accordance with the invention in which the said compound is bonded by a non-covalent bond to another molecule (normally a polar solvent), including especially hydrates and alcoholates.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The terms “patient”, “subject”, “individual”, and the like are used interchangeably herein, and refer to any animal, in some embodiments a mammal, and in some embodiments a human, having a complement system, including a human in need of therapy for, or susceptible to, a condition or its sequelae. As used herein, the terms “patient”, “subject”, “individual”, and the like can be a vertebrate, such as a mammal, a fish, a bird, a reptile, or an amphibian. Thus, the subject of the herein disclosed methods can be a human, non-human primate, monkey, horse, pig, rabbit, dog, sheep, goat, cow, cat, mouse, guinea pig or rodent. The terms do not denote a particular age or sex. Thus, adult and newborn subjects, as well as fetuses, whether male or female, are intended to be covered. In one aspect, the subject is a mammal.

A “therapeutic treatment” is a treatment administered to a subject who exhibits signs of disease or disorder, for the purpose of diminishing or eliminating those signs.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause one decrease in the subject's state of health.

As used herein, “treating a disease or disorder” means reducing the frequency and/or severity of a sign and/or symptom of the disease or disorder is experienced by a subject.

A disease or disorder is “alleviated” if the severity of a sign or symptom of the disease or disorder, the frequency with which such a sign or symptom is experienced by a subject, or both, is reduced.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected/homeostatic) respective characteristic. Characteristics which are normal or expected for one cell, tissue type, or subject, might be abnormal for a different cell or tissue type.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the novel bioorthogonal reaction that can generate steric-free labeling of protein substrates and thereby allow for global profiling of molecular targets. Thus, the present invention provides, in part, methods of labeling, imaging, and/or detecting protein substrates using said bioorthogonal reaction. The present invention also relates, in part, to novel methods of linking amino acid sequences to various compounds (e.g., therapeutic agents, small molecules, etc.) as well as novel methods of making stapled amino acid sequences. The present invention also relates to the said amino acid sequences linked to various compounds and the said stapled amino acid sequences as well as compositions thereof. The present invention further provides methods of treating various disorders and diseases using the said compositions.

### Methods of Making Stapled Amino Acid Sequences

The present invention provides a method of stapling one or more amino acid sequences. In one aspect, the present invention relates to a method of bioorthogonal stapling of one or more amino acid sequences. In one aspect, the method comprises reacting a compound or salt thereof having the structure of Formula (I) and a compound or salt thereof having the structure of Formula (II)

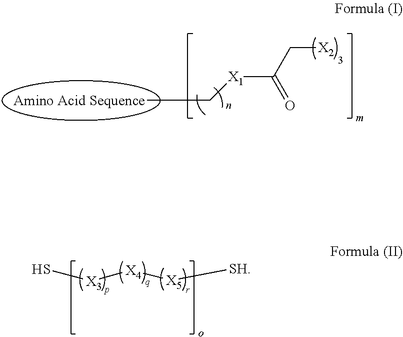

In some embodiments, each occurrence of X₁ is independently O, S, NR₁, CR₁R₂, or C(═R₃). In some embodiments, each X₂ is independently hydrogen, alkyl, substituted alkyl, or halogen. In some embodiments, each occurrence of X₂ is independently H, Br, Cl, F, or I.

In some embodiments, each occurrence of R₁ and R₂ is independently hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, or substituted heteroaryl. In some embodiments, each occurrence of R₃ is independently O, NR₁, or S.

In some embodiments, each occurrence of X₃, X₄, and X₅ is independently O, S, NR₁, CR₁R₂, C(═R₃), cycloalkyl, substituted cycloalkyl, heterocycloalkyl, substituted heterocycloalkyl, aryl, substituted aryl, heteroaryl, substituted heteroaryl, alkyl, substituted alkyl, —Y(R₄)ₐ(R₅)_{b}-cycloalkyl, substituted —Y(R₄)ₐ(R₅)_{b}-cycloalkyl, —Y(R₄)ₐ(R₅)_{b}-heterocycloalkyl, substituted —Y(R₄)ₐ(R₅)_{b}-heterocycloalkyl, alkenyl, substituted alkenyl, cycloalkenyl, substituted cycloalkenyl, —Y(R₄)ₐ(R₅)_{b}-cycloalkenyl, substituted —Y(R₄)ₐ(R₅)_{b}-cycloalkenyl, alkynyl, substituted alkynyl, cycloalkynyl, substituted cycloalkynyl, —Y(R₄)ₐ(R₅)_{b}-cycloalkynyl, substituted —Y(R₄)ₐ(R₅)_{b}-cycloalkynyl, —Y(R₄)ₐ(R₅)_{b}-aryl, substituted —Y(R₄)ₐ(R₅)_{b}-aryl, heteroaryl, substituted heteroaryl, —Y(R₄)ₐ(R₅)_{b}-heteroaryl, substituted —Y(R₄)ₐ(R₅)_{b}-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyalkyl, hydroxyalkenyl, hydroxyalkynyl, hydroxyaryl, alkoxy, carboxyl, carboxylate, ester, —Y(R₄)ₐ(R₅)_{b}-ester, —Y(R₄)ₐ(R₅)_{b}, ═O, —NO₂, —CN, sulfoxy, secondary amide, tertiary amide, or CON—R₆ amide. In some embodiments, each occurrence of Y is independently C, N, O, S, or P. In some embodiments, each occurrence of R₄, R₅, and R₆ is independently H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, natural amino acid, unnatural amino acid, or sulfoxy. In some embodiments, a is an integer represented by 0, 1, or 2. In some embodiments, b is an integer represented by 0, 1, or 2.

In some embodiments, m is an integer from 1 to 10. In some embodiments, each occurrence of n, p, q, and r is independently an integer from 0 to 50. In some embodiments, o is an integer from 0 to 10.

In one embodiments, the compound having the structure of Formula (I) is a compound having the structure of Formula (III)

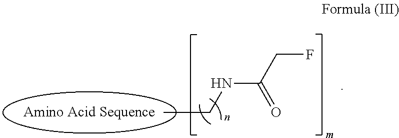

In some embodiments, m is an integer from 1 to 10. For example, in one embodiment, m is an integer represented by 1. In some embodiments, each occurrence of n is independently an integer from 0 to 50. For example, in one embodiment, n is an integer represented by 1.

In one embodiment, the compound having the structure of Formula (II) is a compound having the structure of Formula (IV)

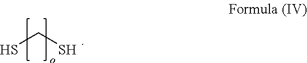

In one embodiment, o is an integer from 1 to 10. For example, in one embodiment, o is an integer represented by 5. In one embodiment, o is an integer represented by 6. In one embodiment, o is an integer represented by 7. In one embodiment, o is an integer represented by 8.

In one embodiment, the compound having the structure of Formula (II) is a compound having the structure of Formula (V)

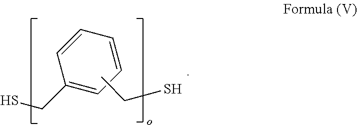

In one embodiment, o is an integer from 1 to 10. For example, in one embodiment, o is an integer represented by 1.

In one aspect, the method comprises reacting the compound or salt thereof having the structure of Formula (I) and the compound or salt thereof having the structure of Formula (II) at ambient temperature.

In one aspect, the method comprises reacting the compound or salt thereof having the structure of Formula (I) and the compound or salt thereof having the structure of Formula (II) in aqueous solutions. In some embodiments, the method comprises reacting the compound or salt thereof having the structure of Formula (I) and the compound or salt thereof having the structure of Formula (II) in the presence of an organic solvent. Examples of organic solvents include, but are not limited to: dimethylformamide (DMF), toluene, xylene, p-xylene, benzene, chloroform, dichloromethane, carbon tetrachloride, diethyl ether, pyridine, triethylamine, methanol, ethanol, propanol, 1-propanol, isopropanol, butanol, 1-butanol, tert-butanol, pentane, hexane, cyclohexane, heptane, n-heptane, acetone, ethyl acetate, acetonitrile, tetrahydrofuran (THF), dioxane, dimethyl sulfoxide (DMSO), and any combination thereof.

In one aspect, the method comprises reacting the compound or salt thereof having the structure of Formula (I) and the compound or salt thereof having the structure of Formula (II) in the presence of a base. Examples of bases include, but are not limited to: an amidine compound, 1,8-Diazabicyclo[5.4.0]undec-7-ene (DBU), carbonate compound, K₂CO₃, Li₂CO₃, Na₂CO₃, Rb₂CO₃, Cs₂CO₃, K₂CO₃, KHCO₃, KOH, NaOH, LiOH, CsOH, RbOH, Ca(OH)₂, Sr(OH)₂, Ba(OH)₂, organic bases, such as ammonia, methylamine, ethylamine, n-propylamine, isopropylamine, cyclohexylamine, dimethylamine, diethylamine, di-n-propylamine, trimethylamine, triethylamine, tri-n-propylamine, N,N-Diisopropylethylamine, aniline, N-methylaniline, N,N-dimethylaniline, p-bromoaniline, p-methoxyaniline, p-nitroaniline, pyrrole, pyrrolidine, imidazole, pyridine, piperidine, phosphazenes, and any combination thereof.

In one embodiment, the method comprises reacting the compound or salt thereof having the structure of Formula (I) and the compound or salt thereof having the structure of Formula (II) in a solution having a pH above 7. In some embodiments, the method comprises reacting the compound or salt thereof having the structure of Formula (I) and the compound or salt thereof having the structure of Formula (II) in a solution having a pH between around 7 and around 14. In some embodiments, the method comprises reacting the compound or salt thereof having the structure of Formula (I) and the compound or salt thereof having the structure of Formula (II) in a solution having a pH between around 7.5 and around 9.0. In one embodiment, the method comprises reacting the compound or salt thereof having the structure of Formula (I) and the compound or salt thereof having the structure of Formula (II) in an aqueous solution having a pH around 8.5. In one embodiment, the method comprises reacting the compound or salt thereof having the structure of Formula (I) and the compound or salt thereof having the structure of Formula (II) in an aqueous solution having a pH of 8.5.

In one aspect, the method comprises reacting the compound or salt thereof having the structure of Formula (I) and the compound or salt thereof having the structure of Formula (II) in the presence of a reducing agent. Examples of reducing agent include, but are not limited to: tris(2-carboxyethyl)phosphine (TCEP), tris(3-hydroxypropyl)phosphine (THPP), sodium cyanoborohydride, lithium aluminum hydride, sodium amalgam (Na(Hg)), sodium borohydride, sulfite reducing agent, dithionate reducing agent, Na₂S₂O₆, thiosulfate reducing agent, Na₂S₂O₃, KI, hydrazine, diisobutylaluminum hydride (DIBAL-H), oxalic acid, formic acid, ascorbic acid, reducing sugars, phosphites, hypophosphites, phosphorous acid, dithiothreitol (DTT), carbon monoxide, and any combination thereof.

In some embodiments, the compound or salt thereof having the structure of Formula (I) and the compound or salt thereof having the structure of Formula (II) are stoichiometric, near stoichiometric, or stoichiometric-like. In some embodiments, the methods comprise a stoichiometric, near stoichiometric, or stoichiometric-like stapling of the compound or salt thereof having the structure of Formula (I) and the compound or salt thereof having the structure of Formula (II). In some embodiments, the provided strategies, reaction mixtures, or synthetic conditions comprise stoichiometric, near stoichiometric, or stoichiometric-like stapling of the compound or salt thereof having the structure of Formula (I) and the compound or salt thereof having the structure of Formula (II).

In one embodiment, the compound or salt thereof having the structure of Formula (I) is reacted with the compound or salt thereof having the structure of Formula (II) in 1:1 molar ratio. In one embodiment, the compound or salt thereof having the structure of Formula (I) is reacted with the compound or salt thereof having the structure of Formula (II) in 1:2 molar ratio. In one embodiment, the compound or salt thereof having the structure of Formula (I) is reacted with the compound or salt thereof having the structure of Formula (II) in 1:3 molar ratio. In one embodiment, the compound or salt thereof having the structure of Formula (I) is reacted with the compound or salt thereof having the structure of Formula (II) in 1:4 molar ratio. In one embodiment, the compound or salt thereof having the structure of Formula (I) is reacted with the compound or salt thereof having the structure of Formula (II) in 1:5 molar ratio.

In one embodiment, the compound or salt thereof having the structure of Formula (I) is reacted with about 10-fold excess of the compound or salt thereof having the structure of Formula (II). In one embodiment, the compound or salt thereof having the structure of Formula (I) is reacted with about 20-fold excess of the compound or salt thereof having the structure of Formula (II). In one embodiment, the compound or salt thereof having the structure of Formula (I) is reacted with about 30-fold excess of the compound or salt thereof having the structure of Formula (II). In one embodiment, the compound or salt thereof having the structure of Formula (I) is reacted with about 40-fold excess of the compound or salt thereof having the structure of Formula (II). In one embodiment, the compound or salt thereof having the structure of Formula (I) is reacted with about 50-fold excess of the compound or salt thereof having the structure of Formula (II). In one embodiment, the compound or salt thereof having the structure of Formula (I) is reacted with about 100-fold excess of the compound or salt thereof having the structure of Formula (II).

In one embodiment, the compound or salt thereof having the structure of Formula (I) is reacted with the compound or salt thereof having the structure of Formula (II) in 2:1 molar ratio. In one embodiment, the compound or salt thereof having the structure of Formula (I) is reacted with the compound or salt thereof having the structure of Formula (II) in 3:1 molar ratio. In one embodiment, the compound or salt thereof having the structure of Formula (I) is reacted with the compound or salt thereof having the structure of Formula (II) in 4:1 molar ratio. In one embodiment, the compound or salt thereof having the structure of Formula (I) is reacted with the compound or salt thereof having the structure of Formula (II) in 5:1 molar ratio. For example, in one embodiment, the compound or salt thereof having the structure of Formula (I) is reacted with the compound or salt thereof having the structure of Formula (II) in 1:1 molar ratio and m is an integer represented by 2.

In one embodiment, the compound or salt thereof having the structure of Formula (II) is reacted with about 10-fold excess of the compound or salt thereof having the structure of Formula (I). In one embodiment, the compound or salt thereof having the structure of Formula (II) is reacted with about 20-fold excess of the compound or salt thereof having the structure of Formula (I). In one embodiment, the compound or salt thereof having the structure of Formula (II) is reacted with about 30-fold excess of the compound or salt thereof having the structure of Formula (I). In one embodiment, the compound or salt thereof having the structure of Formula (II) is reacted with about 40-fold excess of the compound or salt thereof having the structure of Formula (I). In one embodiment, the compound or salt thereof having the structure of Formula (II) is reacted with about 50-fold excess of the compound or salt thereof having the structure of Formula (I). In one embodiment, the compound or salt thereof having the structure of Formula (II) is reacted with about 100-fold excess of the compound or salt thereof having the structure of Formula (I).

In various embodiments, the amino acid sequence comprises two or more amino acids. In some embodiments, the amino acid sequence is a protein or a fragment thereof, peptide or a fragment thereof, polypeptide or a fragment thereof, antigen or a fragment thereof, or any combination thereof. In some embodiments, the peptide or a fragment thereof is an axin peptide or a fragment thereof, HIV peptide or a fragment thereof, or any combination thereof. Examples of amino acid sequences include, but are not limited to: an isolated protein or fragment thereof, isolated peptide or fragment thereof, tyrosinase-related protein or fragment thereof, tyrosinase-related protein 1 (TRP1) or fragment thereof, tyrosinase-related protein 2 (TRP2) or fragment thereof, peptide or fragment thereof derived from one or more proteins related to neuron regeneration or degeneration, peptide or fragment thereof derived from one or more proteins related to immune signaling, such as phosphatase and tension homolog (PTEN), GSK-3β, Akt, protein tyrosine phosphatase (PTP), leukocyte common antigen-related (LAR), chondroitin sulfate proteoglycans (CSPGs), heparan sulfate proteoglycans (HSPGs), netrin-G ligand-3 (NGL-3), neurotrophin receptor TrkC, phosphoinositide 3-kinase (PI3Ks), interleukin (IL), interferon (IFN), tumor necrosis factor alpha (TNF alpha), beta-amyloid, alpha-synuclein and other synucleins, melanocyte lineage/differentiation antigens, tyrosinase, glycoprotein 75 (gp 75), human homologue of the mouse brown locus, glycoprotein 100 (gp100), Pmel17, target for monoclonal antibody HMB45, human homologue of the mouse silver locus, Melan A/MART-1, oncofetal/cancer-testis antigens, melanoma antigen gene (MAGE) family proteins, B melanoma antigen (BAGE) peptides family, GAGE family antigens, esophageal squamous cell carcinoma-1 (NY-ESO-1), cancer-testis antigen 1B (CTAG1B), tumor-specific antigens, peptides with subtle mutations of normal cellular proteins (e.g., coding region mutations), cyclin-dependent kinase 4 or cell division protein kinase 4 (CDK4), 0-catenin, mutated peptides activated as a result of cellular transformation, mutated introns, N-acetylglucosaminyltransferase V gene product, MUM-1, p15, antigens identified by monoclonal antibodies, gangliosides (e.g., GM2, GD2, GM3, and GD3), high molecular weight chondroitin sulfate proteoglycan, p97 melanotransferrin, SEREX antigens, D-1, synovial sarcoma/X breakpoint 2 (SSX-2), ovarian cancer antigens, surviving or baculoviral inhibitor of apoptosis repeat-containing 5 (BIRC5), cancer antigen 125 (CA125), carcinoembryonic antigen (CEA), DEAD-box helicase 43 (DDX43), epithelial cell adhesion molecule (EPCAM), folate Receptor Alpha (FOLR1), human epidermal growth factor receptor 2 (Her-2)/neu, melanoma-associated antigen 1 (MAGE-A1), melanoma-associated antigen 2 (MAGE-A2), melanoma-associated antigen 3 (MAGE-A3), melanoma-associated antigen 4 (MAGE-A4), melanoma-associated antigen 6 (MAGE-A6), melanoma-associated antigen 10 (MAGE-A10), melanoma-associated antigen 12 (MAGE-A12), mucin 1 (MUC-1), preferentially expressed antigen in melanoma (PRAME), tumor protein p53 (p53), trophoblast glycoprotein (TPBG), TRT, Wilms tumor protein (WT1), cancer/testis antigen 45 (CT45), breast cancer antigens, telomerase reverse transcriptase (hTERT), Sialyn-Tn, Wilms' Tumor Gene, antigens associated with cancers (e.g., acute lymphoblastic leukemia (ALL), acute myeloid leukemia (AML), cancer in adolescents, adrenocortical carcinoma, acquired immunodeficiency syndrome (AIDS)-related cancers, kaposi sarcoma, lymphoma, AIDS-related lymphoma, primary central nervous system (CNS) lymphoma, anal cancer, appendix cancer, gastrointestinal carcinoid tumors, astrocytomas, childhood astrocytomas, brain cancer, atypical teratoid/rhabdoid tumor, childhood atypical teratoid/rhabdoid tumor, CNS atypical teratoid/rhabdoid tumor, basal cell carcinoma of the skin, skin cancer, bile duct cancer, bladder cancer, childhood bladder cancer, bone cancer (includes Ewing sarcoma and osteosarcoma and malignant fibrous histiocytoma), brain tumors, breast cancer, bronchial tumors, Burkitt lymphoma, non-Hodgkin lymphoma, carcinoid tumor (gastrointestinal), childhood carcinoid tumors, carcinoma of unknown primary, childhood carcinoma of unknown primary, cardiac (heart) tumors, childhood cardiac (heart) tumors, medulloblastoma and other CNS embryonal tumors, childhood brain cancer, germ cell tumor, primary CNS lymphoma, cervical cancer, childhood cervical cancer, childhood cancers, unusual cancers of childhood, cholangiocarcinoma, chordoma, childhood chordoma, chronic lymphocytic leukemia (CLL), chronic myelogenous leukemia (CML), chronic myeloproliferative neoplasms, colorectal cancer, craniopharyngioma, childhood craniopharyngioma, mycosis fungoides and Sezary syndrome, ductal carcinoma in situ (DCIS), embryonal tumors, medulloblastoma and other childhood CNS brain cancers, endometrial cancer, ependymoma, childhood ependymoma, esophageal cancer, esthesioneuroblastoma, Ewing sarcoma, extracranial germ cell tumor, childhood extracranial germ cell tumor, extragonadal germ cell tumor, eye cancer, retinoblastoma, fallopian tube cancer, fibrous histiocytoma of bone, malignant fibrous histiocytoma of bone, osteosarcoma fibrous histiocytoma of bone, gallbladder cancer, gastric (stomach) cancer, gastrointestinal carcinoid tumor, gastrointestinal stromal tumors (GIST), soft tissue sarcoma, germ cell tumors, childhood CNS germ cell tumors, ovarian germ cell tumors, gestational trophoblastic disease, hairy cell leukemia, head and neck cancer, heart tumors, childhood heart tumors, hepatocellular (liver) cancer, histiocytosis, langerhans cell histiocytosis, Hodgkin lymphoma, hypopharyngeal cancer, Islet cell tumors, pancreatic neuroendocrine tumors, kidney (renal cell) cancer, Langerhans cell histiocytosis, Laryngeal cancer, leukemia, lip and oral cavity cancer, liver cancer, lung cancer, such as non-small cell, small cell, pleuropulmonary blastoma, and tracheobronchial tumor, male breast cancer, melanoma, childhood melanoma, intraocular (eye) melanoma, childhood intraocular melanoma, Merkel cell carcinoma, mesothelioma, malignant mesothelioma, metastatic cancer, metastatic squamous neck cancer with occult primary, midline tract carcinoma with NUT gene changes, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, chronic myeloproliferative neoplasms, nasal cavity and paranasal sinus cancer, nasopharyngeal cancer, neuroblastoma, non-small cell lung cancer, oral cancer, lip and oral cavity cancer and oropharyngeal cancer, oropharyngeal cancer, ovarian cancer, childhood ovarian cancer, pancreatic cancer, pancreatic neuroendocrine tumors, papillomatosis, childhood laryngeal, paraganglioma, childhood paraganglioma, paranasal sinus and nasal cavity cancer, parathyroid cancer, penile cancer, pharyngeal cancer, pheochromocytoma, childhood pheochromocytoma, pituitary tumor, plasma cell neoplasm/multiple myeloma, pleuropulmonary blastoma, pregnancy and breast cancer, primary CNS lymphoma, primary peritoneal cancer, prostate cancer, rectal cancer, recurrent cancer, rhabdomyosarcoma, childhood rhabdomyosarcoma, childhood soft tissue sarcoma, salivary gland cancer, sarcoma, childhood vascular tumors, osteosarcoma, uterine sarcoma, Sezary syndrome, childhood skin cancer, small cell lung cancer, small intestine cancer, squamous cell carcinoma of the skin, squamous neck cancer with occult primary, metastatic squamous neck cancer with occult primary, stomach (gastric) cancer, T-cell lymphoma, cutaneous T-cell lymphoma, testicular cancer, childhood testicular cancer, throat cancer, nasopharyngeal cancer, thymoma and thymic carcinoma, thyroid cancer, tracheobronchial tumors, transitional cell cancer of the renal pelvis and ureter, urethral cancer, uterine cancer, endometrial uterine cancer, vaginal cancer, childhood vaginal cancer, vascular tumors, vulvar cancer, Wilms tumor and other childhood kidney tumors, and cancers in young adults), or any combination thereof.

The peptide of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The invention should also be construed to include any form of a peptide having substantial homology to the peptides disclosed herein. Preferably, a peptide which is “substantially homologous” is about 60% homologous, about 70% homologous, about 80% homologous, about 90% homologous, about 91% homologous, about 92% homologous, about 93% homologous, about 94% homologous, about 95% homologous, about 96% homologous, about 97% homologous, about 98% homologous, or about 99% homologous to amino acid sequence of the peptides disclosed herein.

The peptide may alternatively be made by recombinant means or by cleavage from a longer polypeptide. The composition of a peptide may be confirmed by amino acid analysis or sequencing.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The polypeptides of the invention can be post-translationally modified (i.e., modified after synthesis). For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

A peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of the wild-type BEST1 comprising peptide.

A peptide or protein of the invention may be phosphorylated using conventional methods such as the method described in Reedijk et al. (The EMBO Journal 11(4):1365, 1992).

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

(a) Tags

In a particular embodiment of the invention, the polypeptide of the invention further comprises the amino acid sequence of a tag. The tag includes but is not limited to: polyhistidine tags (His-tags) (for example H6 and H10, etc.) or other tags for use in IMAC systems, for example, Ni²⁺ affinity columns, etc., GST fusions, MBP fusions, streptavidine-tags, the BSP biotinylation target sequence of the bacterial enzyme BIRA and tag epitopes that are directed by antibodies (for example c-myc tags, FLAG-tags, among others). As will be observed by a person skilled in the art, the tag peptide can be used for purification, inspection, selection and/or visualization of the fusion protein of the invention. In a particular embodiment of the invention, the tag is a detection tag and/or a purification tag. It will be appreciated that the tag sequence will not interfere in the function of the protein of the invention.

(b) Leader and Secretory Sequences

Accordingly, the polypeptides of the invention can be fused to another polypeptide or tag, such as a leader or secretory sequence or a sequence which is employed for purification or for detection. In a particular embodiment, the polypeptide of the invention comprises the glutathione-S-transferase protein tag which provides the basis for rapid high-affinity purification of the polypeptide of the invention. Indeed, this GST-fusion protein can then be purified from cells via its high affinity for glutathione. Agarose beads can be coupled to glutathione, and such glutathione-agarose beads bind GST-proteins. Thus, in a particular embodiment of the invention, the polypeptide of the invention is bound to a solid support. In a preferred embodiment, if the polypeptide of the invention comprises a GST moiety, the polypeptide is coupled to a glutathione-modified support. In a particular case, the glutathione modified support is a glutathione-agarose bead. Additionally, a sequence encoding a protease cleavage site can be included between the affinity tag and the polypeptide sequence, thus permitting the removal of the binding tag after incubation with this specific enzyme and thus facilitating the purification of the corresponding protein of interest.

(c) Targeting Sequences

The invention also relates to peptides comprising peptide fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e., are heterologous).

A target protein is a protein that is selected for degradation and for example may be a protein that is mutated or over expressed in a disease or condition. In another embodiment of the invention, a target protein is a protein that is abnormally degraded and for example may be a protein that is mutated or underexpressed in a disease or condition. The targeting domain can be a membrane spanning domain, a membrane binding domain, or a sequence directing the protein to associate with for example vesicles or with the nucleus. The targeting domain can target a peptide to a particular cell type or tissue. For example, the targeting domain can be a cell surface ligand or an antibody against cell surface antigens of a target tissue (e.g. retina tissue). A targeting domain may target the peptide of the invention to a cellular component.

(d) Intracellular Targeting

Combined with certain formulations, such peptides can be effective intracellular agents. However, in order to increase the efficacy of such peptides, the peptide of the invention can be provided a fusion peptide along with a second peptide which promotes “transcytosis”, e.g., uptake of the peptide by epithelial cells. To illustrate, the peptide of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the RLP can be provided a fusion polypeptide with all or a portion of the antenopedia III protein.

To further illustrate, the peptide of the invention can be provided as a chimeric peptide which includes a heterologous peptide sequence (“internalizing peptide”) which drives the translocation of an extracellular form of the peptide across a cell membrane in order to facilitate intracellular localization of the peptide. In this regard, the peptide is one which is active intracellularly. The internalizing peptide, by itself, is capable of crossing a cellular membrane by, e.g., transcytosis, at a relatively high rate. The internalizing peptide is conjugated, e.g., as a fusion protein, to a peptide comprising wild-type BEST1. The resulting chimeric peptide is transported into cells at a higher rate relative to the peptide alone to thereby provide a means for enhancing its introduction into cells to which it is applied.

(e) Peptide Mimetics

In other embodiments, the subject compositions are peptidomimetics of the peptide of the invention. Peptidomimetics are compounds based on, or derived from, peptides and proteins. The peptidomimetics of the present invention typically can be obtained by structural modification of a known sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

Moreover, as is apparent from the present disclosure, mimotopes of the subject peptides can be provided. Such peptidomimetics can have such attributes as being non-hydrolysable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), 3-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modified (Roark et al. in Peptides: Chemistry and Biology, G. R. Marshall ed., ESCOM Publisher: Leiden, Netherlands, 1988, p134). Also, see generally, Session III: Analytic and synthetic methods, in in Peptides: Chemistry and Biology, G. R. Marshall ed., ESCOM Publisher: Leiden, Netherlands, 1988)

In addition to a variety of side chain replacements which can be carried out to generate the peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

Moreover, other examples of mimotopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimotope can be obtained by, for example, screening libraries of natural and synthetic compounds for compounds capable of binding to the peptide of the invention. A mimotope can also be obtained, for example, from libraries of natural and synthetic compounds, in particular, chemical or combinatorial libraries (i.e., libraries of compounds that differ in sequence or size but that have the same building blocks). A mimotope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimotopes by, for example, computer modelling, the predicted mimotope structures can then be produced by, for example, chemical synthesis, recombinant DNA technology, or by isolating a mimotope from a natural source (e.g., plants, animals, bacteria and fungi).

A peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.) By way of example, a RLP or chimeric protein may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (See Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Methods of making and using antibodies are well known in the art. For example, polyclonal antibodies useful in the present invention are generated by immunizing rabbits according to standard immunological techniques well-known in the art. Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, which bind to the specific antigens of interest.

One skilled in the art would appreciate, based upon the disclosure provided herein, that the antibody can specifically bind with any portion of an antigen target, which can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

The antibodies can be produced by immunizing an animal such as, but not limited to, a rabbit, a mouse or a camel, with an antigenic protein of the invention, or a portion thereof, by immunizing an animal using a protein comprising at least a portion of the antigen, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind the antigen of interest.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an antigen of interest can be used to produce antibodies that are specific only for that antigen and do not cross-react non-specifically with other proteins.

The invention encompasses monoclonal, synthetic antibodies, and the like. One skilled in the art would understand, based upon the disclosure provided herein, that the crucial feature of the antibody of the invention is that the antibody bind specifically with an antigen of interest. That is, the antibody of the invention recognizes an antigen of interest or a fragment thereof (e.g., an immunogenic portion or antigenic determinant thereof).

The skilled artisan would appreciate, based upon the disclosure provided herein, that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

In some embodiments, a non-human antibody is humanized, where specific sequences or regions of the antibody are modified to increase similarity to an antibody naturally produced in a human or fragment thereof. A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323.)

In one embodiment, the antibody fragment provided herein is a single chain variable fragment (scFv). In various embodiments, the antibodies of the invention may exist in a variety of other forms including, for example, Fv, Fab, and (Fab′) 2, as well as bi-functional (i.e. bi-specific) hybrid antibodies (e.g., Lanzavecchia et al., Eur. J. Immunol. 17, 105 (1987)). In some embodiments, the antibodies and fragments thereof of the invention bind a cell bearing antigen, TCR, and/or BCR with wild-type or enhanced affinity. In some embodiments, the antibodies and fragments thereof of the invention bind a T cell bearing TCR with wild-type or enhanced affinity. In some embodiments, the antibodies and fragments thereof of the invention bind a B cell bearing BCR with wild-type or enhanced affinity. In various embodiments, a human scFv may also be derived from a yeast display library.

ScFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise flexible polypeptide linker (e.g., a Ser-Gly linker) with an optimized length and/or amino acid composition. The flexible polypeptide linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715.

The scFv can comprise a polypeptide linker sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The flexible polypeptide linker sequence may comprise any naturally occurring amino acid. In some embodiments, the flexible polypeptide linker sequence comprises amino acids glycine and serine. In another embodiment, the flexible polypeptide linker sequence comprises sets of glycine and serine repeats such as (Gly4Ser)n, where n is a positive integer equal to or greater than 1. In one embodiment, the flexible polypeptide linkers include, but are not limited to, (Gly4Ser)4 or (Gly4Ser)3. Variation in the flexible polypeptide linker length may retain or enhance activity, giving rise to superior efficacy in activity studies.

Methods of Linking Amino Acid Sequences with Various Compounds of Interest

In one aspect, the present invention also provides a method of linking one or more amino acid sequences and one or more compounds of interest. In one aspect, the present invention also provides a method of linking one or more amino acid sequences and one or more compounds A. In one aspect, the present invention relates to a method of bioorthogonal linking of one or more amino acid sequences and one or more compounds A. In one embodiment, the method generates steric-free labeling. In one embodiment, the method generates steric-free labeling of a substrate of interest. For example, in one embodiment, the method generates steric-free labeling of protein substrates. Thus, in one aspect of the present invention, the method profiles targets of interest. In one embodiment, the method globally profiles molecular targets.

In various embodiments, the method comprises reacting a compound or salt thereof having the structure of Formula (I) with a compound or salt thereof having the structure of Formula (VI)

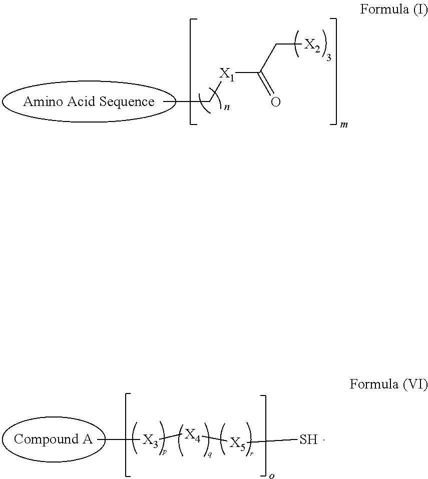

In one embodiment, the compound having the structure of Formula (VI) is a compound having the structure of Formula (VII)

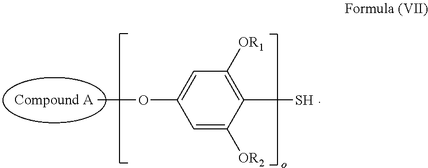

In some embodiments, each occurrence of R₁ and R₂ is independently hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, or substituted heteroaryl. In some embodiments, o is an integer from 0 to 10. For example, in one embodiment, o is an integer represented by 0. In another embodiment, o is an integer represented by 1.

In some embodiments, the compound A is an antibody or a fragment thereof, antigen or a fragment thereof, protein or a fragment thereof, peptide or a fragment thereof, amino acid sequence or a fragment thereof, amino acid or a derivative thereof, small molecule or a derivative thereof, adjuvant, therapeutic agent or a derivative thereof, or any combination thereof.

In one embodiment, the compound A is a biotin. Thus, in some embodiment, the compound having the structure of Formula (VII) is a compound having the structure of Formula (VIII)

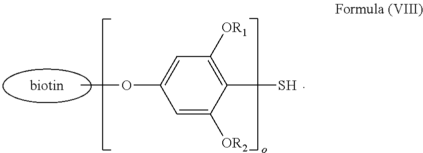

For example, in one embodiment, the compound having the structure of Formula (VIII) is a compound having the structure of Formula (IX)

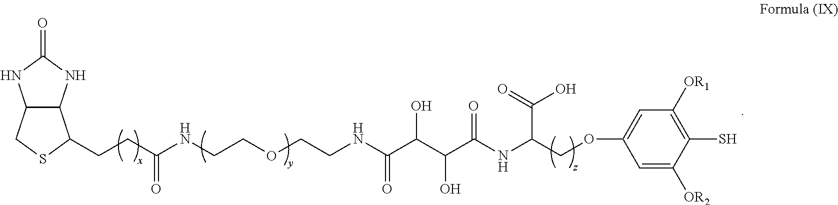

In some embodiments, each occurrence of x, y, and z is independently an integer from 1 to 10.

In one embodiment, the compound A comprises an adjuvant. In one embodiment, the compound A is an adjuvant. Examples of adjuvants include, but are not limited to: muramyl dipeptide derivatives (MDP) or analog thereof, Alum and Emulsions, complete Freund's adjuvant (CFA), incomplete Freund's adjuvant (IFA), pattern recognition receptor (PRR) ligands, cyclic guanosine monophosphate-adenosine monophosphate (2′3′-cGAMP), bis-(3′-5′)-cyclic dimeric adenosine monophosphate (c-di-AMP), Rp,Rp-isomer of the 2′3′-bisphosphorothioate analog of 3′3′-cyclic adenosine monophosphate (2′3′-c-di-AM(PS)2 (Rp,Rp)), cyclic diguanylate monophosphate-stimulator of interferon genes (c-di-GMP STING)-based vaccine adjuvant, CL401, CL413, CL429, Flagellin, Imiquimod, lipopolysaccharide (LPS) from the gram-negative bacteria E. coli 0111:B4 (LPS-EB), monophosphoryl lipid A from Salmonella minnesota R₅₉₅ lipopolysaccharide (MPLA-SM), synthetic monophosphoryl lipid A (MPLA), oligodeoxynucleotides (ODN) 1585, ODN 1826, ODN 2006, ODN 2395, Pam3CSK4, Resiquimod (R848), trehalose-6,6-dibehenate (TDB), or any combination thereof.

In some embodiments, the compound A is at least one therapeutic agent. Examples of such therapeutic agents include, but are not limited to, one or more drugs, metabolites, metabolic inhibitors, proteins, amino acids, peptides, antibodies, medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, anti-tumor agents, including cytotoxic/antineoplastic agents and anti-angiogenic agents, or any combination thereof.

Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2 (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In one embodiment, the compound A comprises one or more non-therapeutic moieties. In one embodiment, the compound A is one or more non-therapeutic moieties. In some embodiments, the compound A comprises folic acid, peptides, proteins, aptamers, antibodies, small RNA molecules, miRNA, shRNA, siRNA, poorly water-soluble therapeutic agents, anti-cancer agents, or any combinations thereof. In some embodiments, the compound A is folic acid, peptides, proteins, aptamers, antibodies, small RNA molecules, miRNA, shRNA, siRNA, poorly water-soluble therapeutic agents, anti-cancer agents, or any combinations thereof.

In one embodiment, the compound A comprises a targeting domain. In one embodiment, the compound A is a targeting domain. In one embodiment, the targeting domain binds to at least one associated with a disease or a disorder. In various embodiments, the targeting domain is an antibody, an antibody fragment, a peptide sequence, aptamer, folate, a ligand, a gene component, or any combination thereof. Examples of targeting domains include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting domains of particular interest include peptidomimetics, peptides, antibodies (e.g., monoclonal antibodies, polyclonal antibodies, recombinant antibodies, human antibodies, humanized antibodies, etc.) and antibody fragments (e.g., the Fab′ fragment).

### Linked and Stapled Amino Acid Sequences

The present invention also relates to methods, compositions, techniques, and strategies for making, purifying, characterizing, and using the compounds prepared by the methods of the present invention. Thus, in one aspect, the present invention provides compounds prepared by the methods of the present invention. In one aspect, the present invention provides a compound prepared by the method of stapling one or more amino acid sequences of the present invention.

In one embodiment, the compound prepared by the methods of the present invention is a compound or salt thereof having the structure of Formula (X)

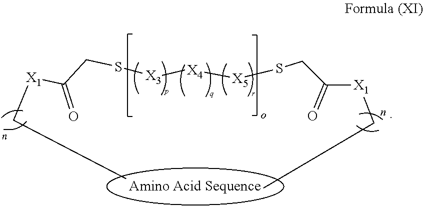

In one embodiment, the compound having the structure of Formula (X) is a compound having the structure of Formula (XI)

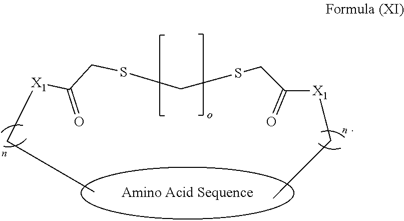

In one embodiment, o is an integer from 1 to 10. For example, in one embodiment, o is an integer represented by 5. In one embodiment, o is an integer represented by 6. In one embodiment, o is an integer represented by 7. In one embodiment, o is an integer represented by 8.

In one embodiment, the compound having the structure of Formula (X) is a compound having the structure of Formula (XII)

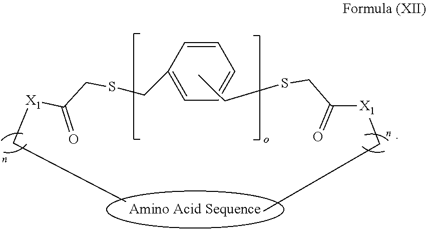

In some embodiments, o is an integer from 1 to 10. For example, in one embodiment, o is an integer represented by 1.

In one embodiment, the compound prepared by the methods of the present invention is a compound or salt thereof having the structure of Formula (XIII)

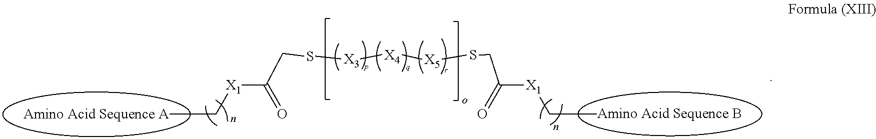

In one embodiment, the compound having the structure of Formula (XIII) is a compound having the structure of Formula (XIV)

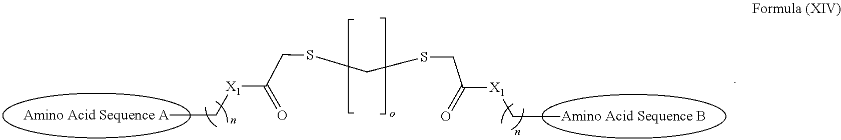

In one embodiment, o is an integer from 1 to 10. For example, in one embodiment, o is an integer represented by 5. In one embodiment, o is an integer represented by 6. In one embodiment, o is an integer represented by 7. In one embodiment, o is an integer represented by 8.

In one embodiment, the compound having the structure of Formula (XIII) is a compound having the structure of Formula (XV)

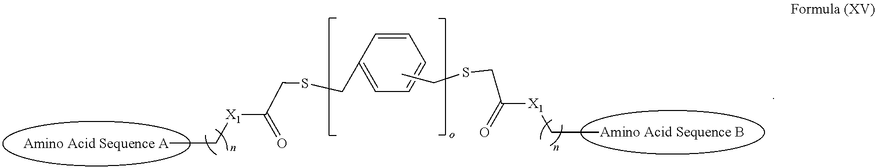

In some embodiments, o is an integer from 1 to 10. For example, in one embodiment, o is an integer represented by 1.

In various embodiments, each occurrence of the amino acid sequence A and amino acid sequence B is independently any amino acid sequence disclosed herein. In one embodiment, the amino acid sequence A is identical to the amino acid sequence B. In one embodiment, the amino acid sequence A is not identical to the amino acid sequence B.

In one aspect, the present invention provides a compound prepared by the method of linking one or more amino acid sequences and one or more compounds of interest of the present invention. Thus, in one aspect, the present invention provides a compound prepared by the method of linking one or more amino acid sequences and one or more compound A.

In one embodiment, the compound prepared by the methods of the present invention is a compound or salt thereof having the structure of Formula (XVI)

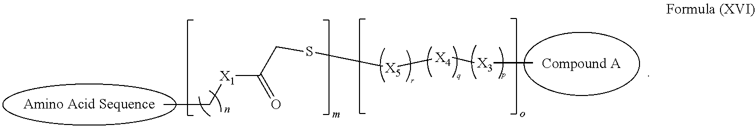

In one embodiment, the compound prepared by the methods of the present invention is a compound or salt thereof having the structure of Formula (XVII)

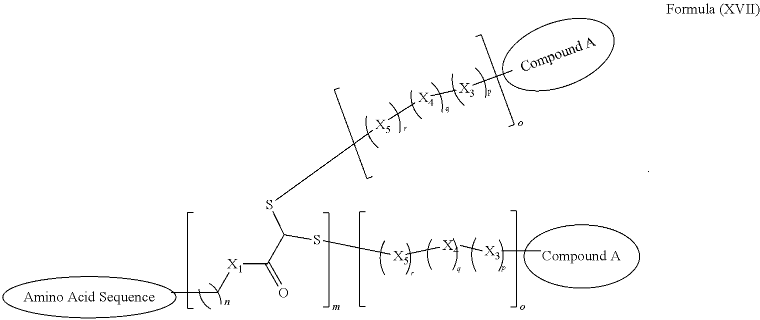

In one embodiment, the compound prepared by the methods of the present invention is a compound or salt thereof having the structure of Formula (XVIII)

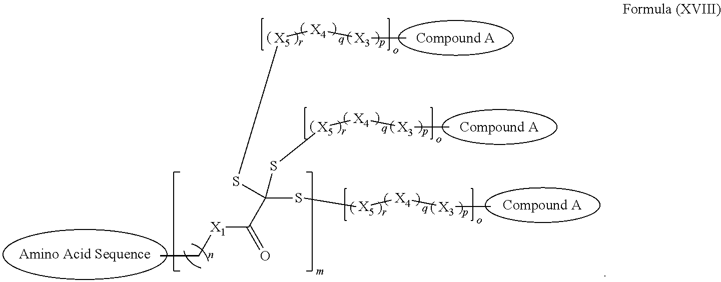

In one embodiment, the compound having the structure of Formula (XVI) is a compound having the structure of Formula (XIX)

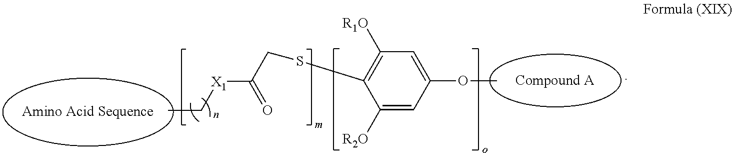

In various embodiments, the amino acid sequence is any amino acid sequence disclosed herein. In various embodiments, the compound A is any compound A disclosed herein. For example, in one embodiment, the compound A is a biotin. Thus, in one embodiment, the compound having the structure of Formula (XIX) is a compound having the structure of Formula (XX)

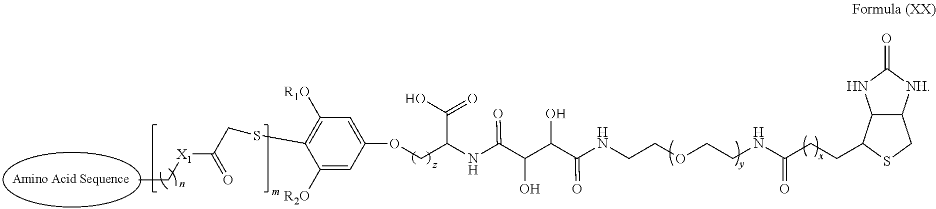

In some embodiments, each occurrence of X₁ is independently O, S, NR₁, CR₁R₂, or C(═R₃). In some embodiments, each occurrence of R₁ and R₂ is independently hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, or substituted heteroaryl. In some embodiments, each occurrence of R₃ is independently O, NR₁, or S.

In some embodiments, each occurrence of X₃, X₄, and X₅ is independently O, S, NR₁, CR₁R₂, C(═R₃), cycloalkyl, substituted cycloalkyl, heterocycloalkyl, substituted heterocycloalkyl, aryl, substituted aryl, heteroaryl, substituted heteroaryl, alkyl, substituted alkyl, —Y(R₄)ₐ(R₅)_{b}-cycloalkyl, substituted —Y(R₄)ₐ(R₅)_{b}-cycloalkyl, —Y(R₄)ₐ(R₅)_{b}-heterocycloalkyl, substituted —Y(R₄)ₐ(R₅)_{b}-heterocycloalkyl, alkenyl, substituted alkenyl, cycloalkenyl, substituted cycloalkenyl, —Y(R₄)ₐ(R₅)_{b}-cycloalkenyl, substituted —Y(R₄)ₐ(R₅)_{b}-cycloalkenyl, alkynyl, substituted alkynyl, cycloalkynyl, substituted cycloalkynyl, —Y(R₄)ₐ(R₅)_{b}-cycloalkynyl, substituted —Y(R₄)ₐ(R₅)_{b}-cycloalkynyl, —Y(R₄)ₐ(R₅)_{b}-aryl, substituted —Y(R₄)ₐ(R₅)_{b}-aryl, heteroaryl, substituted heteroaryl, —Y(R₄)ₐ(R₅)_{b}-heteroaryl, substituted —Y(R₄)ₐ(R₅)_{b}-heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyalkyl, hydroxyalkenyl, hydroxyalkynyl, hydroxyaryl, alkoxy, carboxyl, carboxylate, ester, —Y(R₄)ₐ(R₅)_{b}-ester, —Y(R₄)ₐ(R₅)_{b}, ═O, —NO₂, —CN, sulfoxy, secondary amide, tertiary amide, or CON—R₆ amide. In some embodiments, each occurrence of Y is independently C, N, O, S, or P. In some embodiments, each occurrence of R₄, R₅, and R₆ is independently H, halogen, alkyl, substituted alkyl, cycloalkyl, substituted cycloalkyl, heterocycloalkyl, substituted heterocycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, heteroaryl, substituted heteroaryl, alkoxycarbonyl, linear alkoxycarbonyl, branched alkoxycarbonyl, amido, amino, aminoalkyl, aminoalkenyl, aminoalkynyl, aminoaryl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, natural amino acid, unnatural amino acid, or sulfoxy. In some embodiments, a is an integer represented by 0, 1, or 2. In some embodiments, b is an integer represented by 0, 1, or 2.

In some embodiments, m is an integer from 1 to 10. In some embodiments, each occurrence of n, p q, and r is independently an integer from 0 to 50. In some embodiments, o is an integer from 1 to 10.

In some embodiments, each occurrence of n, x, y, and z is independently an integer from 0 to 50.

In various aspects, the compounds of the present have improved cell-penetrating ability. In other aspects, the compounds of the present have improved chemical stability.

### Compositions

The present invention also provides various compositions comprising the stapled amino acid sequences and/or linked amino acid sequence compounds of the present invention. In one embodiment, the composition is a biodegradable composition. In one embodiment, the composition is a medical biodegradable composition.

In various aspects, the composition comprises: one or more compounds of the present invention and one or more stabilizers. In various embodiments, the stabilizer to compound weight ratio is less than 50%. In one embodiment, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines, carboxylic acids, or combinations thereof.

The compositions are formulated in a pharmaceutically acceptable excipient, such as wetting agents, buffers, disintegrants, binders, fillers, flavoring agents and liquid carrier media such as sterile water, water/ethanol etc. The compositions should be suitable for administration either by topical administration or injection or inhalation or catheterization or instillation or transdermal introduction into any of the various body cavities including the alimentary canal, the vagina, the rectum, the bladder, the ureter, the urethra, the mouth, etc. For oral administration, the pH of the composition is preferably in the acid range (e.g., 2 to 7) and buffers or pH adjusting agents may be used. The contrast media may be formulated in conventional pharmaceutical administration forms, such as tablets, capsules, powders, solutions, dispersion, syrups, suppositories etc.

The compounds or compositions of the invention can be formulated and administered to a subject, as now described. The invention encompasses the preparation and use of pharmaceutical compositions comprising the compound and/or compositions of the invention useful for the delivery of a therapeutic agent to a cell. The invention also encompasses the preparation and use of pharmaceutical compositions comprising the compound and/or compositions of the invention useful for the treatment of a disease or disorder. The invention also encompasses the preparation and use of pharmaceutical compositions comprising the compound and/or compositions of the invention useful for improved cell penetration.

Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The pharmaceutical compositions useful for practicing the invention may be administered to deliver a dose of between about 0.01 ng/kg/day and 500 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate modulator thereof, according to the methods of the invention.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals, patients, and subjects of all sorts. Modification of pharmaceutical compositions suitable for administration to humans in order to render the compositions suitable for administration to various animals and patients is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 nanometers to about 2000 micrometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa.

Typically dosages of the compound of the invention which may be administered to an animal or patient, preferably a human, range in amount from about 0.01 mg to about 100 g per kilogram of body weight of the animal or patient. While the precise dosage administered will vary depending upon any number of factors, including, but not limited to, the type of animal and type of disease state being treated, the age of the animal or patient and the route of administration. Preferably, the dosage of the compound will vary from about 0.01 mg to about 500 mg per kilogram of body weight of the animal or patient. The compound can be administered to an animal or patient as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, patient, etc.

Administration of the compounds of the present invention or the compositions thereof may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the muscle. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by subretinal injection. In other embodiments, the composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. In another embodiments, route of administration is subretinal injection or intravitreal injection.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Methods of Cell Penetration and Treatment of Diseases or Disorders

In various aspects, the compounds and/or compositions of the present invention can be used for improved cell penetration. Thus, in one embodiment, the present invention provides a method to deliver a substrate of interest into a cell. For example, in one embodiment, the present invention provides a method to deliver an amino acid sequence of interest into a cell. In one embodiment, the present invention provides a method to deliver a peptide of interest into a cell. In one embodiment, the present invention provides a method to deliver an antigen of interest into a cell. In one embodiment, the present invention provides a method to deliver a therapeutic agent of interest into a cell.

Thus, in one aspect, the present invention provides a method of treating or preventing a disease or disorder in a subject in need thereof. In one embodiment, the method of treating or preventing a disease or disorder in a subject in need thereof comprises a delivery of a substrate of interest into a cell using the compounds of the present invention. In one embodiment, the method of treating or preventing a disease or disorder in a subject in need thereof comprises a delivery of an amino acid sequence of interest into a cell using the compounds of the present invention. In one embodiment, the method of treating or preventing a disease or disorder in a subject in need thereof comprises a delivery of a peptide of interest into a cell using the compounds of the present invention. In one embodiment, the method of treating or preventing a disease or disorder in a subject in need thereof comprises a delivery of an antigen of interest into a cell using the compounds of the present invention. In one embodiment, the method of treating or preventing a disease or disorder in a subject in need thereof comprises a delivery of a therapeutic agent of interest into a cell using the compounds of the present invention.

In one aspect, the present invention provides a method to modulate the function of a cell. Thus, in various embodiments, the present invention provides a method of treating or preventing a disease or disorder associated with cell function in a subject in need thereof. In one embodiment, the method comprises a delivery of a substrate of interest into a cell using the compounds of the present invention. In one embodiment, the method comprises a delivery of an amino acid sequence of interest into a cell using the compounds of the present invention. In one embodiment, the method comprises a delivery of a peptide of interest into a cell using the compounds of the present invention. In one embodiment, the method comprises a delivery of an antigen of interest into a cell using the compounds of the present invention. In one embodiment, the method comprises a delivery of a therapeutic agent of interest into a cell using the compounds of the present invention.

To obtain additional selectivity, the compounds or compositions of the present invention may be passively or actively targeted to regions of interest, such as cells, organs, vessels, sites of disease, wounds, or a specific organism in a subject. In active targeting, the compounds of the present invention may be attached to biological recognition agents to allow them to accumulate in or to be selectively retained by or to be slowly eliminated from certain parts of the body, such as specific cells, specific organs, organs, parts of organs, bodily structures and disease structures, and lesions. Active targeting is defined as a modification of biodistribution using chemical groups that will associate with species present in the desired tissue or organism to effectively decrease the rate of loss of compounds from the specific tissue or organism.

Active targeting of the compounds of the present invention can be considered as localization through modification of biodistribution of the compounds by means of a targeting domain that is attached to or incorporated into the compounds. The targeting domain can associate or bind with one or more receptor species present in the cell, tissue, or organism of interest. This binding will effectively decrease the rate of loss of compounds from the specific cell, tissue, or organism of interest. In such cases, the compounds can be modified synthetically to incorporate the targeting domain. Targeted compounds can localize because of binding between the ligand and the targeted receptor. Alternatively, the compounds can distribute by passive biodistribution, i.e., by passive targeting, into diseased tissues of interest such as wounds. Thus, even without synthetic manipulation to incorporate a targeting domain that can bind to a receptor site, passively targeted contrast agents can accumulate in a diseased tissue or in specific locations in the subject, such as the skin. The present invention comprises use of a compound that is linked to a targeting domain that has an affinity for binding to a receptor. Preferably the receptor is located on the surface of a diseased cell or wounded tissue in a human or animal subject.

In certain embodiments, the targeting domain may recognize a particular ligand or receptor present in a desired cell and/or tissue type when introduced into a subject. In certain embodiments, the targeting domain may be an antibody that recognizes such a particular ligand or receptor. The use of antibody fragments may also be suitable in the methods of the present disclosure. The choice of a targeting domain may depend upon, among other things, the cell and/or tissue type into which an at least partial increase in uptake of the compositions of the present disclosure is desired, as well as particular ligand(s) present in such cell and/or tissue types.

In certain embodiments, the targeting domain may be chosen, among other things, to at least partially increase the uptake of the compounds of the present disclosure into a desired cell and/or tissue type when introduced into a subject.

In some embodiments, the suitable targeting domain may be a peptide sequence, DNA fragment, aptamer, RNA, folate, polymer, etc. One of ordinary skill in the art, with the benefit of this disclosure, will recognize other targeting domains that may be useful in the compositions of the present disclosure. Such targeting domains are considered to be within the spirit of the present disclosure.

In one aspect, the present invention provides a method of protein target profiling peptide linking site-specific antibody drug conjugates. In one embodiment, the present invention provides a method of protein target profiling peptide stapling site-specific antibody drug conjugates.

In another aspect, the present invention provides a method of targeting a disease or disorder related intracellular targets. In one embodiment, the present invention provides a method of targeting therapeutics for cancer, inflammation, or neuroregeneration (e.g. spinal cord injury treatment). In one embodiment, the present invention provides a method of targeting sensors or antibody mimics to detect at least one biomarker related to a disease or disorder of interest.

Method of Labelling, Imaging, and/or Detecting Compounds of Interest

The present invention also relates, in part, to methods of labeling, imaging, and/or detecting a compound of interest (e.g., an antibody or a fragment thereof, antigen or a fragment thereof, protein or a fragment thereof, peptide or a fragment thereof, amino acid sequence or a fragment thereof, amino acid or a derivative thereof, small molecule or a derivative thereof, therapeutic agent or a derivative thereof, or any combination thereof).

In various aspects, the method of the present invention comprises reacting the compound of interests or salt thereof and a compound or salt thereof having the structure of Formula (XXI)

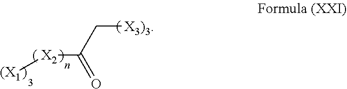

In some embodiment, each occurrence of X₁ and X₃ is independently hydrogen, halogen, OR₁, SR₁, NR₁R₂, CR₁(═R₃), alkyl, substituted alkyl, cycloalkyl, substituted cycloalkyl, heterocycloalkyl, substituted heterocycloalkyl, aryl, substituted aryl, heteroaryl, or substituted heteroaryl.

In some embodiment, each occurrence of X₂ is independently O, S, NR₁, CR₁R₂, C(═R₃), cycloalkyl, substituted cycloalkyl, heterocycloalkyl, substituted heterocycloalkyl, aryl, substituted aryl, heteroaryl, or substituted heteroaryl.

In some embodiment, each occurrence of R₁ and R₂ is independently hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, or substituted heteroaryl.

In some embodiment, each occurrence of R₃ is independently O, NR₁, and S.

In some embodiment, n is an integer from 0 to 10.

In other aspects, the method of the present invention comprises reacting the compound of interest or salt thereof and a compound or salt thereof having the structure of Formula (II)

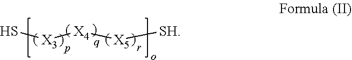

In yet other aspects, the method of the present invention comprises reacting the compound of interest or salt thereof and a compound or salt thereof having the structure of Formula (III)

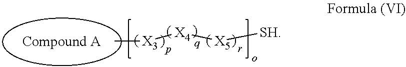

For example, in some embodiments, the method of the present invention comprises reacting a compound or salt thereof having the structure of Formula (XXII)

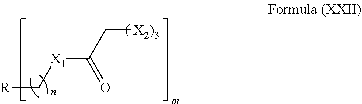

and a compound or salt thereof having the structure of Formula (II)

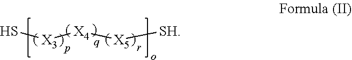

In other embodiments, the method of the present invention comprises reacting a compound or salt thereof having the structure of Formula (XXII)

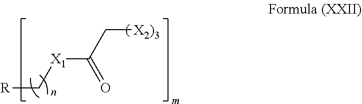

and a compound or salt thereof having the structure of Formula (III)

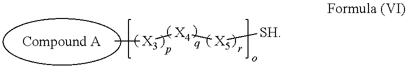

In some embodiments, R is an antibody or a fragment thereof, antigen or a fragment thereof, protein or a fragment thereof, peptide or a fragment thereof, amino acid sequence or a fragment thereof, amino acid or a derivative thereof, small molecule or a derivative thereof, therapeutic agent or a derivative thereof, or any combination thereof.

In some embodiments, the compound A is an antibody or a fragment thereof, antigen or a fragment thereof, protein or a fragment thereof, peptide or a fragment thereof, amino acid sequence or a fragment thereof, amino acid or a derivative thereof, small molecule or a derivative thereof, therapeutic agent or a derivative thereof, or any combination thereof. For example, in one embodiment, the compound A is biotin.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: A Fluorine-Thiol Displacement Reaction (FTDR) for Steric-Free Bioorthogonal Labeling

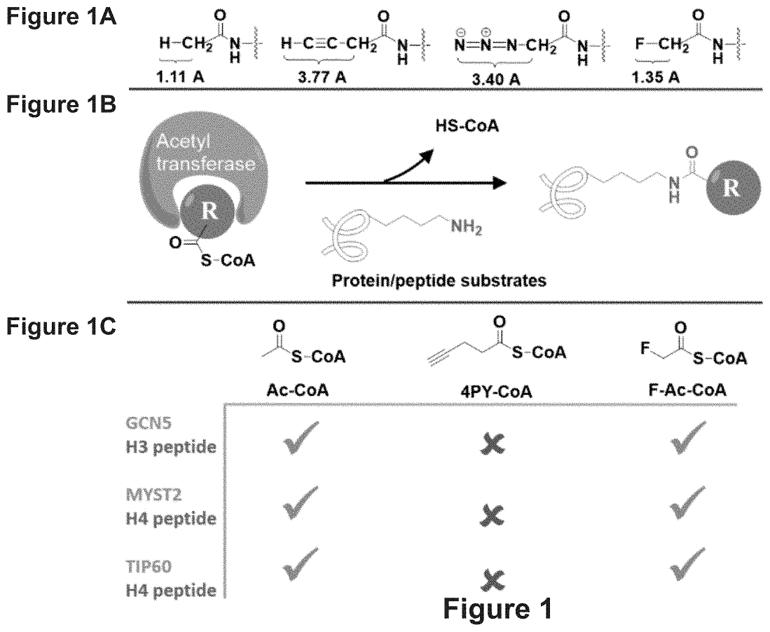

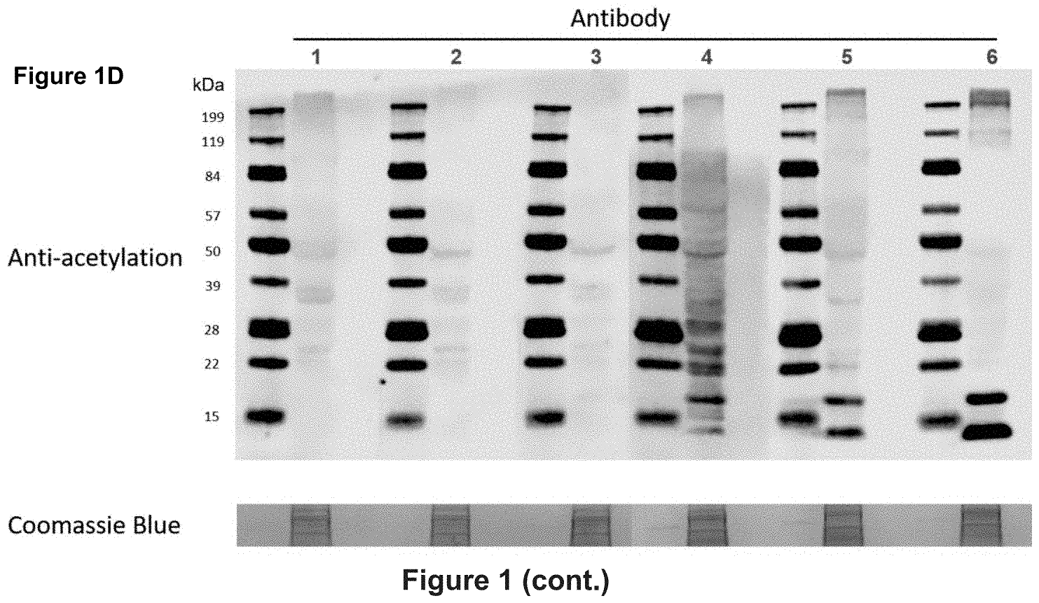

Global profiling of post-translational modification (PTM) substrates, such as those of acetylation can still be achieved by antibody-based detections (Shaw P G et al., 2011, Anal. Chem., 83:3623-3626). However, the enriched substrates and sites varied significantly per antibody (Shaw P G et al., 2011, Anal. Chem., 83:3623-3626). However, in the present example, a list of commercially available anti-lysine acetylation antibodies revealed different sensitivity and substrate specificity (FIG. 1D). Taken together, elucidating the molecular targets of PTMs, such as acetylation, has been thereby compromised, despite being a key step towards the systematic dissection of PTMs and their roles in biological and pathology-related cellular signaling regulation (Yang C et al., 2013, J. Am. Chem. Soc., 135:7791-7794; Markus Grammel Y Y Y et al., 2012, Chemical reporters of protein methylation and acetylation; Buuh Z Y et al., 2018, J. Med. Chem., 61:3239-3252). To this end, the present example focused on the investigation of whether a bioorthogonal reaction can be developed to generate reporters for the steric-free labeling of protein substrates and thereby allow for the global profiling of molecular targets.

The fluorine atom is attractive due to its orthogonality from biological molecules (Tressler C M et al., 2017, Biochemistry, 56:1062-1074; Li C et al., 2010, J. Am. Chem. Soc., 132:321-327), similarity in size to hydrogen (Tressler C M et al., 2017, Biochemistry, 56:1062-1074; Gee C T et al., 2015, Angew. Chem. Int. Ed. Engl., 54:3735-3739; O'Hagan D, 2008, Chem. Soc. Rev., 37:308-319), and the comparable length of the carbon-fluorine bond to the carbon-hydrogen bond (FIG. 1A) (O'Hagan D, 2008, Chem. Soc. Rev., 37:308-319). Fluorinated amino acids have been exploited to label proteins, bringing in minimal perturbations to protein structure and function, as evident by ¹⁹F NMR (Tressler C M et al., 2017, Biochemistry, 56:1062-1074; Li C et al., 2010, J. Am. Chem. Soc., 132:321-327; Gee C T et al., 2015, Angew. Chem. Int. Ed. Engl., 54:3735-3739; Mishra N K et al., 2014, ACS Chem. Biol., 9:2755-2760). Using a representative PTM, acetylation, for proof of concept, initial studies investigated whether fluorinated acetyl-CoA can hijack the CoA metabolism and be used by acetyltransferases to label their protein substrates (FIG. 1B).

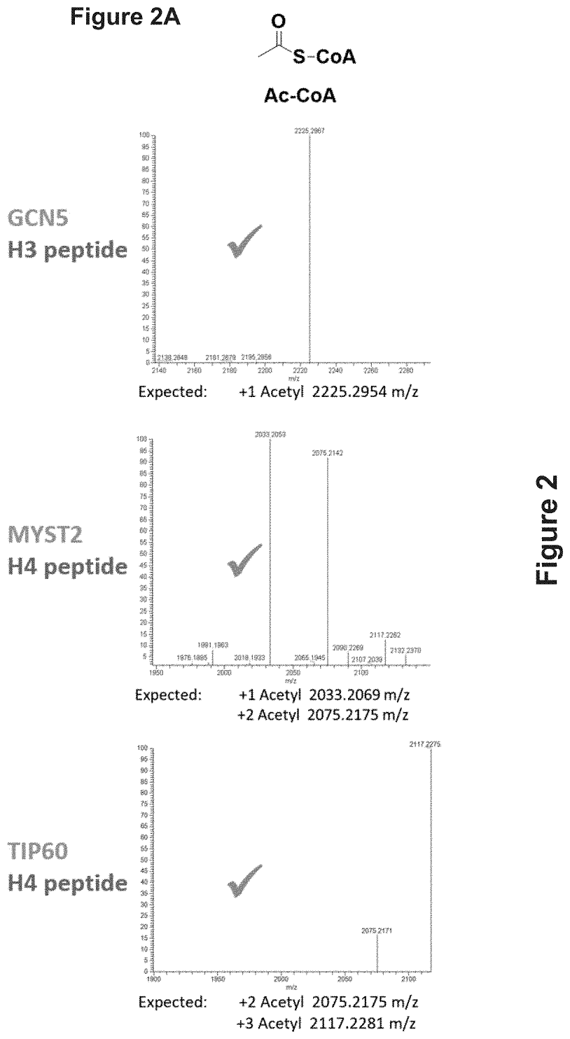

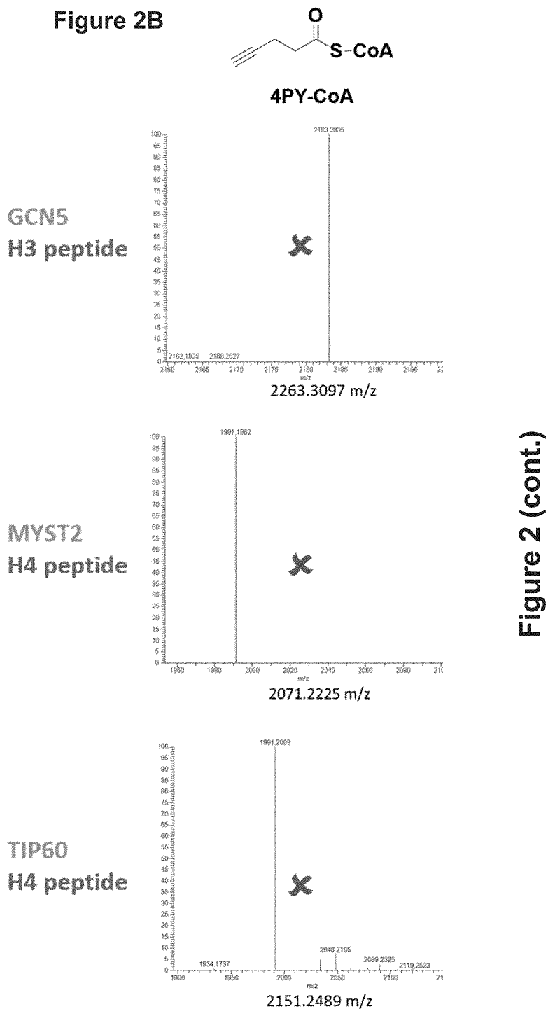

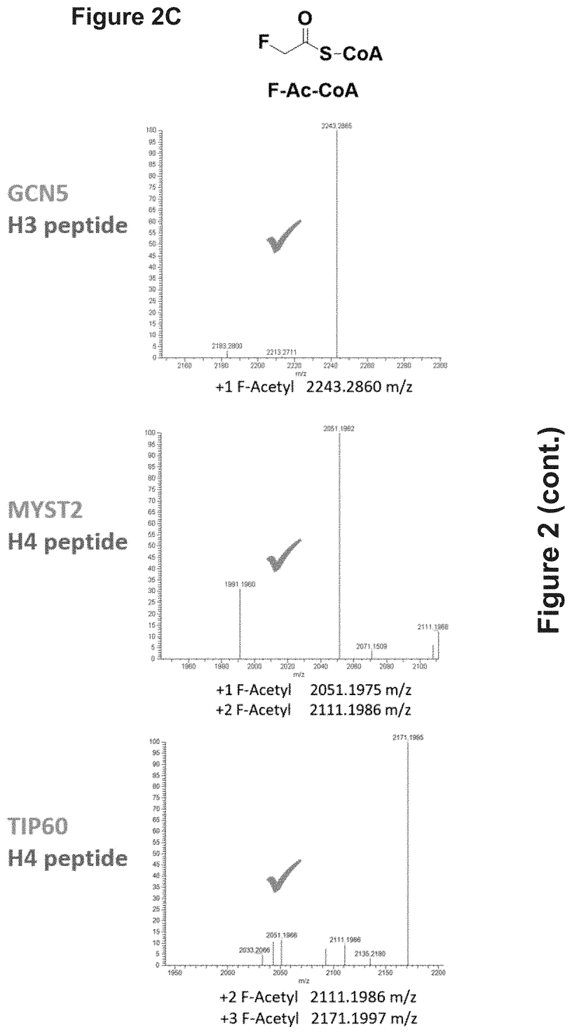

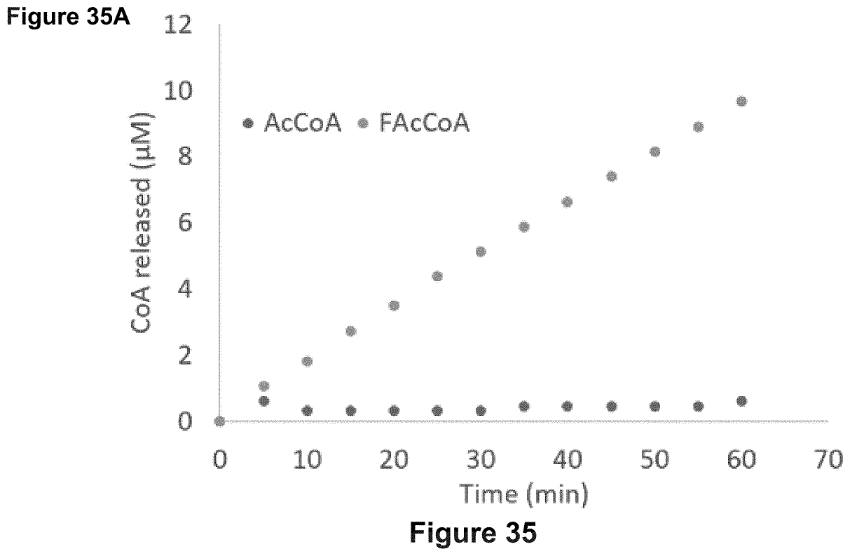

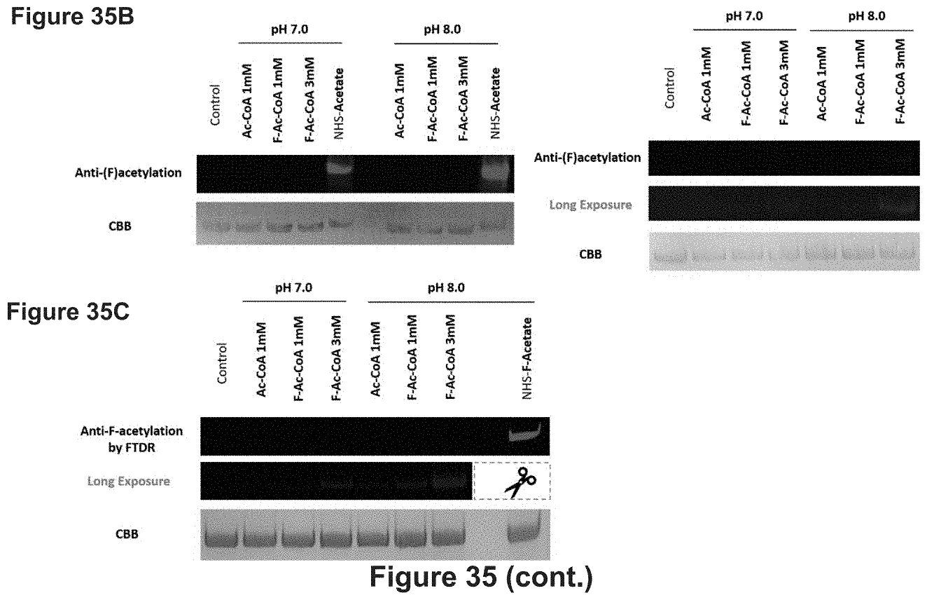

Despite the in vivo toxicity of its pro-metabolite fluoroacetate, only the late-stage metabolite fluorocitrate was found account-able, mostly damaging organ tissues, such as the kidney (Peters R et al., 1952, Proc. R. Soc. Lond. Ser. B. Biol. Sci., 139:143-170). Fluorinated acetyl-CoA and fluoroacetate have not yet been found to inhibit any enzyme, and have been successfully used to study metabolisms (Marcus A et al., 1956, J. Biol. Chem., 218:823-830; Mori T et al., 2009, Nucl. Med. Biol., 36:155-162). Ac-CoA analogs with fluorine (F-Ac-CoA) (Weeks A M et al., 2012, Proc. Natl. Acad. Sci. USA, 109:19667-19672) or alkyne (4-pentynoyl (4PY)-CoA) (Yang C et al., 2013, J. Am. Chem. Soc., 135:7791-7794) functional groups were synthesized, and each mixed with key acetyltransferase GCN5, MYST2, or TIP60 and their corresponding histone peptide substrates (Yang C et al., 2013, J. Am. Chem. Soc., 135:7791-7794; Marmorstein R, 2001, Cell Mol. Life Sci., 58:693-703; Kimura A et al., 198, Genes Cells, 3:789-800). Mass spectrometric analysis (FIG. 2) indicated successful acetyl or acetyl analog labelling after either acetyltransferase was incubated with wild type Ac-CoA or F-Ac-CoA. On the contrary, both acetyltransferases failed to incorporate the alkyne-modified 4-PY-CoA, which is consistent with previous reports (Yang C et al., 2013, J. Am. Chem. Soc., 135:7791-7794; Han Z et al., 2017, ACS Chem. Biol., 12:1547-1555). Substitution with the electron-withdrawing fluorine also endowed a much higher hydrolysis rate to F-Ac-CoA, which somehow did not display significantly greater non-enzymatic reactivity (FIG. 35) (Wagner G R et al., 2017, Cell Metab., 25:823-83). Taken together (FIG. 1C), fluorine modification afforded a relatively steric-free chemical reporter that can satisfactorily label substrates of PTMs, such as acetylation.

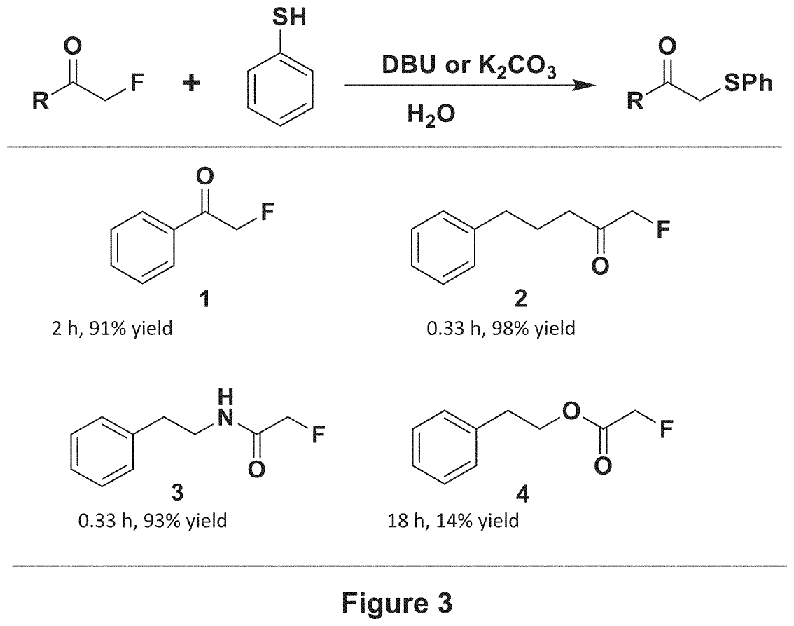

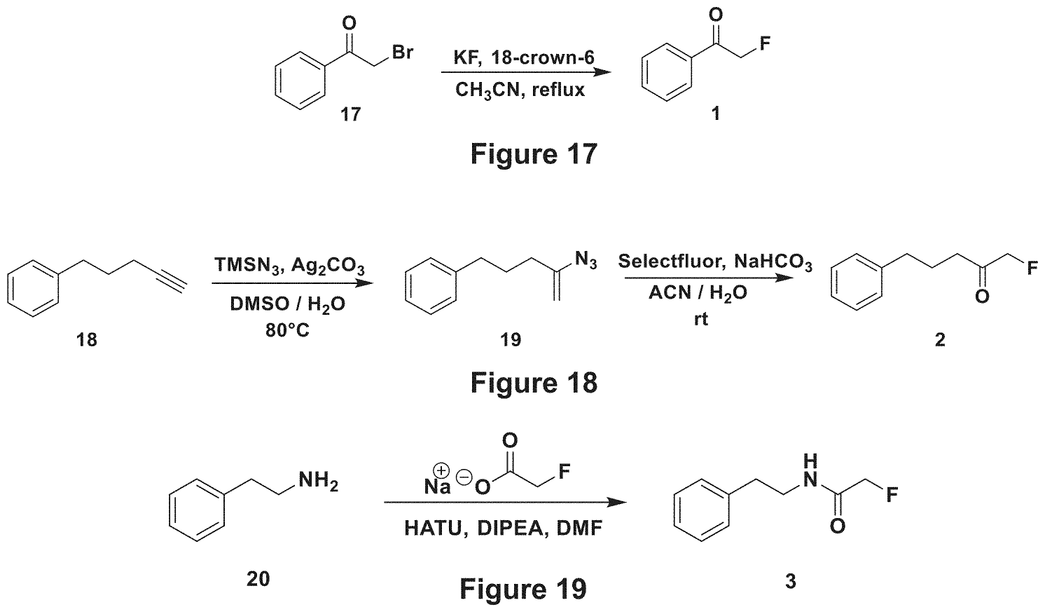

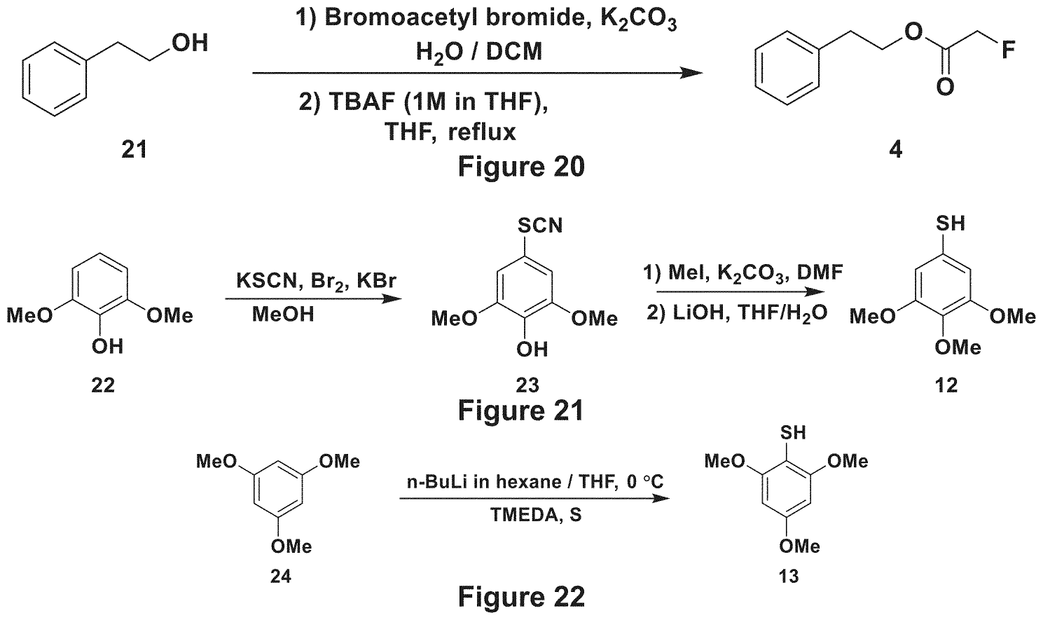

Next, studies investigated whether the substituted fluorine can convert to other tags, such as a fluorescent dye “TAMRA” (Whitaker J E et al., 1992, Anal. Biochem., 207:267-279) or a biotin affinity probe (Yang Y et al., 2013, Mol. Cell Proteomics, 12:237-244) for detection, imaging, and future identification of protein substrates. Despite substantial progress in the development of fluorination methodology, there are few efforts on the replacement of fluorine. One relevant study (Xiang Z et al., 2013, Nat. Methods, 10:885-888) has incorporated alpha-fluorinated acetophenone (1) moiety into proteins and observed its reaction with a proximal cysteine. Without being bound to any particular theory, it was hypothesized that fluorinated acetyl groups may react with thiol derivatives even at the small molecule level. To test this, a representative thiol compound, benzenethiol, was reacted with a couple of fluorinated acetyl substrates in water (FIG. 3). In the presence of a strong base (DBU or K₂CO₃), the fluorine alpha to acetophenone (1), ketone (2), and amide (3), synthesized via methods shown in FIG. 17 through FIG. 19, were all efficiently displaced with decent yields (>90%; FIG. 3). A similar conversion was also observed with the fluoroacetate derivative (4), synthesized via a method shown in FIG. 20, despite a lower yield and efficiency, which was likely caused by hydrolysis of the ester bond given the isolated benzeneethanol side product. The result with (3) were particularly exciting as it is a model substrate for fluorinated peptide/protein substrates. A related report came out at that same time, confirming that similar reactions can happen at the protein level if in close proximity (Kobayashi T et al., 2016, J. Am. Chem. Soc., 138:14832-14835).

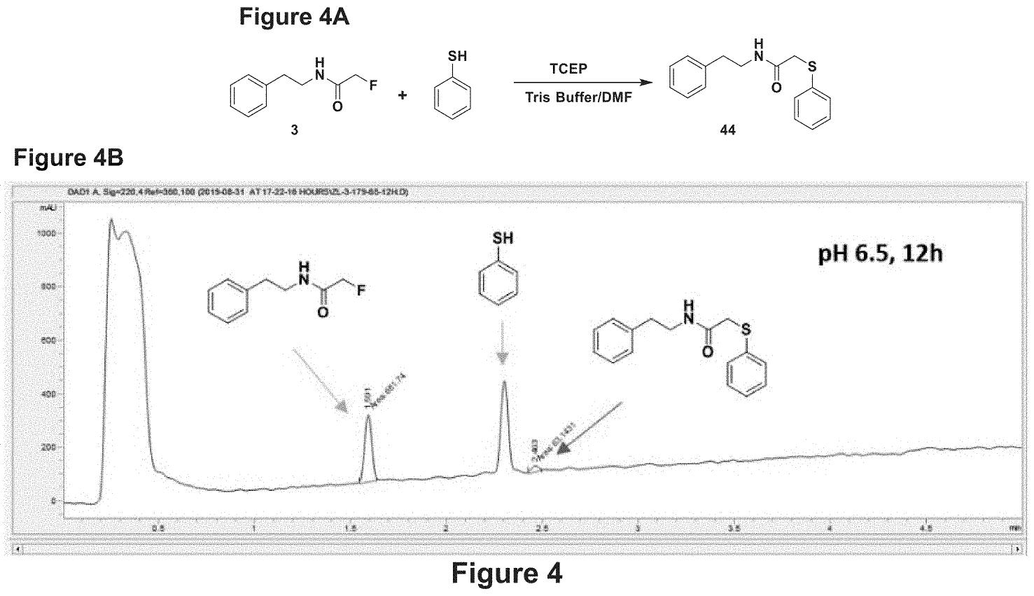

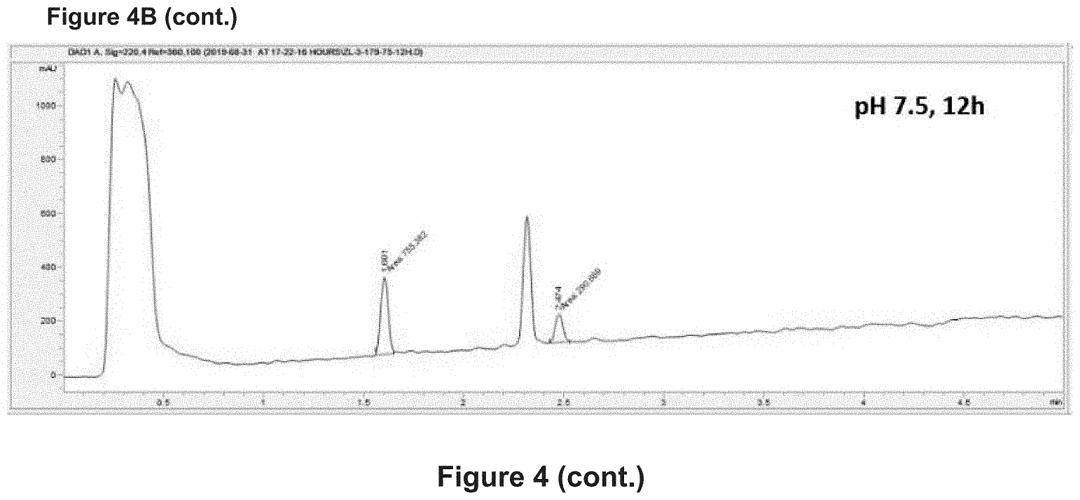

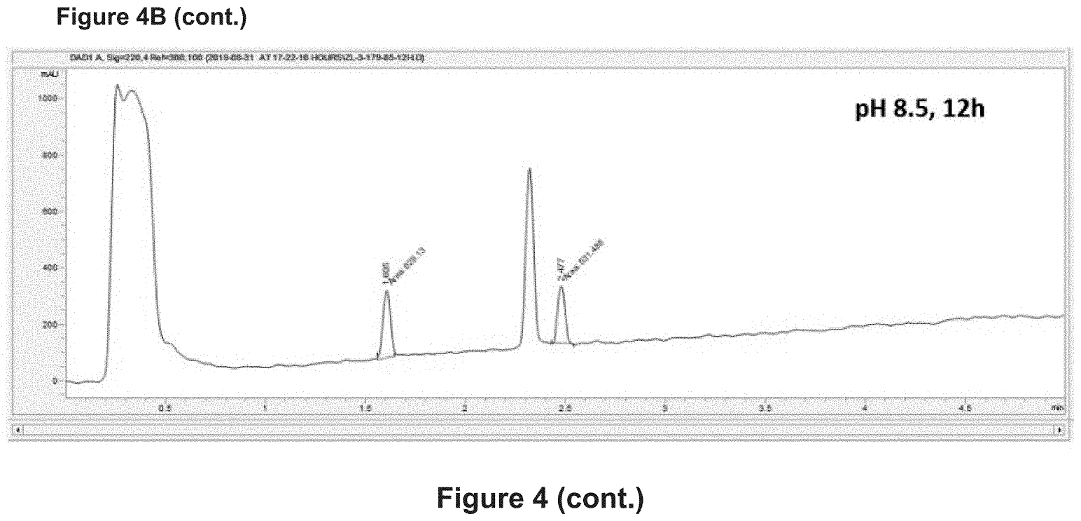

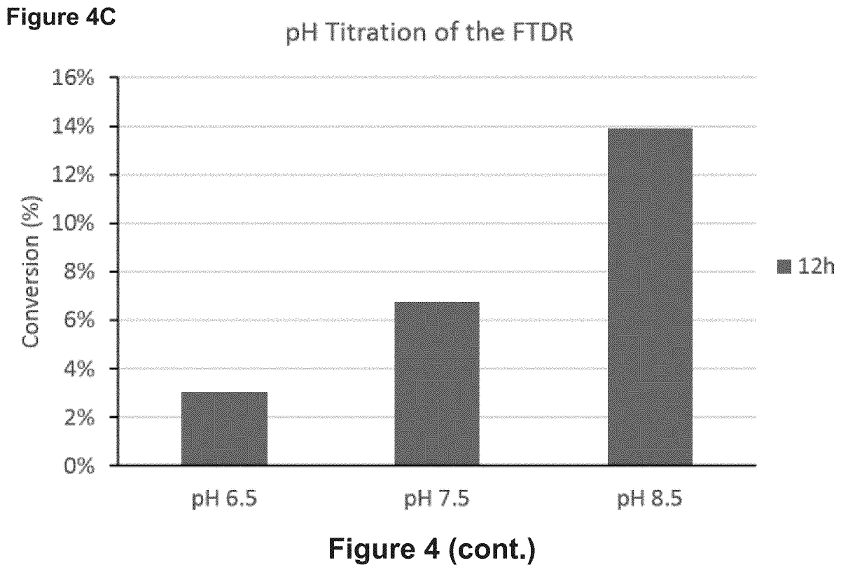

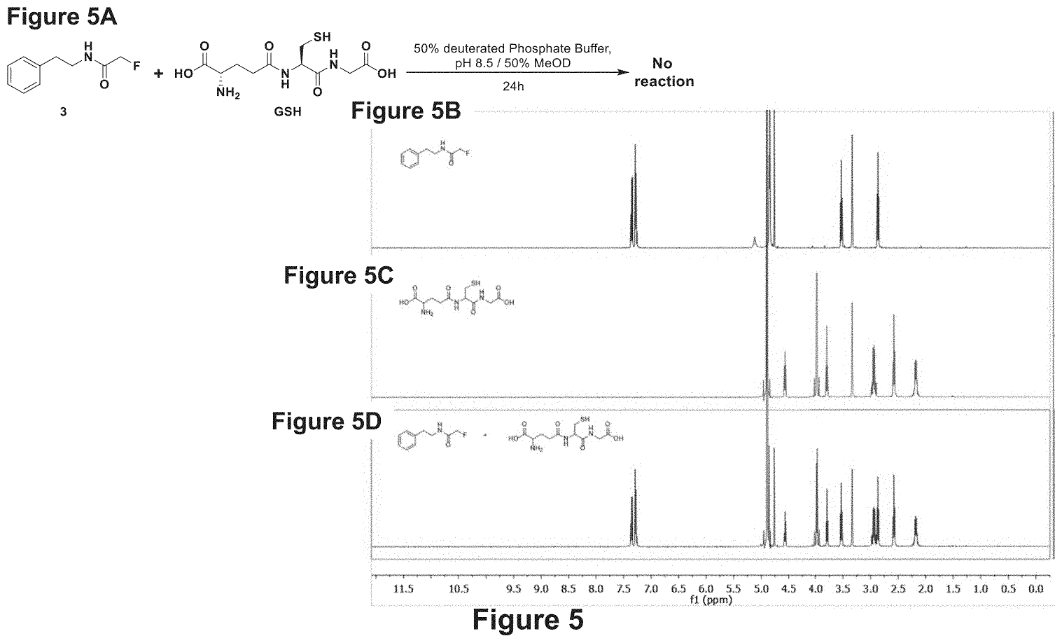

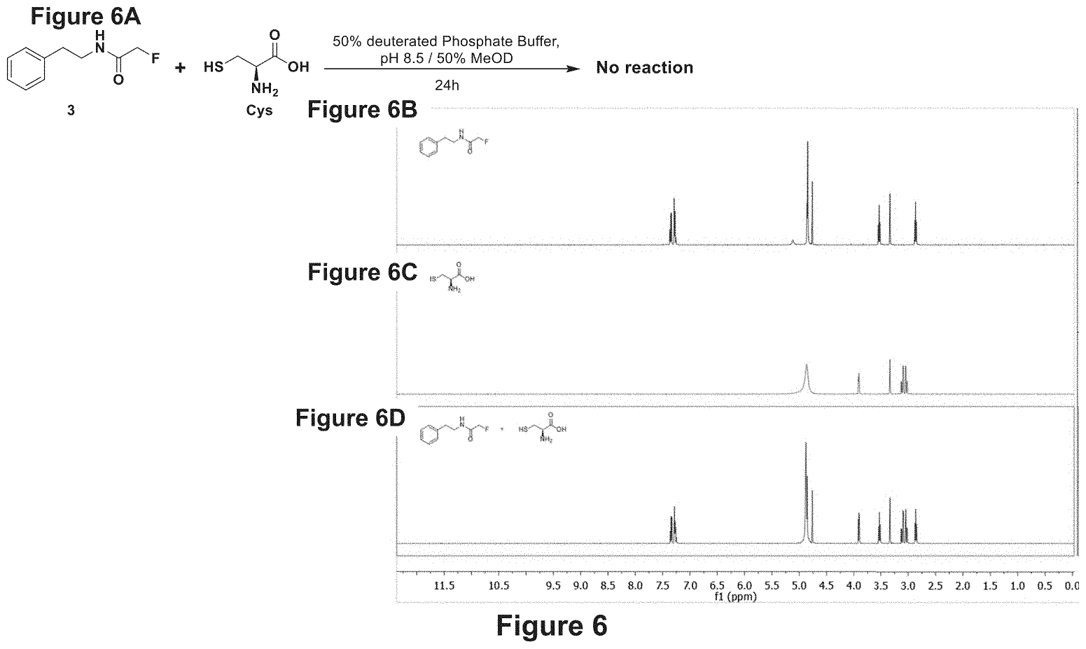

To identify the optimal pH range for this type of reaction, the pH (FIG. 4) was titrated and found that the reaction rate significantly increased with pH. The increased deprotonation of benzenethiol likely contributed to this effect. Using mildly basic condition, pH 8.5, in which the reaction has the best reaction rate, substrate (3)'s reactivity was also examined with strong intrinsic nucleophiles that exist in regular cellular environments, glutathione and cysteine (FIG. 5 and FIG. 6). Surprisingly, no reaction was observed for both cases upon an incubation of 24 h, suggesting that substrate (3) could be bioorthogonal to other nucleophilic species.

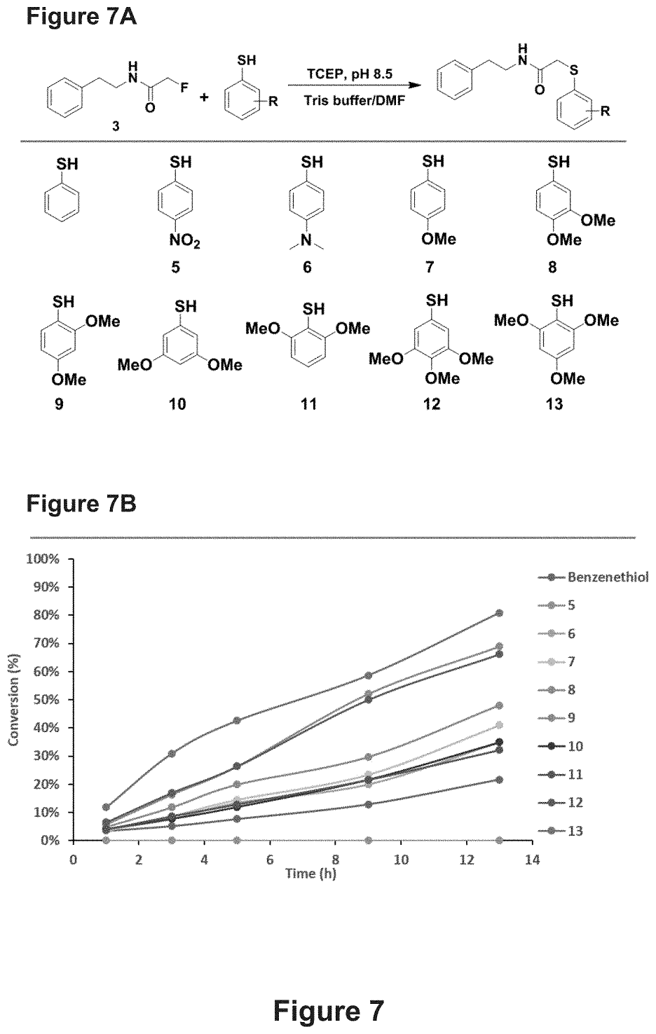

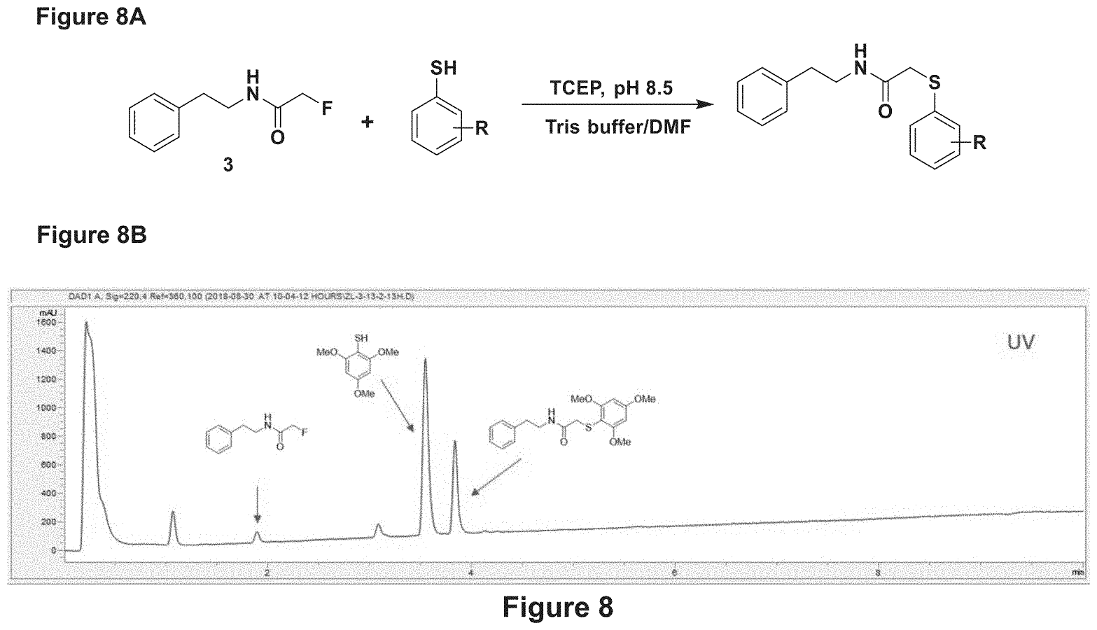

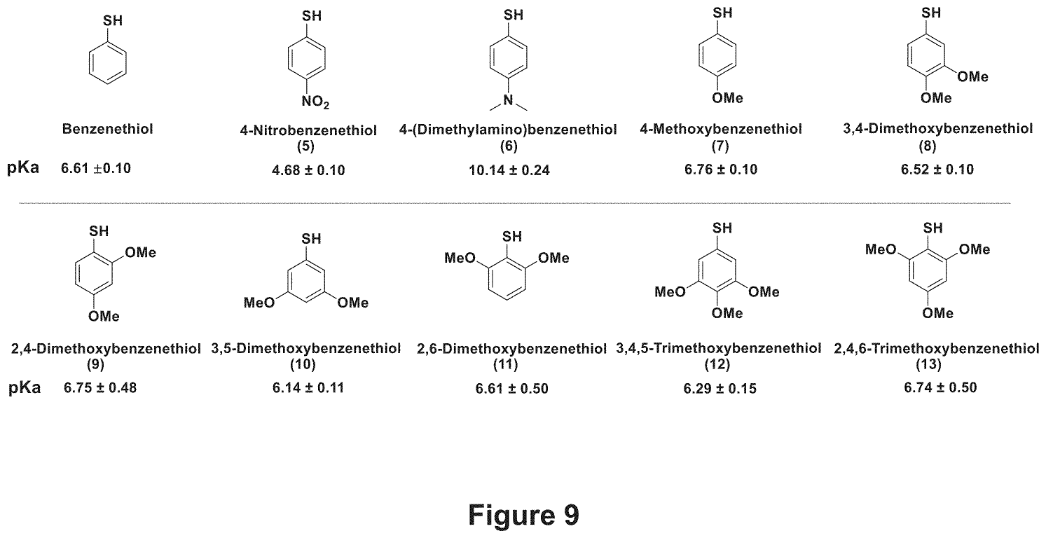

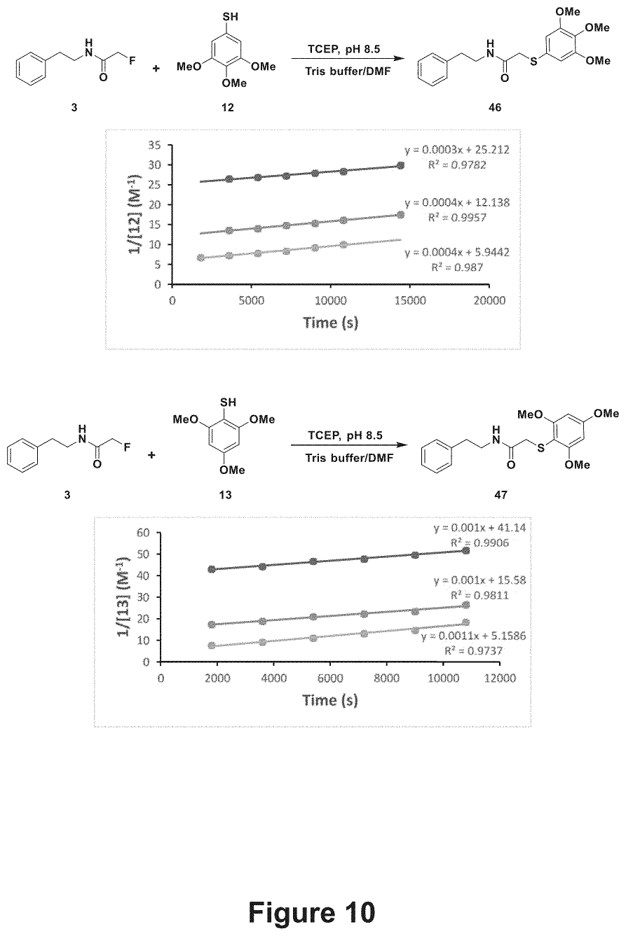

Subsequent studies then investigated substitution effects on benzenethiol in order to efficiently convert fluoroacetamide under the mild physiological reaction condition (Bos J et al., 2018, J. Am. Chem. Soc., 140:4757-4760; Zhu X et al., 2016, New J. Chem., 40:4562-4565). Exploration of a series of benzenethiol derivatives (5)-(13) found that electron donating groups at the ortho- and para-positions facilitate the reaction by increasing the nucleophilicity (FIG. 7 and FIG. 8). Given their theoretical pKa's (<7.0) (FIG. 9), almost all of these derivatives should be fully deprotonated. The most reactive derivative, (13), is a tri-methoxy analog that can reach ˜81% reaction conversion within 13 h. Despite potential issues regarding sterics for the ortho-substitutions, the superior reactivity of (13) to the metasubstituted derivative (12) indicated that the electron donating methoxy groups are not bulky enough to perturb reactivity. Likewise, derivative (9) that only bears two substitutions at the electron donating sites ended up possessing the same reactivity as (12). Follow-up kinetics studies based on the reported procedures (Agard N J et al., 2006, ACS Chem. Biol., 1:644-648) suggested that the bimolecular reaction between (3) and (13) followed second order kinetics, and had an observed rate constant of (1.03±0.06)×10⁻³ M⁻¹ S⁻¹ (FIG. 10), close in rates to the classic Staudinger ligation reaction (Agard N J et al., 2006, ACS Chem. Biol., 1:644-648; Sundhoro M et al., 2017, Angew. Chem. Int. Ed. Engl., 56:12117-12121).

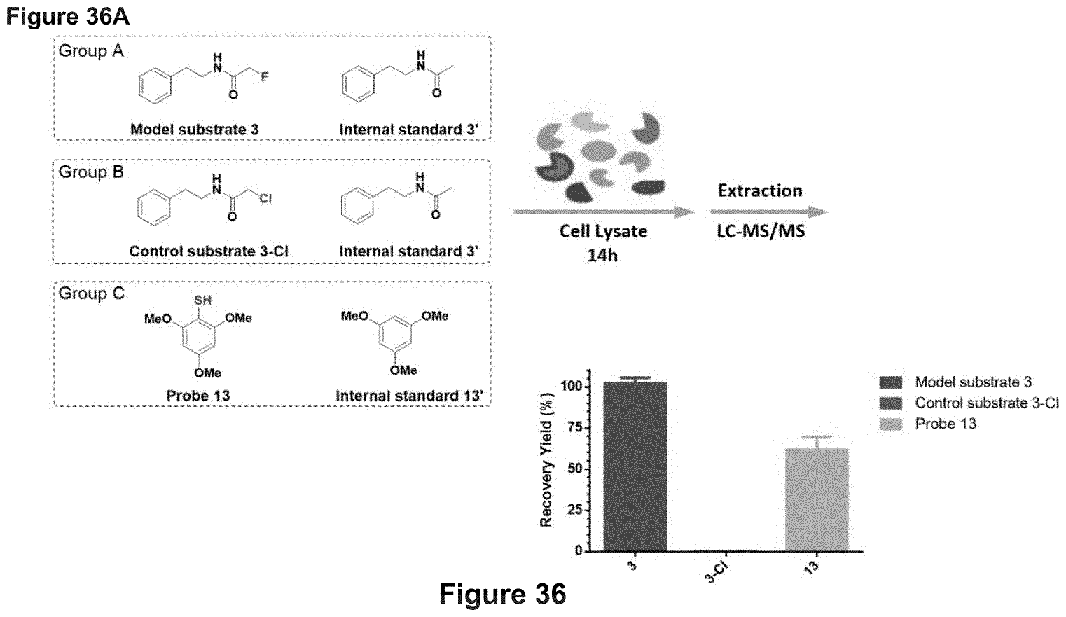

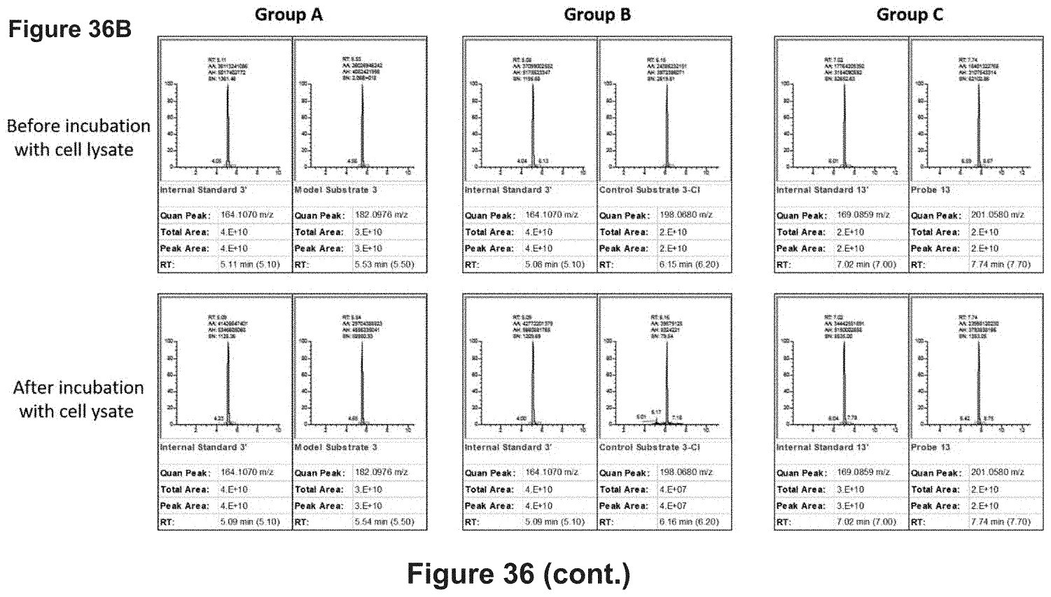

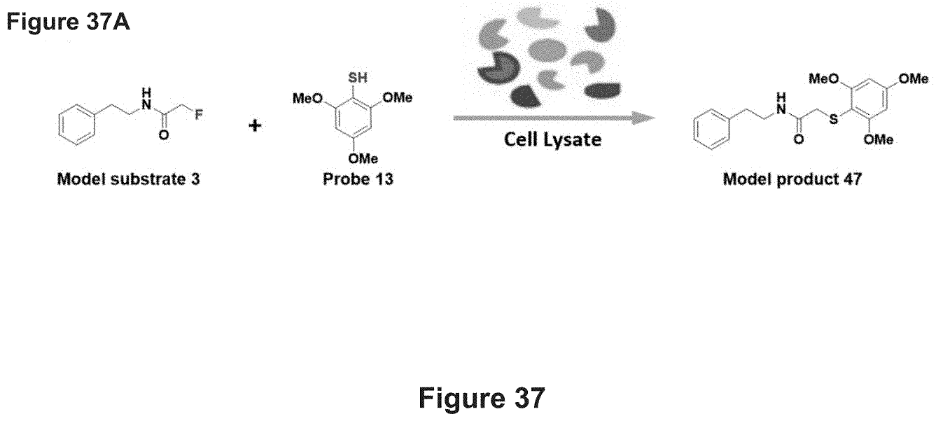

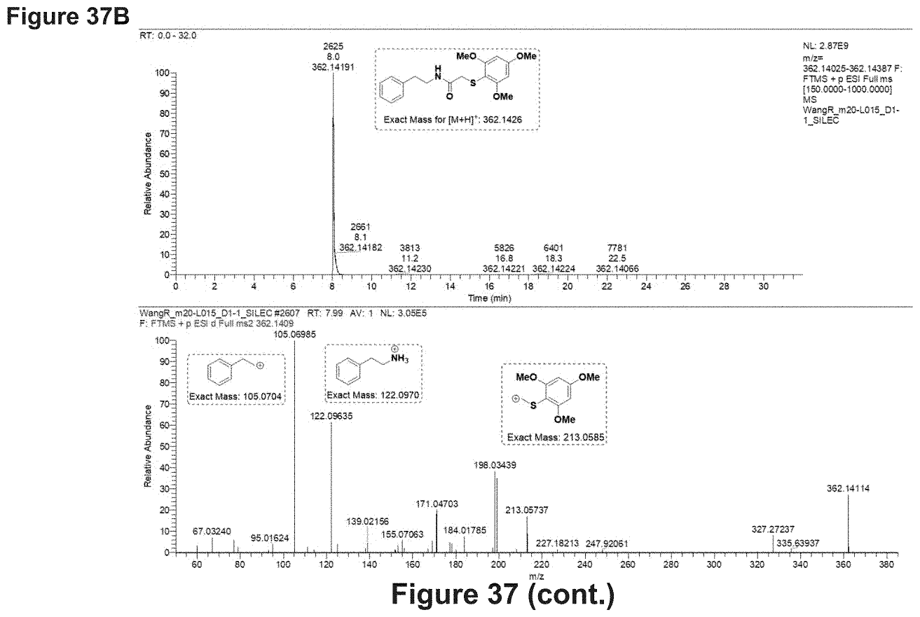

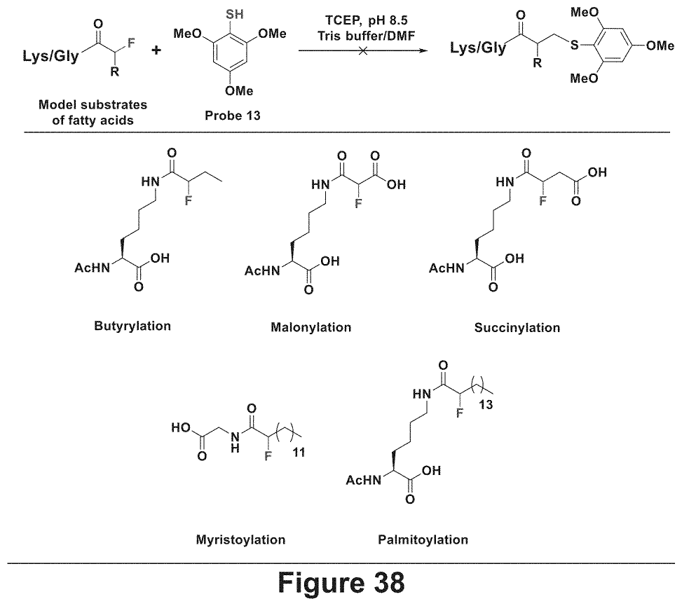

Moreover, the stability of (13) along with substrate (3) were further evaluated in cell lysates (FIG. 36), where they remained mostly intact (61.8% and 100%, respectively). On the contrary, control (3)-Cl possessing the reported chloroacetamide (Yu M et al., 2006, J. Am. Chem. Soc., 128:15356-15357) was not stable, with only 0.15% remained. This result was consistent with previous observations that chloroacetamide readily reacted with cellular proteins (Barglow K T et al., 2004, Chem. Biol., 11:1523-1531). With the aid of LC-MS/MS, the FTDR product of (3) and (13) in cell lysates was confirmed (FIG. 37), further corroborating the bioorthogonality of FTDR. Notably, wild-type Ac-CoA has been regarded as a central metabolite that can be also used for ketogenesis, mevalonate pathway, and fatty acid synthesis (Pietrocola F et al., 2015, Cell Metab., 21:805-821). F-Ac-CoA as a mimic of Ac-CoA was demonstrated to be taken by fatty acid synthetases to make fluoro-fatty acids (Walker M C et al., 2014, Chem. Soc. Rev., 43:6527-6536), most of which are also classic PTMs. The FTDR reaction was thereby tested between probe (13) and the alpha-fluorinated model substrates of fatty acids, such as butyrate, myristic acid, palmitic acid, malonic acid, and succinic acid, etc. (FIG. 38). Surprisingly, no FTDR reaction was observed with these substrates, suggesting that due to the in-creased steric hindrance the secondary fluorides in these fatty acids may not be as readily displaceable as the primary fluoride in fluoroacetamide. On the other hand, these observations also indicated the potential uniqueness of fluoroacetamide and its orthogonality to other fluorine-substituted natural molecules for FTDR-based detection, imaging, and target identification.

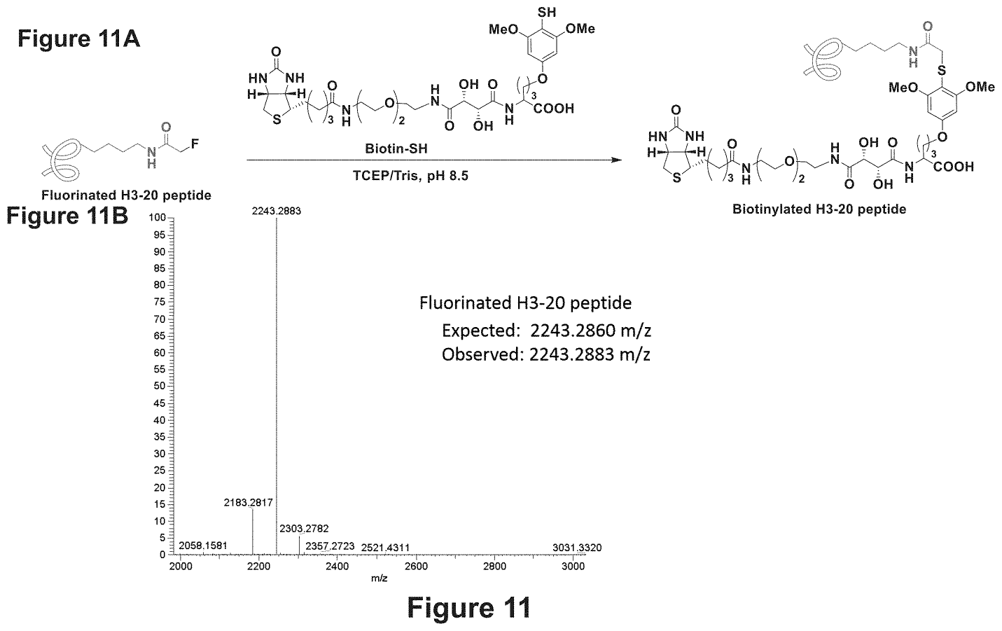

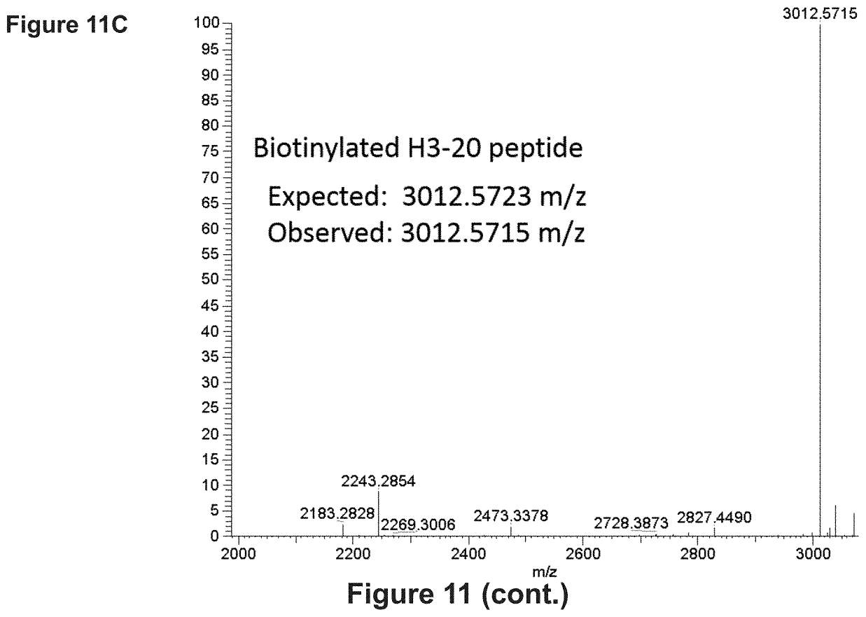

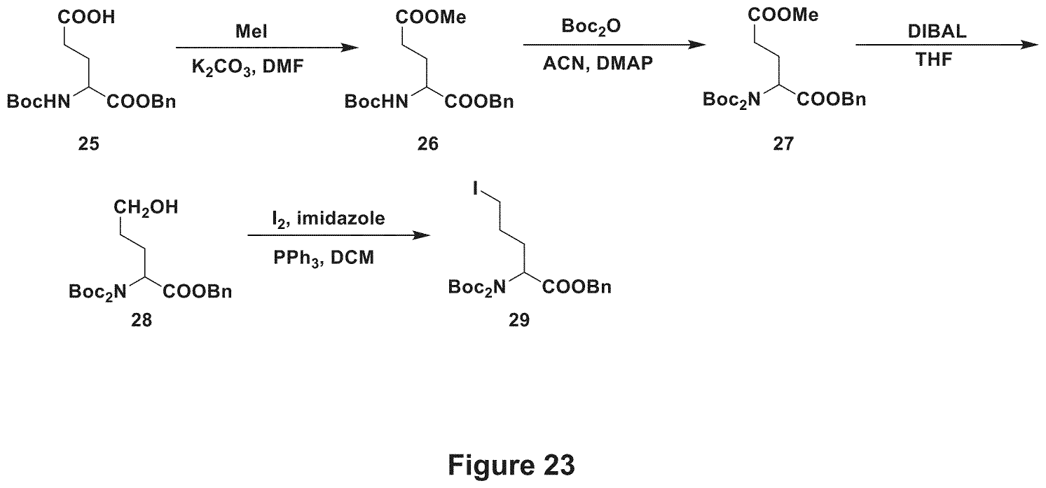

With the most active benzenethiol derivative in hand, converting fluorine to functional detection tags, such as fluorescent dyes and biotin probes, which have been routinely used for detection (Yang Y et al., 2013, Mol. Cell Proteomics, 12:237-244; Andersen K A et al., 2015, J. Am. Chem. Soc., 137:2412-2415) was investigated. It also provided great potential for future affinity pull down studies (Yang Y et al., 2013, Mol. Cell Proteomics, 12:237-244). Thus, the studies started by constructing a biotin probe ((14), Biotin-SH) that contained the benzenethiol structure of (13) as the warhead, and the glutamic acid building block (29) as the connecting unit to improve overall solubility (FIG. 23 through FIG. 25). Due to the ease of ESI-MS characterization on peptides, the successful conversion of the fluorine label to a biotin tag on the aforementioned histone H3-20 peptide substrate was demonstrated first (FIG. 11).

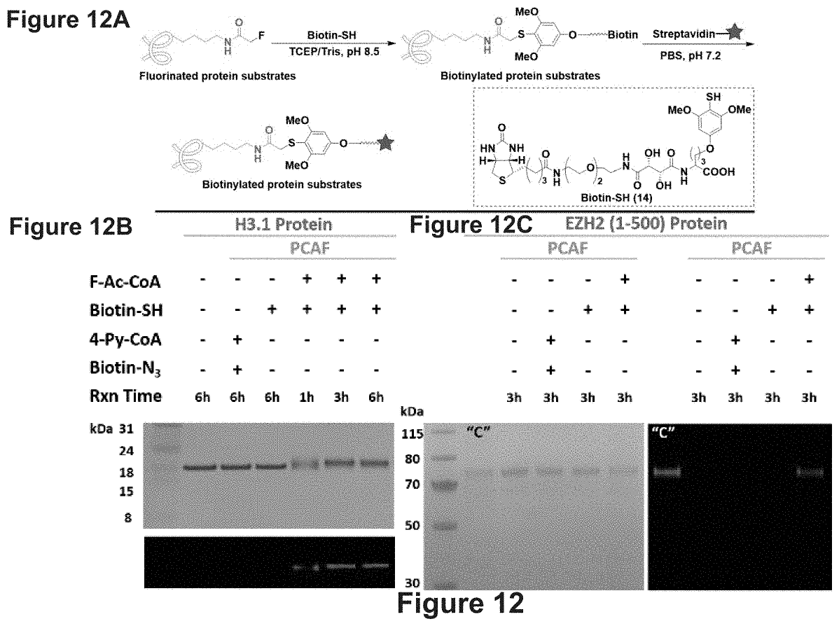

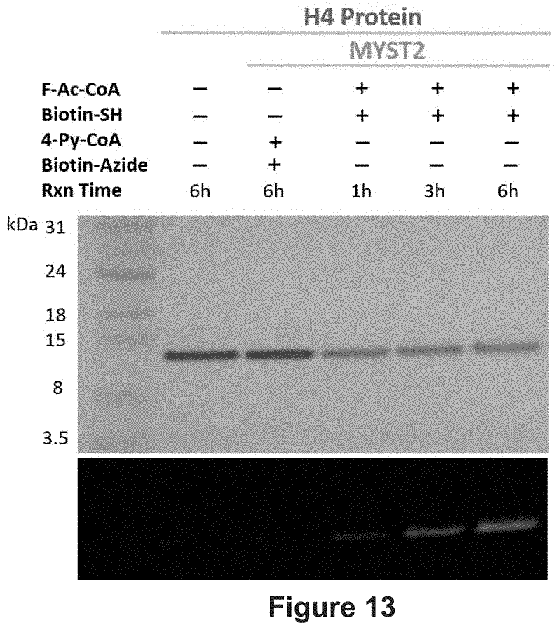

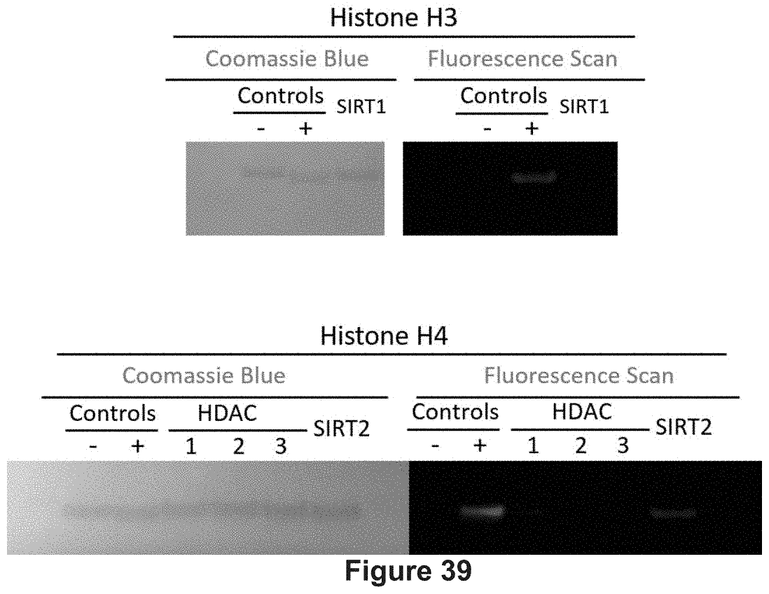

Next, the whole process of labelling and tagging (FIG. 12A) was investigated on histone protein substrates. Histone H3.1 (FIG. 12B) and H4 subunit (FIG. 13) were each incubated with F-Ac-CoA, and the corresponding KAT under standard in vitro enzymatic reaction conditions (Yang C et al., 2013, J. Am. Chem. Soc., 135:7791-7794). The mixtures were then incubated with the Biotin-SH probe for varied time periods, with subsequent in-gel fluorescent imaging to detect the biotin tagging. For both histone proteins, efficient biotinylation occurred within 1 h of incubation, and gradually reached saturation after 3 h. Staining with coomassie brilliant blue (CBB) also revealed a complete change of molecular weight, presumably due to the two-step modification. The minimal signal observed from the control group without prior fluorination also indicated the relative specificity of Biotin-SH. Concurrently, failure of biotinylation was observed for the group using CuAAC labeling, which was consistent with literature reports, and confirmed the fact that many KATs are incapable of uptaking sterically hindered substrates (Yang C et al., 2013, J. Am. Chem. Soc., 135:7791-7794; Han Z et al., 2017, ACS Chem. Biol., 12:1547-1555). To check if the fluoroacetyl label is recognized by histone deacetylases, these fluoroacetylated histone proteins were also incubated with a few reported deacetylases (Seto E et al., 2014, Cold Spring Harb. Perspect. Biol., 6:a018713) before Bio-tin-SH tagging. Interestingly, most fluoroacetylation was efficiently removed (FIG. 39), suggesting that the label can be a substrate for deacetylases as well.

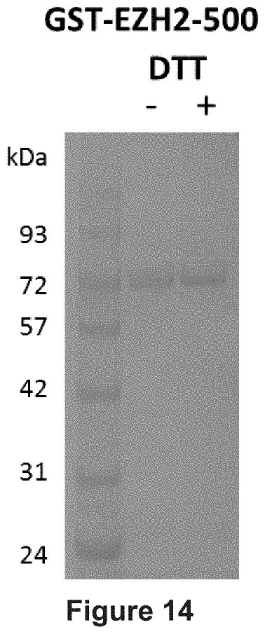

In addition to histones, a growing number of non-histone substrates were recently uncovered, with their roles in protein function regulation being increasingly appreciated (Buuh Z Y et al., 2018, J. Med. Chem., 61:3239-3252). For instance, enhancer of zeste homolog 2 (EZH2) was revealed to be acetylated by PCAF mainly at lysine 348, which improved EZH2's stability and promoted the migration of lung cancer cells (Wan J et al., 2015, Nucleic Acids Res., 43:3591-3604). To evaluate whether the labeling strategy is applicable to non-histone substrates, the truncated form EZH2(1-500) was specifically constructed and expressed in E. coli, at a shake-flask yield of approximately 2 mg/L. The protein's purity and size were confirmed by SDS-PAGE (FIG. 14). Reaction of the EZH2 fragment under the same conditions as those for histone proteins resulted in a similar type of tagging as evident by FIG. 12C. Taken together, these observations displayed the steric-free in vitro labeling and tagging of a range of known protein substrates using the FTDR reaction, which could not be achieved by the known CuAAC method.

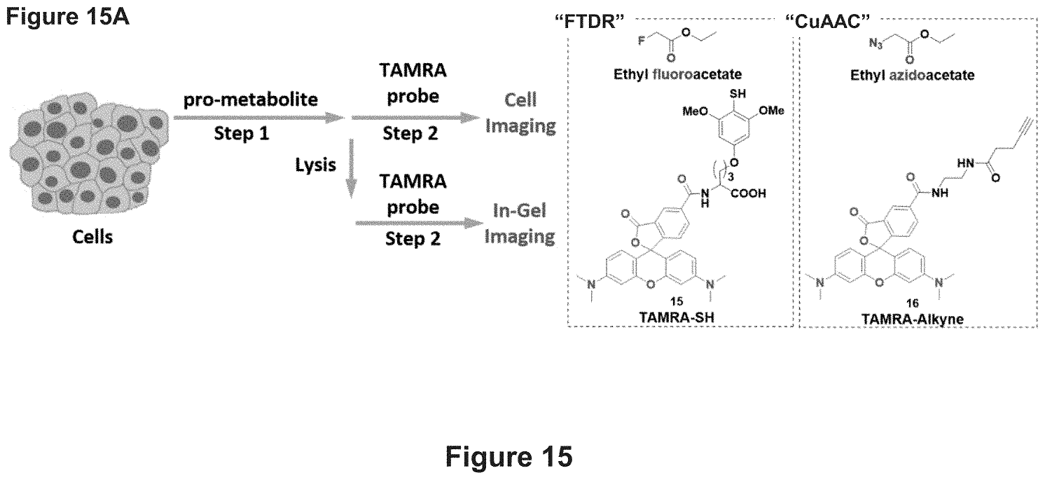

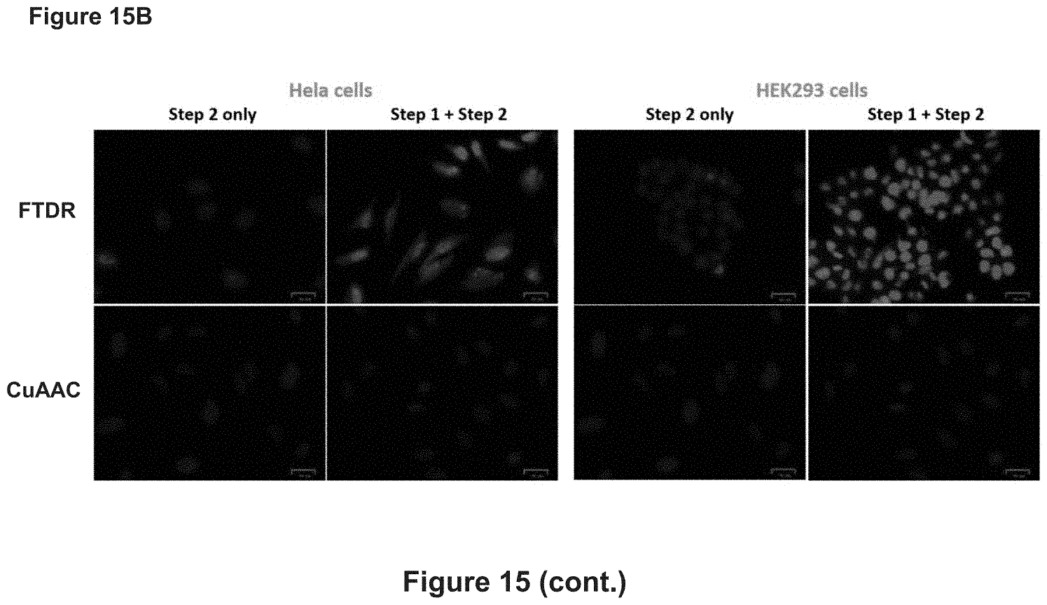

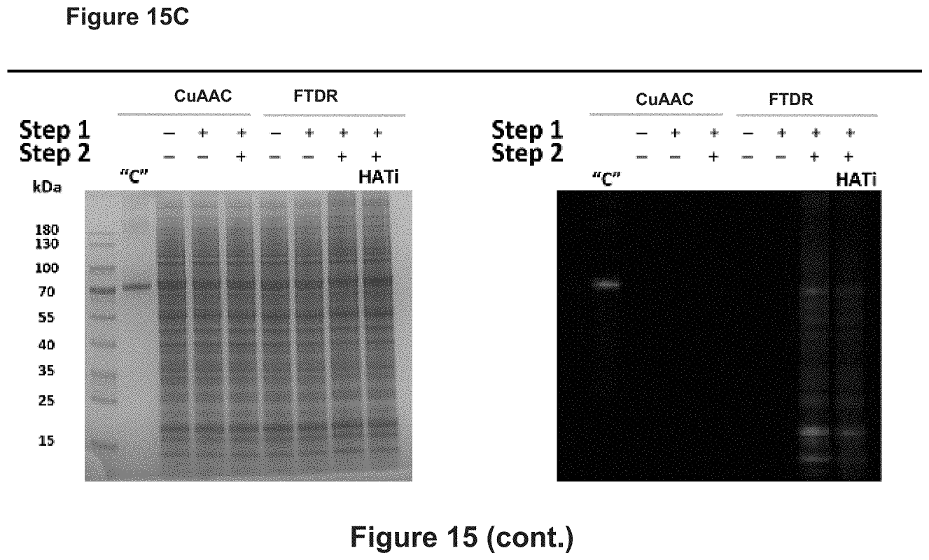

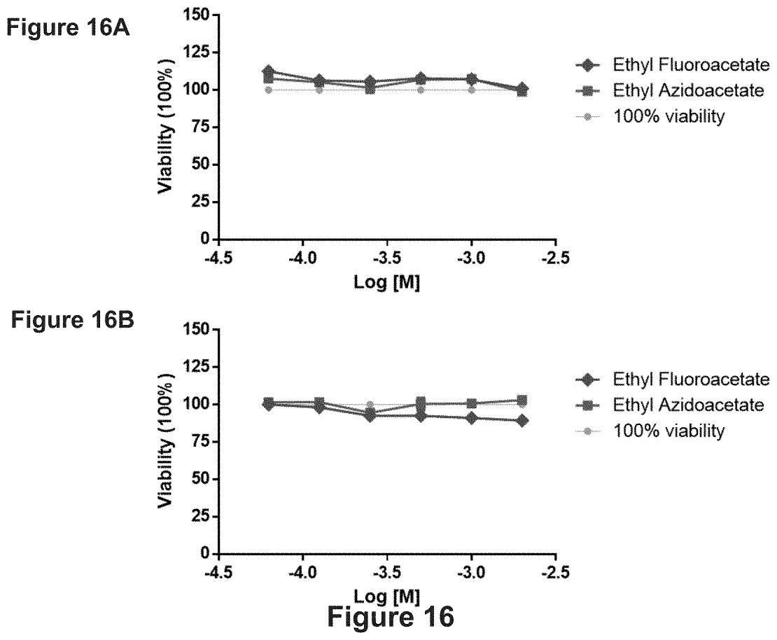

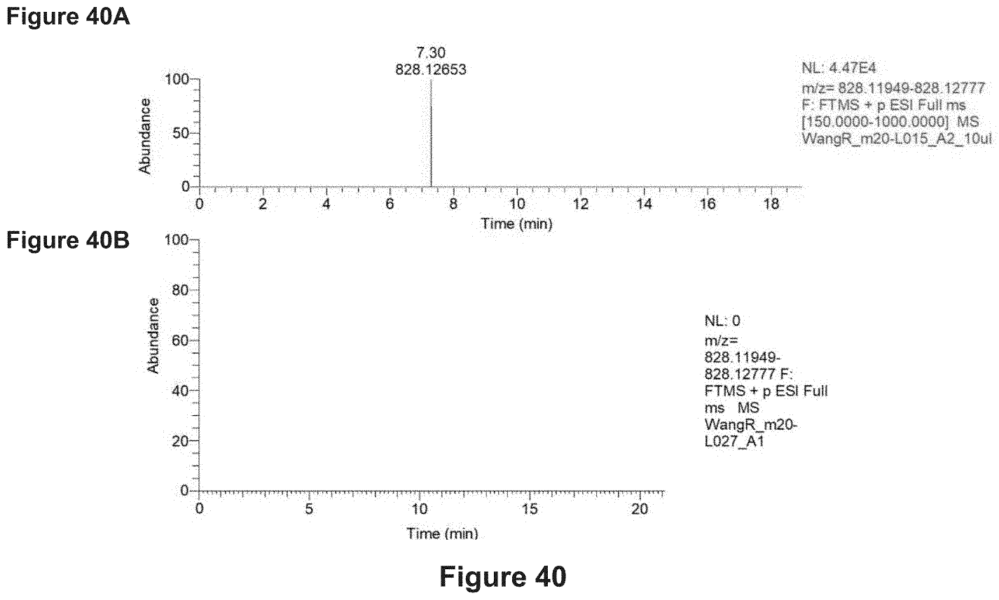

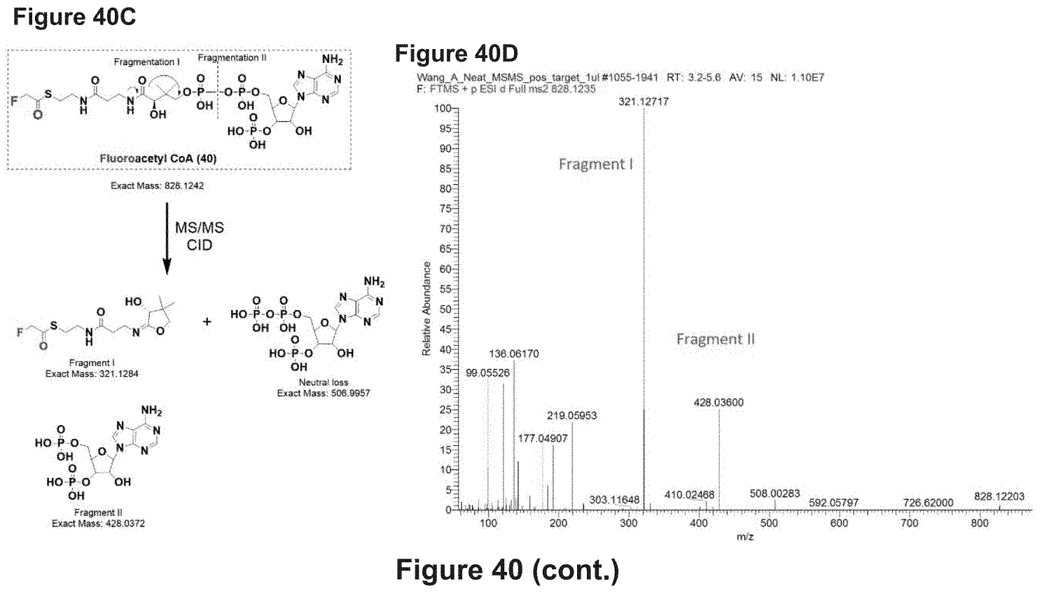

For labeling proteins in living cells, azide or alkyne analogs of fatty acids have been exploited, which were metabolized intracellularly into CoA derivatives (Grammel M et al., 2013, Nat. Chem. Biol., 9:475-484; Saxon E et al., 2000, Science, 287:2007-2010; Chuh K N et al., 2015, Curr. Opin. Chem. Biol., 24:27-37; Yang Y Y et al., 2010, J. Am. Chem. Soc., 132:3640-3641). To increase their cellular delivery, pro-metabolites with esters masking the polar carboxylate group constituted an effective strategy in recent years (Sinclair W R et al., 2018, Chem. Sci., 9:1236-1241). Nevertheless, the intrinsic sterics of these pro-metabolites resulted in varied and suboptimal labeling results (Yang Y Y et al., 2010, J. Am. Chem. Soc., 132:3640-3641; Sinclair W R et al., 2018, Chem. Sci., 9:1236-1241), sometimes requiring extensive structural optimization (Sinclair W R et al., 2018, Chem. Sci., 9:1236-1241). To explore the utility of the probing system for studying acetylation in the cellular level, the fluorinated version of pro-metabolite, ethyl fluoroacetate, was designed (FIG. 15A). Given its reported in vivo toxicity (Peters R A et al., 1952, Proceedings of the Royal Society of London. Series B—Biological Sciences, 139:143-170; Gribble, G W, 1973, J. Chem. Educ., 50:460-462), the cell cytotoxicity was first evaluated and it was found that this pro-metabolite exhibited minimal toxicity with doses up to 2 mM after 12 h of incubation (FIG. 16). Additional LC-MS/MS studies indeed confirmed its conversion by enzymes to fluoroacetyl-CoA in live cells (FIG. 40), which, taken together with the observed minimal toxicity in cell lines, supported the applicability of ethyl fluoroacetate and its CoA metabolite to studies in the cellular level.

With confidence in the safety profile of the pro-metabolite, studies started by treating it with two representative cell lines, HeLa, and HEK293, as the first step, followed by subsequent FTDR with the TAMRA-SH probe (15) as the second step for fluorescent detection (FIG. 15A). Treatment with azido modified pro-metabolite (Sinclair W R et al., 2018, Chem. Sci., 9:1236-1241) and the TAMRA-alkyne (16) for CuAAC chemistry was performed in parallel as a control, wherein weak signals were previously reported (Sinclair W R et al., 2018, Chem. Sci., 9:1236-1241). Direct microscope imaging studies (FIG. 15B) revealed much stronger intracellular labeling and tagging with TAMRA following the FTDR based approach, suggesting a drastically more complete profiling of acetylation substrates. Significant fluorescence was also observed not only in the nucleus but also cytoplasm, which may indicate the successful labeling of both histones and non-histone proteins. The little background signals emitted from the cells treated with only (15) (step 2) further demonstrated the specificity of the developed —SH probes. Following similar procedures, the labeling of the cell lysates was also tested after metabolic incorporation of fluorine reporters. Lysates following FTDR or CuAAC mediated ligation with TAMRA were separated on SDS-PAGE, and were visualized by CBB to confirm the equal amount of protein loading (FIG. 15C). Yet, multiple labelled protein bands spanning a wide range of molecular weights were observed only for the lysates of cells that underwent a complete two-step process of FTDR chemistry (FIG. 15C). This discovery was consistent with the microscope imaging results, and fully supported that FTDR allowed for profiling the proteome-wide substrates of KAT from the cellular contexts.

In conclusion, a fluorine-thiol displacement reaction was developed and used for steric-free labeling of protein substrates of a representative PTM, acetylation. Along with the benzenethiol derived functional tags, the FTDR-based imaging and detection of substrates demonstrated great potential for globally profiling KATs substrates, which are of vital significance for understanding the roles of acetylation in physiology and disease. This tool kit, together with future applications to quantitative proteomics studies, is expected to offer versatile probes for identifying targets of acetylation, and many other PTMs that are mediated by transferases with restricted active sites.

As disclosed herein, a novel bioorthogonal reaction that can selectively displace fluorine substitutions alpha to amide bonds was developed. This FTDR allows for fluorinated cofactors or precursors to be utilized as chemical reporters, hijacking acetyltransferase mediated acetylation both in vitro and in live cells, which cannot be achieved with azide- or alkyne-based chemical reporters. The fluorine labels can be further converted to biotin or fluorophore tags using FTDR, enabling the general detection and imaging of acetyl substrates. This strategy may lead to a steric-free labeling platform for substrate proteins, expanding the chemical toolbox for functional annotation of post-translational modifications (PTMs) in a systematical manner.

Thus, the herein disclosed protein labeling is steric free and can be used to probe a broad spectrum of enzymes, which cannot be achieved by existing labeling techniques (e.g., alkyne- or azide-based click chemistry tags that bulky in size).

The current data supported the application of this reaction for protein labeling. On the basis of this, additional experiments are conducted to develop new methods for peptide stapling (stapled peptides as therapeutics) and protein conjugation (antibody drug conjugates) with potential application as protein target profiling peptide stapling site-specific antibody drug conjugates.

### Example 2: FTDR Activity

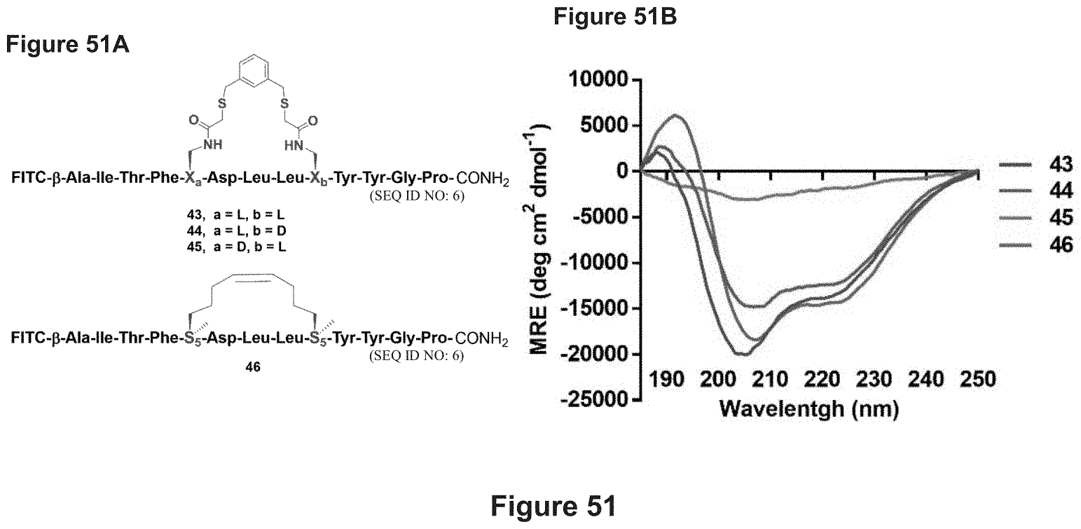

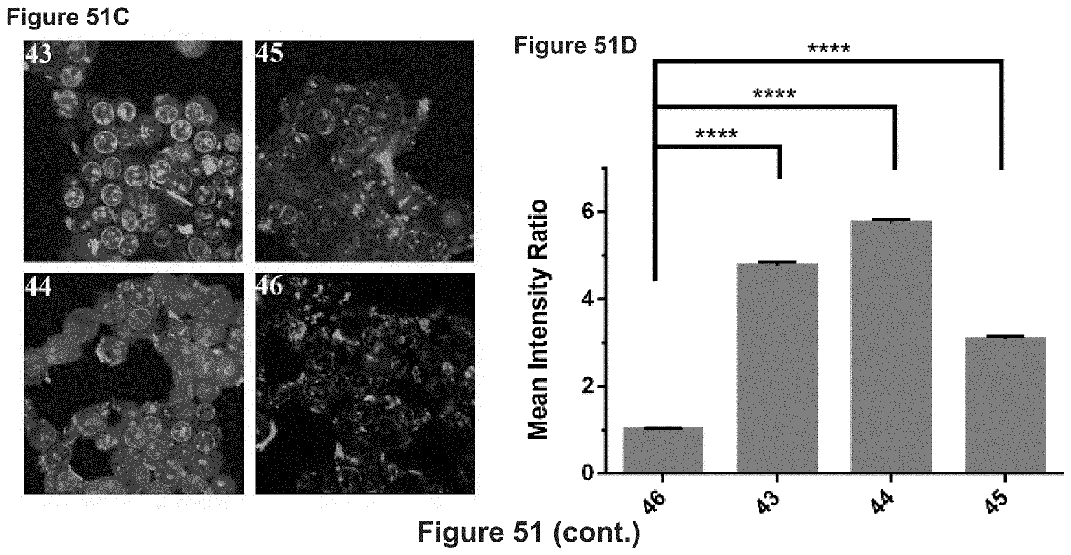

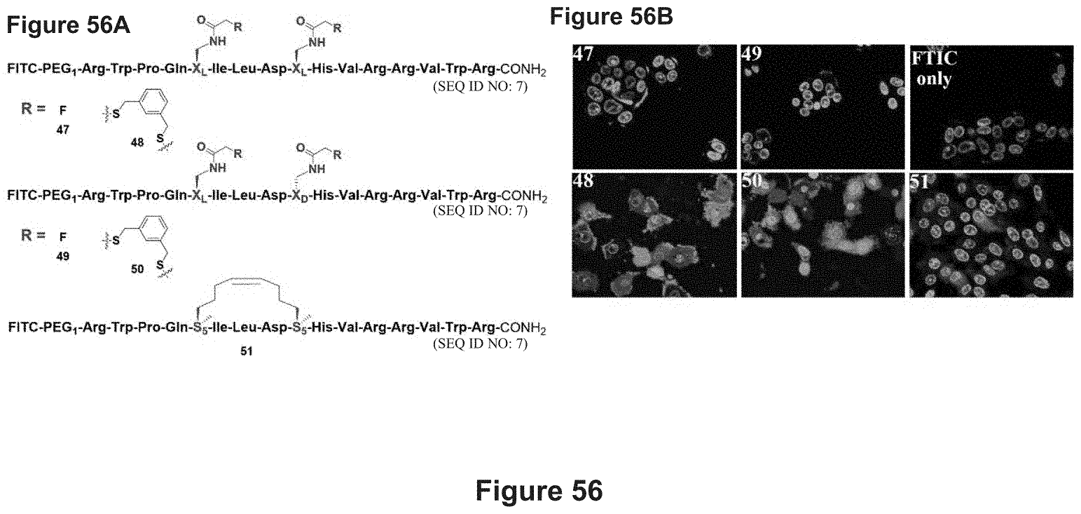

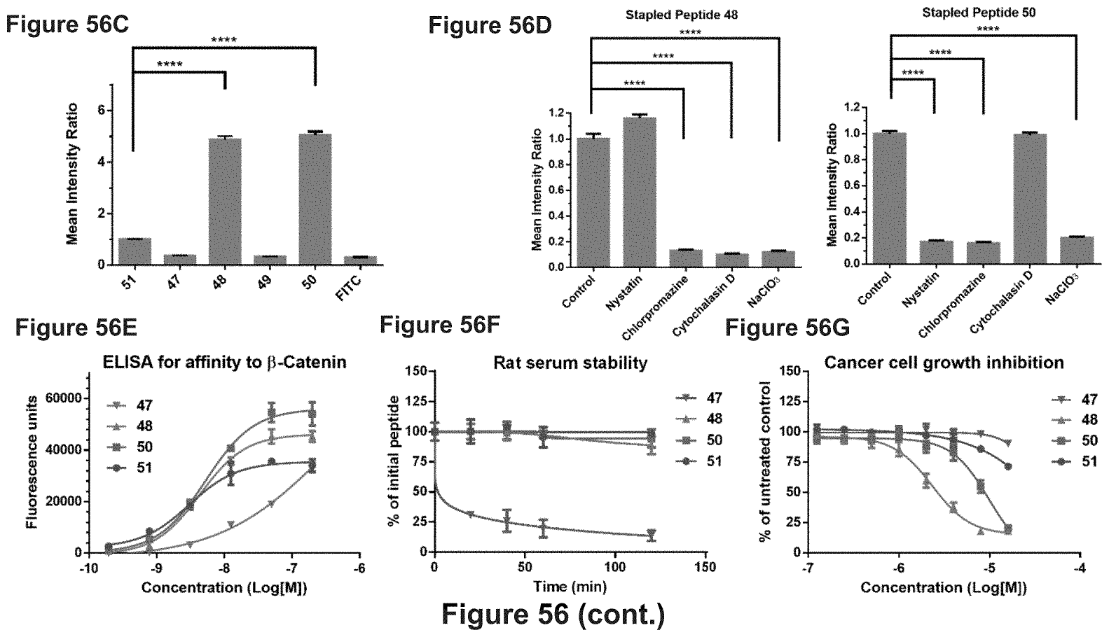

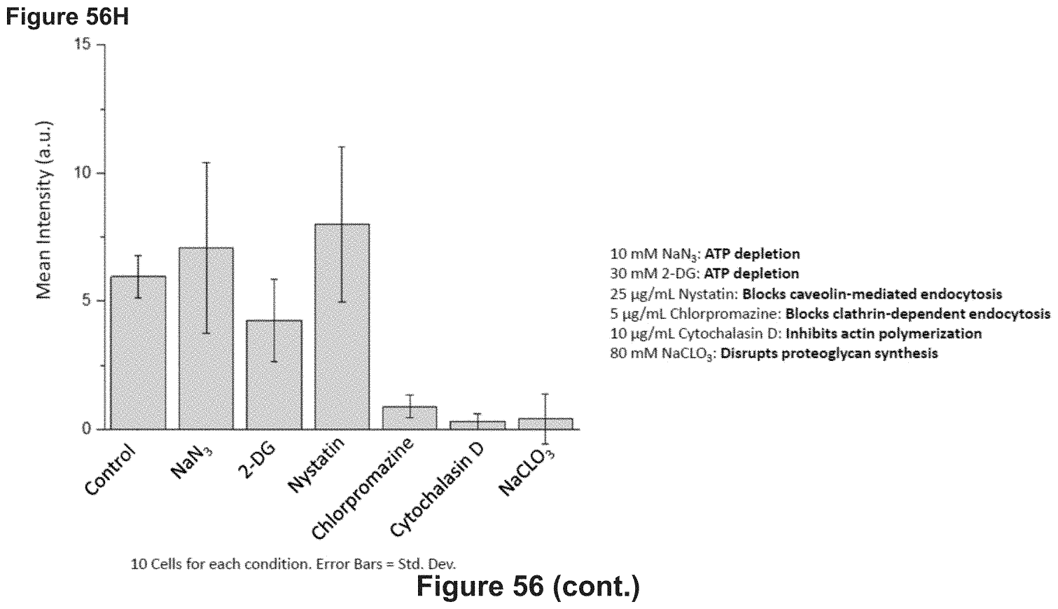

As shown in FIG. 51C, the peptides (43) and (44), which were stapled by the herein disclosed fluorine-thiol reaction, turned out to have much better cell permeability than the peptide (45) that is axin derived therapeutic peptide that has been stapled using known methods and were used in therapeutic studies for treating cancer cells relying on Wnt signaling pathways. The novel peptides (43) and (44) also have bound better to protein target Catenin than the known stapled peptide (43). Peptides (43) and (44) are the ones that were stapled based on the herein disclosed chemical method, which displayed better binding to beta-catenin and much enhanced cell penetration (FIG. 51C). The recent mechanism studies using small molecule modulators also revealed that the peptides stapled by the herein disclosed method (e.g., peptide (43)) permeates mammalian cells using a significantly different mechanism from other known stapled peptides (FIG. 56). It is known that commonly used stapled peptide enter cells through proteoglycan and can be knocked down by NaClO₃ (Chu Q. et al., 2015, Med. Chem. Commun., 6:111-119). However, the peptides stapled by the herein disclosed method actually enter cells via multiple pathways. As demonstrated in FIG. 56, the fluorine-thiol stapled peptides enter cells via ATP-dependent clarthrin-mediated endocytosis, actin polymerization, and proteoglycan, three independent pathways in total. Taken together, this finding explains why the herein disclosed stapled peptides possess significantly better cell permeability than known stapled peptides.

### Example 3: FTDR for Labeling Protein Substrates

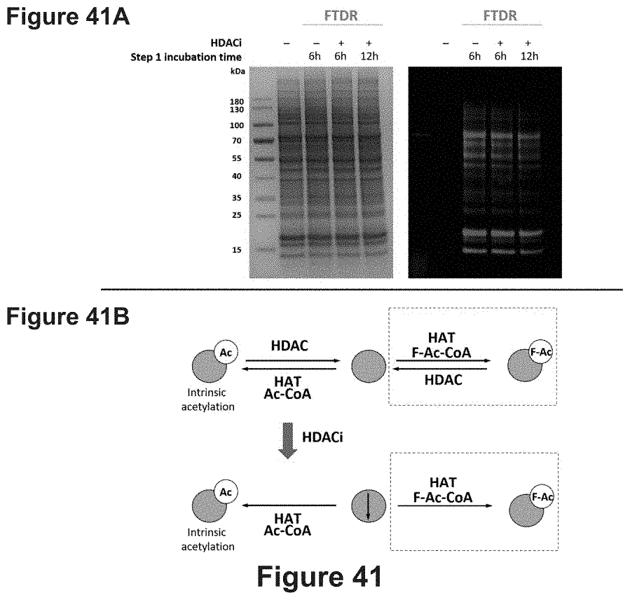

Furthermore, pretreatment of the cells with A-485 (potent and selective p300/CBP inhibitor; Lasko L M et al., 2017, Nature, 550:128-132; Zhang B et al., 2020, Biochem. Pharmacol., 175:113914) (FIG. 15C) before the two-step process of FTDR also resulted in weaker labeling intensity, which was consistent with the previously reported results using HATi (Yang Y Y et al., 2010, J. Am. Chem. Soc., 132:3640-3641; Sinclair W R et al., 2018, Chem. Sci., 9:1236-1241; Legartova S et al., 2013, Epigenomics, 5:379-396; Eliseeva E D et al., 2007, Mol. Cancer Ther., 6:2391-2398; Li S et al., 2019, J. Cell. Mol. Med., 23:2744-2752; Huang H et al., 2018, Mol. Cell, 70:663-678; Weinert B T et al., 2018, Cell, 174:231-244). In addition, the concurrent incubation with HDAC inhibitor cocktails was exploited and slightly decreased labeling was observed (FIG. 41), which was consistent to literature reports (Ourailidou M E et al., 2015, Org. Biomol. Chem., 13:3648-3653; Yang Y Y et al., 2010, J. Am. Chem. Soc., 132:3640-3641; Sinclair W R et al., 2018, Chem. Sci., 9:1236-1241), suggesting that incorporation of F-acetylation needs prior deacetylation of intrinsically acetylated lysine residues in order to make the lysine available. Thus, blocking the removal of wild-type acetylation prevents metabolic incorporation of F-acetylation (Ourailidou M E et al., 2015, Org. Biomol. Chem., 13:3648-3653; Yang Y Y et al., 2010, J. Am. Chem. Soc., 132:3640-3641; Sinclair W R et al., 2018, Chem. Sci., 9:1236-1241). Taken together, these observations fully supported that FTDR allowed for profiling the proteome-wide substrates of acetylation from the cellular contexts.

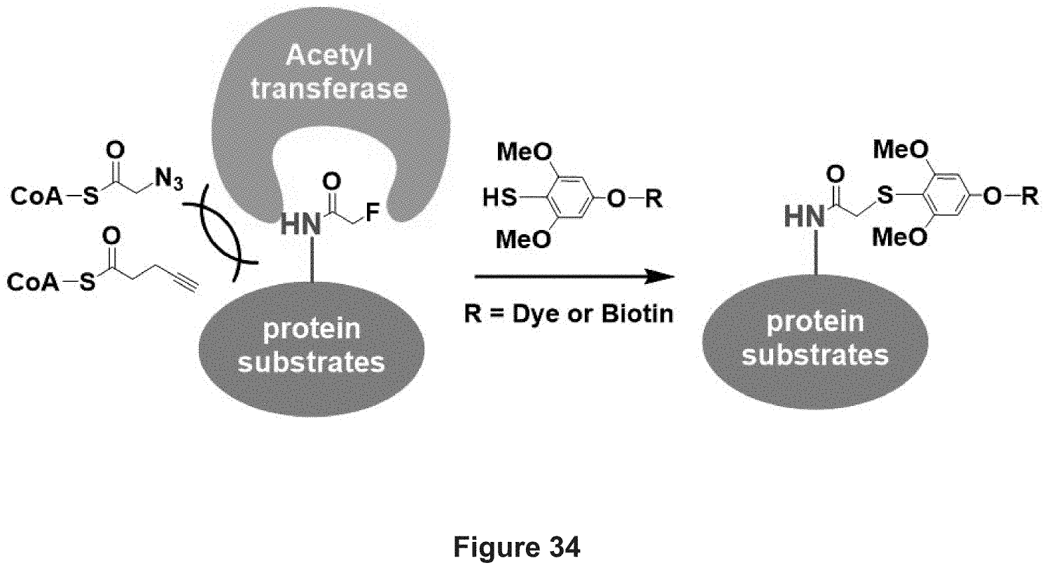

To further validate that the FTDR-based two-step metabolic labeling occurred on acetylation protein substrates, histone extraction were performed (FIG. 34A) and confirmed the existence of TAMRA labeling on the primary acetylation substrates, his-tones (FIG. 34B). As controls, both the treatment with HAT inhibitors and the competition with acetate have resulted in decreased TAMRA-SH probe labeling, suggesting that the FTDR-based two-step labeling is acetyltransferase-dependent, and relied on F-Ac-CoA metabolites. To test if the FTDR-based labeling can be used to enrich these protein substrates, cell lysates were treated with the Biotin-SH probe in step 2, and pulled down the labelled proteins (FIG. 34A). Western blot analysis confirmed the presence of known acetylation substrates, including histones, and alpha-tubulin (Dan W et al., 2018, Cereb. Cortex, 28:3332-3346; Liu N et al., 2015, Sci. Rep., 5:16869) only in the protein pool enriched from the cell lysates that have been subjected to the two-step process (FIG. 34C). Accordingly, pretreatment with HAT inhibitors decreased the amount of proteins enriched, particularly for H3 and H4. The level of alpha-tubulin was not significantly perturbed, likely due to the fact that certain acetylation sites, such as lysine 40 on alpha-tubulin, are mediated by other acetyltransferases (e.g. αTAT1) (Dan W et al., 2018, Cereb. Cortex, 28:3332-3346) that were not targeted by the administered HAT inhibitors.

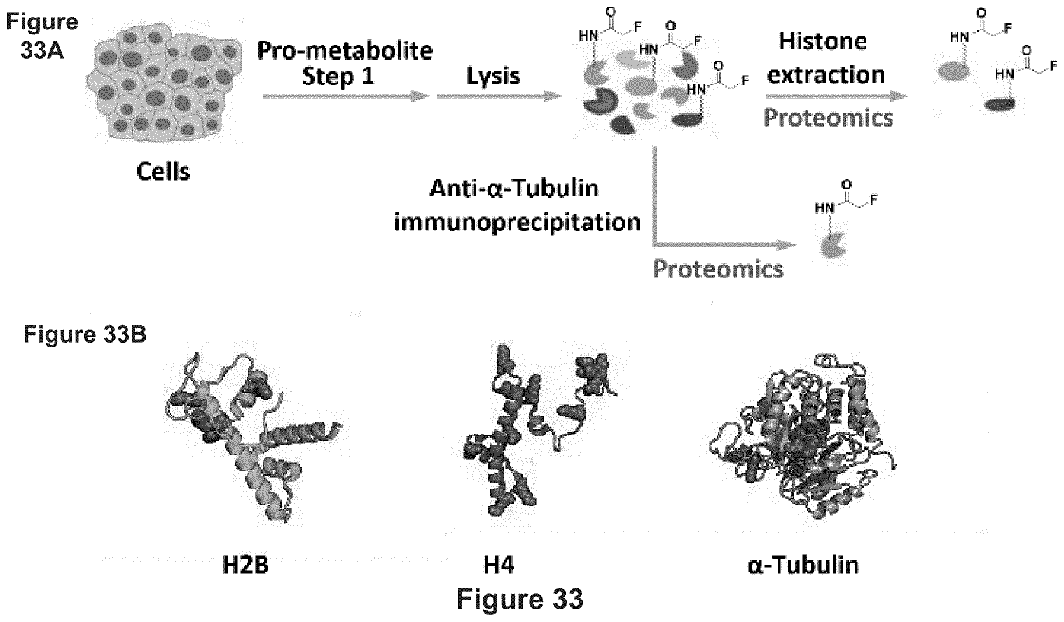

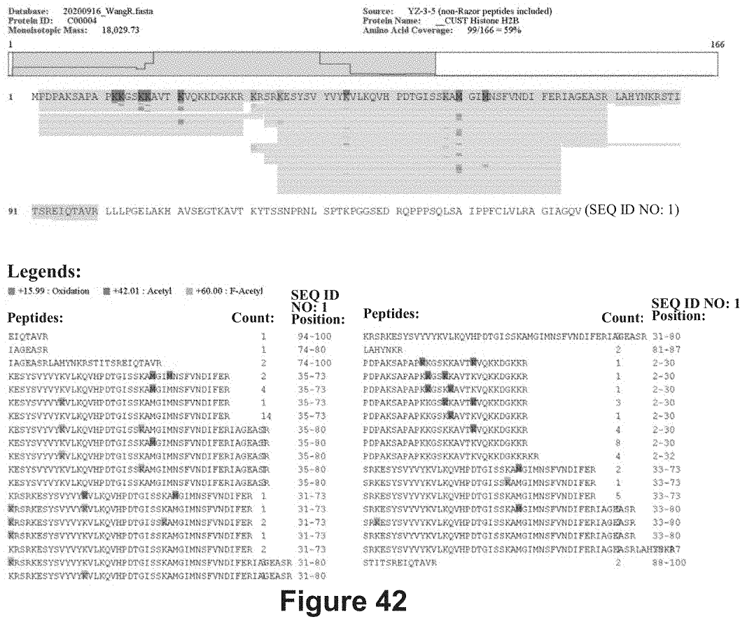

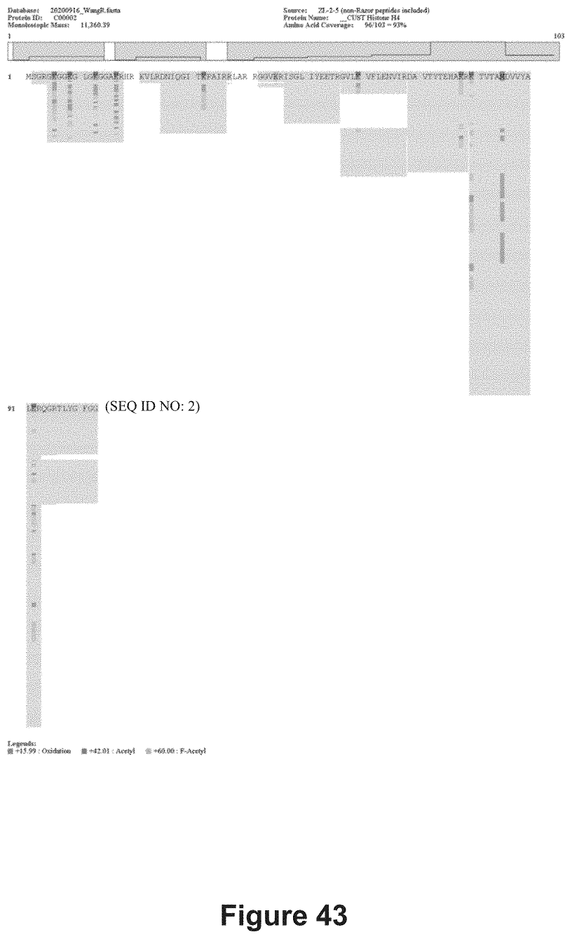

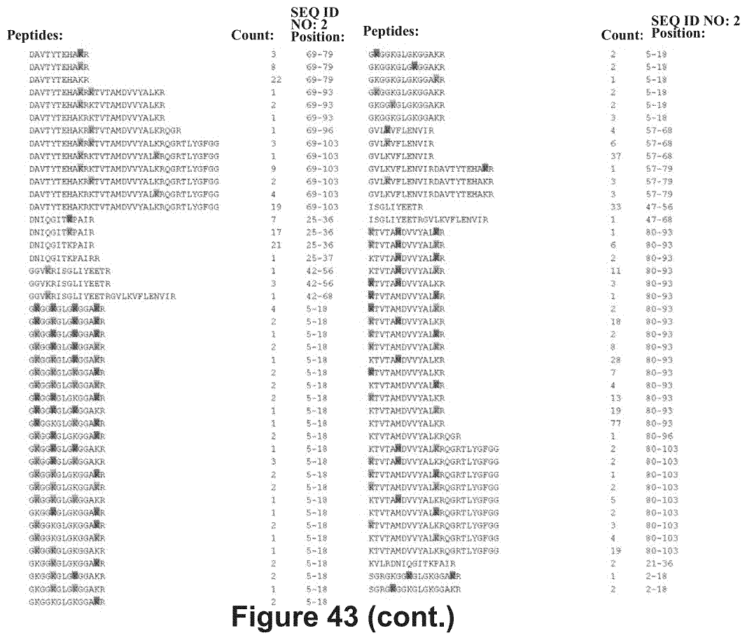

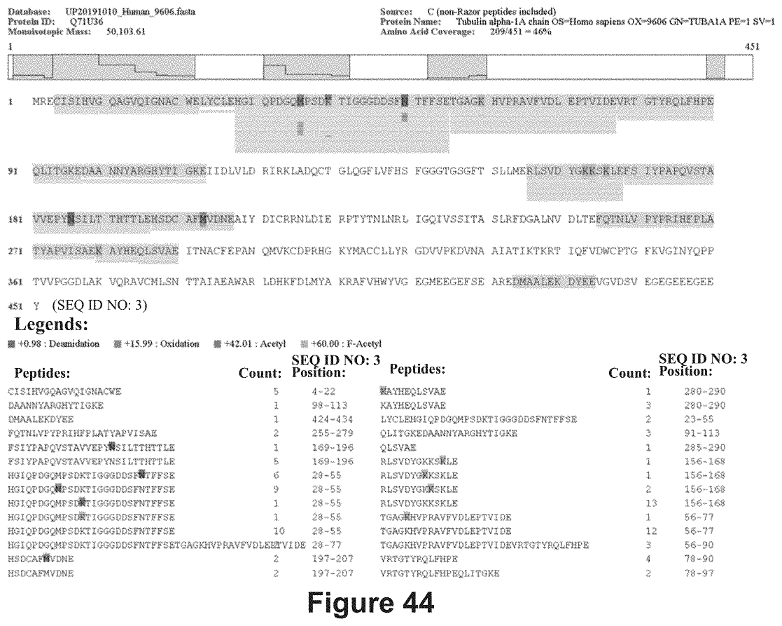

Lastly, to gain insight into the specific labeling sites by the present tagging strategy, histones were extracted out and alpha-tubulin was pulled down from the cells having incorporated the pro-metabolite ethyl fluoroacetate; and proteomics studies were carried out (FIG. 33A). As shown in FIG. 33B and FIG. 42 through FIG. 44, fluoroacetylation has been observed on lysine 30, lysine 34, lysine 43, and lysine 57 of histone H2B (SEQ ID NO: 1); lysine 5, lysine 8, lysine 12, lysine 16, lysine 31, lysine 44, lysine 59, lysine 77, lysine 79, and lysine 91 of histone H4 (SEQ ID NO: 2); lysine 40, lysine 60, lysine 163, lysine 164, lysine 166, and lysine 280 of alpha-tubulin (SEQ ID NO: 3). Almost all of these sites have been consistent with the previous literature reports on acetylation sites (Liu N et al., 2015, Sci. Rep., 5:16869; Hansen B K et al., 2019, Nat. Commun., 10:1055; Barnes C E et al., 2019, Essays Biochem., 63:97-107; Li D et al, 2019, Mol. Genet. Genomic. Med., 7:e1002), further demonstrating that this labeling strategy can probe lysine acetylation in a general manner. Notably, N-terminal acetylation has recently drawn significant research interest, which is catalyzed by a specific class of N-terminal acetyltransferases using Ac-CoA, and has been revealed to play important roles in protein functions and cellular locations, etc. (Ree R et al., 2018, Exp. Mol. Med., 50:1-13; Aksnes H et al., 2016, Trends Biochem. Sci., 41:746-760). During the preliminary proteomics studies of histone H2B and H4 (FIG. 42 and FIG. 43), no F-acetylation at the N-terminus was able to be observed. Although N-terminal acetylation is widespread and common in human proteins, N-terminal acetylation is considered to be irreversible (Ree R et al., 2018, Exp. Mol. Med., 50:1-13), which can block the incorporation of F-acetylation to the intrinsic N-terminus. Thus, the present FTDR-based labeling and imaging can be specifically applied to internal lysine sites.

In summary, a FTDR was developed and its use for the steric-free labeling of protein substrates of a representative PTM via acetylation was demonstrated (FIG. 34). This novel bioorthogonal reaction selectively displaced fluorine substitutions alpha to amide bonds and allowed for fluorinated cofactors or precursors to be utilized as chemical reporters; hijacking acetyltransferase mediated acetylation both in vitro and in live cells, which cannot be achieved with azide- or alkyne-based chemical reporters. The fluoroacetamide labels was further converted to biotin or fluorophore tags using FTDR, enabling the general detection and imaging of acetyl substrates. Along with the benzenethiol derived functional tags, the FTDR-based imaging and detection of substrates demonstrated great potential for globally profiling acetyl transferases' substrates, which are of vital significance for under-standing the roles of acetylation in physiology and disease. Thus, this strategy led to a steric-free labeling platform for substrate proteins, expanding the chemical toolbox for functional annotation of PTMs in a systematic manner. This tool kit, together with the applications to quantitative proteomics studies, offers versatile probes for identifying targets of acetylation, and many other PTMs that are mediated by transferases with restricted active sites.

The materials and methods employed in Examples 1 through 3 are now described.

General Information: Chemical reagents and solvents were purchased from commercial resources such as VWR, Thermo Fisher, and Sigma Aldrich, and were used directly without further purification. Analytical TLC was carried out with Silica Gel 60 F254 plates (EMD Chemicals). The chemicals on TLC were either visualized by UV 254 nm (UV lamp, Chemglass Life Sciences) or stained by phosphomolybdic acid or KMnO₄ oxidation. Compound purification was performed by normal-phase flash column chromatography on columns manually loaded with silica gel grade 60 (230-400 mesh, Fisher Scientific) or by reverse-phase Combi-Flash on prepacked C18 columns (Teledyne ISCO). Further purification by preparative high-performance liquid chromatography (HPLC) was implemented on Waters 1525 series that consist of a 2489 UV/vis detector, 1525 binary pump, and an XBridge Prep C18 column. Routine mass spectrometry analysis was done using liquid chromatography-mass spectrometry (LC-MS) Agilent 1100 series. High resolution LC-MS analysis was performed on an Agilent 6520 Accurate-Mass Quadrupole-Time-of-Flight (Q-TOF) coupled with an electrospray ionization source. For NMR analysis, ¹H NMR and ¹³C NMR spectra were recorded on 400 MHz or 500 MHz Bruker Advance. The raw data were processed with MestReNova, and the chemical shifts were reported in parts per million (ppm) downfield from the internal standard tetramethylsilane (TMS).

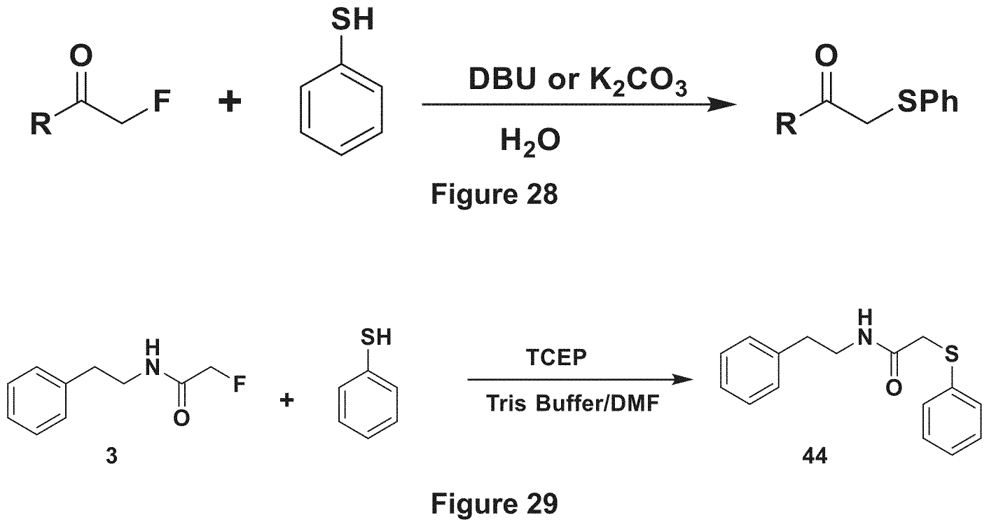

General Procedure A (FIG. 28): The nucleophile thiophenol (0.4 mmol, 44 mg) was mixed with 0.2 mmol α-fluorocarbonyl derivative (compounds (1)-(4)) in 1 mL water. Then 0.6 mmol DBU (91 mg) or 0.4 mmol potassium carbonate (55 mg) was added to the reaction mixture. After stirring at room temperature with indicated time, 5 mL ethyl acetate was added to the flask to quench the reaction. The organic layer was separated, dried with anhydrous sodium sulfate, vacuum concentrated, and subsequently purified via flash column chromatography to provide the desired thiophenol adduct (compounds (42)-(45)).

Optimizing Reaction Conditions—pH Titration (FIG. 29): Substrate (3), 2-fluoro-N-phenethylacetamide, was dissolved in DMF to make a 1 M stock solution. About 0.75 μL of it was mixed with 5 μL of thiophenol stock (300 mM in DMF), and 2 μL of TCEP solution (1.5 M stock in water, pH 7.4). DMF/Tris buffer were added to make a total volume of 30 μL (50% of Tris buffer), during which 2 M HCl or NaOH was slightly added to adjust the final pH value to 6.5, 7.5, or 8.5. Thus, the final concentration of substrate (3), thiophenol, and TCEP was 25 mM, 50 mM, and 100 mM, respectively. The mixture was reacted at 37° C. Approximately 3 μL of the reaction mixture was taken out at indicated time points (12 h) and was mixed with 30 μL 0.5% TFA/ACN to quench the reaction. The samples were analyzed by LC-MS. To reduce the inter-assay variations and errors, the yield of product (44) was determined by comparing the UV peak area ratio of the (product)/(the product+unreacted substrate) in each LC-MS assay with the standard curve. The standard curve was plotted by the known concentration ratios of “[the product]/([the product]+[substrate])” against the corresponding UV peak area ratios. The denominator of the equation equals the very initial concentration of the substrate (25 mM for the current reaction).

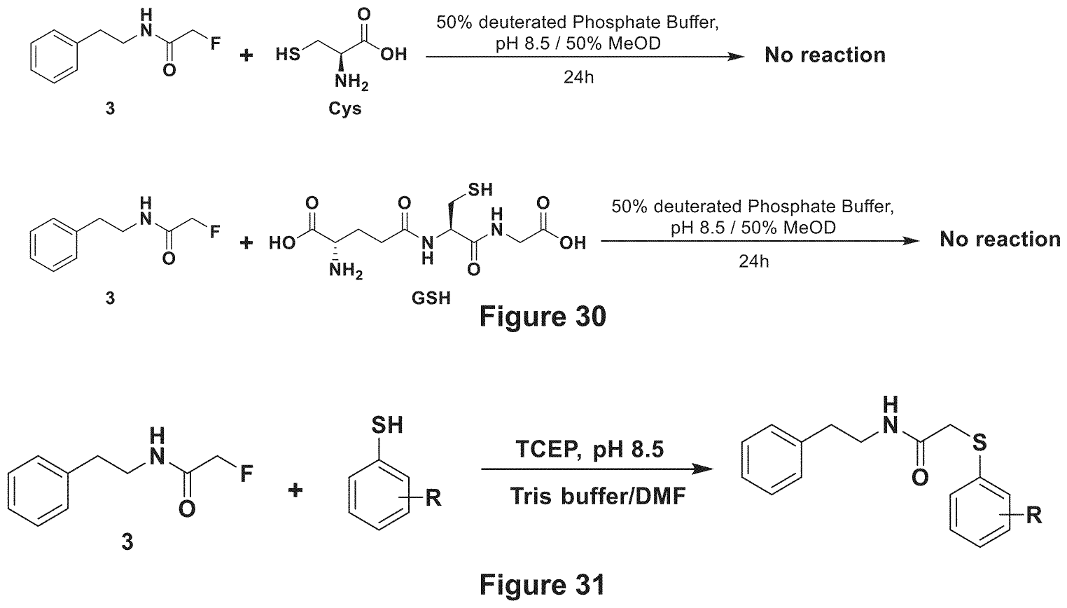

Bioorthogonality of the Fluorine-Thiol Displacement Reaction (FIG. 30) The common substrate 2-fluoro-N-phenethylacetamide (compound (3)) was dissolved in MeOD as a 10× stock solution (250 mM). Sixty microliter of the stock was mixed with another 60 μL of either the reduced glutathione or cysteine 10× stock solution (250 mM in D₂O). The mixture was added with additional deuterated solvents (1:1 mix of deuterated sodium phosphate buffer and MeOD) to make a final volume of 600 μL (pH adjusted to 8.5). The resulting solution was incubated at 37° C. water bath for 24 h, and then analyzed by ¹H NMR spectroscopy.

General Procedure B (FIG. 31): 2-Fluoro-N-phenethylacetamide (3) (25 mM, 5 μL 0.5 M stock in DMF) and substituted benzenethiol (50 mM, 5 μL 1 M stock in DMF) were dissolved in 40 μL DMF and 43 μL Tris buffer (50 mM, pH 8.5). Reducing reagent TCEP (100 mM, 5 μL 2 M stock in water) was added to the mixture and the final pH value was adjusted to 8.5 by adding 2 μL 6M NaOH solution. The reaction mixture was incubated at 37° C. At indicated time points, 5 μL of the reaction mixture was taken out and mixed with 30 μL 0.5% TFA/CH₃CN that was expected to quench the reaction. The sample was analyzed by LC/MS, and the relative product yield was determined the same as mentioned in the section of pH titration.

Measurement of Reaction Kinetics: Reaction kinetics were evaluated similarly to reported procedures (Addy P S et al., 2017, J. Am. Chem. Soc., 139:11670-11673; Sundhoro M et al., 2017, Angew. Chem. Int. Ed. Engl., 56:12117-12121.). Stock solutions of substrate (3) and nucleophiles were prepared in DMF, while TCEP stock (pH 7.4) was dissolved in H₂O. Equal concentrations (40 mM, 80 mM, or 160 mM) of the substrate and the nucleophile (3,4,5-trimethoxybenzenethiol or 2,4,6-trimethoxybenzenethiol) were mixed in 40 μL of DMF/Tris buffer (70/30). Over excess amount of TCEP (100 mM, 200 mM, or 400 mM) was added, and the final pH of the mixture was adjusted to 8.5 to initiate the reaction at 37° C. Time dependent measurements were carried out by taking 2 μL of the reaction mixture at indicated time points (30 min, 60 min, 90 min, 120 min, 150 min, 180 min), and mixing it with 18 μL 0.5% TFA/ACN to quench the reaction. The samples were eventually analyzed by LC/MS and the concentrations of product and reactant were determined by comparing peak area ratios with those of the standard curves. Plotting 1/[X]t against time yielded the desired rate constant (k), based on the second order rate equation “1/[X]ₜ=1/[X]₀+kt” ([X]₀: initial concentration of either reactant; t: reaction time; [X]ₜ: concentration of either reactant at time t).

Antibody-Based Global Profiling of Acetylation: HEK293 cells (American Type Culture Collection) were maintained in 10 cm cell culture dishes with DMEM media and 10% FBS, 100 IU/mL penicillin, and 100 μg/mL streptomycin. Deacetylase inhibitor cocktail (100×, APExBIO) was added to the media overnight, after which the cells were washed with PBS and lysed in the CelLytic M buffer (Sigma Aldrich) that was pre-mixed with protease inhibitor cocktail (EDTA-free, Roche) and the deacetylase inhibitor cocktail (APExBIO). The cell mixture was sonicated for 18 sec (3 sec on, 7 sec off, 20% amplitude) on ice, and subsequently centrifuged at 15,000 rpm at 4° C. for 10 min. The resulting supernatants were collected and the protein concentration was determined by BCA assay (Pierce, Thermo Fisher) to be ˜3 mg/mL. Approximately 50 μg of cell lysates were loaded for each lane and separated by 4-12% Bis-Tris SDS PAGE (Thermo Fisher), which were then transferred to PVDF membranes using a semi-dry blotting apparatus (Bio-Rad). Each lane on the membrane was carefully cut and blocked with 3% BSA in TBST (with 0.1% Tween-20) for 1 h. The piece of the membrane that contained one sample lane was then incubated with a specific anti-acetyl lysine antibody (from Ab21623, Ab190479, Ab80178, Ab61257, Abcam; or CST9441, CST9814, Cell Signaling Technology) overnight at 4° C., followed by washings and subsequent incubation with the IRDye® 680RD secondary antibody (Li-Cor).7 After extensive washing, protein bands were detected via near-infrared fluorescence on the LI-COR Odyssey FC Imaging System (700 nm channel scanning).

Enzymatic Peptide Substrate Modifications with Fluorine: The PCAF assay cocktail was prepared by mixing 4 μL of 5×histone acetyltransferase (HAT) assay buffer (250 mM Tris-HCl, pH 8.0, 0.5 mM EDTA, 5 mM DTT), 1 μL of 2 mM histone H3-20 peptide (AnaSpec), 4.7 μL of 2.1 mM acetyl-CoA (Fisher Scientific) or acetyl CoA analogs, and 5.3 μL H2O, in a total volume of 15 μL. After 5 μL of 1.3 μM PCAF enzyme (Cayman Chemical) was added, the reaction mixture was incubated at 30° C. for 3 h. The sample was then subjected to high resolution LC-MS analysis.

The MYST2 assay cocktail was prepared by mixing 4 μL of 5×HAT assay buffer, 0.5 μL of 2 mM histone H4-20 peptide solution (AnaSpec), 4.7 μL of 2.1 mM acetyl-CoA (Fisher Scientific) or acetyl CoA analogs, and 0.8 μL H₂O, at a total volume of 10 μL. After the addition of 10 μL 0.9 μM KAT7 enzyme (SignalChem), the reaction mixture was incubated at 30° C. for 3 h. The sample was then subjected to high resolution LC-MS analysis.

The TIP60 assay cocktail was prepared by mixing 0.5 μL of 2 mM histone H4-20 peptide (AnaSpec), 4.7 μL of 2.1 mM acetyl-CoA or related analogs, and 2.5 μL Tris buffer (50 mM, pH 8.0). Approximately 2.3 μL of 4.3 μM Tip60 (Cayman Chemical) in the stock buffer (50 mM Tris-HCl, pH 7.5, 100 mM NaCl, 10% glycerol) was then added and the reaction mixture was incubated at 30° C. for 5 h. The sample was finally analyzed by high resolution LC-MS.

Hydrolysis of Acetyl CoA and Fluoroacetyl CoA: The hydrolysis rate of acetyl-CoA and fluoroacetyl CoA were measured in a similar manner to reported procedures (Weeks A M et al., 2010, Biochemistry, 49:9269-9279; Wagner G R et al., 2017, Cell Metab., 25:823-837). Briefly, 10 μM of the acetyl or fluoroacetyl CoA was incubated in 100 mM Tris buffer mixed with 0.5 mM DTNB, pH 7.2. The increase in the solution's absorbance at 412 nm was recorded, as a result of the reaction between DTNB and the free thiol in the released CoA hydrolysis product. A CoA standard curve was generated by measuring the absorbance of serially diluted CoA stock (200 μM) in the same assay buffer.

Non-Enzymatic Acetylation on Bovine Serum Albumin: Following the reported procedures for acyl-CoAs (Wagner G R et al., 2017, Cell Metab., 25:823-837), bovine serum albumin as a model protein (1 mg/mL) was dissolved in 50 mM HEPES and 150 mM NaCl, pH 8.0 or 7.0. For western blot analysis, acetyl-CoA or fluoroacetyl CoA at the desired final concentrations, the buffer as a negative control, or the Sulfo-NHS-acetate (Pierce, Thermo Fisher) as a positive control were added to separate solutions, with the final pH adjusted to pH 8.0 or 7.0. For FTDR-based detection, the aforementioned reagents, the buffer as a negative control, or the NHS-fluoroacetate as a positive control were added to separate solutions of bovine serum albumin. Next, all the reaction mixtures were incubated at 37° C. for 6 h. The reaction samples (2 μL each) were then separated by SDS-PAGE, transferred to PVDF membrane, blocked and washed the same way as the aforementioned anti-acetylation western blot assays. The blot was probed with the MultiMab™ antibody (Ac-K-100, CST9814, Cell Signaling) that is comprised of mixed monoclonal antibodies for recognition of both acetyl-lysine and F-acetyl lysine. For FTDR detection, 20 μL of each reaction sample was further treated with 5 mM TAMRA-SH and 10 mM TCEP, and was incubated at pH 8.5 for 8 h. After SDS-PAGE separation of 2 μg of each sample, in-gel fluorescence detection was achieved on the LI-COR Odyssey FC imager (600 nm channel).

Stability and Reactivity of Model Substrates and Probes in Cell Lysates: The cell lysate experiments were performed similarly to those reported for other previously established bio-orthogonal reactions (Blackman M L et al., 2008, J. Am. Chem. Soc., 130:13518-13519). In general, 5 μL of substrate compound 3, 3-Cl, or the probe 13 mixed with their corresponding internal standards (3′ or 13′) in DMSO (10 mM stock concentration) were added to 25 μL HEK293 cell lysates (˜3 mg/mL protein concentration). TCEP (5 μL, 60 mM stock concentration) was added to each reaction group (A, B, or C, respectively, FIG. 36A) to maintain a reducing environment. The final reaction pH was adjusted to 8.5, and water was added to make a final volume of 50 μL. After incubation at 37° C. for 14 h, the reaction mixture's pH was adjusted to ˜6.

The solution was extracted with ethyl acetate three times and concentrated in vacuo. The resulting residues were dissolved in methanol and analyzed by high resolution LC-MS (Wistar Institute) on a ThermoFisher Scientific Q Exactive HF-X mass spectrometer equipped with a HESIII probe and coupled to a ThermoFisher Scientific Vanquish Horizon UHPLC system. Compounds were separated on a Synergi™ Polar RP column (4 μm, 150×1 mm, Phenomenex). After LC-MS analysis, the peak area for each compound was integrated, and the percent recovery yield was calculated versus the internal standard. The FTDR reaction in cell lysate (FIG. 37) was carried out using the same procedure except that the incubation time was 5 h, and the extracts were analyzed by LC-MS/MS analysis.

Biotinylation of Labelled Peptides Based on the FTDR: The histone peptide H3-20 that previously underwent fluoroacetylation was lyophilized and resuspended in water (˜400 μM). About 1 μL of this stock solution was mixed with 1 μL Tris buffer (1M) and 4 μL H₂O. Then, 1 μL of the Biotin-SH probe stock in DMF (40 mM) was added, along with 1 μL of 50 mM TCEP aqueous solution. After adjusting the pH to 8.5 (with 2 μL of 1 M NaOH solution), the final concentrations of H3-20 substrate, Biotin-SH probe, and TCEP were 40 μM, 4 mM, and 5 mM, respectively. The reaction was incubated at 37° C. overnight, and the sample was subjected to high resolution LC-MS analysis.

Expression, Purification and Characterization of EZH2 Protein: The plasmid expressing GST-fused EZH2 N-terminal domain (1-500) was constructed based on the parent plasmid pGEX-EZH2 that was a gift from Prof. Min-Chie Hung (Addgene plasmid #28060) (Wei Y et al., 2011, Nat. Cell. Biol., 13:87-94). The C-terminal sequence (501-745) was deleted using Q5® Site-Directed Mutagenesis Kit (New England Biolabs) and customized primers (Integrated DNA Technologies). The sequences of the resulting pGEX expression vector were confirmed by DNA sequencing (GENEWIZ). The final vector was transformed into BL21 competent cells via electroporation. The resulting cells were recovered in SOC medium and plated onto an ampicillin-containing LB agar plate. After overnight incubation at 37° C., the colonies were picked, amplified and mixed with 50% glycerol as cell stock for EZH2 (1-500) protein expression. On the day of expression, BL21 stock was inoculated in LB medium supplemented with 100 μg/mL ampicillin, and shaken at 250 rpm, 37° C. (MaxQ 8000, Thermo Scientific). When OD600 reached 0.8, 0.5 mM IPTG was added to induce protein expression. The cell culture was left for ˜12 h at 25° C., 250 rpm (MaxQ 8000, Thermo Scientific). Cells were then harvested and frozen at −80° C. overnight.

The bacteria pellets were lysed in lysis buffer (50 mM Tris-HCl, pH 8.0, 150 mM NaCl, 2 mM EDTA, 20% sucrose, 0.2% TritonX-100, and 5% glycerol) that also contained 1 mg/mL lysozyme (VWR) and protease inhibitor cocktail (Roche cOmplete). After shaking at 250 rpm, 25° C. for 1.5 h, the cell lysate were centrifuged at 9000 rpm for 40 min (FX6100 Rotor, Beckman Coulter), and was filtered through a 0.2 μm filter to remove debris. The filtrate was passed through affinity columns packed with glutathione resin (GenScript), which was later washed with 20 mL PBS buffer (0.02% tween-20). Finally, 15 mL of elution buffer (10 mM glutathione, 50 mM Tris-HCl, pH 8.0) was added, and the eluted EZH2 (1-500) protein was buffer exchanged into storage buffer (50 mM Tris-HCl, pH 8.0, 10% glycerol, 1 mM DTT, 0.1 mM EDTA) by Amicon® Ultra Centrifugal Filters (MilliporeSigma). The protein purity and identity was confirmed by SDS-PAGE.

The fluorination and biotinylation on EZH2 protein were performed following the aforementioned procedures (acetyltransferase assay and FTDR using Biotin-SH probe) for histone substrates. The acetyltransferase used for EZH2 was PCAF (Cayman Chemical). To prepare the positive control for CuAAC (“C”), azidoacetic acid NHS ester (N3-NHS) was synthesized according to published procedures (Loka R S et al., 2010, Bioconjugate Chem., 21:1842-1849). Briefly, 30 μL of EZH2 protein (0.65 mg/mL in DPBS with 0.25 mM TCEP added, pH 7.2) was first reacted with 2.5 μL of N3-NHS (1 mM, DPBS buffer, pH 7.2) at room temperature for 1 h. To the resulting EZH2 mixture with lysines conjugated by azide, 1 μL of THPTA (10 mM), 1 μL of sodium ascorbate (50 mM), 0.5 μL of biotin-alkyne linker (5 mM), and 1 μL of CuSO4 (5 mM) were added to initiate the CuAAC-based modification of EZH2 by biotin. The reaction mixture was incubated at room temperature for 2 h, before loading to SDS PAGE for analysis in parallel with other samples.

Fluorination and Biotinylation of Known Protein Substrates by Acetyltransferase Assay and FTDR: The protein substrates of acetylation such as human histone H3.1 (New England Biolabs), histone H4 (New England Biolabs), or EZH2 (1-500) was dissolved in the aforementioned HAT buffer at a final concentration of 15 μM, and was mixed with acetyl-CoA analogs (450 μM) and the corresponding acetyl transferase (1.3 μM) (PCAF (Cayman Chemical) for histone H3.1 and EZH2; MYST2 (SignalChem) for histone H4). The reaction mixture was incubated at 30° C., pH 7.2 for 6 h, and was quenched by lyophilization.

For biotinylation, the fluorinated protein mixture was redissolved in water (final concentration ˜35 μM), added with 1.75 mM Biotin-SH probe and 2 mM TCEP. The pH was adjusted to 8.5 and the mixture was incubated at 37° C. for indicated time period (1 h, 3 h, or 6 h). Samples after this reaction were then loaded on gels for SDS-PAGE analysis. The control protein mixture (previously treated with 4PY-CoA) was reacted with biotin-azide probe (Thermo Fisher) following the reported copper-catalyzed azide-alkyne cycloaddition (CuAAC) procedures (Yang C et al., 2013, J. Am. Chem. Soc., 135:7791-7794), and was analyzed by SDS-PAGE in parallel to other samples.

For in-gel fluorescent imaging, the separated proteins on the PAGE gels were fixed with 50% isopropanol/45% water/5% acetic acid for 15 min at RT. The gel was washed, and incubated for 1 h at RT in biotin-free Casein blocking buffer (Sigma Aldrich). The blocked gel was then probed with streptavidin-IRDye 680RD (LI-COR) at a dilution of 1/2000 for 45 min at RT, followed by multiple washings with PBST (containing 0.1% Tween-20). Those biotinylated proteins were finally visualized by near-infrared fluorescence detection through the LI-COR Odyssey FC Imaging System (700 nm channel scanning, Ex 685 nm/Em 730 nm).

Removal of Fluoroacetylation on Protein Substrates by Histone Deacetylases: A 5×histone deacetylase assay buffer was prepared, containing 250 mM Tris-HCl (pH 8.0), 685 mM NaCl, 5 mM MgCl₂, 13.5 mM KCl, and 5 mM DTT. The fluoroacetylated histone substrate (˜20 μM final concentration) was mixed with the corresponding histone deacetylase (SIRT1 (R&D Systems) for fluoroacetylated H3; HDAC1, HDAC2, HDAC3, or SIRT2 (BPS Bioscience) for fluoroacetylated H4), and 2 μL of the 5×assay buffer. For assays involving sirtuins, nicotinamide adenine dinucleotide (NAD+, Fisher Scientific) was added at a final concentration of 1 mM. The reaction mixture's pH was adjusted to 8.0, with water added to a final volume of 10 μL. The mixture was incubated at 37° C. for 6 h, and subsequently treated with Biotin-SH to initiate FTDR reaction on any remaining fluoroacetylation. All the samples were finally separated on SDS-PAGE, transferred to PVDF membranes, and probed by streptavidin-IRDye 680RD (LI-COR) as mentioned before.

Cell Cytotoxicity Studies of Cofactor Analogs: HeLa and HEK293 cell lines were obtained from American Type Culture Collection (ATCC), and were cultured in DMEM medium supplemented with 10% FBS, 100 IU/mL penicillin, and 100 μg/mL streptomycin. Both cells were maintained in a cell-culture incubator at 37° C., with 5% CO2. On the night before the assay, these cell lines were plated into 96-well cell culture plates (white, flat bottom, Costar) at 5,000 cells, 90 μL media per well. The next day, cofactor analogs (azido-ethyl acetate, fluoro-ethyl acetate, or DMSO carrier control) were serially diluted in culture media as 10× stock solutions, and then added to the pre-plated cells at 10 μL/well. The final treatment concentrations are 62.5, 125, 250, 500, 1000, and 2000 μM. After thorough mixing, the samples were incubated in the cell culture incubator for 12 h at 37° C., under 5% CO₂. At the end of the treatment, the plates were taken out and cooled down to room temperature. Cell viability was measured by CellTiter Glo assay (Promega) following the published procedure (Lyu Z et al., 2018, ACS Chem. Biol., 13:958-964). Data were processed and plotted using Graphpad Prism (GraphPad Software). Viability of the cells treated with DMSO control was adopted as 100% viability control.

Cellular Metabolism Study of Ethyl Fluoroacetate: HEK293 cells were cultured in a 100 mm×20 mm tissue culture dish (Corning Costar) up to ˜80% confluence. The pro-metabolite ethyl fluoroacetate was added at a final concentration of 1 mM, and the culture was incubated at 37° C. for 2 h. The acyl-CoA extraction was performed following the reported procedures. Briefly, the cells were gently scraped down into the media, and spun down at 1,000 g. The cells were resuspended in 1 mL ice-cold extraction solution (10% trichloroacetic acid in milliQ water), and sonicated on ice for 30 sec (1 pulse per sec). The resulting cell lysate was centrifuged at 15,000 g, 4° C. for 5 min to precipitate the debris. Meanwhile, Oasis HLB SPE columns (Waters) were conditioned by 1 mL methanol and equilibrated with 1 mL water. The supernatant of the cell lysate was loaded to the column, followed by subsequent washing of the column with water. The potential CoA extracts were eluted by 0.5 mL elution solution (25 mM ammonium acetate in methanol), dried in vacuo, and resuspended in 50 uL of 5% 5-sulfosalicyilic acid for LC-MS/MS analysis. As a control, the HEK293 cell lysate was heated at 90° C. for 10 min, and then mixed with 1 mM ethyl fluoroacetate at 37° C. for 2 h. The follow-up extraction and LC-MS/MS analysis were carried out the same as mentioned above.

Intracellular Fluorination of Protein Substrates: HeLa or HEK293 cells were seeded at a density of 20,000 cells/well in a 12-well flat bottom cell culture plate (Corning Costar). When they reached ˜80% confluence, cells were incubated with 1 mM ethyl azidoacetate (for “click chemistry”), ethyl fluoroacetate (for fluorination), or DMSO control at 37° C. in standard medium for 6 h, similar to reported procedures (Sinclair W R et al., 2018, Chem. Sci., 9:1236-1241). The cells were then ready for follow-up intracellular imaging.

Intracellular Dye Labeling and Imaging of Protein Substrates: After incubation with azido- or fluoro-modified ethyl acetate precursors, cells were rinsed for three times with DPBS buffer, and fixed with 3.2% paraformaldehyde for 10 min at RT. With another around of rinses, cells were permeabilized with 0.1% Triton-100 in PBS buffer for 10 min at RT, after which point the intracellular proteins became ready for labeling by TAMRA dyes.

The “click chemistry” labeling on control groups was performed according to published procedures (Soriano Del Amo D et al., 2010, J. Am. Chem. Soc., 132:16893-16899). Cells previously treated with azidoacetate or DMSO were incubated with 100 μM TAMRA-alkyne probe (compound (16)) in the standard medium that also contained 200 μM CuSO₄, 500 μM BTTES, and 2.5 mM freshly prepared sodium ascorbate. The mixture was reacted at 37° C. for 1 h, followed by PBS washing, and a subsequent nucleus staining with 1 μg/mL Hoechst 33342 (Fisher Scientific). For fluorine-thiol displacement labeling, cells previously treated with fluoroacetate or DMSO were washed twice with PBS buffer (pH 8.0) that contained 5 mM TCEP. Each round of washing consisted of a 10 min incubation period. The cells were then incubated with 1 mM TAMRA-SH probe (compound (15)), and 5 mM TCEP (pH 8.5) in the standard medium to ensure the complete displacement of fluorinated intracellular proteins. After an incubation of 6 h at 37° C., cell samples were washed with TCEP containing PBS buffer for three times, and then stained with Hoechst 33342 dye at RT for 10 min. All cell samples were briefly washed after nucleus staining, and examined using the ZOE fluorescent microscope imager (Bio-Rad Laboratories).

In-Gel Fluorescent Imaging of Protein Substrates Labelled from Cell Lysates: HeLa or HEK293 cells were seeded at 20,000 cells/dish in a 100 mm×20 mm cell culture dish (Corning). Upon ˜80% confluence, cells were incubated with 1 mM ethyl azidoacetate (for “click chemistry”), ethyl fluoroacetate (for fluorination), or DMSO control at 37° C. for 6 h. After these treatments, cells were rinsed three times with DPBS buffer. Each cell sample was immediately added 700 μL of celLytic M buffer (Sigma Aldrich), and gently shaken for 15 min at RT. The cell lysates were centrifuged at 15,000 g, 4° C. for 15 min to pellet the debris. Supernatants were collected, and the protein concentrations were quantified via BCA assay kit (Thermo Fisher).

For “click chemistry” labeling of the azido-modified proteins in cell lysates, approximately 300 μL supernatant of each cell lysate (˜2 mg/mL) was incubated with 1 mM TCEP, 100 μM TBTA, 1 mM CuSO₄, and 100 μM TAMRA-alkyne probe (compound (16)) according to published procedures (Sinclair W R et al., 2018, Chem. Sci., 9:1236-1241; Montgomery D C et al., 2014, J. Am. Chem. Soc., 136:8669-8676). For fluorine-thiol displacement labeling, approximately 300 μL supernatant of each cell lysate (˜2 mg/mL) was treated with 100 mM TCEP and 4 mM TAMRA-SH probe (compound (15)) at pH 8.5 for 12 h to ensure the complete displacement. All the protein samples after reaction were each added 1 mL of cold acetone to precipitate out proteins. Proteins were then resuspended in PBS buffer, re-precipitated with methanol, and pelleted by centrifuging at 15,000 g, 4° C. for 10 min. Each protein pellet was redissolved in 100 μL PBS buffer that comprised of 1% SDS and 10% glycerol. Approximately ˜20 μg of each protein sample was mixed with SDS-PAGE sample buffer, and separated by 4%-12% gradient SDS-PAGE analysis. In-gel fluorescence detection of the labelled TAMRA dye was achieved by scanning the gel with the LI-COR Odyssey Fe Imager (600 nm channel, Ex 520 nm/Em 600 nm).

FTDR-Based Labeling and Imaging of Protein Substrates with Concurrent HDAC Inhibition: HEK293 cells were cultured and treated with the pro-metabolite ethyl fluoroacetate, or DMSO control as mentioned above. To probe the effect of HDAC inhibition, 1×deacetylase inhibitor cocktail (APExBIO) was added to the medium concurrently with the addition of the pro-metabolite and incubated for the indicated time (6 h and 12 h, respectively). The cell samples were then lysed, with the proteins labelled by the TAMRA-SH probe based on FTDR. Subsequently, each sample was purified, loaded to SDS-PAGE, and imaged the same as mentioned above.

Histone Extraction: HEK293 cell samples were prepared as mentioned above. For the competition assay, 10 mM ethyl acetate was added to the media concurrently with the pro-metabolite ethyl fluoroacetate. After cell lysis and FTDR, histones were extracted with the EpiQuik Total Histone Extraction kit (Epigentek) following the manufacturer's instructions. Approximately 3 μg of each histone extract was mixed with the sample loading buffer and separated on 12% SDS-PAGE, followed by subsequent in-gel fluorescent imaging and CBB staining as mentioned before.

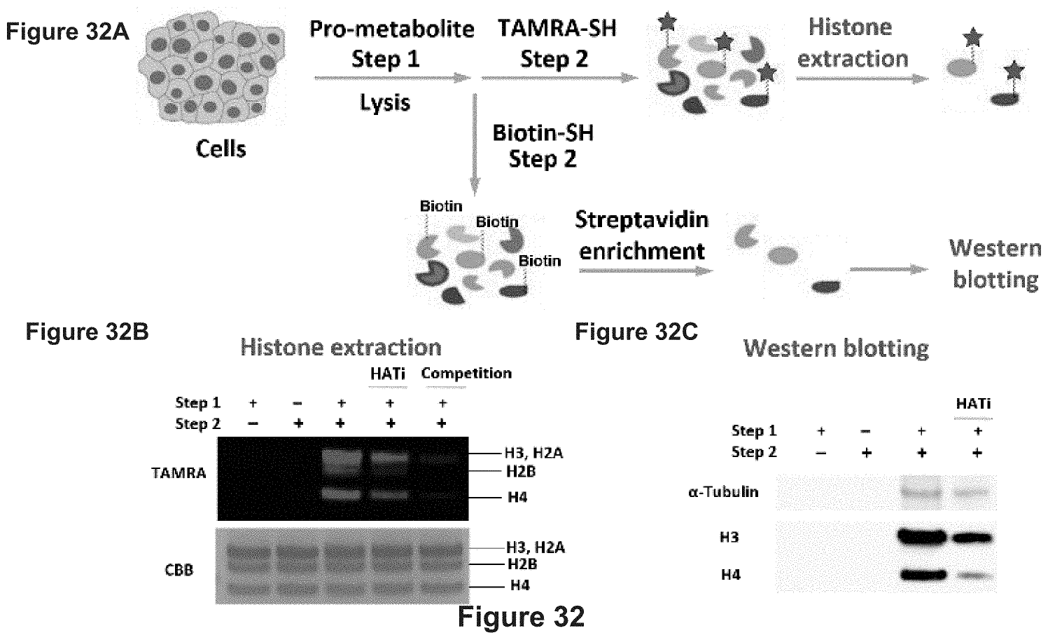

FTDR-Based Pull Down of Protein Substrates Followed by Western Blot Analysis: HEK293 cells after step 1 treatment (FIG. 32A) was lysed and the resulting proteome (˜2 mg/mL, pH 8.5) was added with 100 mM TCEP and 4 mM Biotin-SH probe (compound (14)). The reaction mixture was incubated at 37° C. for 6 h, with the unconjugated probes removed by subsequent methanol precipitation twice. The protein pellet was redissolved in PBS buffer (0.1% SDS, pH 7.2). Approximately 100 μL of the proteins (1 mg/ml) were mixed with 100 μL streptavidin magnetic beads (New England BioLabs) at room temperature for 1 h. The beads were sequentially washed with PBS buffer (0.1% SDS, pH 7.2), PBS buffer (0.2% SDS and 4 M urea, pH 7.2), and PBS buffer (0.2% SDS, pH 7.2). After the final washing with pure PBS buffer three times, the beads were incubated with the elution buffer (10 mM sodium periodate in PBS) for 30 min in the dark. The elution was repeated three times and the combined elute was concentrated on a lyophilizer. The eluted protein samples were finally separated by 4-12% Bis-Tris SDS-PAGE and transferred onto PVDF membranes. The blot was blocked with 3% BSA in TBST for 1 h, followed by the incubation with the anti-Histone H3 antibody (HRP Conjugate, CST #12648), anti-Histone H4 antibody (HRP Conjugate, abcam #ab197517), and the anti-alpha-Tubulin antibody (CST #2144) at 4° C. After overnight incubation, the blot was washed with TBST three times and was further incubated with an anti-rabbit secondary antibody (HRP conjugated) for the detection of alpha tubulin. After 30 min incubation at room temperature, the blot was washed with TBST and imaged using the Clarity Max Western ECL substrate.

Proteomics Study of Fluoroacetylated Histones and Alpha-tubulin: HEK293 cells were treated with ethyl fluoroacetate (FIG. 33A), with subsequent histone extraction carried out the same as mentioned above. The histone proteins were desalted using the C4 columns (The Nest Group), lyophilized, and dissolved in the incubation buffer (50 mM Tris-HCl, 5 mM CaCl₂), 2 mM EDTA, pH 7.6-7.9) for in-solution digestion. After the addition of Arg-C (Promega), the digestion was activated with the 10× activation buffer (50 mM Tris-HCl, 60 mM DTT, 2 mM EDTA, pH 7.6-7.9) that was added to a final concentration of 1×. The reaction mixture was incubated at 37° C. for 7 h, followed by purification using C18 columns (The Nest Group). The elutions were collected, lyophilized, and dissolved in water to be injected into the LC-MS/MS system. For immuno-enrichment of endogenous alpha tubulin, 100 μL of each cell lysate was gently mixed with 10 μL of anti-alpha-tubulin antibody (Cell Signaling, CST3873S) and incubated overnight at 4° C. Approximately 50 μL of Dynabeads Protein G (Invitrogen) was then added and the mixture was incubated at 4° C. for 1 h. The Dynabeads were pulled down and washed with 300 μL PBS buffer (0.05% Tween-20) three times. The targeted proteins were eluted by heating the beads at 90° C. for 10 min within the LDS samples loading buffer (GenScript). The resulting supernatants were loaded onto a 10% SurePAGE Bis-Tris gel (GenScript). After staining by Coomassie brilliant blue R-250, the bands at 50 kDa were cut off for in-gel digestion by Glu-C (Promega). Liquid chromatography tandem mass spectrometry (LC-MS/MS) analysis was performed as previously described16 using a Q Exactive HF mass spectrometer (ThermoFisher Scientific) coupled with a Nano-ACQUITY UPLC system (Waters). Peptide sequences were identified using MaxQuant v1.6.15.0.17 MS/MS spectra were searched against a UniProt human protein database (Oct. 10, 2019) using full enzyme specificity with up to two missed cleavages, static carboxamidomethylation of Cys, and variable Met oxidation (+15.9949 Da), Asn deamidation (0.9840 Da), Lys acetylation (+42.0106 Da) and Lys F-acetylation (+60.0011 Da). Consensus identification lists were generated with false discovery rates of 1% at protein, peptide and site levels.

### Compound Characterization

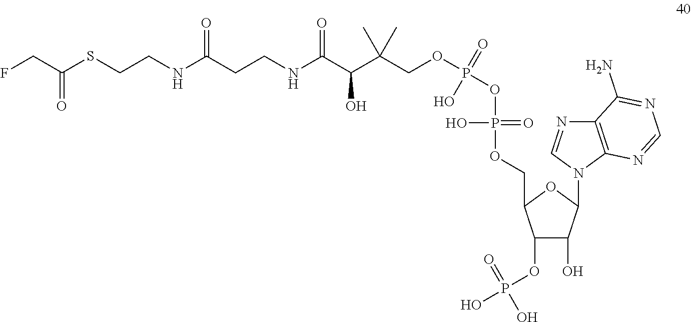 [SMILES: CC(C)(COP(=O)(O)OP(=O)(O)OCC1OC(n2cnc3c(N)ncnc32)C(O)C1OP(=O)(O)O)[C@@H](O)C(=O)NCCC(=O)NCCSC(=O)CF]

The fluoroacetyl-CoA (compound (40)) was synthesized and purified following the reported procedures (Weeks A M et al., 2012, Proc. Natl. Acad. Sci. USA, 109:19667-19672). ¹H NMR (500 MHz, D₂O): δ 8.66 (s, 1H), 8.42 (s, 1H), 6.21 (d, J=5.5 Hz, 1H), 5.01 (d, J=46.5 Hz, 2H), 4.87-4.82 (m, 2H), 4.58 (s, 1H), 4.24 (s, 2H), 4.00 (s, 1H), 3.85-3.82 (m, 1H), 3.59-3.56 (m, 1H), 3.44 (t, J=6.5 Hz, 2H), 3.35 (t, J=6.0 Hz, 2H), 3.08 (t, J=6.5 Hz, 2H), 2.42 (t, J=6.5 Hz, 2H), 0.92 (s, 3H), 0.79 (s, 3H). HRMS (ESI) m/z calculated for C₂₃H₃₈FN₇O₁₇P₃S [M+H]⁺: 828.1236, found 828.1229.

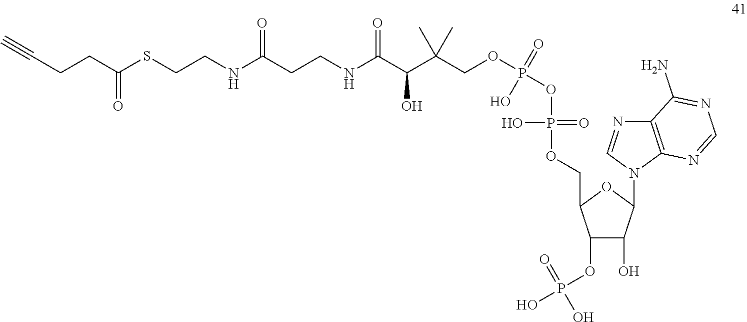 [SMILES: C#CCCC(=O)SCCNC(=O)CCNC(=O)[C@H](O)C(C)(C)COP(=O)(O)OP(=O)(O)OCC1OC(n2cnc3c(N)ncnc32)C(O)C1OP(=O)(O)O]

The 4-pentynoyl CoA (compound (41)) was prepared according to the published synthetic and purification procedures (Yang C et al., 2013, J. Am. Chem. Soc., 135:7791-7794). ¹H NMR (500 MHz, D₂O): δ 8.66 (s, 1H), 8.45 (s, 1H), 6.22 (d, J=6.0 Hz, 1H), 4.61 (s, 1H), 4.28 (s, 2H), 4.02 (s, 1H), 3.90-3.86 (m, 1H), 3.65-3.61 (m, 1H), 3.46 (t, J=6.5 Hz, 2H), 3.35 (t, J=6.0 Hz, 2H), 3.03 (t, J=6.5 Hz, 2H), 2.84 (t, J=7.0 Hz, 2H), 2.52-2.48 (m, 2H), 2.44 (t, J=6.5 Hz, 2H), 2.34 (t, J=2.5 Hz, 2H), 0.94 (s, 3H), 0.82 (s, 3H). HRMS (ESI) m/z calculated for C₂₆H₄₁N₇O₁₇P₃S [M+H]⁺: 848.1487, found 848.1477.

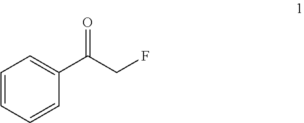

Dried potassium fluoride (580 mg, 10 mmol) was added to a solution of 18-crown-6 (264 mg, 1 mmol) in anhydrous acetonitrile (6 mL) (FIG. 17). After stirred at room temperature for 20 min, 2-bromoacetophenone (17) (398 mg, 2 mmol) in anhydrous acetonitrile (2 mL) was added and then heated to reflux and stirred overnight. After being concentrated under reduced pressure, the mixture was purified via flash column chromatography (hexane/ethyl acetate: 6/1) to afford compound (1) as a light yellow oil (210 mg, 1.52 mmol, 76% yield). ¹H NMR (500 MHz, CDCl₃): δ 7.90 (d, J=8.0 Hz, 2H), 7.64-7.61 (m, 1H), 7.52-7.48 (m, 2H), 5.54 (d, J=47.0 Hz, 2H); ¹³C NMR (126 MHz, CDCl₃): δ 193.4 (d, J=15.5 Hz), 134.2, 133.7, 129.0, 127.8 (d, J=2.5 Hz), 83.6 (d, J=182.8 Hz); ¹⁹F NMR (471 MHz, CDCl₃): δ −230.75; GC-MS m/z calculated for C₈H₇FO [M]⁺: 138.0 found 138.0.

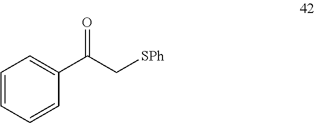

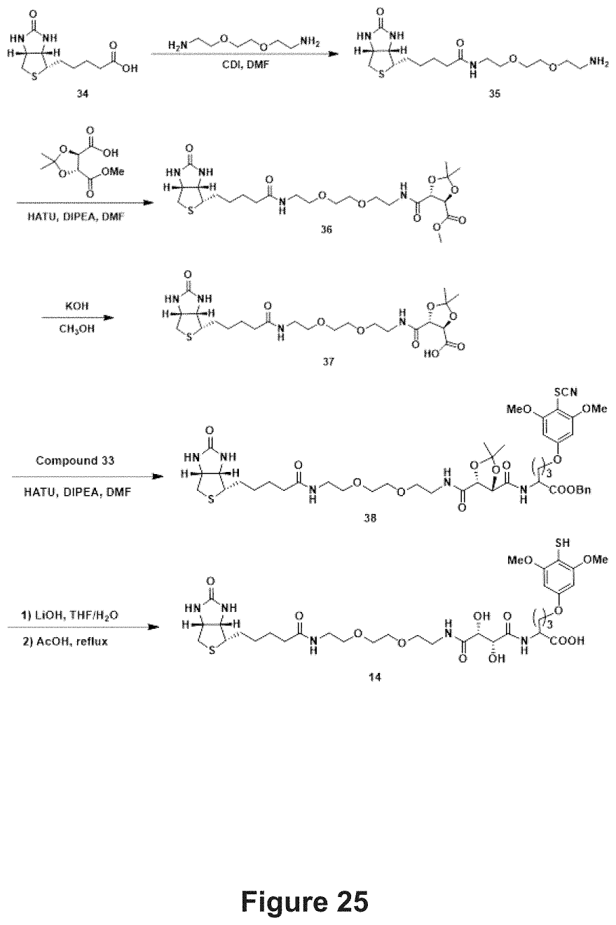

General procedure A (FIG. 25) was followed from 1 (28 mg, 0.2 mmol) in the presence of potassium carbonate to give (42) (42 mg, 91% yield) as a colorless oil after flash column chromatography (hexane/ethyl acetate: 10/1). ¹H NMR (500 MHz, CDCl₃): δ 7.96-7.93 (m, 2H), 7.60-7.56 (m, 1H), 7.48-7.44 (m, 2H), 7.40-7.38 (m, 2H), 7.30-7.26 (m, 2H), 7.24-7.20 (m, 1H), 4.28 (s, 2H); ¹³C NMR (126 MHz, CDCl₃): δ 194.1, 135.4, 134.8, 133.5, 130.6, 129.1, 128.7, 127.2, 41.3; MS (ESI) m/z calculated for C₁₄H₁₃OS [M+H]⁺: 229.1, found 229.1.

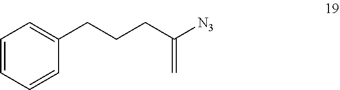

To a solution of pent-4-yn-1-ylbenzene (18) (455 μL, 3 mmol), trimethylsilyl azide (788 μL, 6 mmol), water (108 μL, 6 mmol), DMSO (10 mL), and silver carbonate (83 mg, 0.3 mmol) were added (FIG. 18). The mixture was then stirred at 80° C. for 1 h. After cooled down to room temperature, water was added. The aqueous phase was extracted with ethyl acetate. The combined organic phase was washed with brine, then water, dried over anhydrous sodium sulfate and concentrated under reduced pressure. The residue was purified by flash column chromatography (100% hexane) to provide the desired compound (19) as a colorless oil (338 mg, 1.8 mmol, 60% yield). ¹H NMR (500 MHz, CDCl₃): δ 7.31-7.28 (m, 2H), 7.22-7.18 (m, 3H), 4.67-4.66 (m, 2H), 2.64 (t, J=7.5 Hz, 2H), 2.11 (t, J=7.5 Hz, 2H), 1.86-1.79 (m, 2H); ¹³C NMR (126 MHz, CDCl₃): δ 146.5, 141.8, 128.5, 128.4, 125.9, 98.4, 35.0, 33.2, 28.9; HRMS (ESI) m/z calculated for C₁₁H₁₄N₃ [M+H]⁺: 188.1182, found 188.1180.

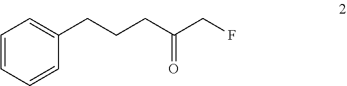 [SMILES: O=C(CF)CCCc1ccccc1]

Vinyl azide (19) (187 mg, 1 mmol) was added to a suspension of Selectfluor (480 mg, 1.5 mmol), sodium bicarbonate (168 mg, 2 mmol), and water (36 μL, 2 mmol) in acetonitrile (10 mL) (FIG. 18). The resulting mixture was stirred at room temperature overnight. After concentrated under reduced pressure, the residue was purified by flash column chromatography (hexane/ethyl acetate: 20/1) to give compound (2) as a colorless oil (86 mg, 0.48 mmol, 48% yield). ¹H NMR (500 MHz, CDCl₃): δ 7.31-7.27 (m, 2H), 7.22-7.17 (m, 3H), 4.75 (d, J=48 Hz, 2H), 2.66 (t, J=7.5 Hz, 2H), 2.55 (dt, J=7.5, 2.5 Hz, 2H), 1.97 (quint, J=7.5 Hz, 2H); ¹³C NMR (126 MHz, CDCl₃): δ 206.8 (d, J=20.2 Hz), 141.2, 128.48, 128.47, 126.1, 85.0 (d, J=185.2 Hz), 37.4, 35.0, 24.1 (d, J=1.8 Hz); ¹⁹F NMR (471 MHz, CDCl₃): δ −227.52; MS (ESI) m/z calculated for C₁₁H₁₄FO [M+Na]⁺: 203.0, found 203.1.

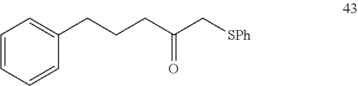

General procedure A (FIG. 25) was followed from (2) (36 mg, 0.2 mmol) in the presence of DBU to give (43) (53 mg, 98% yield) as a colorless oil after flash column chromatography (hexane/ethyl acetate: 10/1). ¹H NMR (500 MHz, CDCl₃): δ 7.37-7.16 (m, 10H), 3.68 (s, 2H), 2.63 (t, J=7.5 Hz, 2H), 2.62 (t, J=7.5 Hz, 2H), 1.94 (quint, J=7.5 Hz, 2H); ¹³C NMR (126 MHz, CDCl₃): δ 205.4, 141.5, 134.9, 129.6, 129.2, 128.5, 128.4, 126.9, 126.0, 44.0, 39.8, 35.0, 25.2; MS (ESI) m/z calculated for C₁₇H₁₉OS [M+H]⁺: 271.1, found 271.1.

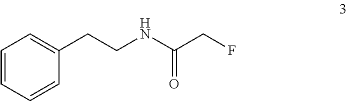 [SMILES: O=C(CF)NCCc1ccccc1]

Sodium fluoroacetate (100 mg, 1 mmol) was mixed with 1-[Bis(dimethylamino)methylene]-1H-1,2,3-triazolo[4,5-b]pyridinium 3-oxid hexafluorophosphate (HATU) (456 mg, 1.2 mmol) and DIPEA (209 μL, 1.2 mmol) in DMF (5 mL) (FIG. 19). After the mixture was stirred at room temperature for 20 min, phenethylamine (20) (251 μL, 2 mmol) was added dropwisely. The resulting mixture was continuously stirred at room temperature overnight, and then quenched by water. Ethyl acetate was added to extract the product from aqueous layer. The organic layer was dried with anhydrous sodium sulfate and concentrated under vacuum. The crude mixture was then purified via flash column chromatography (hexane/ethyl acetate: 3/1) to afford compound (3) as a white solid (147 mg, 0.81 mmol, 81% yield). ¹H NMR (500 MHz, CDCl₃): δ 7.36-7.33 (m, 2H), 7.28-7.22 (m, 3H), 6.37 (br, 1H), 4.79 (d, J=47.5 Hz, 2H), 3.62 (q, J=6.5 Hz, 2H), 2.89 (t, J=7.5 Hz, 2H); ¹³C NMR (126 MHz, CDCl₃): δ 167.5 (d, J=17.1 Hz), 138.4, 128.8, 128.7, 126.7, 80.3 (d, J=186.1 Hz), 40.0, 35.6; ¹⁹F NMR (471 MHz, CDCl₃): δ −227.23; HRMS (ESI) m/z calculated for C₁₀H₁₃FNO [M+H]⁺: 182.0976, found 182.0980.

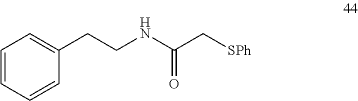

General procedure A (FIG. 25) was followed from (3) (37 mg, 0.2 mmol) in the presence of DBU to generate (44) (50 mg, 93% yield) as a white solid after flash column chromatography (hexane/ethyl acetate: 2/1). ¹H NMR (500 MHz, CDCl₃): δ 7.30-7.17 (m, 8H), 7.06-7.04 (m, 2H), 3.61 (s, 2H), 3.51 (q, J=7.0 Hz, 2H), 2.73 (t, J=6.5 Hz, 2H); ¹³C NMR (126 MHz, CDCl₃): δ 167.7, 138.5, 134.7, 129.3, 128.70, 128.66, 127.8, 126.58, 126.55, 40.9, 37.2, 35.5; MS (ESI) m/z calculated for C₁₆H₁₈NOS [M+H]⁺: 272.1, found 272.1.

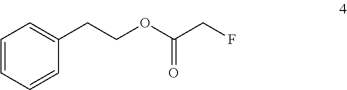 [SMILES: O=C(CF)OCCc1ccccc1]

2-Phenylethanol (21) (359 μL, 3 mmol) was dissolved in DCM (10 mL), and mixed with potassium carbonate (828 mg, 6 mmol) in 2 mL water (FIG. 20). The mixture was cooled in an ice bath, and a solution of bromoacetyl bromide (392 μL, 4.5 mmol) in DCM (3 mL) was dropwisely added. After 30 min of stirring, the reaction solution was warmed to room temperature and continuously stirred for another 2 h. The aqueous layer was then separated and extracted with DCM. The combined organic phase was washed with brine and water. After drying with anhydrous sodium sulfate, the organic layer was vacuum concentrated to yield an oily intermediate. After resuspension in THF, the oily intermediate was mixed with TBAF (6 mL, 6 mmol, 1 M in THF), refluxed for 1 h, and concentrated under reduced pressure. The crude mixture was purified via flash column chromatography (hexane/ethyl acetate: 15/1) to afford colorless oil-like compound (4) (337 mg, 1.85 mmol, 62% yield over two steps). ¹H NMR (500 MHz, CDCl₃): δ 7.33-7.29 (m, 2H), 7.26-7.20 (m, 3H), 4.81 (d, J=47.0 Hz, 2H), 4.43 (t, J=7.5 Hz, 2H), 2.98 (t, J=7.0 Hz, 2H); ¹³C NMR (126 MHz, CDCl₃): δ 167.8 (d, J=21.8 Hz), 137.2, 128.9, 128.6, 126.8, 78.3 (d, one peak overlap with CDCl₃), δ 5.8, 35.0; ¹⁹F NMR (471 MHz, CDCl₃): δ −229.98. HRMS (ESI) m/z calculated for C₁₀H₁₂FO₂ [M+H]⁺: 183.0816, found 183.0862.

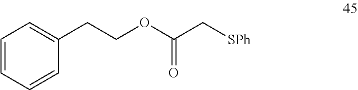

General procedure A (FIG. 25) was followed from (4) (37 mg, 0.2 mmol) in the presence of potassium carbonate to afford (45) (8 mg, 14% yield) as a colorless oil after flash column chromatography (hexane/ethyl acetate: 20/1). ¹H NMR (500 MHz, CDCl₃): δ 7.38-7.35 (m, 2H), 7.30-7.26 (m, 4H), 7.24-7.17 (m, 4H), 4.32 (t, J=7.5 Hz, 2H), 3.63 (s, 2H), 2.90 (t, J=7.0 Hz, 2H); ¹³C NMR (126 MHz, CDCl₃): δ 169.7, 137.5, 135.0, 129.9, 129.1, 128.9, 128.6, 127.0, 126.6, 66.0, 36.7, 34.9; MS (ESI) m/z calculated for C₁₆H₁₇O₂S [M+H]⁺: 273.1, found 273.1.

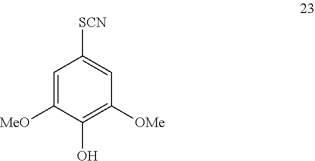

Bromine (774 μL, 15 mmol) was slowly added to the saturated solution of sodium bromide in methanol (10 mL) at 0° C. (FIG. 21). After stirring for 15 min, the solution was added dropwisely to a mixture of 2,6-dimethoxyphenol(22) (1.54 g, 10 mmol) and potassium thiocyanate (1.46 g, 15 mmol) in 0° C. methanol (30 mL). The reaction was left on for 1 h, and then quenched by water. The methanol in the mixture was evaporated under reduced pressure, and the remaining aqueous phase was extracted with ethyl acetate. The combined organic layer was washed with brine, and water, dried with anhydrous Na₂SO₄, and eventually concentrated to afford an orange colored oil-like mixture. The crude mixture was purified via flash column chromatography (hexane/ethyl acetate: 5/1) to result in compound (23) as an orange colored solid (1.14 g, 5.4 mmol, 54% yield). ¹H NMR (500 MHz, CDCl₃): δ 6.80 (s, 2H), 5.74 (s, 1H), 3.91 (s, 6H); ¹³C NMR (126 MHz, CDCl₃): δ 147.9, 136.9, 112.7, 111.6, 109.3, 56.8; MS (ESI) m/z calculated for C₉H₁₀NO₃S [M+H]⁺: 212.0, found 212.0.

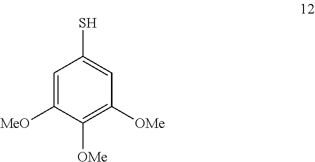 [SMILES: COc1cc(S)cc(OC)c1OC]

Potassium carbonate (276 mg, 2 mmol) and intermediate (23) (211 mg, 1 mmol) were dissolved in DMF (20 mL) and cooled to 0° C. (FIG. 23). Methyl iodide (125 μL, 2 mmol) was added dropwisely, after which the reaction was stirred at room temperature until TLC analysis confirmed the completion of reaction. The mixture was then diluted with ethyl acetate and water. After extraction and separation, the organic layer was washed with brine and water, dried with anhydrous sodium sulfate, and finally concentrated under reduced pressure to afford the methylated intermediate. The crude mixture was dissolved in THE (20 mL), added with the aqueous solution of lithium hydroxide (1M, 2 mL). After stirring at room temperature for 2 h, the reaction was quenched by water, and the crude product was extracted by ethyl acetate. The organic layer was dried with anhydrous sodium sulfate, vacuum concentrated, and subsequently purified via flash column chromatography (hexane/ethyl acetate: 5/1) to afford compound (12) in yellow colored solid form (72% yield over two steps). ¹H NMR (500 MHz, CDCl₃): δ 6.73 (s, 2H), 3.81 (s, 3H), 3.80 (s, 6H); ¹³C NMR (126 MHz, CDCl₃): δ 153.4, 138.0, 132.0, 106.5, 60.9, 56.2; MS (ESI) m/z calculated for C₉H₁₃O₃S [M+H]⁺: 201.0, found 201.1.

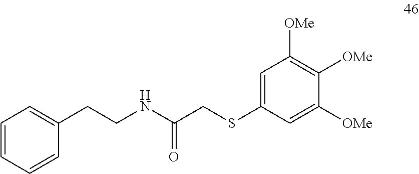 [SMILES: COc1cc(SCC(=O)NCCc2ccccc2)cc(OC)c1OC]

Prepared according to General Procedure B (FIG. 31) using 3,4,5-trimethoxybenzenethiol (12) (20 mg, 0.1 mmol) to give product (46) (16 mg, 88% yield) as white solid after flash column chromatography (hexane/ethyl acetate: 1/1). ¹H NMR (500 MHz, CDCl₃): δ 7.25-7.17 (m, 3H), 7.07-7.05 (m, 2H), 6.77 (t, J=6.0 Hz, 1H), 6.45 (s, 1H), 3.82 (s, 3H), 3.81 (s, 6H), 3.59 (s, 2H), 3.52 (q, J=7.0 Hz, 2H), 2.75 (t, J=7.0 Hz, 2H); ¹³C NMR (126 MHz, CDCl₃): δ 167.9, 153.7, 138.5, 129.5, 128.66, 128.64, 126.6, 105.37, 105.34, 61.0, 56.2, 41.0, 37.9, 35.6; HRMS (ESI) m/z calculated for C₁₉H₂₄NO₄S [M+H]⁺: 362.1421, found 362.1413.

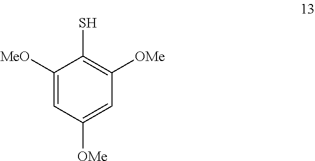 [SMILES: COc1cc(OC)c(S)c(OC)c1]

To a solution of 2,4,6-trimethoxybenzene (24) (504 mg, 3.0 mmol) in THE (10 mL), n-butyllithium in hexane (2.5 M, 1.2 mL, 3.0 mmol) was added at 0° C., followed with a catalytic amount of tetramethylethylenediamine (TMEDA) (23 μL, 0.15 mmol) (FIG. 22). The reaction mixture was warmed up to room temperature, and stirred for 1 h until the suspension turned an orange color. Elemental sulfur (96 mg, 3 mmol) in warm toluene (3 mL) was added dropwisely, and the reaction mixture was stirred at room temperature. Upon reaction completion as monitored by TLC, 10 mL of water was added to quench the reaction, and the aqueous layer was acidified by 1 M HCl. The products were extracted by ethyl acetate, washed with water, brine, and dried over sodium sulfate. After vacuum concentration, the crude mixture was purified via flash column chromatography (hexane/ethyl acetate: 4/1) to afford the final product (13) as a crystalline yellow solid (360 mg, 60% yield). ¹H NMR (500 MHz, CDCl₃): δ 6.17 (s, 2H), 3.87 (s, 6H), 3.80 (s, 3H), 3.77 (s, 1H); ¹³C NMR (126 MHz, CDCl₃): δ 158.7, 156.2, 99.7, 91.2, 56.1, 55.5; MS (ESI) m/z calculated for C₉H₁₃O₃S [M+H]⁺: 201.1, found 201.1.

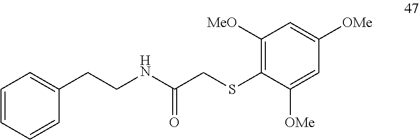 [SMILES: COc1cc(OC)c(SCC(=O)NCCc2ccccc2)c(OC)c1]

Prepared according to General Procedure B (FIG. 31) using 2,4,6-trimethoxybenzenethiol (13) (20 mg, 0.1 mmol) to generate product (47) (15 mg, 83% yield) as white solid after flash column chromatography (hexane/ethyl acetate: 2/1). ¹H NMR (500 MHz, CDCl₃): δ 7.88 (s, 1H), 7.30-7.27 (m, 2H), 7.23-7.20 (m, 1H), 7.18-7.16 (m, 2H), 6.10 (s, 2H), 3.82 (s, 3H), 3.78 (s, 6H), 3.47 (s, 2H), 3.44 (q, J=7.0 Hz, 2H), 2.76 (t, J=7.0 Hz, 2H); ¹³C NMR (126 MHz, CDCl₃): δ 169.1, 162.4, 161.5, 139.1, 128.7, 128.6, 126.4, 100.1, 91.2, 56.1, 55.5, 41.3, 38.6, 35.9; HRMS (ESI) m/z calculated for C₁₉H₂₄NO₄S [M+H]⁺: 362.1421, found 362.1415.

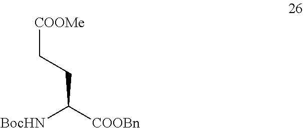

Boc-L-glutamic acid 1-benzyl ester (25) (6.0 g, 17.7 mmol) and sodium bicarbonate (3.68 g, 26.7 mmol) were dissolved in DMF (60 mL) and cooled to 0° C. (FIG. 23). Methyl iodide (2.21 mL, 35.6 mmol) was added dropwisely, after which the reaction was stirred at room temperature until TLC analysis confirmed reaction completion. Upon completion, the reaction mixture was diluted 10-fold with water and extracted with ethyl acetate. The organic layer was washed with a 10% sodium bicarbonate solution, followed by brine and was subsequently dried with anhydrous sodium sulfate. After vacuum concentration, the crude mixture was purified via flash column chromatography (hexane/ethyl acetate: 2/1) to afford compound (26) as a clear oil (6.25 g, quantitative yield). ¹H NMR (500 MHz, CDCl₃): δ 7.38-7.31 (m, 5H), 5.16 (d, J=4.0 Hz, 2H), 5.12 (m, 1H), 4.37 (d, J=5.0 Hz, 1H), 3.66 (s, 3H), 2.44-2.31 (m, 2H), 2.23-2.17 (m, 1H), 2.00-1.92 (m, 1H), 1.42 (s, 9H); ¹³C NMR (126 MHz, CDCl₃): δ 173.2, 172.1, 155.4, 135.3, 128.7, 128.5, 128.3, 80.1, 67.3, 53.0, 51.8, 30.1, 28.3, 27.8; MS (ESI) m/z calculated for C₁₈H₂₅NNaO₆ [M+Na]⁺: 374.2, found 374.2.

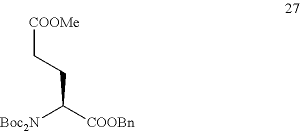

To a solution of intermediate (26) (6.25 g, 17.7 mmol) and 4-dimethylaminopyridine (DMAP)(435 mg, 3.5 mmol) in acetonitrile was added di-tert-butyl dicarbonate (7.76 g, 35.4 mmol) (FIG. 23). The reaction mixture was stirred overnight and directly vacuum concentrated upon completion as monitored by TLC. The concentrated crude mixture was purified via flash column chromatography (hexane/ethyl acetate: 4/1) to afford compound (27) as a clear oil (7.63 g, 95% yield). ¹H NMR (500 MHz, CDCl₃): δ 7.33-7.27 (m, 5H), 5.14 (d, J=2.5 Hz, 2H), 4.97 (q, J=5.0 Hz, 1H), 3.66 (s, 3H), 2.53-2.46 (m, 1H), 2.43-2.35 (m, 2H), 2.24-2.16 (m, 1H), 1.44 (s, 18H); ¹³C NMR (126 MHz, CDCl₃): δ 173.1, 170.2, 152.0, 135.6, 128.5, 128.2, 128.0, 83.3, 66.9, 57.5, 51.7, 30.6, 27.9, 24.8; MS (ESI) m/z calculated for C₂₃H₃₃NNaO₈ [M+Na]⁺: 474.2, found 474.2.

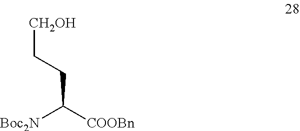

In a flame dried flask under a nitrogen atmosphere, a solution of intermediate (27) (7.63 g, 16.9 mmol) in THE (80 mL) was cooled to −80° C. (FIG. 23). Diisobutylaluminum hydride solution (DIBAL)(1.0 M in hexanes) (33.8 mL) was added dropwisely over 30 min. The reaction mixture was stirred at −80° C. for at least 2 h. Upon completion as monitored by TLC, the reaction was quenched with a saturated Rochelle salt solution in water (200 mL), and was stirred at room temperature overnight. On the next day, the reaction mixture was diluted further with water (100 mL) and extracted with ethyl acetate. The organic layer was then dried with sodium sulfate and vacuum concentrated. The crude reaction mixture was purified via flash column chromatography (hexane/ethyl acetate: 2/1) to afford compound (28) as a clear oil (5.0 g, 70% yield). ¹H NMR (500 MHz, CDCl₃): δ 7.34-7.29 (m, 5H), 5.14 (q, J=12.5 Hz, 2H), 4.91 (d, J=9.5, 5.5 Hz, 1H), 3.66 (t, J=6.5 Hz, 2H), 2.28-2.21 (m, 1H), 1.99-1.91 (m, 1H), 1.66-1.1.62 (m, 2H), 1.44 (s, 18H); ¹³C NMR (126 MHz, CDCl₃): δ 170.8, 152.3, 135.7, 128.5, 128.1, 128.0, 83.2, 66.8, 62.3, 58.0, 29.4, 27.9, 26.0; HRMS (ESI) m/z calculated for C₁₂H₁₈NO₃ (without di-Boc) [M+H]⁺: 224.1281, found 224.1279.

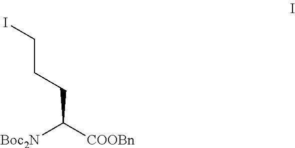

Intermediate (28) (5.0 g, 11.8 mmol), triphenylphosphine (4.64 g, 17.7 mmol), and imidazole (1.2 g, 17.7 mmol) were dissolved in DCM (60 mL) and stirred (FIG. 23). Once dissolved, iodine (5.99 g, 23.6 mmol) was added, and the reaction mixture was stirred overnight. Upon completion, the reaction was quenched with saturated sodium sulfite (75 mL), and the organic products were extracted with dichloromethane. The organic layer was dried with anhydrous sodium sulfate, concentrated under reduced pressure; and the resulting crude mixture was purified using flash column chromatography (hexane/ethyl acetate: 4/1). Compound (29) was finally obtained as a clear oil (5.04 g, 80% yield). ¹H NMR (500 MHz, CDCl₃): δ 7.39-7.27 (m, 5H), 5.15 (q, J=12.5 Hz, 2H), 4.90 (q, J=5.0 Hz, 1H), 3.27-3.14 (m, 2H), 2.27-2.19 (m, 1H), 2.09-2.01 (m, 1H), 1.95-1.85 (m, 2H), 1.46 (s, 18H); ¹³C NMR (126 MHz, CDCl₃): δ 170.4, 152.2, 135.6, 128.5, 128.2, 128.0, 83.3, 66.9, 57.2, 30.5, 30.2, 28.0, 5.7; HRMS (ESI) m/z calculated for C₂₂H₃₃INO₆ [M+H]⁺: 534.1347, found 534.2258.

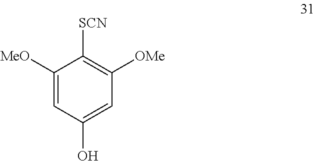

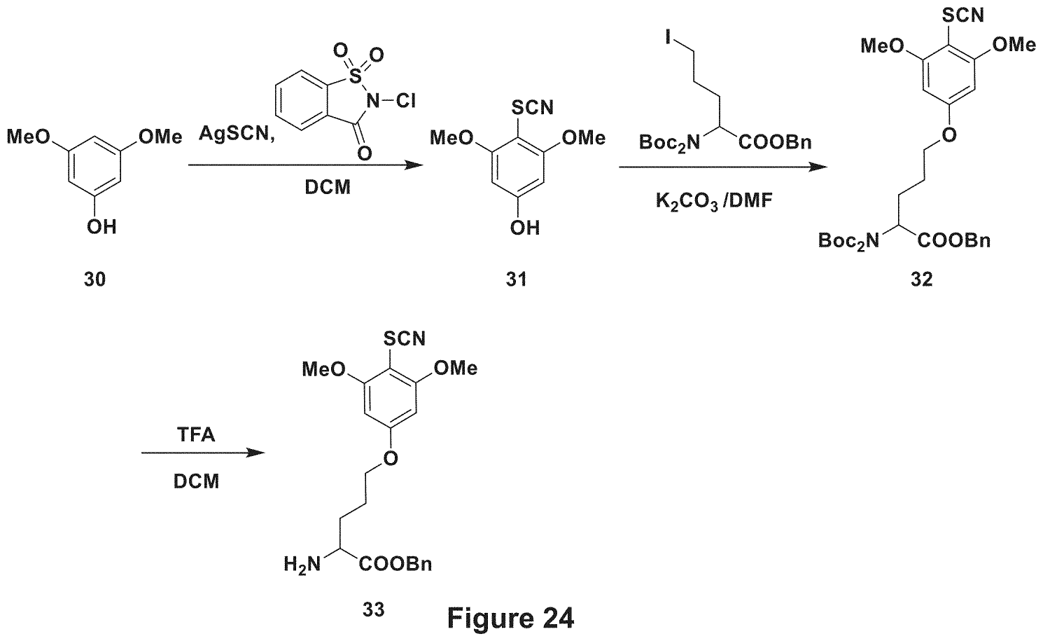

To a solution of N-chlorosaccharin (5.15 g, 23.7 mmol) in dichloromethane (90 mL) was added silver thiocyanate (3.93 g, 23.7 mmol) (FIG. 24). A white solid crashed out upon addition and the reaction mixture was kept stirring for 1 h. 3,5-dimethoxyphenol (compound (30), 3.04 g, 19.6 mmol) was then added, and the reaction mixture was stirred for another 3 h, at which point the reaction was confirmed to be complete by TLC analysis. The dark red heterogeneous mixture was vacuum filtered, and the filtrate was vacuum concentrated to afford a dark red oil. The crude oil mixture was purified via flash column chromatography (hexane/ethyl acetate: 3/1) to afford compound (31) as an orange solid (2.50 g, 60% yield). ¹H NMR (500 MHz, CDCl₃): δ 6.10 (s, 2H), 3.84 (s, 6H); ¹³C NMR (126 MHz, CDCl₃): δ 161.5, 160.9, 112.6, 92.9, 89.1, 56.4; MS (ESI) m/z calculated for C₉H₁₀NO₃S [M+H]⁺: 212.0, found 212.0.

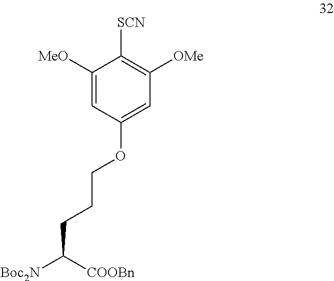

Intermediate (29) (5.0 g, 9.37 mmol), (31) (3.96 g, 18.7 mmol) and potassium carbonate (1.94 g, 14.0 mmol) were dissolved in DMF (50 mL) and stirred at room temperature for 8 h (FIG. 24). Upon reaction completion, the mixture was diluted 10-fold with water, and the organic products were extracted by ethyl acetate. The organic layer was dried with sodium sulfate, vacuum concentrated; and the crude oil was purified via flash column chromatography (hexane/ethyl acetate: 3/1) to afford compound (32) as a yellowish oil (4.05 g, 70% yield). ¹H NMR (500 MHz, CDCl₃): δ 7.34-7.28 (m, 5H), 6.13 (s, 1H), 5.15 (q, J=12.5 Hz, 2H), 4.95 (dd, J=9.5, 5.5 Hz, 1H), 4.00 (t, J=6.0 Hz, 2H), 3.89 (s, 6H), 2.38-2.30 (m, 1H), 2.12-2.05 (m, 1H), 1.91-1.84 (m, 2H), 1.45 (s, 18H); ¹³C NMR (126 MHz, CDCl₃): δ 170.6, 163.6, 161.4, 152.3, 135.6, 128.5, 128.2, 128.0, 111.9, 91.8, 83.3, 67.6, 66.9, 57.8, 56.4, 27.9, 26.0, 26.0; HRMS (ESI) m/z calculated for C₃₁H₄₁N₂O₉S [M+H]⁺: 617.2527, found 617.2522.

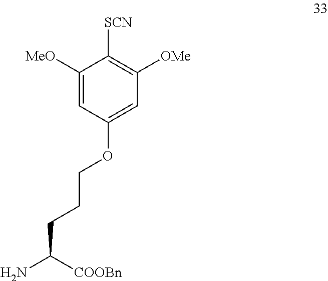

Intermediate (32) (4.0 g, 6.49 mmol) and triisopropylsilane (1.59 mL, 7.78 mmol) were dissolved in trifluoroacetic acid/dichloromethane (10 mL/10 mL) and stirred for 2 h (FIG. 24). The reaction mixture was directly vacuum concentrated. The resulting crude oil was suspended in water and basified to pH=8 with saturated sodium bicarbonate (˜40 mL). The organic products were extracted with ethyl acetate and dried with anhydrous sodium sulfate. The organic layer was concentrated under reduced pressure, and the crude oil was purified via flash column chromatography (dichloromethane/methanol: 50/1) to afford compound (33) are an orange oil (2.16 g, 80% yield). ¹H NMR (500 MHz, CDCl₃): δ 7.30-7.27 (m, 5H), 6.05 (s, 2H), 5.09 (s, 2H), 3.91 (t, J=6.0 Hz, 2H), 3.81 (s, 6H), 3.51-3.48 (m, 1H), 1.89-1.71 (m, 4H); ¹³C NMR (126 MHz, CDCl₃): δ 175.7, 163.7, 161.5, 135.7, 128.8, 128.6, 128.4, 112.0, 91.9, 67.9, 66.9, 56.5, 543, 31.2, 25.5; HRMS (ESI) m/z calculated for C₂₁H₂₅N₂O₅S [M+H]⁺: 417.1479, found 417.1469.

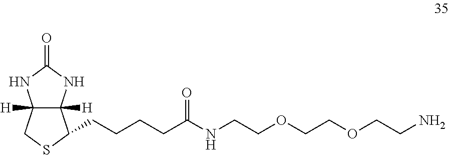 [SMILES: NCCOCCOCCNC(=O)CCCC[C@@H]1SC[C@@H]2NC(=O)N[C@@H]21]

Intermediate (35) was synthesized according to published procedures (Qi K et al., 2004, J. Am. Chem. Soc., 126:6599-6607) (FIG. 25). Briefly, a solution of D-biotin (2.5 g, 10.2 mmol) (compound (34)) in DMF (60 mL) was stirred and heated at 60° C. until fully dissolved. The coupling reagent 1,1′-Carbonyldiimidazole (CDI)(3.32 g, 20.5 mmol) was then added, and the reaction mixture was kept stirring at 60° C. for 3 h, after which the linker 2,2′-(Ethylenedioxy)bis(ethylamine) (5.96 mL, 40.9 mmol) was added. The reaction mixture was stirred overnight at room temperature, and then vacuum concentrated. The crude oil was purified via flash column chromatography (dichloromethane/methanol: 5/1, plus 1% triethylamine) to render compound (35) (3.65 g, 95% yield) as a yellowish oil. ¹H NMR (500 MHz, D₂O): δ 4.49 (dd, J=8.0, 5.0 Hz, 1H), 4.30 (dd, J=7.5, 4.5 Hz, 1H), 3.59-3.50 (m, 7H), 3.41 (t, J=5.5 Hz, 1H), 3.37 (t, J=5.5 Hz, 1H), 3.23-3.19 (m, 1H), 2.93 (dd, J=13.0, 5.0 Hz, 1H), 2.86 (t, J=5.0 Hz, 2H), 2.70 (d, J=13.0 Hz, 1H), 2.22 (t, J=7.5 Hz, 2H), 1.78-1.56 (m, 4H), 1.47-1.41 (m, 2H). MS (ESI) m/z calculated for C₁₆H₃₁N₄O₄S [M+H]⁺: 375.2, found 375.2.

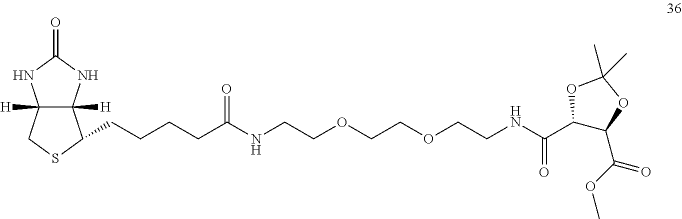 [SMILES: COC(=O)[C@@H]1OC(C)(C)O[C@H]1C(=O)NCCOCCOCCNC(=O)CCCC[C@@H]1SC[C@@H]2NC(=O)N[C@@H]21]

The coupling agent HATU (8.38 g, 22.0 mmol) was mixed with the commercially available building block (4R,5R)-5-(Methoxycarbonyl)-2,2-dimethyl-1,3-dioxolane-4-carboxylic acid (3.0 g, 14.7 mmol) in 40 mL DMF (FIG. 25). Compound (35) (8.25 g, 22.0 mmol) was added, and the reaction mixture was stirred for 10 minutes, followed by the addition of N,N-Diisopropylethylamine (5.12 mL, 29.4 mmol) and the reaction mixture was stirred overnight. The reaction mixture was directly vacuum concentrated and purified via flash chromatography (dichloromethane/methanol: 20/1) to afford compound (36) as an orange oil (6.16 g, 75% yield). ¹H NMR (500 MHz, CD₃OD) δ 4.72 (s, 2H), 4.51 (dd, J=8.0, 4.5 Hz, 1H), 4.32 (dd, J=8.0, 4.5 Hz, 1H), 3.82 (s, 3H), 3.67-3.62 (m, 4H), 3.61 (t, J=5.5 Hz, 2H), 3.56 (t, J=5.5 Hz, 2H), 3.53-3.47 (m, 1H), 3.46-3.41 (m, 1H), 3.38 (q, J=6.0 Hz, 2H), 3.25-3.21 (m, 1H), 2.94 (dd, J=13.0, 5.0 Hz, 1H), 2.72 (d, J=13.0 Hz, 1H), 2.24 (t, J=7.5 Hz, 2H), 1.80-1.58 (m, 4H), 1.50 (s, 3H), 1.47 (s, 3H), 1.12 (d, J=6.5 Hz, 4H); ¹³C NMR (126 MHz, CD₃OD) δ 174.8, 170.8, 170.5, 164.7, 113.3, 78.0, 77.2, 69.9, 69.9, 69.2, 69.0, 62.0, 60.2, 55.6, 51.8, 41.3, 39.6, 38.9, 38.6, 35.3, 28.4, 28.1, 25.5, 25.2, 22.1; HRMS (ESI) m/z calcd for C₂₄H₄₁N₄O₉S [M+H]⁺ 561.2589, found 561.2583.

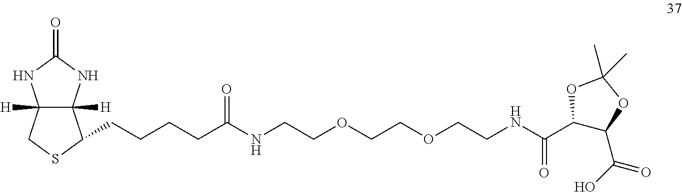 [SMILES: CC1(C)O[C@@H](C(=O)O)[C@H](C(=O)NCCOCCOCCNC(=O)CCCC[C@@H]2SC[C@@H]3NC(=O)N[C@@H]32)O1]

Lithium hydroxide (2 M, 3.2 mL) was slowly added into a solution of compound (36) (3.0 g, 5.35 mmol) in 20 mL methanol at 0° C. (FIG. 25). The reaction mixture was stirred for 2 h and acidified with 1 M HCl (˜5 mL). The crude mixture was vacuum concentrated and purified via high-performance liquid chromatography (HPLC) to afford compound (37) (2.62 g, 90% yield) as a white solid. For HPLC purification (flow rate: 10 mL/min), solvent A is 0.1% TFA containing water while solvent B is 0.1% TFA containing acetonitrile. After the initial 5 min post sample injection, solvent B percentage was increased linearly to 100% within 35 min. The system was continuously flushed with 100% solvent B for another 5 min before the run stopped. Compound peak retention time on HPLC: ˜21 min. ¹H NMR (500 MHz, CD₃OD): δ 4.58 (dd, J=6.0 Hz, 2H), 4.39 (dd, J=8.0, 4.5 Hz, 1H), 4.21 (dd, J=8.0, 4.5 Hz, 1H), 3.54-3.50 (m, 4H), 3.49 (t, J=5.5 Hz, 2H), 3.44 (t, J=5.5 Hz, 2H), 3.41-3.36 (m, 1H), 3.34-3.29 (m, 1H), 3.26 (t, J=5.5 Hz, 2H), 3.13-3.09 (m, 1H), 2.83 (dd, J=13.0, 5.0 Hz, 1H), 2.60 (d, J=12.5 Hz, 1H), 2.13 (t, J=7.5 Hz, 2H), 1.68-1.47 (m, 4H), 1.38 (s, 3H), 1.36 (s, 3H); ¹³C NMR (126 MHz, CD₃OD): δ 174.8, 172.2, 170.9, 164.7, 112.9, 77.9, 77.3, 69.92, 69.88, 69.2, 69.0, 62.0, 60.2, 55.6, 39.6, 38.9, 38.7, 35.3, 28.4, 28.1, 25.4, 25.2; HRMS (ESI) m/z calculated for C₂₃H₃₉N₄O₉S [M+H]⁺: 547.2432, found 547.2424.

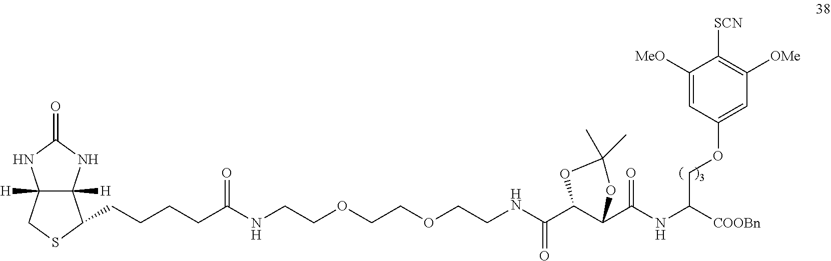

Compound (37) (2.0 g, 3.66 mmol) and HATU (2.1 g, 5.49 mmol) were dissolved in DMF (18 mL) (FIG. 25). Intermediate (33) (0.91 g, 2.19 mmol) was then added and the reaction mixture was stirred for 10 min, followed by the addition of N, N-diisopropylethylamine (DIPEA) (0.57 mL, 3.29 mmol). The reaction mixture was stirred overnight. Upon completion, the mixture was diluted 10-fold with water, and the organic products were extracted with ethyl acetate. The organic layer was dried with sodium sulfate, vacuum concentrated; and the crude mixture was eventually purified via flash column chromatography (dichloromethane/methanol: 30/1)) to afford compound (38) in light-orange solid form (1.64 g, 80% yield). ¹H NMR (500 MHz, CD₃OD): δ 7.58 (d, J=8.5 Hz, 1H), 7.36-7.29 (m, 5H), 6.67 (t, J=5.5 Hz, 1H), 6.11 (s, 2H), 5.21-5.12 (m, 2H), 4.75-4.70 (m, 1H), 4.63 (d, J=6.5 Hz, 1H), 4.54 (d, J=6.5 Hz, 1H), 4.48-4.45 (m, 1H), 4.29-4.26 (m, 1H), 3.97 (t, J=6.0 Hz, 2H), 3.87 (s, 6H), 3.59-3.50 (m, 8H), 3.42-3.37 (m, 2H), 3.13-3.08 (m, 1H), 2.88-2.84 (m, 1H), 2.69 (d, J=13.0 Hz, 1H), 2.20 (t, J=7.5 Hz, 2H), 2.14-2.08 (m, 2H), 1.95-1.88 (m, 1H), 1.86-1.76 (m, 2H), 1.73-1.61 (m, 5H), 1.47 (s, 3H), 1.45 (s, 3H); ¹³C NMR (126 MHz, CD₃OD): δ 173.8, 171.4, 170.2, 169.9, 164.0, 163.5, 161.4, 135.1, 128.7, 128.6, 128.4, 112.9, 111.9, 91.8, 70.1, 70.0, 69.6, 67.5, 67.3, 61.8, 60.3, 56.4, 55.5, 55.4, 53.5, 51.7, 50.7, 40.5, 39.2, 39.1, 35.8, 28.9, 28.1, 28.0, 26.2, 26.1, 25.6, 25.0; HRMS (ESI) m/z calculated for C₄₄H₆₁N₆O₁₃S₂ [M+H]⁺: 945.3733, found 945.3722.

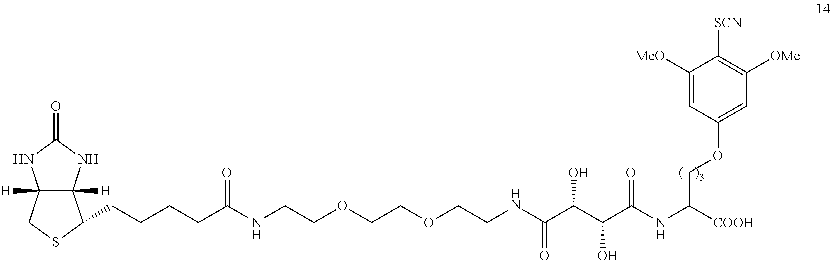

Compound (38) (1.0 g, 1.06 mmol) was dissolved in acetic acid/water (9 mL/1 mL) and refluxed for ˜24 h, at which point the complete diol deprotection was confirmed by LC-MS analysis (FIG. 25). The reaction mixture was then vacuum concentrated to afford the diol intermediate as a white solid. The diol intermediate was resuspended in THE (8 mL) at 0° C., and 2 M lithium hydroxide (2.09 mmol, ˜1.1 mL) in water was added dropwisely. The reaction mixture was stirred for ˜2 h, at which point the hydrolysis was confirmed complete by TLC. The mixture was acidified with 1 M HCl (˜3 mL), vacuum concentrated, and finally purified by HPLC. The HPLC purification utilized water (0.1% TFA) as solvent A and acetonitrile (0.1% TFA) as solvent B. The flow rate was 10 mL/min. For the first 5 min post injection, 100% solvent A was used in the program. After that, solvent B percentage was linearly increased to 50% within 55 min. Then the HPLC flow was changed to 100% solvent B within the next 20 min, followed by additional 5 min flushing with solvent B percentage remaining at 100%. Compound peak retention time on HPLC was ˜58 min. The eluted fraction was lyophilized to afford compound (14) as a white solid (334 mg, 40% yield). ¹H NMR (500 MHz, DMSO-d₆): δ 7.84 (t, J=5.5 Hz, 1H), 7.80 (d, J=8.0 Hz, 1H), 7.64 (t, J=6.0 Hz, 1H), 6.29 (s, 2H), 4.35-4.28 (m, 3H), 4.24 (d, J=2.0 Hz, 1H), 4.12 (dd, J=7.5, 4.5 Hz, 1H), 3.97 (t, J=6.0 Hz, 1H), 4.48-4.45 (m, 1H), 4.29-4.26 (m, 1H), 3.97 (t, J=6.0 Hz, 2H), 3.80 (s, 6H), 3.53-3.48 (m, 4H), 3.44 (t, J=6.0 Hz, 2H), 3.39 (t, J=6.0 Hz, 2H), 3.35-3.28 (m, 1H), 3.26-3.22 (m, 1H), 3.21-3.14 (m, 2H), 3.13-3.07 (m, 1H), 2.82 (dd, J=7.5, 5.0 Hz, 1H), 2.59-2.54 (m, 1H), 2.07-2.04 (m, 2H), 1.97-1.91 (m, 1H), 1.83-1.75 (m, 1H), 1.74-1.67 (m, 2H), 1.63-1.56 (m, 1H), 1.52-1.41 (m, 3H), 1.33-1.28 (m, 2H); ¹³C NMR (126 MHz, DMSO-d₆): δ 173.6, 172.6, 172.3, 172.3, 163.2, 158.0, 156.0, 92.5, 73.0, 72.8, 70.0, 69.7, 69.4, 67.7, 61.5, 59.7, 56.6, 55.9, 54.1, 51.8, 38.9, 38.8, 35.6, 28.7, 28.5, 25.7, 25.4, 18.6, 17.2; HRMS (ESI) m/z calculated for C₃₃H₅₂N₅O₁₃S₂ [M+H]⁺: 790.2998, found 790.2988.

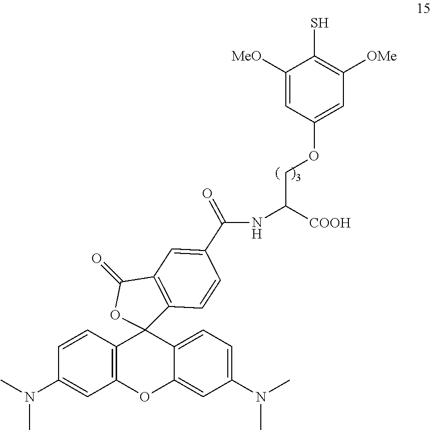

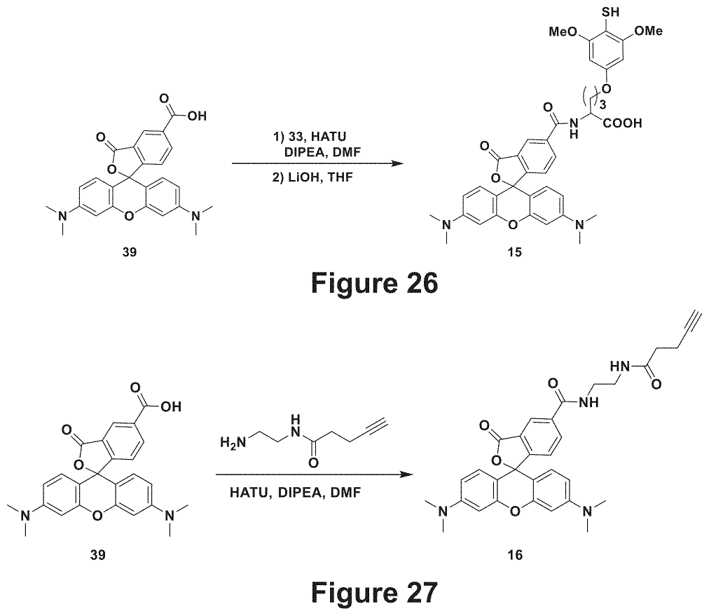

5-carboxytetramethylrhodamine (5-TAMRA, compound (39), Thermo Fisher, 43 mg, 0.10 mmol) was dissolved in 2 mL DMF, and mixed with HATU (46.0 mg, 0.12 mmol) (FIG. 26). The reaction mixture was stirred for 10 min to ensure it was fully dissolved. The previously prepared (33) (42.0 mg, 0.10 mmol) was added to the solution, followed by DIPEA (53.0 μL, 0.30 mmol). The resulting mixture was stirred at RT for 3 h and was vacuum concentrated upon completion as confirmed by TLC. The crude mixture was purified via flash column chromatography (dichloromethane/methanol: 20/1) to afford the conjugated intermediate as purple oil, which was directly dissolved in THE (2 mL) and cooled down to 0° C. Lithium hydroxide (2 M, 0.06 mL) in water was added dropwisely, and the solution was stirred at RT for 2 hours at which point the hydrolysis was near complete as confirmed by TLC. The mixture was then acidified with 1M HCL (˜200 μL), and vacuum concentrated. The crude mixture was purified via HPLC that implemented water (0.1% TFA) as solvent A and acetonitrile (0.1% TFA) as solvent B, with a flow rate of 10 mL/min. The purification program was run as the following: 5.0% solvent B for the first minute, followed by a liner progression to 70.0% solvent B for the next 39 minutes, ending with the last 5 minutes of 100.0% solvent B for a total of 45 minutes per HPLC run. The product peak came out at 40 min, and lyophilization of the collected fraction rendered compound (15) (39.6 mg, 56% yield) as a pink solid. ¹H NMR (500 MHz, CD₃OD): δ 8.76-8.74 (m, 1H), 8.23-8.19 (m, 1H), 7.48 (d, J=8.0 Hz, 1H), 7.14-7.10 (m, 2H), 7.06-7.03 (m, 2H), 6.97-6.93 (m, 2H), 6.25 (s, 2H), 4.78-4.73 (m, 1H), 4.10-4.04 (m, 2H), 3.80 (s, 6H), 3.28 (s, 12H), 2.30-2.22 (m, 1H), 2.12-2.03 (m, 1H), 2.00-1.94 (m, 2H); ¹³C NMR (126 MHz, CD₃OD): δ 175.3, 168.4, 167.3, 160.7, 159.2, 159.1, 159.0, 157.3, 138.3, 137.3, 132.8, 132.5, 132.0, 132.0, 131.9, 131.6, 115.6, 114.7, 101.2, 97.4, 93.1, 68.5, 56.6, 54.3, 40.9, 29.2, 27.1; MS (ESI) m/z calculated for dimerized probe C₇₆H₇₈N₆O₁₈S₂ [M+2H]²+: 713.2402, found 713.2416.

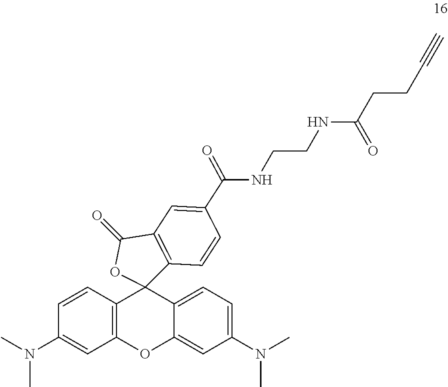 [SMILES: C#CCCC(=O)NCCNC(=O)c1ccc2c(c1)C(=O)OC21c2ccc(N(C)C)cc2Oc2cc(N(C)C)ccc21]

Compound (39) (43.0 mg, 0.10 mmol) was mixed with HATU (46.0 mg, 0.12 mmol) in 2 mL DMF, and the mixture was stirred for 10 min (FIG. 27). N-(2-aminoethyl)pent-4-ynamide (17.0 mg, 0.12 mmol) was then added, followed by 53.0 μL of N,N-diisopropylethylamine (0.30 mmol). The reaction mixture was stirred for 3 h and was vacuum concentrated upon completion by TLC. The crude reaction mixture was purified by HPLC using water (0.1% TFA) as solvent A and acetonitrile (0.1% TFA) as solvent B. The flow at a rate of 10 mL/min used the program same as that used for purifying compound (15). Compound (16) (38.6 mg, 70% yield) was eventually obtained as a pink solid with a retention time of 30 min. ¹H NMR (500 MHz, CD₃OD): δ 8.67 (d, J=1.5 Hz, 1H), 8.15 (dd, J=8.0, 1.5 Hz, 1H), 7.43 (d, J=8.0 Hz, 1H), 7.04 (d, J=9.5 Hz, 2H), 6.96 (dd, J=9.5, 2.5 Hz, 2H), 6.88 (d, J=2.0 Hz, 2H), 3.50-3.47 (m, 2H), 3.42-3.38 (m, 2H), 3.21 (s, 12H), 2.40-2.36 (m, 2H), 2.35-2.31 (m, 2H), 2.16 (t, J=2.5 Hz, 1H); ¹³C NMR (126 MHz, CD₃OD): δ 174.7, 168.5, 167.5, 160.7, 159.1, 159.0, 138.1, 137.6, 133.1, 132.3, 131.94, 131.91, 131.3, 115.6, 114.7, 97.4, 83.5, 70.3, 41.3, 40.9, 39.9, 36.1, 15.7; MS (ESI) m/z calculated for C₃₂H₃₃N₄O₅ [M+H]⁺: 553.2445, found 553.2446.

### Example 4: A FTDR-Based Peptide Stapling Strategy

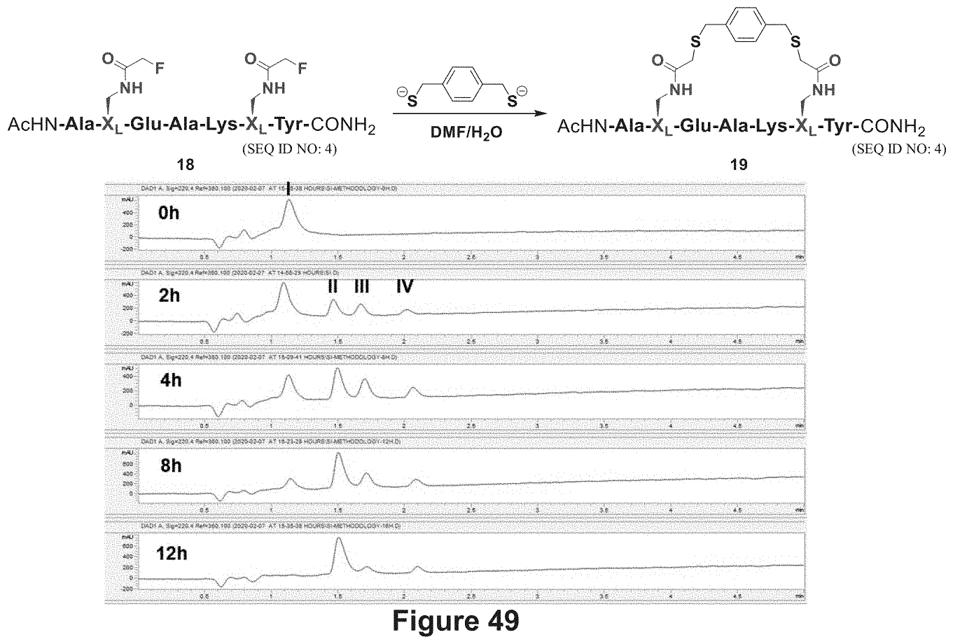

Protein-protein interactions (PPIs) mediate important cellular processes including certain pathways that may lead to diseases, such as cancer. Recently, mimics of alpha helices that are the most common secondary structural motifs found in PPIs can act as competitive inhibitors and are thereby drawing significant attention as potential therapeutics against previously undruggable targets. Yet, most short alpha helixes as single units are unstructured and random coils with consequent loss of binding affinities. Peptide stapling has emerged as an efficient approach to retain the native conformation and binding capabilities, and sometimes with even improved proteolytic stability and cell permeability. Herein, a FTDR-based novel peptide stapling methodology that uses a pair of alpha fluoroimide containing amino acids is reported (FIG. 49 through FIG. 51). Both S and R configurations of the unnatural amino acid have been explored by incorporating them into Axin-derived catenin-targeting or HIV-targeting peptides. A wide range of dithiol-containing linkers have been tested to crosslink the linear peptides. Among them, the cyclic Si, i+4R and Si, i+4S analog with 1,3-benzenedimethanethiol promoted highest alpha helicity. These stapled Axin-derived peptides maintained the binding affinities against beta-catenin protein that regulates the Wnt-signaling needed by cancer cells, and were stable in rat plasma for at least 2 h. Better cell permeability of these stapled peptides in DLD1 cell lines has been observed compared to the hydrocarbon stapling counterpart. Compared to the proteoglycan aided cell penetration by peptides stapled with ring-closing metathesis (RCM), the preliminary mechanistic studies of the FTDR-stapled peptides revealed that the thiolated linkers rendered peptides multiple pathways (clathrin-dependent endocytosis, actin polymerization, and proteoglycan-aided) to take for entering cells. In summary, the FTDR-based stapling approach may provide a novel class of cell permeable peptides to probe intracellular targets.

Currently, stapled peptides emerged as a promising class of peptide therapeutics, due to the retained folding as alpha-helix, the enhanced biostability towards protease, etc. A representative company that has been promoting this technology into clinical drugs is Aileron Therapeutics, Inc. Hereby, a novel stapling strategy based on FTDR chemical reaction was invented. The resulting peptides possessed improved cell permeability while maintaining comparable stability, helicity, etc.

As disclosed herein, the stapling strategy resulted in peptides with improved cell permeability, due to the novel and additional cell entry pathways this class of peptides undertake.

Targeting any disease-related intracellular targets. It can be used as therapeutics for cancer, inflammation, or neuroregeneration (e.g. spinal cord injury treatment). It can be also used as sensors or antibody mimics to detect any biomarkers related to disease.

A novel peptide stapling strategy based on the fluorine-thiol displacement reaction (FTDR), which could generate a class of stapled peptides that maintain decent folding, stability, binding affinity, but possess better cell penetrating ability. The resulting peptides could be a novel class of therapeutic leads to target intracellular proteins or protein-protein interactions that are key to the onset or relapse of diseases, such as cancer, inflammatory disorders, and neuron regeneration/degeneration.

### Example 5: Unprotected Peptide Macrocyclization and Stapling Via a Fluorine-Thiol Displacement Reaction

PPIs also regulate important molecular processes including gene replication, transcription activation, translation, and transmembrane signal transduction (Wan J et al., 2016, Lead Generation, 259-306; Scott D E et al., 2016, Nat. Rev. Drug Discov., 15:533-550; Zinzalla G et al., 2009, Future Med. Chem., 1:65-93). Aberrant PPIs have been consequently implicated in the development of diseases such as cancer, infections, and neurodegenerative diseases (Zinzalla G et al., 2009, Future Med. Chem., 1:65-93; Ryan D P et al., 2005, Curr. Opin. Struct. Biol., 15:441-446). Targeting PPIs has since emerged as a promising therapeutic strategy that specifically inhibits specific molecular pathways without compromising other functions of the involved proteins (Bakail M et al., 2016, Comptes Rendus Chimie, 19:19-27). Yet this avenue is challenging due to the generally flat, shallow, and extended nature of PPI interfaces (Wan J et al., 2016, Lead Generation, 259-306; Pelay-Gimeno M et al., 2015, Angew. Chem. Int. Ed Engl., 54:8896-8927; Tian Y et al., 2016, Chem. Sci., 7:3325-3330). In this regard, efficient mimicking of peptides involved in PPIs is a long-standing direction in PPI inhibitor development (Pelay-Gimeno M et al., 2015, Angew. Chem. Int. Ed Engl., 54:8896-8927). Driven by entropy and the interactions with water, short peptides are mostly unstructured random coils in aqueous solutions (Smith L J et al., 1996, Fold. Des., 1:R₉₅-106). Given that over fifty percent of PPIs involve α-helices (Bullock B N et al., 2011, J. Am. Chem. Soc., 133:14220-14223; Jochim A L et al., 2009, Mol. Biosyst., 5:924-926), one common approach used chemical stapling to restore and stabilize the bioactive helical conformation of short peptides (Lau Y H et al., 2015, Chem. Soc. Rev., 44:91-102). By introducing side-chain to side-chain crosslinking of residues positioned at the same face (an i, i+4 or i, i+7 fashion) of the a helix, the structure of peptides are locked in folded conformation due to the reduced entropy penalty (Lau Y H et al., 2015, Chem. Soc. Rev., 44:91-102).

Using olefin-containing unnatural amino acids and RCM mediated chemical crosslinking, Grubbs, Verdine, Walensky, and others. have done seminal work to develop RCM-stapled peptides as a promising class of therapeutics (Blackwell H E et al., 1998, Angew Chem Int Ed Engl, 37:3281-3284; Walensky L D et al., 2004, 2004, Science, 305:1466-1470; Grossmann T N et al., 2012, Proc Natl Acad Sci USA, 109:17942-17947; Bird G H et al., 2014, ACS Chem Biol, 9:831-837; Chapuis H et al., 2012, Amino Acids, 43:2047-2058; Cromm P M et al., 2015, ACS Chem Biol, 10:1362-1375; Walensky L D et al., 2014, J Med Chem, 57:6275-6288). The hydrocarbon stapled peptides not only maintained the desired tertiary structures and the associated targeting specificities, but also possessed increased binding affinity and protease resistance (Wan J et al., 2016, Lead Generation, 259-306; Bird G H et al., 2014, ACS Chem Biol, 9:831-837; Walensky L D et al., 2014, J Med Chem, 57:6275-6288). One hydrocarbon stapled peptide, a lead p53 mimic (ALRN-6924), has been in clinical testing to treat a diverse set of tumors including acute myeloid leukemia (AML) (Scott D E et al., 2016, Nat. Rev. Drug Discov., 15:533-550). Yet, RCM-based stapling necessitates the use of metal-based catalysts that were sometimes incompatible with other functional groups present in peptides such as a thiourea moiety (Verdine G L et al., 2012, Methods Enzymol., 503:3-33). The increased hydrophobicity brought by hydrocarbon linkers may also incur issues in targeting specificity and aqueous solubility(Verdine G L et al., 2012, Methods Enzymol., 503:3-33). To date, a number of other stapling strategies based on different crosslinking chemistry have been established (Tian Y et al., 2016, Chem. Sci., 7:3325-3330; Lau Y H et al., 2015, Chem. Soc. Rev., 44:91-102; Muppidi A et al., 2012, J Am Chem Soc, 134:14734-14737; Kumita J R et al., 2000, Proc Natl Acad Sci USA, 97:3803-3808; Jo H et al., 2012, J Am Chem Soc, 134:17704-17713; Wang Y et al., 2015, Angew Chem Int Ed Engl, 54:10931-10934; Mendive-Tapia L et al., 2015, Nat Commun, 6:7160; Lau Y H et al., 2014, Chem Sci, 5:1804-1809; Spokoyny A M et al., 2013, J Am Chem Soc, 135:5946-5949; Kawamoto S A et al., 2012, J Med Chem, 55:1137-1146), each bearing different strengths and weaknesses (Tian Y et al., 2016, Chem. Sci., 7:3325-3330; Mendive-Tapia L et al., 2015, Nat Commun, 6:7160; Lau Y H et al., 2014, Chem Sci, 5:1804-1809). For instance, many approaches required additional catalysts such as metal complexes or photoinitiators. Direct crosslinking based on bromo or iodo-alkyl/benzyl chemistry, on the other hand, could produce over-alkylated species due to their increased cross-reactivity (Muppidi A et al., 2012, J Am Chem Soc, 134:14734-14737; Kumita J R et al., 2000, Proc Natl Acad Sci USA, 97:3803-3808; Jo H et al., 2012, J Am Chem Soc, 134:17704-17713; Lau Y H et al., 2014, Chem Sci, 5:1804-1809). More importantly, the vast majority of stapled peptides still had limited cell permeability (Dougherty P G et al., 2019, Journal of Med Chem, 62:10098-10107). As a result, the development of stapled peptides with designable cell permeability remains challenging and requires a delicate balance between positive charges, hydrophobicity, alpha-helicity, and staple position (Dougherty P G et al., 2019, Journal of Med Chem, 62:10098-10107; Chu Q et al., 2015, MedChemComm, 6:111-119; Bird G H et al., 2016, Nat Chem Biol, 12:845-852).

Thus, novel strategies capable of stapling unprotected peptides in a straightforward, chemoselective, and clean manner, as well as promoting cellular uptake are highly sought (Lau Y H et al., 2015, Chem. Soc. Rev., 44:91-102). Unlike other readily reacting α-haloacetamides, a fluoroacetamide functional group has been considered biologically inert due to the poor leaving capability of fluorine (Kobayashi T et al., 2016, J Am Chem Soc, 138:14832-14835). A number of probes consisting of the radiolabeled [¹⁸F]fluoroacetamide have been thereby developed as positron emission tomography (PET) agents for in vivo diagnostics (Seo Y J et al., 2013, Bioorg Med Chem Lett, 23:6700-6705; Reid A E et al., 2009, Nucl Med Biol, 36:247-258; Zeglis B M et al., 2011, Nucl Med Biol, 38:683-696), indicating the biorthogonality of the fluoroacetamide functional group. Nevertheless, fluoroacetamide was recently incorporated into proteins as the side-chain of an unnatural amino acid, and was revealed to react with the thiol group of cysteine within protein-confined close proximity (Kobayashi T et al., 2016, J Am Chem Soc, 138:14832-14835).

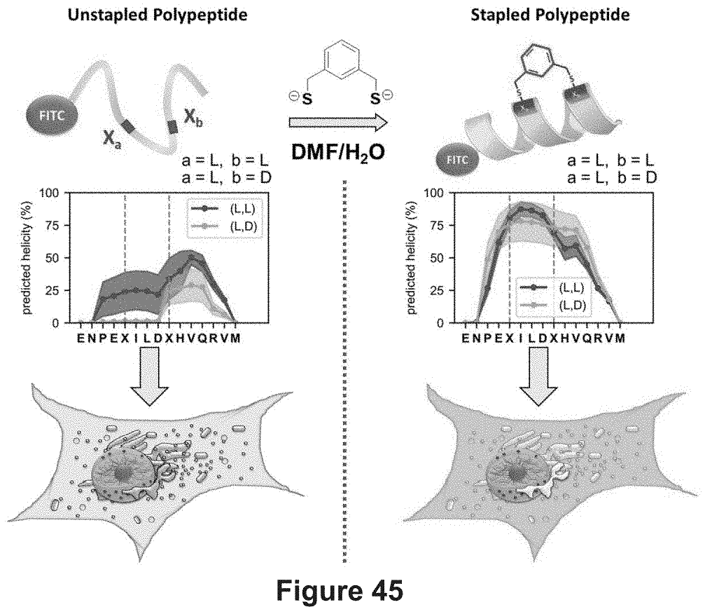

The present example discloses the discovery of a fluorine displacement reaction using different thiol-containing linkers, which were used for the mild functionalization of unprotected peptides. This versatile approach allowed the facile preparation of constrained macrocyclized peptides of different linker sizes, and led to the identification of stapled peptides that possessed improved target binding and cellular permeability compared to hydrocarbon stapled peptides (FIG. 45). The peptide analogues stapled via FTDR using the 1,3-benzenedimethanethiol linker in general demonstrated approximately five-fold enhanced cellular uptake than the hydrocarbon stapled ones. The corresponding Axin analogues also showed enhanced growth inhibition of cancer cell growth as a result of increased cellular uptake, demonstrating the potential of FTDR-stapled peptides as probes for targeting intracellular compartments.

FTDR-Based Coupling with a Model Compound/Peptide

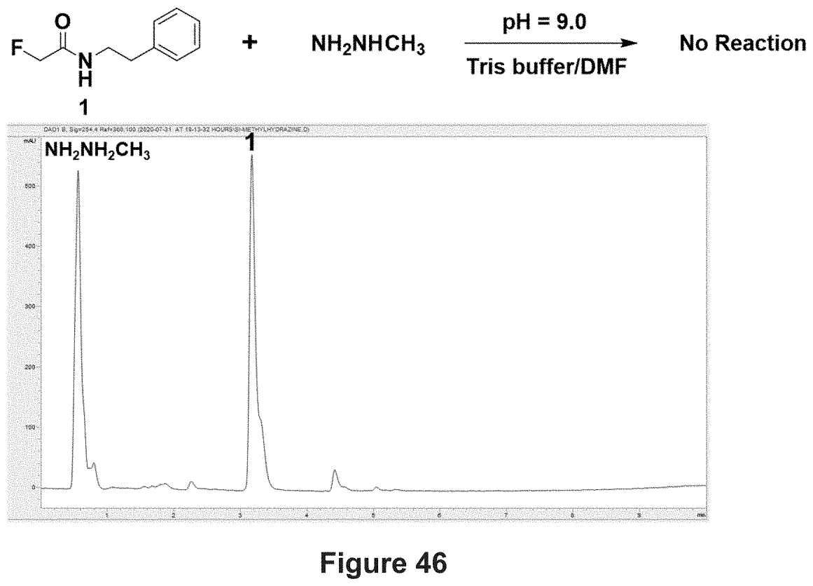

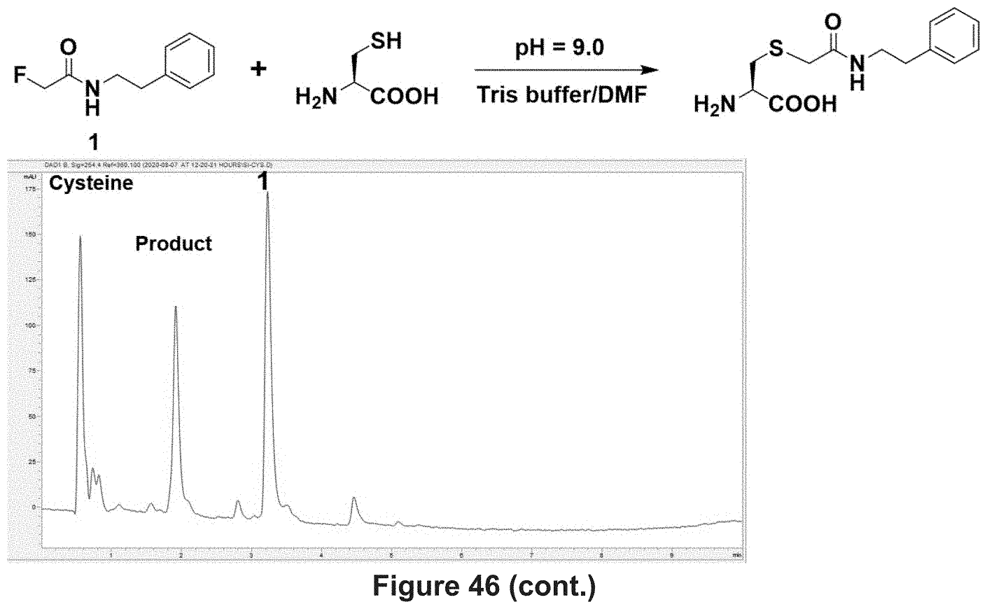

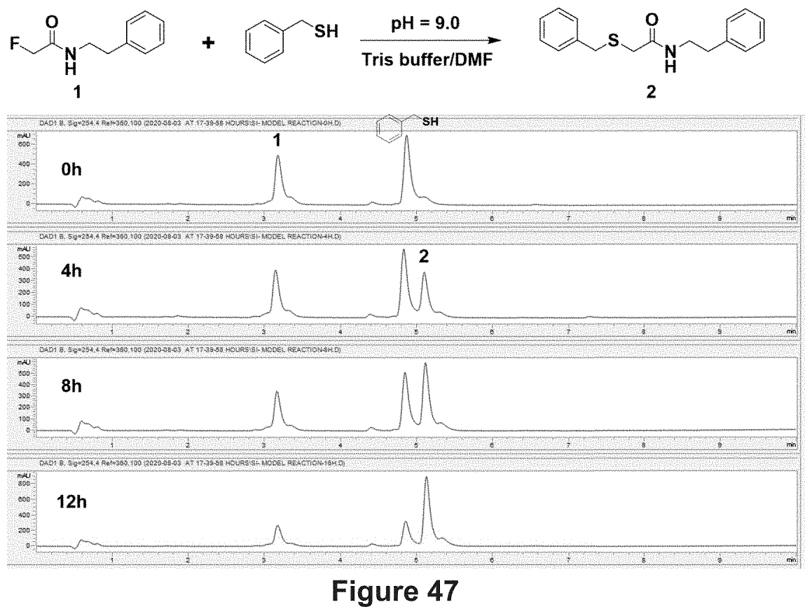

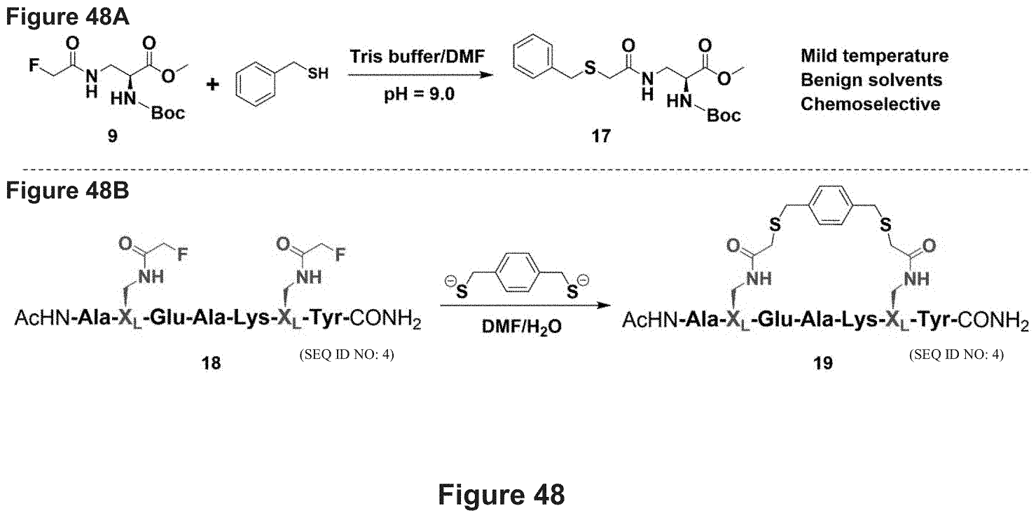

Initially, the previously reported fluoroacetamide (1) (Miscevic S et al., 1992, J Fluor Chem, 59:239-247) was used as a model compound that was UV active and allows the facile monitoring of reaction progress on LC-MS (FIG. 46). Evaluation of its reaction with common nucleophiles, such as methyl hydrazine and cysteine at a mildly basic Tris (tris(hydroxymethyl)-aminomethane) buffer (FIG. 46) revealed that although there was no reaction between fluoroacetamide and methyl hydrazine after 12 h of incubation at 37° C., approximately 40.4% of fluoroacetamide has been converted by cysteine to the fluorine-displaced adduct. This indicated that the fluoroacetamide functional group reacted specifically to thiols at high concentrations. Subsequent studies investigated the fluorine thiol displacement reaction with a more nucleophilic benzyl thiol (Shigeru O et al., 1965, Bulletin of the Chemical Society of Japan, 38:1381-1385; Wu M H et al., 1998, J Org Chem, 63:5252-5254), and have observed a further enhanced reaction (FIG. 47), rendering a relative yield of 52.7%. With this encouraging result, further studies investigated the reaction on a protected amino acid building block (9), which had a natural amino acid backbone but possessed a fluoroacetamide functional group in the side chain (FIG. 48A). Consistent with the previously observed FTDR results, the alpha fluoride was efficiently replaced by benzyl thiol, giving the desired product (17) at a yield of 73% after purification. Additional studies then focused on the evaluation of the chemoselectivity of this FTDR as a general macrocyclization method using a seven amino acid long model peptide (18; SEQ ID NO: 4) that bore multiple unprotected functional groups (FIG. 48B). Given the documented ability of the sulfur to stabilize the negative charge (Spokoyny A M et al., 2013, J Am Chem Soc, 135:5946-5949), 1,4-benzenedimethanethiol, a previously reported bifunctional thiol linker (Wu M H et al., 1998, J Org Chem, 63:5252-5254), was deprotonated with a stoichiometric amount of base and was then incubated with the model peptide (18; SEQ ID NO: 4) in a mixture of water/DMF. The reaction was monitored by LC-MS analysis (FIG. 49), which indicated significant conversion of the starting material and the mono-substituted intermediate into the macrocyclized peptide product (19; stapled SEQ ID NO: 4). The observed transformation was >90% completed after 12 h, and the yield was approximately 62% after HPLC purification. Given the observed conversion with benzenedimethanethiol, the FTDR based coupling appeared to be chemoselective and orthogonal to functional groups in natural amino acid side chains such as carboxylic acids, amines, and phenolic alcohols.

Substrate Scope with Various Linkers

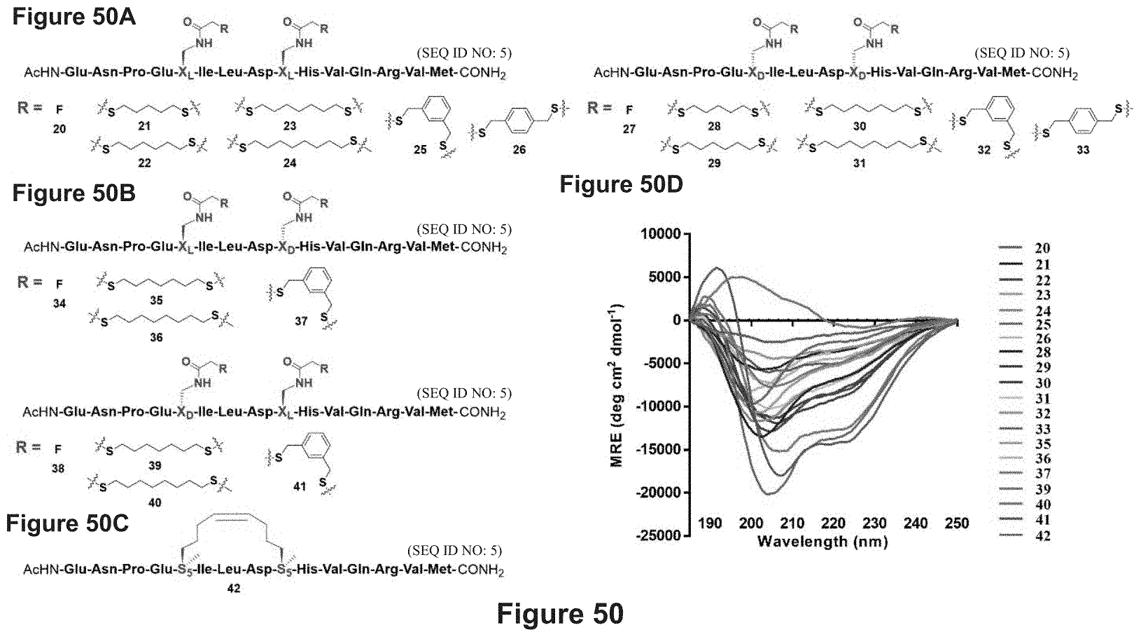

Subsequent studies then focused on the evaluation of the FTDR-based cyclization on varied peptides of interesting chemical and biological properties. The Axin mimetic analogue first caught attention as it was a classic peptide used for stapling (Grossmann T N et al., Proc Natl Acad Sci USA, 109:17942-17947; Wang Y et al., 2015, Angew Chem Int Ed Engl, 54:10931-10934), and its blocking of the Axin-β-catenin PPI has been shown to inhibit the Wnt signaling pathway that is crucial for the development of colorectal cancers and acute myeloid leukemia (Grossmann T N et al., Proc Natl Acad Sci USA, 109:17942-17947; Ysebaert L et al., 2006, Leukemia, 20:1211-1216; Wang Y et al., 2010, Science, 327:1650-1653; Chung E J et al., 2002, Blood, 100:982-990). A pair of fluoroacetamide-containing amino acids with a combination of possible chirality (X_{L} or X_{D}) were incorporated into the i and i+4 positions to render the fifteen amino acid long fluoroacetamide containing Axin analogues (SEQ ID NO: 5) (X_{L},X_{L} for peptide (20), X_{D},X_{D} for (27), X_{L},X_{D} for (34), and X_{D},X_{L} for (38)) (FIG. 50A and FIG. 50B). With the optimized reaction conditions, FTDR-based macrocyclization were investigated on these peptides with aliphatic dithiols of various lengths or more rigid and reactive benzenedimethanethiol linkers. As demonstrated in FIG. 50, most linkers cyclized fluoroacetamide-containing peptides in satisfactory yields (Table 1).

TABLE 1
Summary of the molecular weights (MWs) and yields
of stapled Axin analogues.

| | | Observed | Observed | |
|---|---|---|---|---|
| | Theoretical | MW | MW | % |
| Peptide | MW in Da | [M + 2H]/2 | [M + 3H]/3 | yield |
| 21 | 2007.9 | 1005.1 | 670.5 | 54.8% |
| 22 | 2021.9 | 1012.2 | 675.2 | 59.2% |
| 23 | 2036.0 | 1019.2 | 679.9 | 48.6% |
| 24 | 2050.0 | 1026.1 | 684.5 | 51.9% |
| 25 | 2041.9 | 1022.2 | 681.9 | 62.6% |
| 26 | 2041.9 | 1022.1 | 681.8 | 52.2% |
| 28 | 2007.9 | 1005.2 | 670.5 | 58.2% |
| 29 | 2021.9 | 1012.2 | 675.2 | 61.7% |
| 30 | 2036.0 | 1019.1 | 679.9 | 60.4% |
| 31 | 2050.0 | 1026.1 | 684.6 | 55.3% |
| 32 | 2041.9 | 1022.2 | 681.9 | 59.3% |
| 33 | 2041.9 | 1022.2 | 681.9 | 64.9% |
| 35 | 2036.0 | 1019.1 | 679.9 | 53.9% |
| 36 | 2050.0 | 1026.1 | 684.6 | 43.2% |
| 37 | 2041.9 | 1022.2 | 681.9 | 58.4% |
| 39 | 2036.0 | 1019.1 | 679.9 | 39.4% |
| 40 | 2050.0 | 1026.1 | 684.5 | 49.8% |
| 41 | 2041.9 | 1022.2 | 681.9 | 50.9% |
| 42* | 1870.0 | 936.2 | 624.5 | 73.4% |

(*Control peptides stapled by RCM)

Efficient macrocyclization was observed on both X_{L} (20) or X_{D} (27) enantiomer combination with aliphatic linkers from 5-carbon to 8-carbon long, and benzenedimethanethiol linkers at meta or para positions. The oppositely angled fluoroacetamide-containing side chains in the X_{L},X_{D} (34) or X_{D},X_{L} (38) substrates seemed to bring in more rigid conformations and thereby were only crosslinked by 7-carbon and 8-carbon long aliphatic linkers as well as 1,3-benzenedimethanethiol as the only tolerated aromatic linker. For all the substrates being tested, macrocyclization appeared to proceed more efficiently than those in small molecule model reactions, presumably due to the complete deprotonation of thiols in advance that ensured the generation of more nucleophilic thiolate anions (Nair D P et al., 2014, Chemistry of Materials, 26:724-744). Taken together, FTDR-based coupling represents an efficient macrocyclization approach in synthesizing cyclic unprotected peptides with flexible linker choices.

Linker and Chirality Requirement in FTDR-Based Stapling

With the library of cyclized Axin peptide analogues on hand, subsequent studies focused on to evaluate the stapling effect of these dithiol linkers crosslinked at the i, i+4 positions. As a positive control, the RCM-stapled Axin analogue (42) (stapled SEQ ID NO: 5) (FIG. 50C) that had a reported helicity of around 51% was prepared (Grossmann T N et al., Proc Natl Acad Sci USA, 109:17942-17947). Circular dichroism (CD) experiments were performed to measure the alpha helicity of these peptides (FIG. 50D). As demonstrated in Table 2, the highest helicity was achieved with peptides (25) (stapled SEQ ID NO: 5) and (37) (stapled SEQ ID NO: 5), with a mean value of 46% and 44%, respectively, which were close to the measured helicity of the RCM stapled control (42) (stapled SEQ ID NO: 5). Although (25) (L,L) and (37) (L,D) possessed different substrate chirality's, both analogues were stapled well by the 1,3-benzenedimethanethiol linker, indicating this aromatic linker universally promoted the alpha helicity of fluoroacetamide-containing peptides. In comparison, cyclized peptides with chirality combinations of D,D ((28)-(33) (stapled SEQ ID NO: 5)) or D,L ((39)-(41) (stapled SEQ ID NO: 5)) all failed to display enhanced alpha helicity to a comparable extent, even for the ones ((32) with a helicity of 13%, (41) with a helicity of 8%) stapled by the 1,3-benzenedimethanethiol linker. Consistent to the literature report (Schafmeister C E et al., 2000, Journal of the American Chemical Society, 122:5891-5892), substitution with both D-configured amino acids at i, i+4 positions largely destabilized the intrinsic helix conformation. Yet, the inclusion of D-amino acids at the i position has been well documented, with the resulting D,L crosslinked peptide substrates usually inheriting enhanced stabilization of alpha helixes and improved binding affinity in comparison to the L,L crosslinked peptides (Muppidi A et al., 2012, J Am Chem Soc, 134:14734-14737; Schafmeister C E et al., 2000, Journal of the American Chemical Society, 122:5891-5892; Jackson D Y et al., 1991, Journal of the American Chemical Society, 113:9391-9392; Muppidi A et al., 2011, Chemical Communications, 47:9396-9398; Leduc A M et al., 2003, Proc Natl Acad Sci USA, 100:11273-11278). On the other hand, there was little observation of D-amino acid's beneficial effects at the i+4 position either, as most crosslinked L,D analogues resulted in a negative effect to the peptides' alpha helix conformation (Jackson D Y et al., 1991, Journal of the American Chemical Society, 113:9391-9392; Leduc A M et al., 2003, Proc Natl Acad Sci USA, 100:11273-11278). Nevertheless, D-propargylglycine was once substituted at i+4 (Kawamoto S A et al., 2012, J Med Chem, 55:1137-1146), and compared to its L-enantiomer, Copper-mediated Huisgen 1,3-dipolar cycloaddition between L-azido norleucine and D-propargylglycine at i, i+4 positions resulted in much less distortion to the peptide backbone conformation (Kawamoto S A et al., 2012, J Med Chem, 55:1137-1146). These data demonstrated that for stapling-induced alpha helical stabilization the chirality requirements of substrate side chains largely depend on the chemistry used for stapling, and specifically the chemical structures desired for both side chains and crosslinkers.

TABLE 2
Summary of the helicity of peptides
stapled at i, i+4 positions.

| | Fluoro | Helicity |
|---|---|---|
| Peptide | substrates | (%) |
| 21 | Lᵢ, ᵢ₊₄L | 24% |
| 22 | Lᵢ, ᵢ₊₄L | 29% |
| 23 | Lᵢ, ᵢ₊₄L | 18% |
| 24 | Lᵢ, ᵢ₊₄L | 25% |
| 25 | Lᵢ, ᵢ₊₄L | 46% |
| 26 | Lᵢ, ᵢ₊₄L | 17% |
| 28 | Dᵢ, ᵢ₊₄D | 14% |
| 29 | Dᵢ, ᵢ₊₄D | 28% |
| 30 | Dᵢ, ᵢ₊₄D | 31% |
| 31 | Dᵢ, ᵢ₊₄D | 25% |
| 32 | Dᵢ, ᵢ₊₄D | 13% |
| 33 | Dᵢ, ᵢ₊₄D | 19% |
| 35 | Lᵢ, ᵢ₊₄D | 25% |
| 36 | Lᵢ, ᵢ₊₄D | 14% |
| 37 | Lᵢ, ᵢ₊₄D | 44% |
| 39 | Dᵢ, ᵢ₊₄L | 19% |
| 40 | Dᵢ, ᵢ₊₄L | 5% |
| 41 | Dᵢ, ᵢ₊₄L | 8% |
| 42* | S₅, S₅ | 48% |
| 43 | Lᵢ, ᵢ₊₄L | 50% |
| 44 | Lᵢ, ᵢ₊₄D | 45% |
| 45 | Dᵢ, ᵢ₊₄L | 9% |
| 46* | S₅, S₅ | 52% |

(*Control peptides stapled by RCM)

To explore if the L,D versus D,L substrate preference for FTDR-based stapling is generally applicable to different peptide sequences, additional studies focused on another model peptide, which bonded to the C-terminal region of an HIV-1 capsid assembly polyprotein (HIV C-CA) that is key to viral assembly and core condensation (Sticht J et al., 2005, Nat Struct Mol Biol, 12:671-677). The 12-mer long peptide was previously demonstrated to have efficiently stabilized alpha-helical conformation once RCM-based stapling was performed at the indicated i and i+4 positions ((46) (stapled SEQ ID NO: 6), FIG. 51) (Spokoyny A M et al., 2013, J Am Chem Soc, 135:5946-5949; Zhang H et al., 2008, Journal of molecular biology, 378:565-580). Using the optimized linker (1,3-benzenedimethanethiol), FTDR-based stapling were performed on the same sequence positions, and obtained analogues (43)-(45) (stapled SEQ ID NO: 6) that have fluoroacetamide substrates of different chirality combinations (FIG. 51A, Table 3). FITC labeling was applied to the N-terminal during solid-phase synthesis in order to facilitate follow-up biological characterizations. Notably, stapling of the unprotected FITC-labelled peptides (43)-(45) proceeded smoothly despite the presence of FITC's thiourea moiety. As shown in CD spectra (FIG. 51B) and Table 2, stapling with L,L or L,D substrates generated peptides (43) and (44) that possessed similar helicity to the control peptide stapled by RCM. On the contrary, the crosslinked D,L substrate-containing analogue (45) only displayed a minimal level of helicity.

TABLE 3
Summary of the molecular weights (MWs) and yields of stapled peptides
for cell permeability studies (*Control peptides stapled by RCM).

| | | Observed | Observed | Observed | Observed | Observed | |
|---|---|---|---|---|---|---|---|
| | Theoretical | MW | MW | MW | MW | MW | |
| Peptide | MW in Da | [M + 2H]/2 | [M + 3H]/3 | [M + 4H]/4 | [M + 5H]/5 | [M + 6H]/6 | % yield |
| 43 | 2081.8 | 1042.2 | | | | | 59.4% |
| 44 | 2081.8 | 1042.2 | | | | | 51.1% |
| 45 | 2081.8 | 1042.3 | | | | | 55.7% |
| 46* | 1909.9 | 956.4 | | | | | 51.2% |
| 48 | 2871.3 | | 958.5 | 719.2 | 575.6 | 479.8 | 53.6% |
| 50 | 2871.3 | | 958.5 | 719.1 | 575.6 | 479.7 | 52.9% |
| 51* | 2699.4 | | 901.3 | 676.2 | 542.5 | 451.1 | 62.8% |

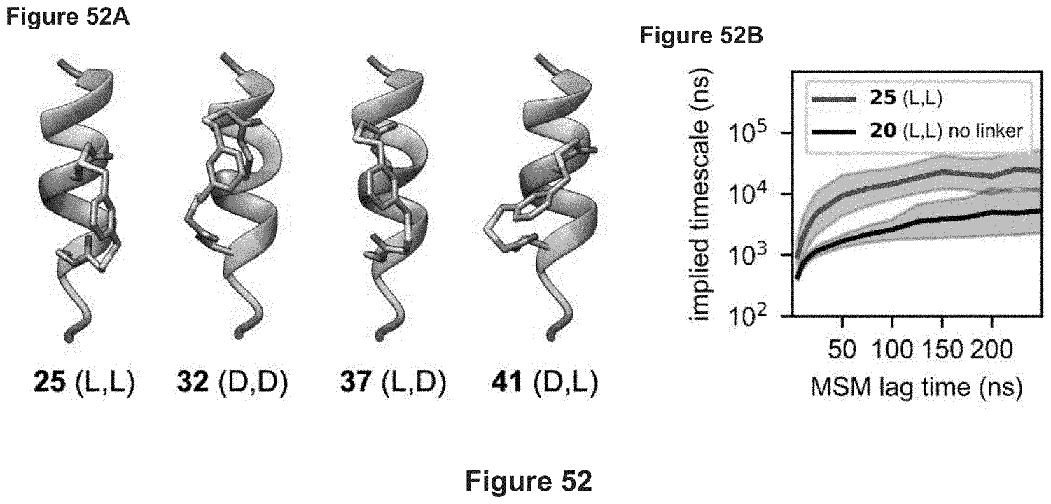

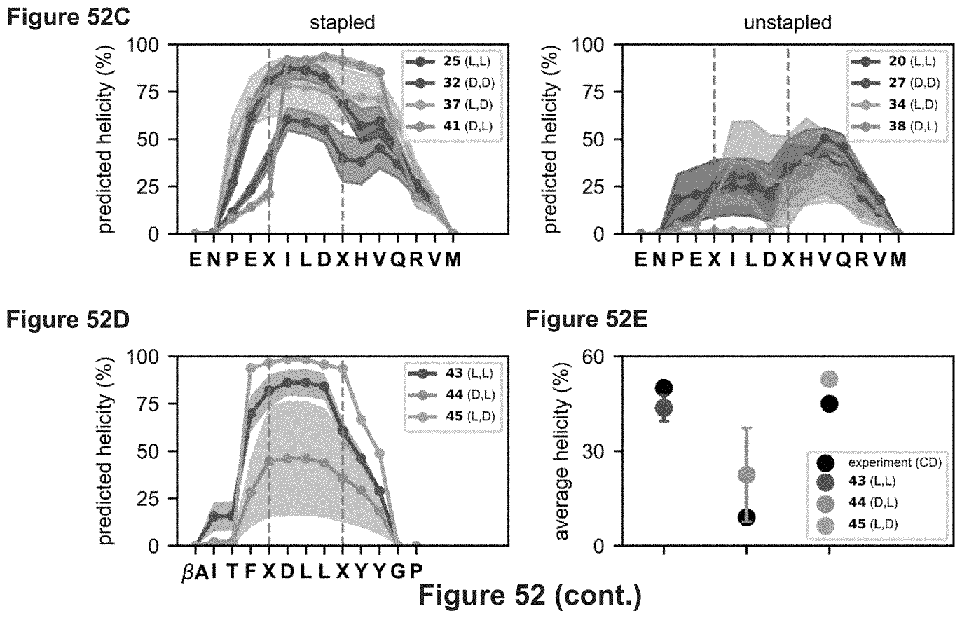

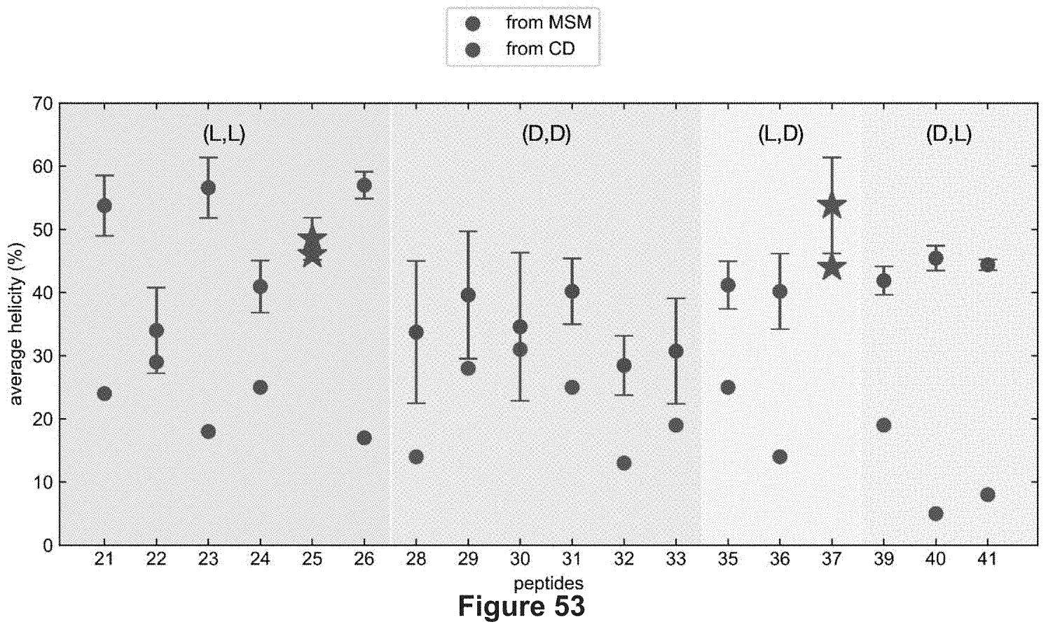

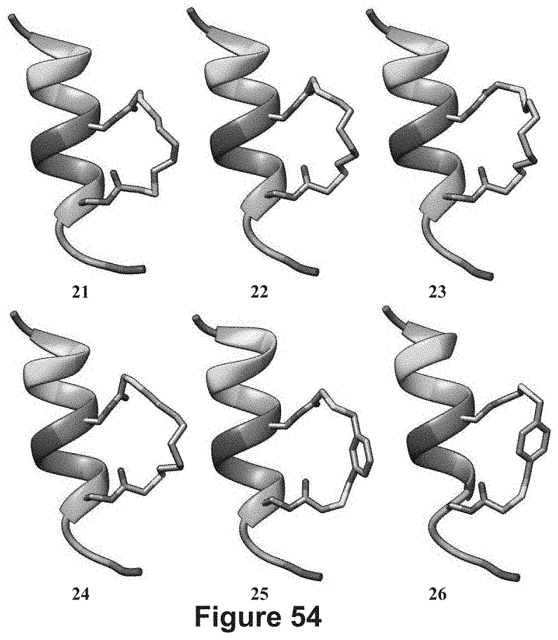

Taken together, FTDR-based stapling worked most efficiently with the rigid meta-benezenemethane dithiol linker, and generally preferred the L,L or L,D fluoroacetamide substrates on different peptide targets, with the D,L substrates least tolerated. To gain insight to the molecular mechanisms driving this preference, extensive molecular dynamics simulations were performed for stapled Axin peptides (21)-(41) and HIV C-CA binding peptides (43)-(45), generating an aggregate of 1.8 ms of trajectory data (Table 4) on the Folding@home distributed computing platform (Shirts M et al., 2000, Science, 290:1903-1904; Zhou G et al., 2017, Biophysj, 113:785-793; Zhou G et al., 2016, Journal of Physical Chemistry B, 120:926-935). Markov State Models (Chodera J D et al., 2014, Current opinion in structural biology, 25:135-144; Prinz J.-H. et al., 2011, Journal of chemical physics, 134:174105) of the conformational dynamics showed that peptide stapling stabilized helical conformations (FIG. 52A and FIG. 52C), and slowed folding by an order of magnitude to the ˜10 μs time scale (FIG. 52B). Predicted helicity profiles for stapled and unstapled Axin peptides (FIG. 52C) and stapled HIV C-CA binding peptides (FIG. 52D) also showed that crosslinked D,L substrate-containing peptide analogues disrupt helicity at the D-amino acid position more than those crosslinked L,D substrate-containing peptides. This was likely due to subtle differences in linker strain, combined with a differential helix nucleation propensities in the N-vs-C-terminal direction for stapled peptides (Acharyya A et al., 2019, Journal of Physical Chemistry B, 123:1797-1807). In summary, predicted helicities from simulations compared well with experimental values from CD spectroscopy (FIG. 52E, FIG. 53, and FIG. 54)

TABLE 4
Summary of simulation trajectory data for stapled/unstapled
Axin and HIV peptides.

| peptide | Fluoro substrates | Simulation time (μs) |
|---|---|---|
| 20 | L_{i, i+4}L | 77.40 |
| 21 | L_{i, i+4}L | 76.25 |
| 22 | L_{i, i+4}L | 82.10 |
| 23 | L_{i, i+4}L | 76.90 |
| 24 | L_{i, i+4}L | 84.35 |
| 25 | L_{i, i+4}L | 75.20 |
| 26 | L_{i, i+4}L | 82.25 |
| 27 | Dᵢ, ᵢ₊₄D | 62.60 |
| 28 | Dᵢ, ᵢ₊₄D | 74.10 |
| 29 | Dᵢ, ᵢ₊₄D | 71.85 |
| 30 | Dᵢ, ᵢ₊₄D | 83.45 |
| 31 | Dᵢ, ᵢ₊₄D | 76.55 |
| 32 | Dᵢ, ᵢ₊₄D | 76.75 |
| 33 | Dᵢ, ᵢ₊₄D | 76.40 |
| 34 | Lᵢ, ᵢ₊₄D | 68.10 |
| 35 | Lᵢ, ᵢ₊₄D | 78.40 |
| 36 | Lᵢ, ᵢ₊₄D | 84.10 |
| 37 | Lᵢ, ᵢ₊₄D | 65.25 |
| 38 | Dᵢ, ᵢ₊₄L | 66.50 |
| 39 | Dᵢ, ᵢ₊₄L | 79.45 |
| 40 | Dᵢ, ᵢ₊₄L | 82.05 |
| 41 | Dᵢ, ᵢ₊₄L | 84.80 |
| 43 | Lᵢ, ᵢ₊₄L | 51.65 |
| 44 | Lᵢ, ᵢ₊₄D | 60.00 |
| 45 | Dᵢ, ᵢ₊₄L | 63.30 |
| | total | 1859.75 |
| | average | 74.39 |

Cell Permeability of FTDR-Stapled Peptide Analogues

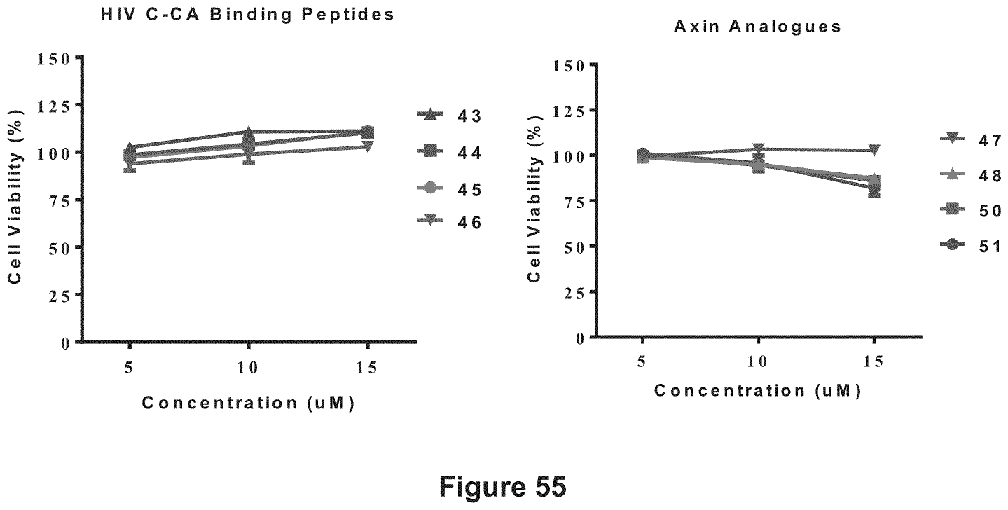

Cellular uptake of peptides has been revealed to be a complicated process driven by hydrophobicity, positive charge, and alpha-helicity, etc. (Dougherty P G et al., 2019, Journal of Medicinal Chemistry, 62:10098-10107) The RCM-based stapling was previously reported to endow enhanced cellular permeability to HIV C-CA binding peptide (46) due to the stabilized secondary structure and the increased hydrophobicity (Zhang H et al., 2008, Journal of Molecular Biology, 378:565-580). Particularly for stapled peptides, the staple type also served as one of the deciding factors for cell penetration (Chu Q et al., 2015, MedChemComm, 6:111-119; Bird G H et al., 2016, Nat Chem Biol, 12:845-852). Thus, additional studies examined whether peptides stapled by FTDR render at least comparable cell permeability. The studies first investigated the dose-dependent cytotoxicity of analogues ((43)-(46)) after incubation with HEK293T cells for 12 h, and did not observe any significant effects on viability with up to 15 μM of peptides (FIG. 55). Toward this end, all the FITC-labelled HIV C-CA binding analogues ((43)-(46)) at 10 μM concentration were incubated with HEK293T cells for 4 h and were subsequently analyzed by confocal fluorescence microscopy (FIG. 51C). Significant cellular uptake of the FTDR-stapled peptides (43) and (44) were observed, with peptides not only spreading in the cytosol but also existing in the endosomes showing punctuated greenish fluorescence. In comparison, much less fluorescence was observed from cells treated with the RCM stapled control (46). A similar trend has been previously reported with HIV C-CA binding peptides stapled by perfluoroarylation of cysteines (Spokoyny A M et al., 2013, J Am Chem Soc, 135:5946-5949), indicating that the aromatic part in the linkers enhance the cellular uptake. Additionally, the weaker uptake of the D,L-stapled peptide (45) also corroborated the previously reported positive correlation between helicity and cell permeability (Muppidi A et al., 2011, Chemical Communications, 47:9396-9398).

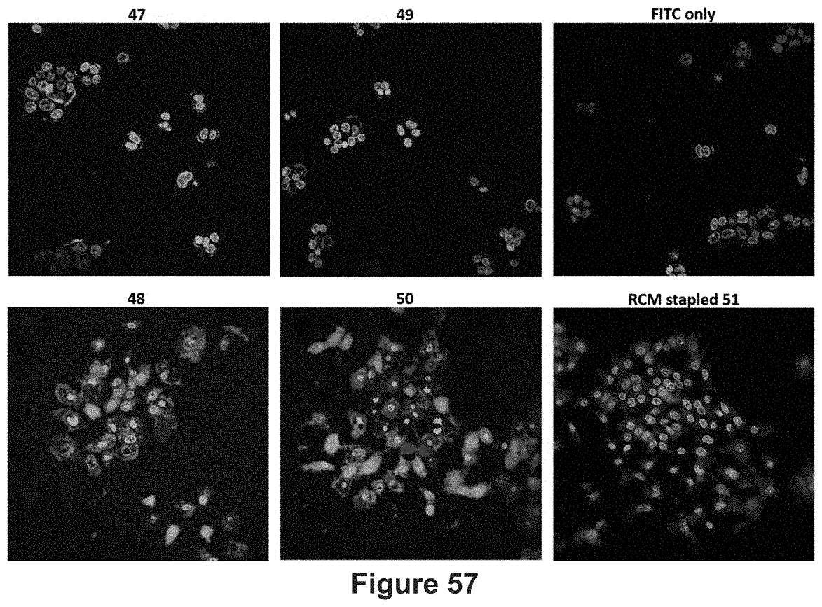

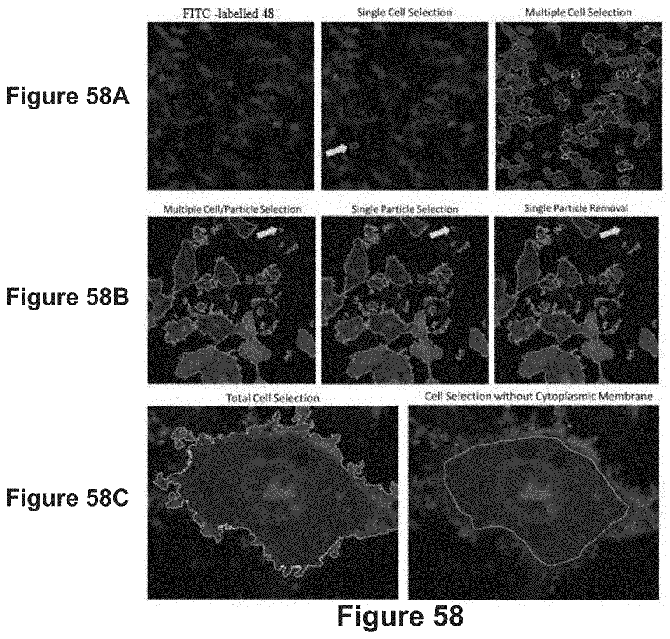

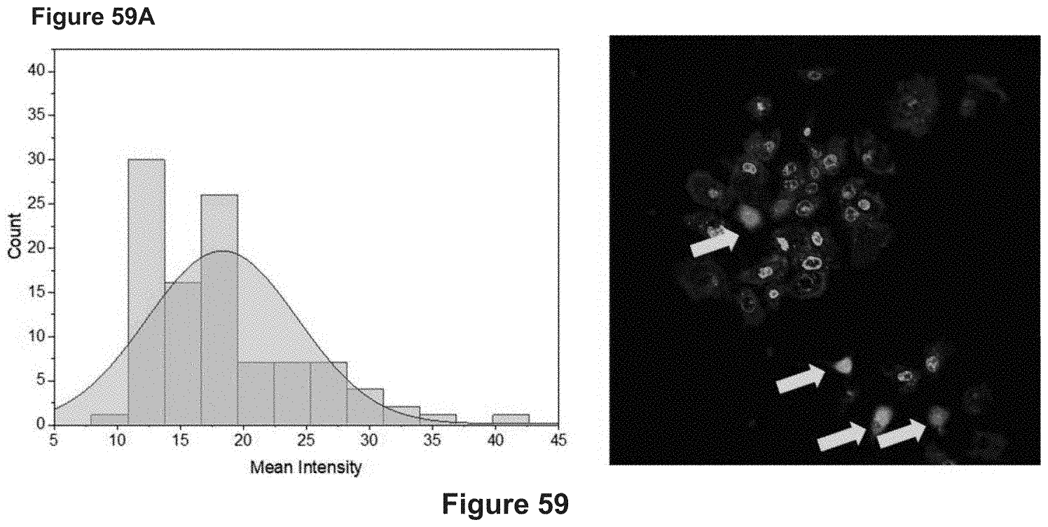

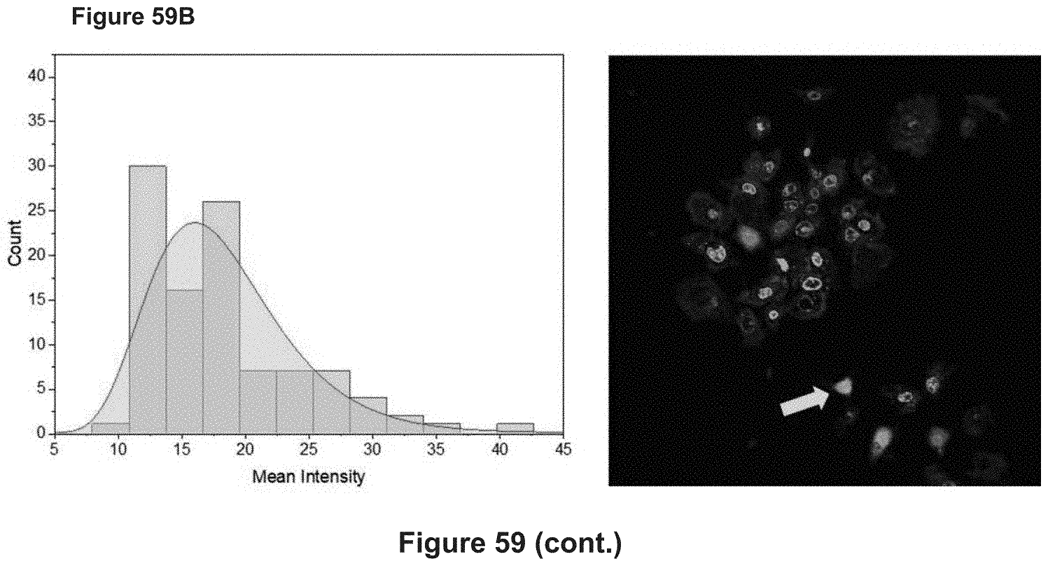

Next, studies investigated whether FTDR-based stapling on other peptide sequences led to enhanced cell penetration as well. To observe the broader applications of FTDR-based stapling on other peptides and cell lines, another Axin derived peptide analog ((51) (stapled SEQ ID NO: 7), FIG. 56A), which displayed single-digit nM Kd after i, i+4 stapling by RCM, but showed limited cell permeability to DLD-1 cells was chosen (Grossmann T N et al., 2012, Proc Natl Acad Sci USA, 109:17942-17947). Previously, other amino acids in this sequence had to be mutated into arginine in order to increase the overall positive charges. The resulting lead analogue had improved cellular uptake at the expense of losing 6-7 folds of affinity towards the protein target β-catenin (Grossmann T N et al., 2012, Proc Natl Acad Sci USA, 109:17942-17947). The FITC labelled fluoroacetamide containing L,L substrate (47) (SEQ ID NO: 7) and L,D substrate (49) (SEQ ID NO: 7) were thereby synthesized, and studies focused on the DLD-1 cell line as reported in order to facilitate direct comparison. FTDR-based stapling with 1,3-benzenedimethanethiol proceeded smoothly, and the resulting analogues ((48) (stapled SEQ ID NO: 7), (50) (stapled SEQ ID NO: 7), FIG. 56A, Table 3) did not affect the viability of DLD-1 cells at 10 μM concentration after 12 h incubation (FIG. 55). With confidence that there was no cytotoxicity, DLD-1 cells were treated with each of these peptides, along with the unstapled or RCM-stapled control peptide in parallel. As shown in FIG. 56B and FIG. 57, significant cellular uptake was observed from cells incubated with 10 μM FTDR stapled (48) or (50), while there was much weaker fluorescence in other control treatment groups including the cells incubated with (51). The uptake pattern of (48) and (50) were similar to those observed earlier for HIV C-CA binding peptides (43) and (44). A quantitative analysis was achieved by applying the lognormal fitting to a histogram of the individual cell mean intensity (FIG. 58 and FIG. 59), and the resulting mean cellular fluorescence revealed that the FTDR stapled L,L HIV C-CA binding peptide (43) had 4.76±0.09 fold of mean intensity compared to that of the RCM control peptide intracellularly, and the stapled L,D mimetic (44) had 5.75±0.07 fold mean intensity (FIG. 51D and Table 5). Quite consistently, the L,L Axin derivative (48) showed a 4.86±0.15 fold of mean intensity compared to the RCM control one, while the L,D Axin mimetic (50) showed a 5.05±0.14 fold increase (FIG. 56C and Table 6). In both cases, stapled L,D analogues demonstrated slightly stronger cellular uptake than the stapled L,L analogues. Moreover, the diminished uptake from unstapled peptides (e.g. 0.36±0.02 fold for 47, 0.33±0.01 fold for (49)) further indicated that the FTDR stapling was an essential requirement for cell permeability.

TABLE 5
Quantification of the cell penetration of HIV C-CA binding peptides.

| | Mean | N |
|---|---|---|
| | Intensity | (cell |
| Peptide | Ratio | number) |
| 46* | 1.00 ± 0.04 | 108 |
| 43 | 4.76 ± 0.09 | 81 |
| 44 | 5.75 ± 0.07 | 121 |
| 45 | 3.07 ± 0.08 | 115 |

(*Control peptides stapled by RCM)

TABLE 6
Quantification of the cell penetration of Axin analogues.

| | Mean | N |
|---|---|---|
| | Intensity | (cell |
| Peptide | Ratio | number) |
| 51* | 1.00 ± 0.02 | 91 |
| 47 | 0.36 ± 0.02 | 102 |
| 48 | 4.86 ± 0.15 | 101 |
| 49 | 0.33 ± 0.01 | 102 |
| 50 | 5.05 ± 0.14 | 110 |
| FITC only | 0.30 ± 0.02 | 104 |

(*Control peptides stapled by RCM)

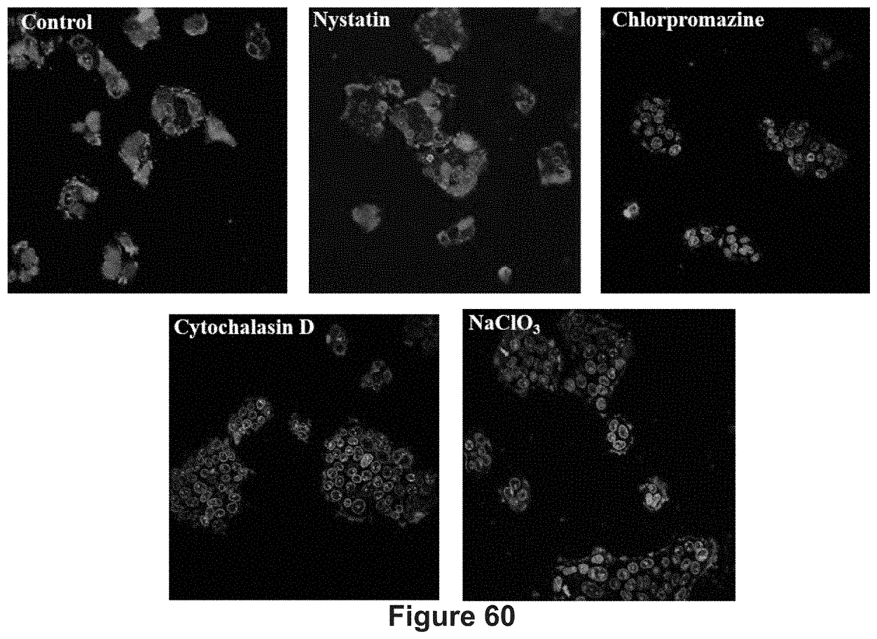

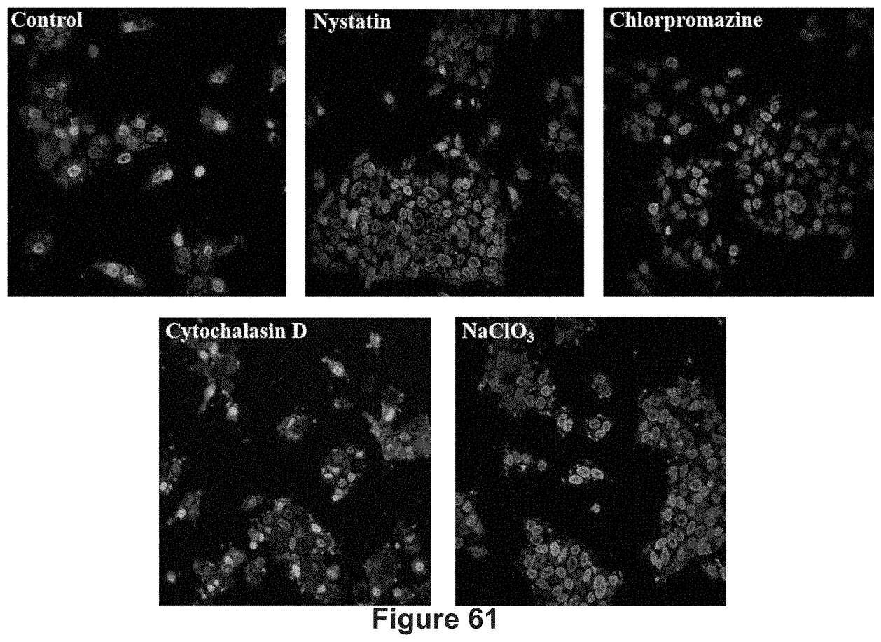

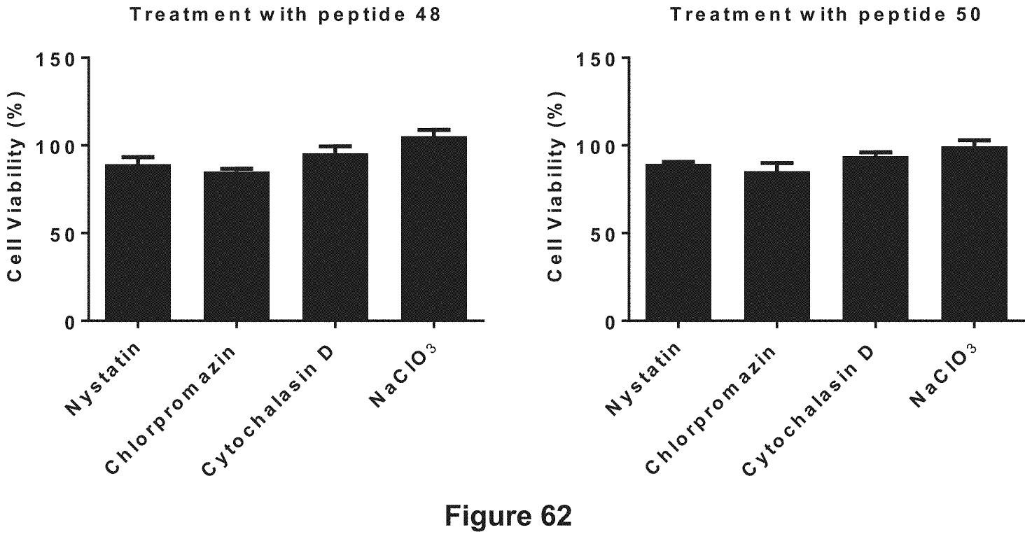

Despite intensive studies, the uptake mechanisms for cell-penetrating peptides remained ambiguous and largely varied due to their complicated nature (Silhol M et al., 2002, Eur J Biochem, 269:494-501; Deshayes S et al., 2005, Cell Mol Life Sci, 62:1839-1849; Kaplan I M et al., 2005, J Control Release, 102:247-253). Small molecule inhibitors specific for each endocytic pathway have been routinely utilized to interrogate the cellular uptake mechanisms of many transporters (Halvorsen B et al., 1998, Biochem J, 331:743-752; Wang L H et al., 1993, J Cell Biol, 123:1107-1117; Gottlieb T A et al., 1993, J Cell Biol, 120:695-710; Zhu X D et al., 2011, J Biol Chem, 286:8231-8239). For example, nystatin as a sterol-binding agent was used to selectively block caveolin-dependent endocytosis (Zhu X D et al., 2011, J Biol Chem, 286:8231-8239, while chlorpromazine selectively inhibited clathrin-mediated endocytosis (Wang L H et al., 1993, J Cell Biol, 123:1107-1117; Zhu X D et al., 2011, J Biol Chem, 286:8231-8239). Cytochalasin D specifically induced depolymerization of actin and ceased the subsequent apical endocytosis (Gottlieb T A et al., 1993, J Cell Biol, 120:695-710). Sodium chlorate, on the other hand, aborted the decoration of cell membranes with sulfated proteoglycans, affecting certain other uptake pathways (Halvorsen B et al., 1998, Biochem J, 331:743-752). With those, RCM-stapled peptides were recently revealed to penetrate cells via clathrin- and caveolin-independent pathways that were partially mediated by cell surface proteoglycans (Chu Q et al., 2015, MedChemComm, 6:111-119). To understand the mechanisms behind the enhanced cellular uptake of peptides stapled by FTDR, similar experimental investigations were performed using the lead Axin derivatives (48) and (50). The cell penetration experiments were repeated under conditions that blocked a different endocytotic pathway each round (FIG. 60 and FIG. 61). Cell viabilities were measured immediately after the imaging experiments to ensure that the observed results were due to the active cellular uptake (FIG. 62). As quantitatively summarized in FIG. 56D and Table 7 and Table 8, both peptides had their uptake partially blocked by more than one pathway-specific inhibitors. Like RCM-stapled peptides, both FTDR-stapled peptides internalized partially through sulfated proteoglycans, as indicated by the reduced uptake in cells treated by sodium chlorate. Yet unlike hydrocarbon-stapled peptides, the L,L stapled analogue (48) also partially penetrated cells via endocytosis depending on clathrin (inhibited by chlorpromazine) and actin polymerization (inhibited by cytochalasin D). The L,D stapled (50) appeared to enter cells additionally through clathrin- and caveolin-dependent (blocked by nystatin) endocytosis. Interestingly, the chirality difference in the i+4 position between the stapled peptide (48) and (50) seemed to result in different peptide backbone conformations, thereby affecting their intake through distinguished endocytosis pathway. Taken together, the present data demonstrated that FTDR-stapled peptides penetrate cells through multiple endocytotic pathways in a distinct pattern compared to the RCM-stapled peptides, which accounts for their enhanced cellular uptake than those observed for the RCM-stapled controls.

TABLE 7
Quantification of the cell penetration of stapled Axin
analogue 48 during pathway blocking studies.

| | Mean | |
|---|---|---|
| Pathway | Intensity | |
| Blocker | Ratio | N |
| Control* | 1.00 ± 0.04 | 52 |
| Nystatin | 1.16 ± 0.03 | 48 |
| Chlorpromazine | 0.13 ± 0.01 | 61 |
| Cytochalasin D | 0.10 ± 0.01 | 60 |
| NaClO₃ | 0.12 ± 0.01 | 63 |

(*Control: solvent vehicle only)

TABLE 8
Quantification of the cell penetration of stapled Axin
analogue 50 during pathway blocking studies.

| | Mean | |
|---|---|---|
| Pathway | Intensity | |
| Blocker | Ratio | N |
| Control* | 1.00 ± 0.02 | 54 |
| Nystatin | 0.17 ± 0.01 | 59 |
| Chlorpromazine | 0.16 ± 0.01 | 53 |
| Cytochalasin D | 0.99 ± 0.02 | 56 |
| NaClO₃ | 0.20 ± 0.01 | 49 |

(*Control: solvent vehicle only)

Activity of FTDR-Stapled Peptide Analogues

In addition to cell permeability, additional studies investigated whether the structural features brought by the 1,3-benzenedimethanethiol crosslinker translates to other functional relevance. Thus, ELISA assay were performed to quantify the binding affinity of the lead Axin derivatives towards the target protein β-catenin (FIG. 56E). The EC₅₀ was 4.36±1.75 nM for stapled analogue (48), and 5.27±2.29 nM for analogue (50), which were similar to the EC₅₀ of RCM-stapled (51) (3.03±1.8 nM) and were at least 100-fold more potent than unstapled peptide (47). Notably, a direct comparison of the fluorescence signals for all the peptides at 50 μM or 200 μM doses pinpointed the potentially better binding of FTDR stapled peptides than the RCM control (51). Given that staples and the peptide conformational changes after stapling usually rendered the structures less prone to protease-mediated cleavage (Bird G H 2014, ACS Chem Biol, 9:831-837; Walensky L D et al., 2014, J Med Chem, 57:6275-6288; Spokoyny A M et al., 2013, J Am Chem Soc, 135:5946-5949), the serum stability of these lead peptides in 100% rat serum were also tested. As shown in FIG. 56F, all the stapled peptides remained mostly intact while unstapled control (47) was rapidly degraded, with only 30.9% left after 20 min of incubation. In order to see if their improved cellular uptake translate to enhanced cellular activity, subsequent studies examined these analogues' inhibition of the growth of a Wnt-driven colorectal cancer cell line (Grossmann T N et al., 2012, Proc Natl Acad Sci USA, 109:17942-17947) DLD-1 over a 5-day period (FIG. 56G). FTDR stapled (48) and (50) potently impeded the cell growth with EC₅₀s of 2.3±0.4 μM, and 9.8±2.0 μM, respectively. On the contrary, neither the unstapled control (47) nor the RCM-stapled analogue (51) displayed a significant growth inhibition until they were administered at the 16 μM concentration. Together, unprotected peptides stapled by FTDR appeared to recapitulate the advantages in biological functions seen in the classic RCM-stapled peptides, but also possessed enhanced cell permeability and the correlated growth inhibition of the targeted cancer cells.

In summary, the data described herein have demonstrated a new, mild, and clean synthetic strategy to cyclize and/or staple unprotected peptides. The developed FTDR approach operated at mild temperature in aqueous solutions and offered excellent chemoselectivity and functional group tolerance. Its application was first exemplified as a general macrocyclization platform that was compatible with a variety of linkers. Then its use in stapling peptides at i, i+4 positions was demonstrated, and further showed that the lead stapled peptides retained the structure features and biological properties reported in literature. The identification of the 1,3-benzenedimethanethiol as the optimal linker for stapling demonstrated that certain aromatic rigidity in the crosslinker region was required to maintain the alpha-helical conformation of peptide substrates stapled by FTDR. Further, both the experimental results and the molecular dynamics simulation consistently pinpointed the preference of L,L and L,D substrate chirality for the folding (alpha helicity) of the FTDR-stapled peptides, implicating the distinct helix nucleation propensities in the N-vs-C-terminal direction for this class of stapled peptides.

In terms of biological functions, the enhanced cellular uptake of i, i+4 stapled peptides and the associated distinct penetration mechanism demonstrated that this FTDR-based stapling approach expand the toolbox of chemical transformations to generate a new class of probes or therapeutic leads for intracellular targets. This was also the first reported effort to elucidate the cellular uptake mechanism of peptides stapled by strategies other than RCM. The present findings confirmed the previous observations that there could be more than one uptake mechanisms existing for stapled peptides (Chu Q et al., 2015, MedChemComm, 6:111-119). Further, they were consistent to the previous discovery that internalization of stapled peptides mainly correlated with the staple type (Chu Q et al., 2015, MedChemComm, 6:111-119). Accordingly, the present approach in many aspects was complementary to the RCM-mediated peptide stapling. Current efforts are focused on optimizing the FTDR reaction conditions to further improve its efficiency, and expanding the substrate scope towards i, i+7 positions. Application of the FTDR-based stapling to probe protein-protein interactions related to the key signaling events in prostate cancer and neurodegenerative diseases are also under active investigation.

In summary, stapled peptides serve as a powerful tool for probing protein-protein interactions, but its application has been largely impeded by the limited cellular uptake. The present example reported the discovery of a facile peptide macrocyclization and stapling strategy based on a FTDR, which rendered a class of peptide analogues with enhanced stability, affinity, and cell permeability. This new approach enabled selective modification of the orthogonal fluoroacetamide side chains in unprotected peptides, with the identified 1,3-benzenedimethanethiol linker promoting alpha helicity of a variety of peptide substrates, as corroborated by molecular dynamics simulations. The cellular uptake of these stapled peptides was universally enhanced compared to the classic RCM stapled peptides. Pilot mechanism studies suggested that the uptake of FTDR-stapled peptides may involve multiple endocytosis pathways. Consistent with the improved cell permeability, the FTDR-stapled lead Axin analogues demonstrated better inhibition of cancer cell growth than the RCM-stapled analogues.

The materials and methods employed in Examples 4 and 5 are now described.

General Information: All chemicals and solvents used were purchased from either Fisher Scientific, Sigma Aldrich or VWR and were used directly without any further purification. All small molecules and building blocks were synthesized following traditional organic chemistry procedures. Regular phase flash column chromatography with manually loaded silica gel (grade 60, 230-400 mesh, Fisher Scientific) was used to purify synthesized compounds. High resolution ESI-MS was obtained at the Wistar Institute, using a ThermoFisher Scientific Q Exactive HF-X mass spectrometer coupled to a ThermoFisher Vanquish Horizon UHPLC system. NMR data was recorded on a 500 MHz Bruker Advance with TMS as an internal standard. Peptides were synthesized on solid-phase using Fmoc chemistry. After cleavage from resins, the crude mixtures were precipitated out by diethyl ether, resuspended, and purified using Waters 1525 series preparative high-performance liquid chromatography (HPLC) loaded with the XBridge Prep C18 column (25 cm×19 mm, particle size 5 μm). All peptide mass spectra data were recorded on the Agilent 1100 series liquid chromatography-mass spectrometry (LC-MS) that was equipped with an Ascentis® Express C8 analytical column (5 cm×2.1 mm, particle size 2.7 μm). The LC-MS program ran with mobile phase A (0.1% formic acid in water) and mobile phase B (0.1% formic acid in acetonitrile). For each run, the acetonitrile was linearly increased from 5% to 95% within 10 min.

Peptides Synthesis: Peptides were synthesized on rink amide resins following traditional Fmoc-based solid-phase chemistry. Most >10 mer peptides were synthesized on an automatic peptide synthesizer at the Macromolecular core facility of the Pennsylvania State University. Generally, approximately four equivalents of Fmoc-protected amino acid building blocks, four equivalents of HATU and eight equivalents of DIPEA were added to the resins resuspended in DMF for every round of coupling. The N-terminal of peptides were capped with acetic anhydride or FITC in DMF using DIPEA as the base. After capping, peptides were cleaved by incubating the beads with reagent H (trifluoroacetic acid (81% w/w), phenol (5% w/w), thioanisole (5% w/w), 1,2-ethanedithiol (2.5% w/w), dimethylsulfide (2% w/w), ammonium iodide (1.5% w/w), and water (3% w/w))61 at room temperature for 3 h. The supernatant was collected and added diethyl ether to precipitate out the crude product.

General Procedures for Peptide Stapling: For the controls that required RCM, RCM-based stapling was performed with protected peptides on resins following the reported procedures (Grossmann T N et al., 2012, Proc Natl Acad Sci USA, 109:17942-17947). The resins were mixed with approximately 0.5 equivalent of Grubbs I catalyst (5 mM) in dichloromethane and were incubated for 2 h at room temperature before the solvents were drained off. The coupling process was repeated three times, followed by subsequent N-terminal deprotection and capping. The crude products were cleaved from resins as mentioned before, and precipitated out by 15-fold ice-cold diethyl ether. The crude mixture was re-dissolved in a water and methanol mix, and purified by reverse phase semi-preparative HPLC that operated at a flow rate of 10 mL/min, used water/0.1% TFA as solvent A and acetonitrile/0.1% TFA as solvent B.

For stapling based on the FTDR, 126 μL of the dithiol-containing linker (250 mM in DMF) was premixed with 210 μL of sodium hydroxide solution (250 mM) and 189 μL of DMF at room temperature for 40 min to completely deprotonate dithiols. After this, 105 μL of peptide solution (50 mM in water or DMF) was added to start the stapling based on FTDR. The reaction mixture was incubated at 37° C. for around 12 h until the reaction was complete as determined by LC-MS, and was then quenched with water and acetic acid. Subsequently, the solution was extracted by ethyl acetate three times to remove excess linkers. The remaining aqueous phase was lyophilized, resuspended in methanol, and added diethyl ether to precipitate out the stapled peptides. The crude product was further purified on HPLC as mentioned before. The yields for all the stapled peptides (Table 1 and Table 3) were determined after recovery from HPLC purification.

Circular Dichroism: Axin- and p53-derived peptides were dissolved in water, and HIV-targeting peptides were dissolved in 25% acetonitrile/water to ensure 100% solubility. The final concentration of the peptide samples was 70 μM. Circular dichroism (CD) measurements were performed on a Jasco model J-815 spectropolarimeter with a 1 mm Jasco quartz cell over the wavelength range of 180-250 nm. The scans were carried out at 0.2 nm resolution with 4 sec average time at 25° C. Data from three scans were averaged, base line corrected, and normalized to the mean residue ellipticity (MRE) following the equation: [θ]_{λ}=[θ]_{obs}/(10×1×C×n). [θ]_{λ} is MRE in deg×cm²×dmol⁻¹; [θ]_{obs} is the measured ellipticity; C is the concentration of peptides in M; 1 is the optical pathlength in cm; and n is the number of residues in peptides. The % alpha helicity was calculated from the MRE values at 222 nm, using the equation % helicity=([θ]₂₂₂−[θ]₀)/([θ]ₘₐₓ−[θ]₀) based on the previously reported method (de Araujo A D et al., 2014, 2014, Angew Chem Int Ed Engl, 53:6965-6969). [θ]₂₂₂ is the MRE value at 222 nm; [θ]ₘₐₓ is the maximum theoretical MRE value for a helix of n residues; and [θ]₀ is the MRE value of the peptide in random coil conformation that usually equals to (2220-53T) (de Araujo A D et al., 2014, 2014, Angew Chem Int Ed Engl, 53:6965-6969).

Molecular Dynamics Simulation: Molecular dynamics (MD) simulations of stapled/unstapled Axin peptides and HIV peptides were performed using the OpenMM 7.0 simulation package (Eastman P et al., 2017, PLOS Computational Biology, 13:e1005659) on the Folding@home distributed computing platform (Shirts M et al., 2000, Science, 290:1903-1904). The AMBER ff14SB force field (Maier J A et al, 2015, Journal of Chemical Theory and Computation, 11:3696-3713) was used for the peptide residues, while non-natural residues and linkers used GAFF (Wang J et al., 2004, Journal of Computational Chemistry, 25:1157-1174) with partial charges from AM1-BCC (Jakalian A et al., 2002, Journal of Computational Chemistry, 23:1623-1641), parameterized using the antechamber package of AmberTools17 (Case D A et al., 2017 AMBER, University of California, San Francisco). Axin peptide simulations were initiated from helical conformations taken from crystal structure PDB:1QZ7. These structures were solvated in a ˜(55 Å)³ cubic periodic box with TIP3P water molecules and Na⁺ and Cl⁻ counterions at 100 mM to neutralize charge, for a total of ˜16K atoms. HIV peptide simulations were initiated from helical conformations taken from the crystal structure PDB:3V3B. These structures were solvated in a ˜(45 Å)³ cubic periodic box with Na⁺ and Cl⁻ counterions, for a simulation size of around 9K atoms.

Trajectory production runs were performed using stochastic (Langevin) integration at 300 K with a 2-fs time step. Covalent hydrogen bond lengths were constrained using the LINCS algorithm. PME electrostatics were used with a nonbonded cutoff of 9 Å. The NVT ensemble was enforced using a Berendsen thermostat. About 50 trajectories were generated for each peptide design, reaching an average trajectory length of ˜1.0 μs. In total, 1.8 milliseconds of aggregate simulation data were generated for all designs, with about ˜70 μs of trajectory data per design (Table 4). Trajectory snapshots for the peptide coordinates were saved for every 500 μs. For all analysis described below, the first 100 ns was discarded from each trajectory to help remove systematic bias.

Markov State Model Construction: To describe the conformational dynamics of each peptide, Markov state models (MSMs) were constructed from the trajectory data using the MSMBuilder 3.8.0 software package (Harrigan M P et al., 2017, Biophysical Journal, 112:10-15). This involved the following steps: (1) performing dimensionality reduction using time-structure-based independent component analysis (tICA) (Pérez-Hernández G et al., 2013, Journal of Chemical Physics, 139:015102; Schwantes C R et al., 2013, Journal of Chemical Theory and Computation, 9:2000-2009), (2) conformational clustering in the reduced space to define and assign the trajectory data to discrete metastable states, and (3) estimating the transition rates between metastable states from the observed transitions between states.

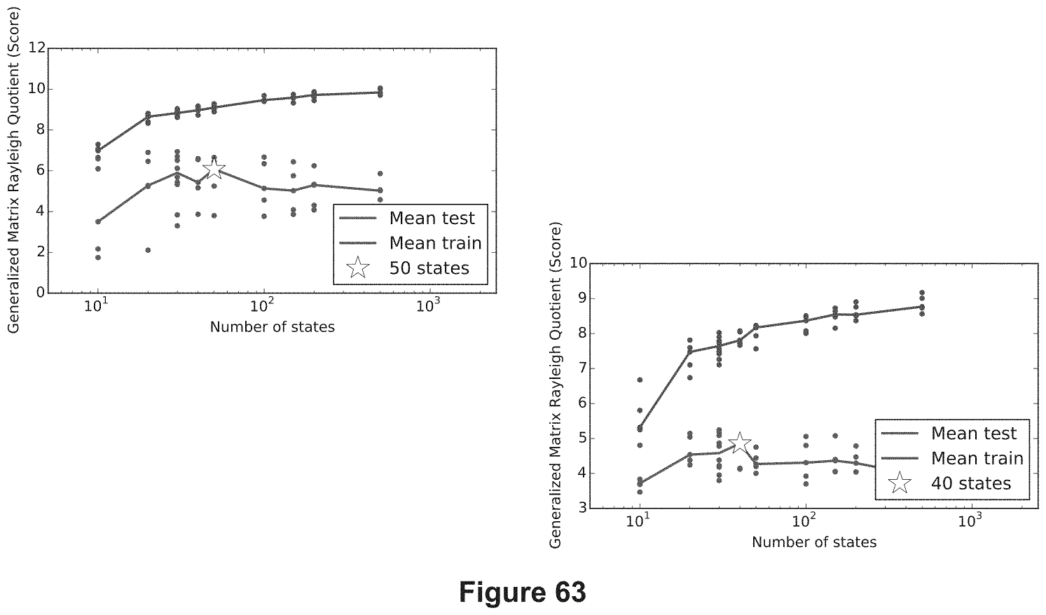

The tICA method is a popular approach to project protein coordinates to a low-dimensional subspace representing the degrees of freedom along which the slowest motions occur. In tICA, structural features fᵢ are computed for each trajectory frame, and the time-lagged correlation matrix C(Δt) of elements Cᵢⱼ=<fᵢ(t) fⱼ(t+Δt)>ₜ of all pairs of features i and j are computed, where t is time, and Δt is the tICA lag time. The tICA components (tICs) are linear combinations of features that capture the greatest time-lagged variance, which can be found by maximizing the objective function <α|C(Δt)|α> subject to the constraint that each component has unit variance (i.e. <αᵢ|Σ|αᵢ>=1). As features, all pairwise distances between Ca and CP atoms (435 and 300 distance pairs for Axin and HIV C-CA binding peptides, respectively) were computed using the MDTraj package (McGibbon R T et al., 2015, Biophysical Journal, 109:1528-1532). A tICA lag time of Δt=5 ns was chosen. After projecting trajectory data to the four largest tICs, conformational clustering was performed using the k-centers algorithm to define 50 discrete states for the construction of Markov State Models (MSMs). The GMRQ cross-validation algorithm was used to determine the optimal number of states (FIG. 63).

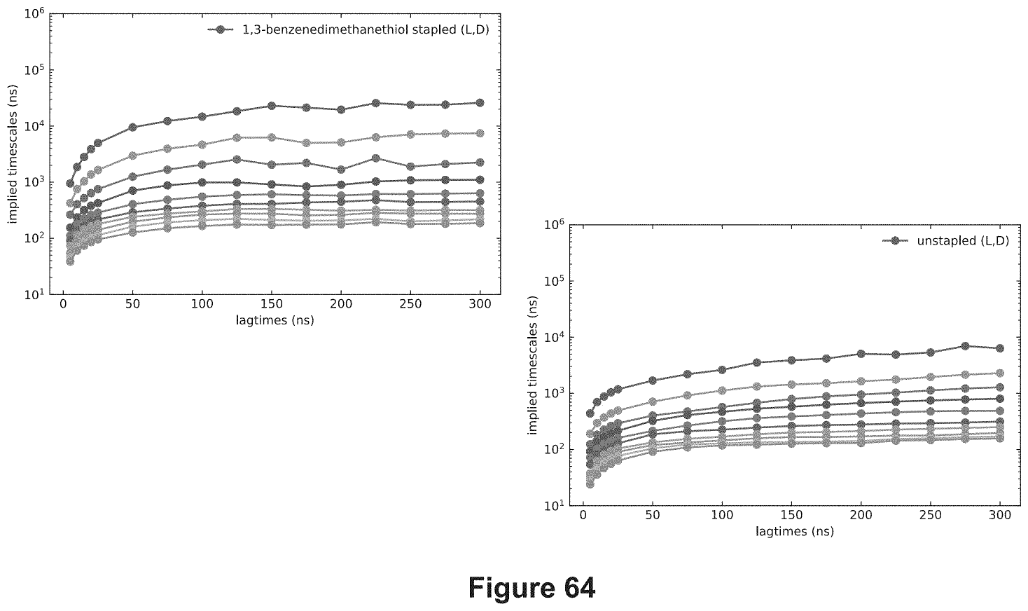

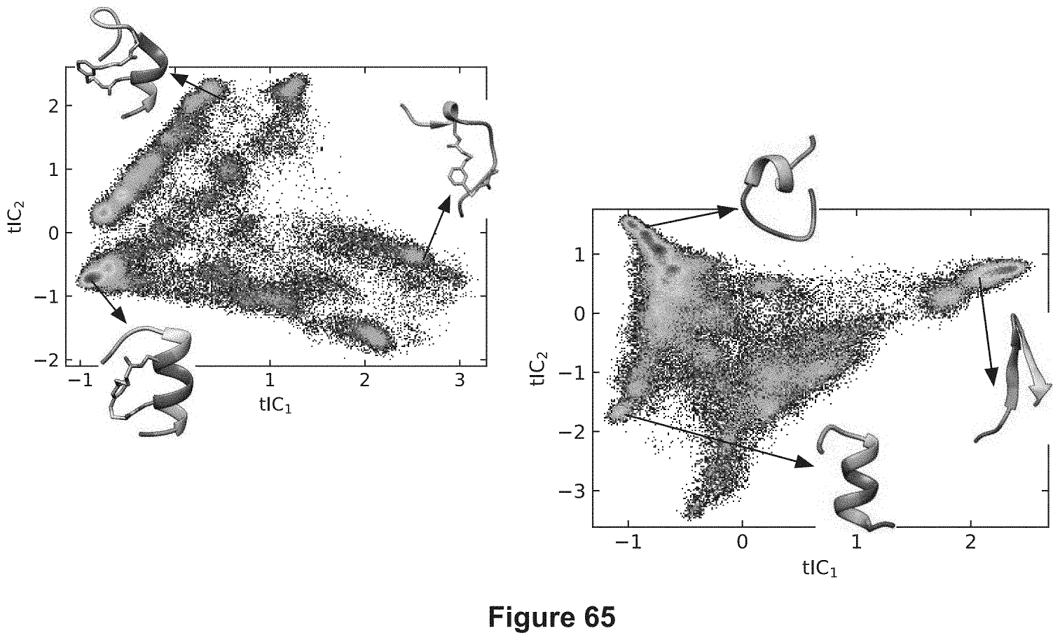

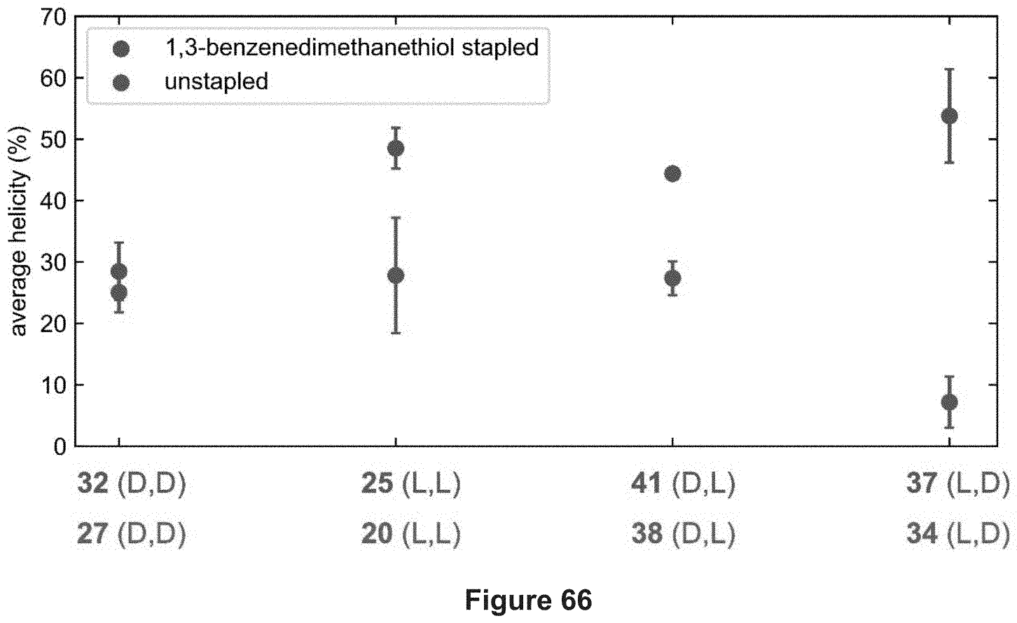

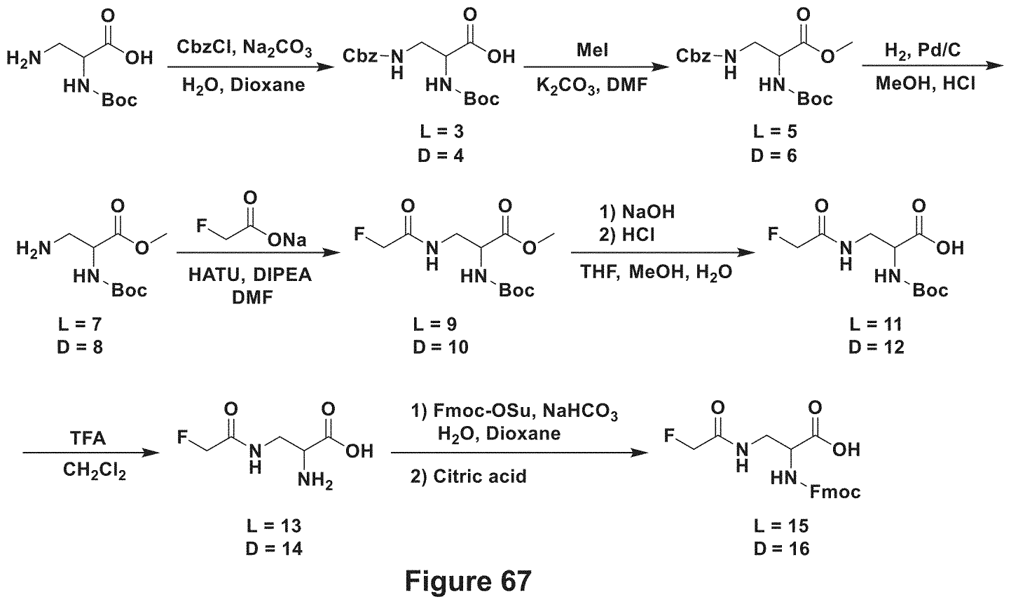

MSM transition matrices T^{(τ)} were constructed at lag times τ ranging from 1 to 300 ns, using a maximum likelihood estimator. Transition matrix elements Tᵢⱼ^{(τ)} contain the probability of transitioning from state i to state j in time τ. MSM implied timescales are computed as tᵢ=τ/ln λᵢ where λᵢ are the eigenvalues of the MSM transition matrix. The implied timescales plateau with increasing lag time, indicating that dynamics is approximately Markovian (i.e. memory-less) beyond a lag time of 100 ns (FIG. 64). The slowest implied timescale can be interpreted as the folding/unfolding relaxation time of the peptide, which generally occurs along the principal tICA component, tIC₁. Example projections of trajectory data to the first two tICs are shown in (FIG. 65).

Analysis of Secondary Structure: The secondary structure content of simulation snapshots was calculated using the DSSP algorithm implemented in MDTraj (Pelay-Gimeno M et al., 2015, Angew Chem Int Ed Engl, 54:8896-8927). The ensemble average of helicity <h> were computed from the trajectory data for each design, using the equilibrium populations πᵢ of each microstate i predicted by the MSM, as <h>=Σᵢ πᵢ hᵢ, where hᵢ is the average helicity of snapshots belonging to state i.

Cell Viability Assay: Wild type DLD-1 cells were cultured in complete growth media (RPMI, 10% FBS and 1% penicillin/streptomycin) at 37° C./5% CO₂. After harvesting, cells were placed onto 96-well flat bottom white plates at 10e4 cells/well, and were incubated overnight. The next morning, serially diluted axin analogues (final concentration 0 μM, 5 μM, 10 μM, and 15 μM) were added to the cell culture media and incubated for 12 h. The cells were then treated with CellTiter-Glo® reagent that had been prepared following the manufacturer's protocol at 100 μL/well. The chemiluminescent signals from the control sample (treated with 0 μM peptides) were used as the 100% control. For HIV-1 peptide analogues, the samples were incubated with HEK293T cells instead, and the rest of the procedure was the same as mentioned before. For DLD-1 cells, after confocal imaging for the penetration pathway studies, viability measurements were carried out similarly via directly mixing with the CellTiter-Glo reagent. Cells treated with vehicle groups were also analyzed and counted as the 100% control.

Confocal Imaging: All images were captured on an Olympus FV3000 confocal laser scanning microscope with a NA 1.05 30× silicone-immersion objective (UPLSAPO 30XS). Hoechst 33342 dye and FITC-labeled peptides were excited with 405 nm and 488 nm lasers (Coherent OBIS), respectively. Images were analyzed with NIH ImageJ software. Using the “analyze particles” function, the area and intensity of individual cells were measured (FIG. 58A) (Schindelin J et al., 2012, Nat Methods, 9:676-682). Particles in the extracellular matrix that the “analyze particle” function selected were manually deselected and removed from the data set (FIG. 58B). Cells with cytoplasmic membrane aggregation of the FITC-labeled peptide had the cytoplasmic membrane excluded from the mean intensity measurement (FIG. 58C). From a normal distribution curve applied to a histogram of the individual cell mean intensity, some outliers in the data collected were found (FIG. 59A). However, from the distribution of data it was found the best fit curve to be lognormal. Outliers were then removed using a Grubbs test (FIG. 59B) (Grubbs F E et al., 1969, Technometrics, 11:1-21). The optimal bin size for the histogram was determined using the Freedman-Draconis rule (Freedman D et al., 1981, Zeitschrift für Wahrscheinlichkeitstheorie und Verwandte Gebiete, 57: 453-476).

Cell Penetration Assay: DLD-1 cells were seeded on 35 mm optical dishes at 3e5 per well and were incubated overnight at 37° C./5% CO₂. FITC-labelled Axin analogues were added and incubated for 12 h at 10 μM final concentration (Grossmann T N et al., 2012, Proc Natl Acad Sci USA, 109:17942-17947). To visualize the nucleus, the cells were incubated with Hoechst 33342 (1 μg/mL, PBS, ThermoFisher) for 10 min. Cells were imaged in buffer mimicking the physiological conditions in the cytoplasm (20 mM HEPES, 110 mM KOAc, 5 mM NaOAc, 2 mM MgOAc, 1 mM EGTA, pH 7.3). FITC labelled HIV-1 C-CA peptides were imaged similarly, except that the incubation was with HEK293T cells for 4 h (SpokoynyAM et al., 2013, J Am Chem Soc, 135:5946-5949).

Cell Penetration Pathway Study: The plated DLD-1 cells were incubated for 1 h with 25 μg/mL nystatin for blocking caveolin-mediated endocytosis, 5 μg/mL chlorpromazine for blocking clathrin-dependent endocytosis, 10 μg/mL cytochalasin D for inhibiting actin polymerization, and 80 mM NaClO₃ for disrupting proteglycan synthesis according to the conditions used by literature (Chu Q et al., 2015, MedChemComm, 6:111-119). After this, the FITC-labelled peptide analogues were added at a final concentration of 10 μM, and the mixture was incubated at 37° C./5% CO₂ for 4 h. The cells were washed with PBS and incubated with Hoechst 33342 (1 μg/mL, PBS) for 10 min. After another round of washing, cells were imaged in the buffer as aforementioned. Subsequent CellTiter-Glo viability measurements were also performed right after confocal imaging.

ELISA Assay for Binding Affinity: The beta-catenin protein (Abcam, ab63175) (1 μg/mL, 50 μL/well) was coated onto a 96-well flat bottom black plate (Nunc, MaxiSorp) at room temperature for 2 h. The wells were washed with 0.05% tween containing PBS buffer, and then blocked with 1% BSA containing PBS buffer at room temperature for 2 h. The FITC labelled Axin peptide derivatives (including the unstapled control) were each serially diluted (200 μM, 50 μM, 12.5 μM, 3.125 μM, 0.781 μM, 0.195 μM, and 0) in 100 μL of blocking solution (1% BSA, PBS buffer), added to the wells, and incubated at room temperature for another 2 h. The wells were then washed three times with PBS buffer (0.05% tween) and treated with HRP-conjugated anti-FITC antibody (100 μL/well, Abcam, ab196968) for 1 h at room temperature. After this final incubation, the wells were washed 5 times with 300 μL of PBS buffer (0.05% tween), and then treated with the QuantaBlu fluorogenic peroxidase substrates that had been prepared following the manufacturer's protocol (100 μL/well). The fluorescence signals were recorded by the H1 synergy plate reader (Biotek) at the excitation wavelength of 325 nm and the emission wavelength of 420 nm.

Serum Stability Assay: Based on the ELISA-based assay of EC₅₀'s (˜4 nM) for all the stapled peptides' binding with beta-catenin, FITC-labelled peptide samples (L_{i, i+4}L, L_{i, i+4}D or the RCM control) were dissolved in 100% rat serum (Sigma Aldrich) at an initial concentration of 4 μM. Given the EC₅₀ value (˜300 nM) of the unstapled peptide, a concentration of 300 μM was initially used for incubation with rat serum. Each sample mixture was then equally aliquoted into 15 tubes, and were incubated at 37° C. At every specified time point (0, 20, 40, 60 and 120 min), three of the tubes were collected, and immediately diluted 1/1000 with PBS buffer (1% BSA), followed by flash freezing in liquid nitrogen and short-term storage at −80° C. On the day of the ELISA assay, all the samples were slowly thawed on ice, and the remaining percentage of active peptides were determined by the ELISA assay following the procedure described above. The signals from the peptide sample mixture at 0 min were used as the 100% control.

Cell Growth Inhibition Assay: DLD-1 cells were cultured and maintained as mentioned before. Right before the assay, cells were resuspended in fresh RPMI media supplemented with 10% FBS, 100 IU/mL penicillin, and 100 μg/mL streptomycin, and plated onto 96-well white flat-bottom plates at 1000 cells per well (90 μL media/well). After overnight incubation, peptides samples were serially diluted as 10× stock in PBS buffer (10% DMSO), and were added to the plated cells at 10 μL stock solution/well in triplicate to make a final treatment concentration of 0 μM, 0.0625 μM, 0.125 μM, 0.25 μM, 0.5 μM, 1 μM, 2 μM, 4 μM, 8 μM, 16 μM. The wells on the edges of plates were filled with PBS buffer to avoid unwanted evaporation of samples. The samples on the plates were incubated at 37° C., 5% CO₂ for 5 days, and the cell growth was evaluated by CellTiter Glo (Promega). The luminescence signals were recorded by the H1 synergy plate reader (Biotek) and were normalized against the control groups (100%) for which cells were only treated with the vehicle (PBS/1% DMSO).

Small Molecule Model Reactions: To a mixture of 20 μL of Tris buffer (3M) and 20 μL of DMF was added 10 μL of the stock DMF solution for the model compound (1) (640 mM). After 10 min of incubation at room temperature, 10 μL of methyl hydrazine, cysteine, or benzyl thiol (640 mM, DMF) together with 20 μL of water (for methyl hydrazine) or TCEP solution (2M) (for cysteine or benzyl thiol) were added. After adjusting its final pH to 9.0, the mixture was aliquoted equally into 4 sample vials (20 μL each), which were subsequently incubated at 37° C. At the desired time point (0, 4 h, 8 h, and 12 h) of incubation, one vial each was collected to run LC-MS analysis of the sample. To determine the reaction progress, the UV peak areas (254 nm) of the model compound (1) before and after the 12 h incubation were integrated and the relative yields of the reaction were calculated based on the decreased percentage of the peak areas for compound (1).

Compound Characterization

Compound 1. In a flame-dried 50 mL round bottom flask, sodium fluoroacetate (200 mg, 2 mmol) and HATU (912 mg, 2.4 mmol) were mixed with DIPEA (418 μL, 2.4 mmol) in 15 mL anhydrous DMF. Around 10 min later, phenylethylamine (251 μL, 2 mmol) was added to the mixture. After overnight stirring, the reaction was quenched with water and the product was extracted with ethyl acetate. The collected organic layer was dried over anhydrous sodium sulfate, concentrated, and purified through silica gel column chromatography (hexane/ethyl acetate: 3/1) to afford the desired product as a white solid (277 mg, 1.52 mmol, 76% yield). ¹H NMR (500 MHz, CDCl₃): δ 7.34-7.31 (m, 2H), 7.26-7.20 (m, 3H), 4.76 (d, J=47.5 Hz, 2H), 3.60 (q, J=6.5 Hz, 2H), 2.86 (t, J=7.5 Hz, 2H); ¹³C NMR (126 MHz, CDCl₃): δ 167.6 (d, J=17.1 Hz), 138.4, 128.8 (d, J=5.0 Hz), 126.7, 80.2 (d, J=186.1 Hz), 40.0, 35.6; ¹⁹F NMR (471 MHz, CDCl₃): δ −224.78; HRMS (ESI) m/z calculated for C₁₀H₁₃FNO [M+H]⁺: 182.0976, found 182.0971.

Compound 2. The conversion of model compound 1 to the fluorine displaced product 2 was scaled up in 5 mL final volume following the procedure described in “Small Molecule Model Reactions”. The reaction was quenched with 20 mL brine and the product was extracted with 30 mL of ethyl acetate three times. The organic layer was combined, dried, and concentrated under reduced pressure. The crude mixture was purified by flash column chromatography (25% ethyl acetate/hexane) to afford 80 mg white solid (70.7% yield). ¹H NMR (500 MHz, CDCl₃): δ 7.12-7.39 (m, 10H), 6.73 (s, 1H), 3.57 (s, 2H,), 3.46 (q, 2H), 3.09 (s, 2H), 2.79 (t, 2H); ¹³C NMR (126 MHz, CDCl₃): δ 168.39, 138.65, 137.06, 128.97, 128.87, 128.77, 128.74, 127.46, 126.67, 40.71, 37.30, 35.43, 35.36. ESI-MS m/z calculated for C₁₇H₁₉NOS [M+H]⁺: 286.1, found 286.1.

Compound 3. Boc-Dap-OH (1 g, 4.9 mmol) was dissolved in the mixture of water (17 mL) and dioxane (47 mL). Na₂CO₃ (1.04 g, 9.8 mmol) in water (5 mL) was added to the flask, and the mixture was cooled on ice bath. Cbz-Cl (1.07 mL, 7.5 mmol) was then added dropwise, after which the mixture was stirred at room temperature overnight. The next day, dioxane was removed under reduced pressure, and the solution's pH was adjusted to approximately 9.0 with 1 M sodium hydroxide. The solution was extracted twice with ethyl acetate to remove the unreacted Cbz-Cl. The pH was subsequently adjusted to 4.0 with 1 M HCl and the acidic solution was then extracted three times by ethyl acetate, with the organic layers combined and dried over sodium sulfate. The organic phase was finally vacuum dried to afford 1.6 gram of crude product (96.5% yield) and was used directly for the next step without purification.

Compound 4. Compound 4 was synthesized following the same procedure for the enantiomer, compound 3, with a 97.4% yield.

Compound 5. The previously synthesized compound 3 (1.6 g, 4.73 mmol) was dissolved in 20 mL of dry DMF. Potassium carbonate (2.29 g, 16.56 mmol) was added, and stirred at room temperature for 20 min. The reaction mixture was then cooled in an ice bath followed by the addition of methyl iodide (353 μL, 5.676 mmol). After overnight stirring at room temperature, the mixture was diluted with 200 mL water, and extracted three times with 60 mL ethyl acetate. The organic layers were combined, dried over anhydrous sodium sulfate, and concentrated under reduced pressure. The crude mixture was purified by flash column chromatography (ethyl acetate/hexane: ¼) to finally afford 1.58 gram of product in viscous oil form (94% yield). ¹H NMR (500 MHz, CDCl₃): δ 7.36-7.30 (m, 5H), 5.46 (s, 1H, br), 5.20 (s, 1H, br), 5.08 (s, 2H), 4.37 (s, 1H, br), 3.73 (s, 3H,), 3.58 (s, 2H, br) 1.42 (s, 9H); ¹³C NMR (126 MHz, CDCl₃): δ 171.18, 156.72, 155.43, 136.24, 128.56, 128.23, 128.15, 80.31, 67.03, 54.04, 52.71, 42.97, 28.30; HRMS(ESI) m/z calculated for [C₁₂H₁₇N₂O₄, M−Boc+H]⁺: 253.1188, found 253.1181.

Compound 6. Compound 6 has been synthesized following the same procedure for compound 5, with a 93.5% yield. ¹H NMR (500 MHz, CDCl₃): δ 7.35-7.28 (m, 5H), 5.63 (s, 1H, br), 5.47 (s, 1H, br), 5.07 (s, 2H), 4.37 (s, 1H, br), 3.71 (s, 3H,), 3.56 (s, 2H, br) 1.43 (s, 9H); ¹³C NMR (126 MHz, CDCl₃): δ 171.29, 156.83, 155.56, 136.33, 128.52, 128.17, 128.12, 80.19, 66.94, 54.08, 52.63, 42.85, 28.29; HRMS(ESI) m/z calculated for [C₁₂H₁₇N₂O₄, M−Boc+H]⁺: 253.1188, found 253.1182.

Compound 7. The previously synthesized compound 5 (1.58 g, 4.48 mmol) was dissolved in 20 mL of methanol and cooled on ice bath. A small amount of hydrogen chloride (370 μL) in methanol (5 mL) was added dropwise in order to quench the nucleophilic amine generated in situ during follow up hydrogenolysis. For hydrogenolysis, 10% Pd/C (200 mg) was added with subsequent stirring at room temperature under the hydrogen atmosphere. After overnight stirring, the reaction mixture was filtered through celite to remove Pd/C. The solvent was also evaporated under reduced pressure to afford crude compound 7 with almost quantitative yield. The crude product was used for the next step without any purification.

Compound 8. As an enantiomer to compound 7, compound 8 has been synthesized following the same procedure for compound 7.

Compound 9. Sodium fluoroacetate (100 mg, 1 mmol), HATU (418.3 mg, 1.1 mmol), and compound 7 (436.4 mg, 2 mmol) were dissolved in 10 mL of DMF and stirred at room temperature for 20 min. DIPEA (608.5 μL, 3.5 mmol) was then added. The mixture was stirred overnight, before the reaction was quenched with 100 mL of brine. After extraction (three times) with 50 mL ethyl acetate, the organic layers were combined and dried over anhydrous sodium sulfate. The crude was vacuum concentrated, loaded onto silica column and purified by ethyl acetate/hexane (1:2) to afford 215 mg of oil-like product with a 77.3% yield. ¹H NMR (500 MHz, CDCl₃): δ 7.03 (s, 1H, br), 5.63 (s, 1H, br), 4.69-4.79 (d, J=50 Hz, 2H,), 4.39 (s, 1H, br), 3.71 (s, 3H,), 3.66 (t, J=5.0 Hz, 2H,), 1.38 (s, 9H); ¹³C NMR (126 MHz, CDCl₃): δ 170.95, 168.47, 155.69, 80.84, 80.35, 79.36, 53.54, 52.72, 40.92, 28.19; HRMS(ESI) m/z calculated for [C₆H₁₂FN₂O₃, M−Boc+H]⁺: 179.0832, found 179.0824.

Compound 10. Compound 10 was prepared similarly to compound 9, with a 75% yield. ¹H NMR (500 MHz, CDCl₃): δ 6.94 (s, 1H, br), 5.55 (s, 1H, br), 4.81-4.72 (d, J=45 Hz, 2H,), 4.41 (s, 1H, br), 3.74 (s, 3H,), 3.68 (t, J=7.5 Hz, 2H,), 1.41 (s, 9H); ¹³C NMR (126 MHz, CDCl₃): δ 170.90, 168.41, 155.69, 80.87, 80.46, 79.40, 53.49, 52.81, 41.07, 28.23; HRMS(ESI) m/z calculated for [C₆H₁₂FN₂O₃, M−Boc+H]⁺: 179.0832, found 179.0825.

Compound 11. Compound 9 (225 mg, 0.81 mmol) was dissolved in 4 mL of tetrahydrofuran and 2 mL of methanol. The solution was cooled in an ice-bath and 0.89 mL of 1 M sodium hydroxide (0.89 mmol) was added. After 15 min of stirring, the ice-bath was removed followed by subsequent stirring at room temperature for 1 h. The reaction was then quenched with 60 mL of water, and the mixture was washed once with 30 mL of ethyl acetate. The remaining mixture in the aqueous phase was cooled on ice-bath again, with the pH adjusted to 4.0 by 1 M HCl. At this point, the solution was extracted three times with 50 mL of ethyl acetate each. The organic layers were combined and dried over anhydrous sodium sulfate. The solvent was removed under reduced pressure by rotavapor, to afford 192.3 mg of crude product (˜90% yield) that was used directly for the next step.

Compound 12. Compound 12 was synthesized following the same procedure for the enantiomer, 11, with a 91% yield.

Compound 13. For Boc deprotection, compound 11 (200 mg, 0.76 mmol) was dissolved in 5 mL of dichloromethane and cooled in an ice-bath. Trifluoroacetic acid (2.5 mL) was added dropwise, and the mixture was kept stirring on the ice-bath for 10 min, after which the stirring was continued at room temperature for 1 h. The solvent was removed under vacuum, to afford 111 mg of crude product with an 89.5% yield.

Compound 14. Compound 14 was prepared similarly from compound 12, with an 88% yield.

Compound 15. Towards 200 mg compound 13 (1.22 mmol) in 3.5 mL of ice-cold 10% sodium carbonate solution, Fmoc-OSu (452 mg, 1.34 mmol) in 4 mL of dioxane was added dropwise. The mixture was left stirring overnight at room temperature. Dioxane was removed by vacuum, followed by the addition of 15 mL of water. The solution was washed with 10 mL of diethyl ether once. The aqueous phase was cooled with ice bath, and the solution's pH was adjusted to 3.5 by citric acid. Ethyl acetate was then used to extract the solution for three times (30 mL each). The combined organic phase was dried by anhydrous sodium sulfate, vacuum concentrated, and purified by flash column chromatography (2.5% Methanol/96.5% DCM/1% acetic acid). Finally, 396 mg of product in white solid form was obtained with a 91.5% yield. ¹H NMR (500 MHz, CD₃OD): δ 7.72 (d, J=5 Hz, 2H), 7.60 (s, 2H, br), 7.32 (t, J=7.5 Hz, 2H), 7.25 (t, J=7.5 2H,), 4.70-4.79 (d, 1H), 4.35 (s, 1H, br), 4.27 (d, J=10 Hz, 2H), 4.15 (t, J=7.5 Hz, 1H,), 3.71-3.54 (m, 2H); ¹³C NMR (126 MHz, CD₃OD): δ 169.73, 169.58, 157.16, 143.86, 141.16, 127.39, 126.77, 124.85, 119.53, 80.35, 78.89, 66.77, 48.14, 39.63; HRMS(ESI) m/z calculated for [C₂₀H₂₀FN₂O₅, M+H]⁺: 387.1356, found 387.1351.

Compound 16. Compound 16 has been synthesized following the same procedure for compound 15, with a 90.7% final yield. ¹H NMR (500 MHz, CD₃OD): δ 7.76 (d, J=10 Hz, 2H), 7.64 (m, 2H), 7.36 (t, J=7.5 Hz, 2H), 7.28 (t, J=7.5 Hz, 2H), 4.82-4.72 (dd, J=50 and 2.5 Hz, 2H), 4.37 (m, 1H), 4.32 (m, 2H), 4.20 (t, J=7.5 Hz, 1H), 3.74-3.56 (m, 2H); ¹³C NMR (126 MHz, CD₃OD): δ 171.99, 169.73, 157.17, 143.87, 141.18, 127.39, 126.77, 124.86, 119.52, 80.34, 78.83, 66.77, 48.12, 39.60; HRMS(ESI) m/z calculated for [C₂₀H₂₀FN₂O₅, M+H]⁺: 387.1356, found 387.1350.

Compound 17. Towards a mixture of 2 mL of DMF and 4 mL of Tris (3M solution) were added 470 μL of compound 9 (600 mM in DMF), 500 μL of benzyl thiol (600 mM in DMF), and 525 μL of TCEP solution (600 mM). After the final pH was adjusted to 9.0, the mixture was left stirring at 37° C. for 12 h. The reaction was then quenched with 20 mL of brine and the product was extracted with 30 mL of ethyl acetate three times. The organic layers were combined, dried over anhydrous sodium sulfate, and concentrated under reduced pressure. The crude mixture was purified by flash column chromatography (25% ethyl acetate/hexane) to finally afford 50.2 mg of compound 17 as a white solid (73% yield). ¹H NMR (500 MHz, CDCl₃): δ 7.24-7.34 (m, 5H), 7.08 (s, 1H), 5.49 (s, 1H), 4.39 (m, 1H,), 3.77 (s, 3H), 3.72 (s, 2H), 3.58 (m, 2H), 3.11 (s, 2H), 1.45 (s, 9H); ¹³C NMR (126 MHz, CDCl₃): δ 171.09, 169.48, 155.57, 136.95, 129.07, 128.75, 127.90, 80.39, 53.94, 52.78, 41.62, 36.93, 35.04, 28.31; HRMS(ESI) m/z calculated for [C₁₃H₁₉N₂O₃S, M−Boc+H]⁺: 0.283.1116, found 283.1109.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of stapling one or more amino acid sequences, wherein the method comprises reacting a compound or salt thereof having the structure of Formula (I) and a compound or salt thereof having the structure of Formula (II) 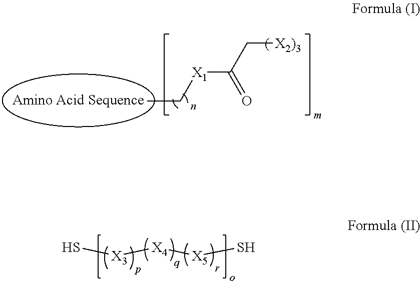
wherein each occurrence of X₁ is independently selected from the group consisting of O, S, NR₁, CR₁R₂, and C(═R₃);
each occurrence of X₂ is independently selected from the group consisting of H, Br, Cl, F, and I;
each occurrence of X₃, X₄, and X₅ is independently selected from the group consisting of O, S, NR₁, CR₁R₂, C(═R₃), cycloalkyl, substituted cycloalkyl, heterocycloalkyl, substituted heterocycloalkyl, aryl, substituted aryl, heteroaryl, and substituted heteroaryl;
each occurrence of R₁ and R₂ is independently selected from the group consisting of hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, and substituted heteroaryl;
each occurrence of R₃ is independently selected from the group consisting of O, NR₁, and S;
m is an integer from 1 to 10;
each occurrence of n, p, q, and r is independently an integer from 0 to 50; and
is an integer from 1 to 10.

2. The method of claim 1, wherein the compound having the structure of Formula (I) is a compound having the structure of Formula (III) 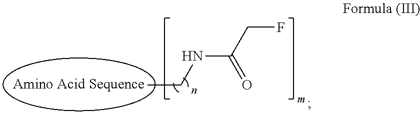
wherein m is an integer from 1 to 10; and
each occurrence of n is independently an integer from 0 to 50.

3. The method of claim 1, wherein the compound having the structure of Formula (II) is a compound having the structure of Formula (IV) 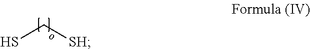
or a compound having the structure of Formula (V) 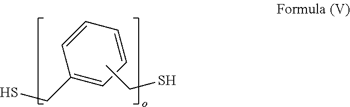
wherein o is an integer from 1 to 10.

4. The method of claim 1, wherein the amino acid sequence comprises two or more amino acids.

5. The method of claim 1, wherein the amino acid sequence is selected from the group consisting of: a protein or a fragment thereof, peptide or a fragment thereof, antigen or a fragment thereof, and any combination thereof.

6. (canceled)

7. The method of claim 1, wherein the method comprises reacting the compound or salt thereof having the structure of Formula (I) and the compound or salt thereof having the structure of Formula (II) in the presence of a base, reducing agent, or a combination thereof.

8. (canceled)

9. The method of claim 1, wherein the method comprises reacting the compound or salt thereof having the structure of Formula (I) and the compound or salt thereof having the structure of Formula (II) in a solution having a pH above 7.

10.-12. (canceled)

13. The method of claim 1, wherein the compound or salt thereof having the structure of Formula (I) is reacted with the compound or salt thereof having the structure of Formula (II) in 100:1, 50:1, 10:1, 5:1, 2:1, 1:1, 1:2, 1:5, 1:10, 1:50, or 1:100 molar ratio.

14. (canceled)

15. A method of linking one or more amino acid sequences and one or more compounds A, wherein the method comprises reacting a compound or salt thereof having the structure of Formula (I) with a compound or salt thereof having the structure of Formula (VI) 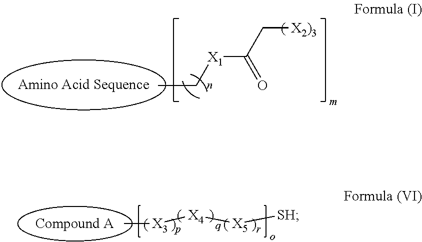
wherein each occurrence of X₁ is independently selected from the group consisting of O, S, NR₁, CR₁R₂, and C(═R₃);
each occurrence of X₂ is independently selected from the group consisting of H, Br, Cl, F, and I;
each occurrence of X₃, X₄, and X₅ is independently selected from the group consisting of O, S, NR₁, CR₁R₂, C(═R₃), cycloalkyl, substituted cycloalkyl, heterocycloalkyl, substituted heterocycloalkyl, aryl, substituted aryl, heteroaryl, and substituted heteroaryl;
each occurrence of R₁ and R₂ is independently selected from the group consisting of hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, and substituted heteroaryl;
each occurrence of R₃ is independently selected from the group consisting of O, NR₁, and S;
m is an integer from 1 to 10;
each occurrence of n, p, q, and r is independently an integer from 0 to 50; and
o is an integer from 0 to 10.

16. The method of claim 15, wherein the compound having the structure of Formula (I) is a compound having the structure of Formula (III) 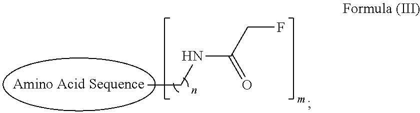
wherein m is an integer from 1 to 10; and
each occurrence of n is independently an integer from 0 to 50.

17. The method of claim 15, wherein the compound A is selected from the group consisting of an antibody or a fragment thereof, antigen or a fragment thereof, protein or a fragment thereof, peptide or a fragment thereof, amino acid sequence or a fragment thereof, amino acid or a derivative thereof, small molecule or a derivative thereof, therapeutic agent or a derivative thereof, and any combination thereof.

18. The method of claim 15, wherein the compound having the structure of Formula (VI) is a compound having the structure of Formula (VII) 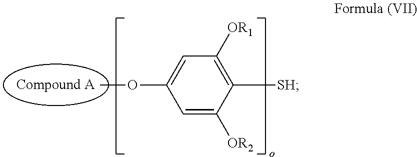
wherein each occurrence of R₁ and R₂ is independently selected from the group consisting of hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, and substituted heteroaryl; and
is an integer from 1 to 10.

19.-20. (canceled)

21. A compound prepared by the method of claim 1.

22. The compound of claim 21, wherein the compound is a compound having the structure selected from the group consisting of 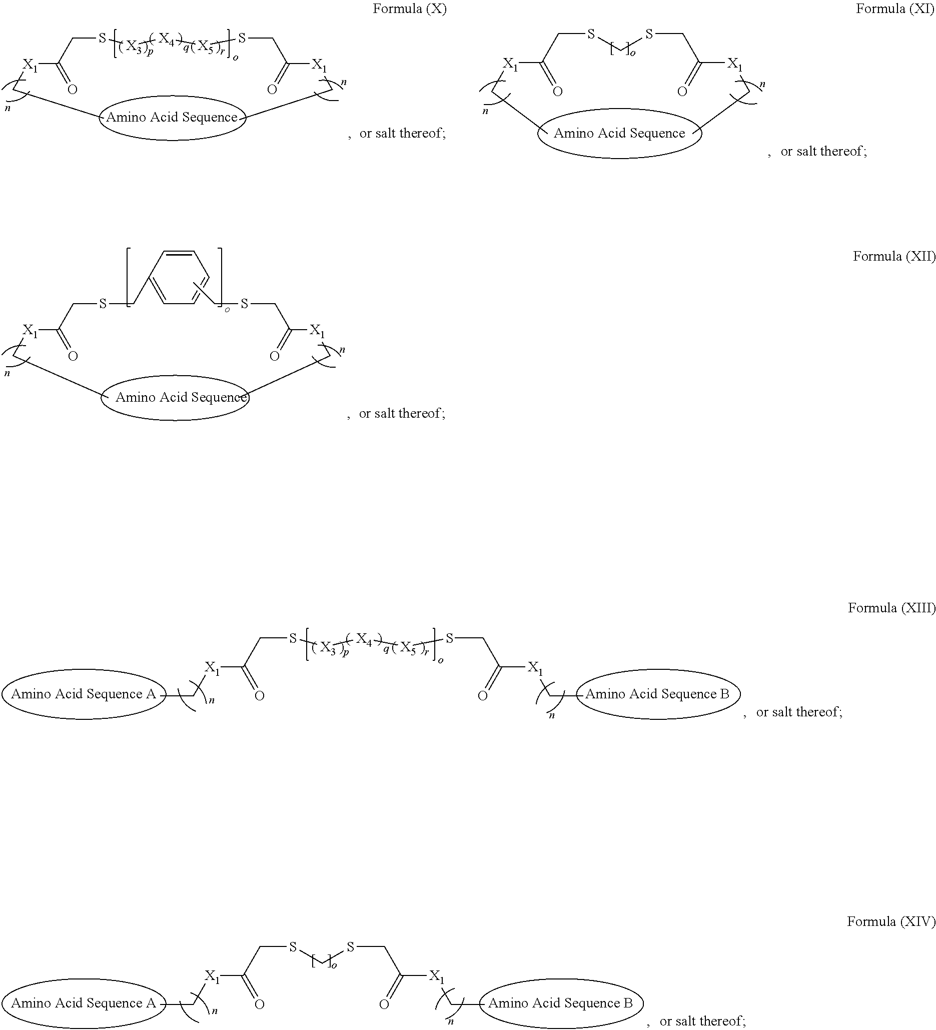 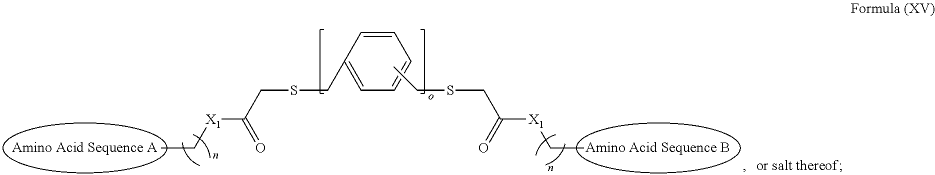
wherein each occurrence of X₁ is independently selected from the group consisting of O, S, NR₁, CR₁R₂, and C(═R₃);
each occurrence of X₃, X₄, and X₅ is independently selected from the group consisting of O, S, NR₁, CR₁R₂, C(═R₃), cycloalkyl, substituted cycloalkyl, heterocycloalkyl, substituted heterocycloalkyl, aryl, substituted aryl, heteroaryl, and substituted heteroaryl;
each occurrence of R₁ and R₂ is independently selected from the group consisting of hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, and substituted heteroaryl;
each occurrence of R₃ is independently selected from the group consisting of O, NR₁, and S;
each occurrence of n, p, q, and r is independently an integer from 0 to 50; and
o is an integer from 1 to 10.

23.-27. (canceled)

29. A compound prepared by the method of claim 15.

30. The compound of claim 29, wherein the compound is a compound having the structure selected from the group consisting of 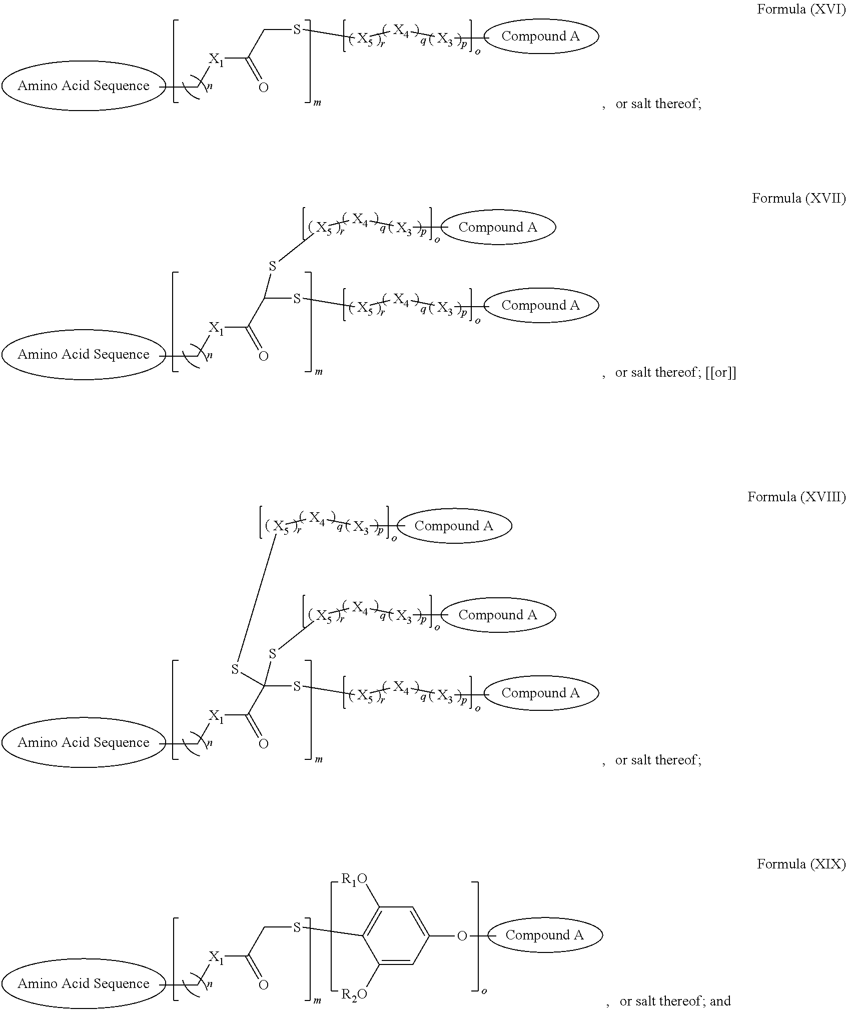 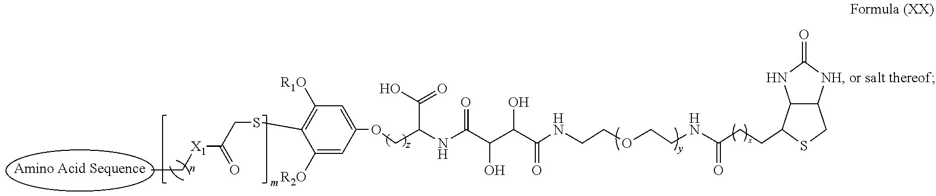
wherein each occurrence of X₁ is independently selected from the group consisting of O, S, NR₁, CR₁R₂, and C(═R₃);
each occurrence of X₃, X₄, and X₅ is independently selected from the group consisting of O, S, NR₁, CR₁R₂, C(═R₃), cycloalkyl, substituted cycloalkyl, heterocycloalkyl, substituted heterocycloalkyl, aryl, substituted aryl, heteroaryl, and substituted heteroaryl;
each occurrence of R₁ and R₂ is independently selected from the group consisting of hydrogen, halogen, hydroxyl, carboxyl, alkyl, substituted alkyl, cycloalkyl, substituted cycloalkyl, heterocycloalkyl, substituted heterocycloalkyl, aryl, substituted aryl, heteroaryl, and substituted heteroaryl;
each occurrence of R₃ is independently selected from the group consisting of O, NR₁, and S;
m is an integer from 1 to 10;
each occurrence of n, p, q, and r is independently an integer from 0 to 50; and
o is an integer from 0 to 10.

31.-33. (canceled)

34. A method of delivering a stapled amino acid sequence into a subject in need thereof, the method comprising administering the compound of claim 21 to the subject, wherein the compound penetrates a cell.

35. A method of delivering an amino acid sequence, a compound A, or a combination thereof into a subject in need thereof, the method comprising administering the compound of claim 29 to the subject, wherein the compound penetrates a cell.

36. A method of treating a disease or disorder in a subject, the method comprising administering the compound of claim 21 to the subject, wherein the compound penetrates a cell.

37. A method of treating a disease or disorder in a subject, the method comprising administering the compound of claim 29 to the subject, wherein the compound penetrates a cell.

38.-41. (canceled)


SEQUENCE LISTING (7 sequences)

SEQ ID NO: 1 - protein, 166 aa, Artificial Sequence
  Histone H2B
MPDPAKSAPAPKKGSKKAVTKVQKKDGKKRKRSRKESYSVYVYKVLKQVHPDTGISSKAMGIMNSFVNDIFERIAGEASRLAHYNKRSTITSREIQTAVRLLLPGELAKHAVSEGTKAVTKYTSSNPRNLSPTKPGGSEDRQPPPSQLSAIPPFCLVLRAGIAGQV

SEQ ID NO: 2 - protein, 103 aa, Artificial Sequence [sequence omitted]

SEQ ID NO: 3 - protein, 451 aa, Artificial Sequence
  Human TUBA1A full-length
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 4 - protein, 7 aa, Artificial Sequence
  Amino Acid Sequence of Peptide 18 through Peptide 19
  misc_feature (2)..(2): Xaa can be any L-amino acid or any D-amino acid
  misc_feature (6)..(6): Xaa can be any L-amino acid or any D-amino acid
AXEAKXY

SEQ ID NO: 5 - protein, 15 aa, Artificial Sequence
  Amino Acid Sequence of Peptide 20 through Peptide 42
  misc_feature (5)..(5): Xaa can be any L-amino acid or any D-amino acid
  misc_feature (9)..(9): Xaa can be any L-amino acid or any D-amino acid
ENPEXILDXHVQRVM

SEQ ID NO: 6 - protein, 13 aa, Artificial Sequence
  Amino Acid Sequence of Peptide 43 through Peptide 46
  misc_feature (5)..(5): Xaa can be any L-amino acid or any D-amino acid
  misc_feature (9)..(9): Xaa can be any L-amino acid or any D-amino acid
AITFXDLLXYYGP

SEQ ID NO: 7 - protein, 16 aa, Artificial Sequence
  Amino Acid Sequence of Peptide 47 through Peptide 51
  misc_feature (5)..(5): Xaa can be any L-amino acid or any D-amino acid
  misc_feature (9)..(9): Xaa can be any L-amino acid or any D-amino acid
RWPQXILDXHVRRVWR